(19) United States
(12) Patent Application Publication
(10) Pub. No.: US 2017/0326229 A1
(43) Pub. Date: Nov. 16, 2017

(54) SWARM IMMUNIZATION WITH 54 ENVELOPES FROM CH505

(71) Applicants: Duke University, Durham, NC (US); Los Alamos National Security, LLC, Los Alamos, NM (US)

(72) Inventors: Barton F. HAYNES, Durham, NC (US); Bette T. KORBER, Los Alamos, NM (US); Peter T. HRABER, Los Alamos, NM (US); Hua-Xin LIAO, Durham, NC (US)

(21) Appl. No.: 15/514,265

(22) PCT Filed: Sep. 29, 2015
(86) PCT No.: PCT/US15/53004

§ 371 Date: Mar. 24, 2017

Related U.S. Application Data
(60) Provisional application No. 62/056,822, filed on Sep. 29, 2014.
(60) Provisional application No. 62/150,019, filed on Apr. 20, 2015.

(51) Int. Cl. A61K 39/21; C12N 7/00; C07K 14/005; A61K 39/00

(52) U.S. Cl. CPC A61K 39/21; C12N 7/00; C07K 14/005; C12N 2740/16022; A61K 2039/5256; A61K 2039/53; A61K 2039/575; C12N 2740/16034; C12N 2740/16043

(57) ABSTRACT

In certain aspects the invention provides HIV-1 immunogens, including envelopes (CH505) and selections therefrom, and methods for swami immunizations using combinations of HIV-1 envelopes.

This application claims the benefit of and priority to the U.S. Provisional Patent Application No. 62/056,822, filed on Sep. 29, 2014, and U.S. Provisional Patent Application No. 62/150,019, filed on Apr. 20, 2015, the contents of each of which are hereby incorporated by reference in their entirety.

This invention was made with government support under Center for HIV/AIDS Vaccine Immunology-Immunogen Design grant UM1-AI100645 from the NIH, NIAID, Division of AIDS. The government has certain rights in the invention.

All patents, patent applications and publications cited herein are hereby incorporated by reference in their entirety. The disclosure of these publications in their entireties are hereby incorporated by reference into this application in order to more fully describe the state of the art as known to those skilled therein as of the date of the invention described herein.

### TECHNICAL FIELD

The present invention relates in general, to a composition suitable for use in inducing anti-HIV-1 antibodies, and, in particular, to immunogenic compositions comprising envelope proteins and nucleic acids to induce cross-reactive neutralizing antibodies and increase their breadth of coverage. The invention also relates to methods of inducing such broadly neutralizing anti-HIV-1 antibodies using such compositions.

### BACKGROUND

The development of a safe and effective HIV-1 vaccine is one of the highest priorities of the scientific community working on the HIV-1 epidemic. While anti-retroviral treatment (ART) has dramatically prolonged the lives of HIV-1 infected patients, ART is not routinely available in developing countries.

### SUMMARY OF THE INVENTION

In certain embodiments, the invention provides compositions and method for induction of immune response, for example cross-reactive (broadly) neutralizing Ab induction. In certain embodiments, the methods use compositions comprising “swarms” of sequentially evolved envelope viruses that occur in the setting of bnAb generation in vivo in HIV-1 infection.

In certain aspects the invention provides compositions comprising a selection of HIV-1 envelopes and/or nucleic acids encoding these envelopes as described herein for example but not limited to Selections as described herein. Without limitations, these selected combinations comprise envelopes which provide representation of the genetic (sequence) and antigenic diversity of the HIV-1 envelope variants which lead to the induction and maturation of the CH103 and CH235 antibody lineages. In certain embodiments, these compositions are used in immunization methods as a prime and/or boost as described in Selections as described herein.

In one aspect the invention provides selections of envelopes from individual CH505, which selections can be used in compositions for immunizations to induce lineages of broad neutralizing antibodies. In certain embodiments, there is some variance in the immunization regimen; in some embodiments, the selection of HIV-1 envelopes may be grouped in various combinations of primes and boosts, either as nucleic acids, proteins, or combinations thereof. In certain embodiments the compositions are pharmaceutical compositions which are immunogenic. In certain embodiments, the compositions comprise amounts of envelopes which are therapeutic and/or immunogenic.

In one aspect the invention provides a composition comprising any one of the envelopes described herein, or any combination thereof (selections in Examples). In some embodiments, CH505 transmitted/founder (T/F) Env is administered first as a prime, followed by a mixture of a next group of Envs, followed by a mixture of a next group(s) of Envs, followed by a mixture of the final Envs. In some embodiments, grouping of the envelopes is based on their binding affinity for the antibodies expected to be induced. In some embodiments, grouping of the envelopes is based on chronological evolution of envelope viruses that occurs in the setting of bnAb generation in vivo in HIV-1 infection. In some embodiments Loop D mutants could be included in either prime and/or boost. In some embodiments, the composition comprises an adjuvant. In some embodiments, the composition and methods comprise use of agents for transient modulation of the host immune response.

In one aspect the invention provides a composition comprising an HIV-1 envelope polypeptide or a nucleic acid encoding an HIV-1 envelope selected from the group consisting of M5, M6 and M11, or any combination thereof, wherein the HIV-1 envelope is a loop D mutant envelope.

In another aspect the invention provides a method of inducing an immune response in a subject comprising administering a composition comprising HIV-1 envelope M11, M6 and/or M5 as a prime in an amount sufficient to induce an immune response, wherein the envelope is administered as a polypeptide or a nucleic acid encoding the same. A method of inducing an immune response in a subject comprising administering a composition comprising any one of the HIV-1 envelopes in Table 1 or any combination as a prime in an amount sufficient to induce an immune response, wherein the envelope is administered as a polypeptide or a nucleic acid encoding the same.

In certain embodiments the methods comprise administering a composition comprising any one of HIV-1 envelopes polypeptides selected from the group consisting of w000.TF, w020.15, w030.13,w020.25, w004.54, w020.11, w078.15, w053.22, w136.B23, w053.31, w136.B2, w100.A13, w100.B4, w160.T4, w030.21, w053.15, w078.17, w136.B10, w053.29, w078.33, w136.B5, w030.36, w030.17, w078.9, w030.20, w100.B2, w078.6, or any combination thereof as a prime.

In certain embodiments the methods comprise administering a composition comprising any one of a nucleic acid encoding HIV-1 envelope selected from the group consisting of w000.TF, w020.15, w030.13,w020.25, w004.54, w020.11, w078.15, w053.22, w136.B23, w053.31, w136.B2, w100.A13, w100.B4, w160.T4, w030.21, w053.15, w078.17, w136.B10, w053.29, w078.33, w136.B5, w030.36, w030.17, w078.9, w030.20, w100.B2, w078.6, or any combination thereof as a prime.

In certain embodiments the methods comprise administering a composition comprising any one of HIV-1 envelopes in Table 1 or any combination thereof as a boost, wherein the envelope is administered as a polypeptide or a nucleic acid encoding the same.

In certain embodiments the methods comprise administering a composition comprising any one of HIV-1 envelopes polypeptides selected from the group consisting of w000.TF, w020.15, w030.13,w020.25, w004.54, w020.11, w078.15, w053.22, w136.B23, w053.31, w136.B2, w100.A13, w100.B4, w160.T4, w030.21, w053.15, w078.17, w136.B10, w053.29, w078.33, w136.B5, w030.36, w030.17, w078.9, w030.20, w100.B2, w078.6, or any combination thereof as a boost.

In certain embodiments the methods comprise administering a composition comprising any one of a nucleic acid encoding HIV-1 envelope selected from the group consisting of w000.TF, w020.15, w030.13,w020.25, w004.54, w020.11, w078.15, w053.22, w136.B23, w053.31, w136.B2, w100.A13, w100.B4, w160.T4, w030.21, w053.15, w078.17, w136.B10, w053.29, w078.33, w136.B5, w030.36, w030.17, w078.9, w030.20, w100.B2, w078.6, or any combination thereof as a boost.

In certain embodiments, the compositions contemplate nucleic acid, as DNA and/or RNA, or proteins immunogens either alone or in any combination. In certain embodiments, the methods contemplate genetic, as DNA and/or RNA, immunization either alone or in combination with envelope protein(s).

In certain embodiments the nucleic acid encoding an envelope is operably linked to a promoter inserted an expression vector. In certain aspects the compositions comprise a suitable carrier. In certain aspects the compositions comprise a suitable adjuvant.

In certain embodiments the induced immune response includes induction of antibodies, including but not limited to autologous and/or cross-reactive (broadly) neutralizing antibodies against HIV-1 envelope. Various assays that analyze whether an immunogenic composition induces an immune response, and the type of antibodies induced are known in the art and are also described herein.

In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides an immunogenic composition comprising the expression vector.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

In certain aspects the invention provides a composition comprising at least one nucleic acid encoding HIV-1 envelope from FIG. 2 or any combination thereof. Non-limiting examples of combinations are shown in Example 2.

In certain aspects, the invention provides a composition comprising any one or at least one nucleic acid encoding HIV-1 envelope selected from the group consisting of w000.TF, w004.31, w004.54, w007.8, w007.21, w007.25, w007.34, w008.20, w009.19, w010.7, w020.15, w020.11, w020.24, w020.25, w022.6, w022.5, w022.9, w022.22, w030.20, w030.17, w030.21, w030.36, w030.26, w030.13, w030.32, w053.15, w053.29, w053.22, w053.8, w053.31, w053.9, w078.6, w078.36, w078.9, w078.26, w078.29, w078.30, w078.33, w078.17, w078.15, w078.27, w100.T3, w100.B10, w100.B2, w100.B4, w100.A11, w100.A13, w136.B10, w136.B5, w136.B2, w136.B23, w160.C1, w160.T3, w160.T4, or any combination thereof.

In certain aspects, the invention provides a composition comprising any one or at least one of the nucleic acids encoding HIV-1 envelope selected from the envelopes in FIG. 34 (57 envelopes from CH505).

In certain aspects, the invention provides a composition comprising any one of or at least one an HIV-1 envelope polypeptide selected from the group consisting of w000.TF, w004.31, w004.54, w007.8, w007.21, w007.25, w007.34, w008.20, w009.19, w010.7, w020.15, w020.11, w020.24, w020.25, w022.6, w022.5, w022.9, w022.22, w030.20, w030.17, w030.21, w030.36, w030.26, w030.13, w030.32, w053.15, w053.29, w053.22, w053.8, w053.31, w053.9, w078.6, w078.36, w078.9, w078.26, w078.29, w078.30, w078.33, w078.17, w078.15, w078.27, w100.T3, w100.B10, w100.B2, w100.B4, w100.A11, w100.A13, w136.B10, w136.B5, w136.B2, w136.B23, w160.C1, w160.T3, w160.T4, or any combination thereof.

In certain aspects, the invention provides a composition comprising any one of or at least one an HIV-1 envelope polypeptide from the envelopes in FIG. 34 (57 envelopes).

In certain embodiments, the compositions and methods employ an HIV-1 envelope as polypeptide instead of a nucleic acid sequence encoding the HIV-1 envelope. In certain embodiments, the compositions and methods employ an HIV-1 envelope as polypeptide, a nucleic acid sequence encoding the HIV-1 envelope, or a combination thereof.

The envelope used in the compositions and methods of the invention can be a gp160, gp150, gp145, gp140, gp120, gp41, N-terminal deletion variants as described herein, cleavage resistant variants as described herein, or codon optimized sequences thereof. In certain embodiments, the envelope used in the compositions and methods of the invention is gp160. In certain embodiments, the envelope used in the compositions and methods of the invention is gp150. In certain embodiments, the envelope used in the compositions and methods of the invention is gp145. In certain embodiments, the envelope used in the compositions and methods of the invention is gp120. In certain embodiments, the envelope used in the compositions and methods of the invention is gp41. In certain embodiments, the envelope used in the compositions and methods of the invention is a gp120 variant. In certain embodiments, the envelope used in the compositions and methods of the invention is gp120D8 variant.

The polypeptide contemplated by the invention can be a polypeptide comprising any one of the polypeptides described herein. The polypeptide contemplated by the invention can be a polypeptide consisting essentially of any one of the polypeptides described herein. The polypeptide contemplated by the invention can be a polypeptide consisting of any one of the polypeptides described herein. In certain embodiments, the polypeptide is recombinantly produced. In certain embodiments, the polypeptides and nucleic acids of the invention are suitable for use as an immunogen, for example to be administered in a human subject.

In certain embodiments the envelope is any of the forms of HIV-1 envelope. In certain embodiments the envelope is gp120, gp140, gp145 (i.e. with a transmembrane). In certain embodiments, the envelope is in a liposome and transmembrane with a cytoplasmic tail in a liposome. In certain embodiments, the nucleic acid comprises a nucleic acid sequence which encode a gp120, gp140, gp145, gp150 or gp160.

In certain embodiments, where the nucleic acids are operably linked to a promoter and inserted in a vector, the vectors is any suitable vector. Non-limiting examples, include, the VSV, replicating rAdenovirus type 4, MVA, Chimp adenovirus vectors, pox vectors, and the like. In certain embodiments, the nucleic acids are administered in NanoTaxi block polymer nanospheres. In certain embodiments, the composition and methods comprise an adjuvant. Non-limiting examples include, AS01 B, AS01 E, gla/SE, alum, Poly I poly C, TLR agonists, TLR7/8 and 9 agonists, or a combination of TLR7/8 and TLR9 agonists (see Moody et al. (2014) J. Virol. March 2014 vol. 88 no. 6 3329-3339), or any other adjuvant. Non-limiting examples of TLR7/8 agonist include TLR7/8 ligands, Gardiquimod, Imiquimod and R848 (resiquimod). A non-limiting embodiment of a combination of TLR7/8 and TLR9 agonist comprises R848 and oCpG in STS (see Moody et al. (2014) J. Virol. March 2014 vol. 88 no. 6 3329-3339).

In certain aspects the invention provides a method for selecting a swarm of HIV-1 envelopes, among a population of HIV-1 envelopes isolated over a period of time from an individual who develops bnAbs against HIV-1 wherein the swarm mimics the envelope diversity in a person who made a good antibody response during natural infection, by representing the relevant HIV diversity, capturing evolution of representative sites from within subject diverse populations.

### BRIEF DESCRIPTION OF THE DRAWINGS

To conform to the requirements for PCT patent applications, many of the figures presented herein are black and white representations of images originally created in color. In the below descriptions and the examples, the colored images are described in terms of its appearance in black and white. Different colors are described by different shades of white to grey with an attempt to match the description the descriptions of the color as closely as possible to that of the figures. The original color versions of some of the Figures can be viewed in Liao, et al., Co-evolution of a broadly neutralizing HIV-1 antibody and founder virus. Nature. 2013; 496 (7446): 469-76 (including the accompanying Supplementary Information) and Haynes et al., B-cell-lineage immunogen design in vaccine development with HIV-1 as a case study. Nat. Biotechnol 2012; 30: 423-33 (including the accompanying Supplementary Information). For the purposes of the PCT, contents of Liao, et al. (2013), including the accompanying “Supplementary Information,” and the contents of Haynes et al. (2012), including the accompanying “Supplementary Information,” are each herein incorporated by reference.

FIG. 1 Shows show Heat Map of Binding (log Area Under the Curve, AUC) of Sequential Envs to CH103 CD4 Binding Site Broadly Neutralizing Antibody Lineage members. Numerical data corresponding to the graphic representations in these figures are shown in Table 1.

FIG. 2 shows 27 (bolded) envelopes, among a selection of 54 envelopes, tested for binding to CH103 CD4 Binding Site Broadly Neutralizing Antibody Lineage members as shown in FIG. 1.

FIGS. 3A-3C shows the genotype variation (FIG. 3A), neutralization titers (FIG. 3B), and Envelope phylogenetic relations (FIG. 3C) among CH505 Envelope variants. The vertical position in each panel corresponds to the same CH505 Env clone named on the right side of the tree. Distance from the Transmitted/Founder form generally increases from top towards bottom of the figure. In the FIG. 3A, sites not colored correspond to the Transmitted/Founder virus, dark grey sites show mutations, and black sites correspond to insertions or deletions relative to the Transmitted/Founder virus. Additional annotation indicates the known CD4 binding-site contacts (short, vertical black bars towards top), CH103 binding-site contacts for the resolved structure (short, vertical grey bars with a horizontal line to indicate the region resolved by X-Ray Crystallography), gp120 landmarks (vertical grey rectangular regions, V1-V5 hypervariable loops, Loop D, and CD4 Loops), a dashed vertical line delineating the gp120/gp41 boundary, and results from testing for CTL epitopes with ELISpot assays (light grey horizontal bands at top and bottom show where peptides were tested and negative, and a light grey rectangle for the tested positive region outside the C-terminal end of V4). FIG. 3B depicts IC50 (50% inhibitory concentrations, in μg/ml) values from autologous neutralization assays against 13 monoclonal antibodies (MAbs) of the CH103 lineage and each of 134 CH505 Env-pseudotyped viruses. Grey Color-scale values indicate neutralization potency and range from gray (no neutralization detected) through dark grey (potent neutralization, i.e. <0.2 μg/ml; empty cells correspond to absence of information). The cumulative progression of neutralization potency from left to right, corresponding to developmental stages in the CH103 lineage, indicates accumulation of neutralization potency. Similarly, increased presence neutralization signal from top to bottom corresponds to increasing neutralization breadth per MAb in the CH103 lineage. In FIG. 3C is the phylogeny of CH505 Envs, with the x-axis indicating distance from the Transmitted-Founder virus per the scale bar (units are mutations per site). The tree is ordered vertically such that lineages with the most descendants appear towards the bottom. Each leaf on the tree corresponds to a CH505 autologous Env, with the name of the sequence depicted (‘w’ and symbol color indicate the sample time-point; ‘M’ indicates a synthetic mutant Env). The color of text in each leaf name indicates its inclusion in a possible embodiment. Three long, vertical lines to the left of the tree depict the phylogenetic distribution of envelopes in three distinct alternative embodiments (identified as “Vaccination Regimes 1-3”), with diamonds used to identify each.

FIG. 4 shows nucleic acid sequences of 26 six CH505 envelopes encoding gp160s. The nucleotide sequences of w004.31 (SEQ ID NO: 01), w007.8 (SEQ ID NO: 02), w007.21 (SEQ ID NO: 03), w007.25 (SEQ ID NO: 04), w007.34 (SEQ ID NO: 05), w008.20 (SEQ ID NO: 06), w009.19 (SEQ ID NO: 07), w010.7 (SEQ ID NO: 08), w022.6 (SEQ ID NO: 09), w022.5 (SEQ ID NO: 10), w022.9 (SEQ ID NO: 11), w022.22 (SEQ ID NO: 12), w030.26 (SEQ ID NO: 13), w030.32 (SEQ ID NO: 14), w053.8 (SEQ ID NO: 15), w053.9 (SEQ ID NO: 16), w078.36 (SEQ ID NO: 17), w078.26 (SEQ ID NO: 18), w078.29 (SEQ ID NO: 19), w078.30 (SEQ ID NO: 20), w078.27 (SEQ ID NO: 21), w100.T3 (SEQ ID NO: 22), w100.B10 (SEQ ID NO: 23), w100.A11 (SEQ ID NO: 24), w160.C1 (SEQ ID NO: 25), and w160.T3 (SEQ ID NO: 26) are shown. These are n26-nucleic acids-not-included in a previously described set of 90 envelopes.

FIG. 5 shows nucleic acid sequences of 28 CH505 envelopes encoding gp160s. The nucleotide sequences of w000.TF (SEQ ID NO: 27),w004.10 (SEQ ID NO: 28), w020.15 (SEQ ID NO: 29), w020.11 (SEQ ID NO: 30), w020.24 (SEQ ID NO: 31), w020.13 (SEQ ID NO: 32)n, w030.20 (SEQ ID NO: 33), w030.17 (SEQ ID NO: 34), w030.21(SEQ ID NO: 35), w030.36 (SEQ ID NO: 36), w030.13 (SEQ ID NO: 37), w053.3 (SEQ ID NO: 38), w053.29 (SEQ ID NO: 39), w053.31 (SEQ ID NO: 40), w053.16 (SEQ ID NO: 41), w078.6 (SEQ ID NO: 42),w078.9 (SEQ ID NO: 43), w078.33 (SEQ ID NO: 44), w078.17 (SEQ ID NO: 45), w078.15 (SEQ ID NO: 46), w100.B2 (SEQ ID NO: 47), w100.B4 (SEQ ID NO: 48), w100.A13 (SEQ ID NO: 49), w136.B10(SEQ ID NO: 50), w136.B5 (SEQ ID NO: 51), w136.B2(SEQ ID NO: 52), w136.B18(SEQ ID NO: 53), and w160.T4 (SEQ ID NO: 54) are shown. These are 28-nucleic acids included in a previously described set of 90 envelopes.

FIG. 6 shows sequences amino acids sequences of the nucleic acids of FIG. 4. The amino acid sequences of w004.31 (SEQ ID NO: 55), w007.8(SEQ ID NO: 56), w007.21(SEQ ID NO: 57), w007.25(SEQ ID NO: 58), w007.34 (SEQ ID NO: 59), w008.20 (SEQ ID NO: 60), w009.19(SEQ ID NO: 61), w010.7(SEQ ID NO: 62), w022.6(SEQ ID NO: 63), w022.5(SEQ ID NO: 64), w022.9(SEQ ID NO: 65), w022.22(SEQ ID NO: 66), w030.26(SEQ ID NO: 67), w030.32(SEQ ID NO: 68), w053.8(SEQ ID NO: 69), w053.9(SEQ ID NO: 70), w078.36 (SEQ ID NO: 71), w078.26(SEQ ID NO: 72), w078.29 (SEQ ID NO: 73), w078.30(SEQ ID NO: 74), w078.27(SEQ ID NO: 75), w100.T3(SEQ ID NO: 76), w100.B10(SEQ ID NO: 77), w100.A11 (SEQ ID NO: 78), w160.C1(SEQ ID NO: 79), and w160.T3(SEQ ID NO: 80) are shown.

FIG. 7 shows sequences of amino acids of the nucleic acid sequences of FIG. 5. The amino acid sequences of w000.TF(SEQ ID NO: 81), w004.10(SEQ ID NO: 82), w020.15 (SEQ ID NO: 83), w020.11(SEQ ID NO: 84), w020.24(SEQ ID NO: 85), w020.13 (SEQ ID NO: 86), w030.20(SEQ ID NO: 87), w030.17(SEQ ID NO: 88), w030.21(SEQ ID NO: 89), w030.36(SEQ ID NO: 90), w030.13(SEQ ID NO: 91), w053.3(SEQ ID NO: 92), w053.29(SEQ ID NO: 93), w053.31(SEQ ID NO: 94), w053.16(SEQ ID NO: 95), w078.6(SEQ ID NO: 96), w078.9(SEQ ID NO: 97), w078.33(SEQ ID NO: 98), w078.17(SEQ ID NO: 99), w078.15(SEQ ID NO: 100), w100.B2(SEQ ID NO: 101), w100.B4(SEQ ID NO: 102), w100.A13(SEQ ID NO: 103), w136.B10(SEQ ID NO: 104), w136.B5(SEQ ID NO: 105), w136.B2(SEQ ID NO: 106), w136.B18(SEQ ID NO: 107), and w160.T4(SEQ ID NO: 108) are shown.

FIG. 8 shows nucleic acid sequences of several M mutants encoding gp160. The sequences of >M5 (SEQ ID NO: 109), >M19(SEQ ID NO: 110), >M10(SEQ ID NO: 111), >M11(SEQ ID NO: 112), >M9(SEQ ID NO: 113), >M7(SEQ ID NO: 114), >M20(SEQ ID NO: 115), >M8(SEQ ID NO: 116), >M21(SEQ ID NO: 117), and >M6(SEQ ID NO: 118)nt are shown.

FIG. 9 shows amino acid sequences of several M mutants as gp160. The sequences of >M5(SEQ ID NO: 119), >M19(SEQ ID NO: 120), >M10 (SEQ ID NO: 121), >M11(SEQ ID NO: 122), >M9(SEQ ID NO: 123), >M7(SEQ ID NO: 124), >M20(SEQ ID NO: 125), >M8(SEQ ID NO: 126), >M21(SEQ ID NO: 127), and >M6(SEQ ID NO: 128) are shown.

FIG. 10 shows examples of amino acid sequences of CH505 D8gp120 constructs. The sequences of >HV1300531_v2 (M5) (SEQ ID NO: 129), >HV1300532_v2 (M6) (SEQ ID NO: 130), >HV1300533_v2 (M7) (SEQ ID NO: 131), >HV1300534_v2 (M8) (SEQ ID NO: 132), >HV1300535_v2 (M9) (SEQ ID NO: 133), >HV1300536_v2 (M10) (SEQ ID NO: 134), >HV1300537_v2 (M11) (SEQ ID NO: 135), >HV1300538_v2 (M19) (SEQ ID NO: 136), >HV1300539_v2 (M20) (SEQ ID NO: 137), >HV1300540_v2 (M20) (SEQ ID NO: 138), and >HV1300541_v2 (T/F) (SEQ ID NO: 139) are shown.

FIG. 11A shows nucleic acid sequence of T/F virus from individual CH505 (>CH0505.TF or SEQ ID NO: 140). FIG. 11B shows CH505 HIV-1 gene sequences. The nucleic acid sequences of GAG (SEQ ID NO: 141), POL (SEQ ID NO: 142), VIF (SEQ ID NO: 143), VPR (SEQ ID NO: 144), TAT (SEQ ID NO: 145), REV (SEQ ID NO: 146), VPU (SEQ ID NO: 147), ENV (SEQ ID NO: 148), NEF (SEQ ID NO: 149) are shown.

FIG. 12 shows loss of ancestral transmitted-founder (TF) amino acids in Envs from CH505. For 953 aligned Env sites, TF loss is proportion of non-TF variants per time-point sampled from the study participant CH505. TF loss is computed for each of 14 time-points sampled longitudinally, weeks 4 through 160, with the number of Envs sequenced (n) per time-point as shown. Bar colors vary over time to indicate 35 sites with at least 80% TF loss in any time-point, whether at peak TF loss, below peak but above the 80% cutoff, or below 80%. Sites not selected for further consideration, which remained below 80% TF loss throughout the study period, are also depicted (black bars). Grey boxes identify hypervariable loops and other gp120 landmarks; a grey line marks the boundary between gp120 and gp41.

FIG. 13 shows diversity of variant frequency dynamics within sites. The single TF virus (dashed lines) yields to putative escape mutations. Shaded regions show 95% confidence intervals for variant frequencies, computed from the binomial probability distribution, given the number of sequences sampled per time-point. Letters below each panel list variants in order of appearance. Numbers above each panel denote TF form, HXB2 position, and alignment column. For instance, “N279 [357]” indicates HXB2 279 (alignment column 357) and depicts loss of the transmitted asparagine. Lower-case letters denote insertions at the C-terminal end of the HXB2 site given. Colors indicate positive (medium grey) and negative (light grey) charges and “O” indicates a potentially gylcosylated asparagine (lighter grey). Hyphens (grey) indicate an insertion or deletion (indel). For clarity, variants that never reach 20% in a sample are not shown.

FIG. 14 shows cumulative distribution of peak TF loss over 953 aligned Env sites. Peak TF loss is the greatest proportion of non-TF variants in any time-point sampled, which corresponds to the minimum for each dashed line in FIG. 13. Of 953 aligned sites, 365 (38.3%) are invariant. 35 sites with at least 80% peak TF loss were selected for further study. Other cutoff values would yield more sites, e.g. 48 with 60% TF loss, or fewer sites, e.g. 15 with 100% TF loss.

FIGS. 15A-15G show selected sites that are localized to immunogenic regions on the BG505 SOSIP trimer (PDB 4TVP [46] Pancera M, Zhou T, Druz A, Georgiev I S, Soto C, Gorman J, et al. Structure and immune recognition of trimeric pre-fusion HIV-1 Env. Nature. 2014; 514(7523):455-61. doi: 10.1038/nature13808. PubMed PMID: 25296255). Selected sites are depicted as spheres, colored to indicate the timing of their emergence. FIG. 15A shows the side view, oriented with viral membrane towards bottom. FIG. 15B shows the addition of known immunogenic regions. FIG. 15C shows selected sites that are colored to show which immune pressures are known to have induced TF loss. FIG. 15D-15F show the top view, as seen from host cell membrane. FIG. 15G shows the key to the color scheme. Table 2 lists colored symbols for each selected site.

FIGS. 16A-16C show the variant frequency across 35 sites selected from CH505 Env gp160. FIG. 16A shows the variant frequencies among all 398 sequence sampled. Symbol height is proportional to amino acid frequency. Colors correspond to FIG. 13, and indels appear as grey boxes. Site order follows ranks listed in Table 2. FIG. 16B shows variant frequency, stratified by time. To emphasize TF loss progression, frequency of the TF form below the first row is blank. Each row corresponds to one time-point sampled for the three-year interval studied, weeks 0-160 (w000 through w160). FIG. 16C shows variant frequencies among swarm set of 54 selected Envs.

FIG. 17 shows the swarm selection algorithm. From a sequence alignment and list of selected sites, a greedy, deterministic approach identifies viable Envs and tabulates variants among selected sites. This table tracks which mutations remain to be included. Rare mutations, i.e. mutations detected fewer times than the minimum variant count over the entire sampling period, are disregarded. Selection among multiple sequences that carry a mutation is resolved by minimizing a series of distance criteria, first to minimize Hamming distance (number of mutations, gaps included) to the TF form among selected sites, then distance to the full-length TF sequence, and finally to minimize average distance to sequences in the current swarm set. The selected Env is included in the swarm set, counts in the table of needed variants are set to zero, and iteration continues. This produces a “swarm” of Envs, which represents variant diversity as it developed within the subject. Stacked boxes signify iteration.

FIGS. 18A-18B show the selected swarm set is distinct from randomly selected sets. FIG. 18A shows the number of distinct concatamers, mutations included, and clustering coefficients from dendrograms of concatamer distances differ for the selected swarm of 54 Envs (red) and the null distribution from 1,000 sets of 54 Envs, randomly selected without replacement from the non-redundant set of 260 viable full-length Envs, with the TF form always included. Values have jitter added for less overplotting. FIG. 18B shows the clustering coefficient quantifies sequence differences among as the average distance over which each sequence is merged into a cluster (horizontal grey bars in bottom row), compared for the selected and swarm set two extreme randomly sampled sets (min and max, circled points in the right-hand part of panel 18A.).

FIG. 19 shows env variants in phylogenetic context. A pixel plot is paired with the maximum-likelihood phylogeny, such that each row depicts one of 396 Envs sequenced by limiting-dilution PCR. The top row corresponds to the TF virus. In the pixel plot (left), sites that match the TF are blank and mutations are shaded indicate gain of negatively (light grey) or positively charged amino acids (medium grey), addition of an N-linked glycosylation motif (lighter grey), indels (black), or other mutations (grey). Stripes correspond roughly to TF loss. Env landmarks appear as vertical bands throughout the pixel plot (light grey), and dashed lines delineate the signal peptide and gp41. Tree branches and symbols are color-coded to indicate sample time-point, and the 54 selected Envs are marked by a black circle and horizontal bar.

FIG. 20 shows selected Envs that represent diverse binding phenotypes. Among the swarm of 54 Envs selected, 27 were synthesized as gp120s for ELISA binding assays (light grey text). Another four of the antigens tested contained selected sites that matched with those in selected Envs (w020.9; w100.B7; w160.C11; w160.D1). Binding data are shown as shades of grey to indicate log-transformed area under the curve (AUC) from dilution series, which summarized experimental results better than EC50s. Both assays tested Env constructs against monoclonal antibodies of the CH103 lineage, from mAb isolates (e.g., CH103) to the unmutated ancestor (UCA) via intermediate ancestors IA1-IA8 [15] (Liao H X, Lynch R, Zhou T, Gao F, Alam S M, Boyd S D, et al. Co-evolution of a broadly neutralizing HIV-1 antibody and founder virus. Nature. 2013; 496(7446):469-76. doi: 10.1038/nature12053. PubMed PMID: 23552890; PubMed Central PMCID: PMC3637846). Blank entries indicate no binding was detected. Selected Env sites correspond to concatamers in Table 3. An “X” appears for gp41 sites not in the gp120 antigens tested.

FIG. 21 shows selected Envs that represent diverse neutralization phenotypes. Among the swarm of 54 Envs, 26 were cloned into pseudovirus backbones for TZM-bl neutralization assays (red text). Another four of the Env-pseudotyped virus constructs tested contained selected sites that matched with those in selected Envs (w004.27; w004.10; w004.15; w020.27). Neutralization IC50s are depicted as shades of grey to indicate sensitivity of each virus to neutralization by each mAb in the CH103 lineage. Selected Env sites correspond to concatamers in Table 3.

FIGS. 22A-22D show structural mapping of selected and non-selected Env sites in CH505 sequences. (FIG. 22A) Sites selected by high TF loss are depicted by beads, in shades of grey to indicate when each site exceeded 10% TF loss, as listed in Table 2. For structural context, the immunologically relevant mutations and regions described in FIG. 15 are also shown. V1 sites missing from the structure are illustrated schematically (top left). (FIG. 22B) Sites that mutated only once among 398 CH505 Env sequences (0.25% TF loss over all time-points), and present in the structure, are identified by light grey beads. These sites were not selected, due to low TF loss. (FIG. 22C) Sites with two or more mutations among 398 CH505 Env sequences (at least 0.5% TF loss over all time-points), but less than 80% peak TF loss, are marked by light grey beads. (FIG. 22D) The symbols for each selected site in shades of grey.

FIG. 23 shows the number of sites varying with cutoff in chronically infected donor.

FIGS. 24A-24C show variant frequencies among selected sites in chronic infection. Frequencies from CH457, computed among (FIG. 24A) all sequences, pooled; (FIG. 24C) sequences stratified by time; and (FIG. 24B) 44 selected Envs. Medium grey indicates negative charge and “O” indicates a potentially gylcosylated asparagine (lighter grey). Indels appear as grey boxes.

FIG. 25 shows diversity in chronic infection. CH457 Env mutations (left), neutralization ID50 titers against autologous contemporaneous plasmas (center), and maximum-likelihood Env phylogeny (right). The 44 selected Envs are emphasized among all Envs sampled. The divergent clade appears above the “Time Sampled” legend. This representation follows FIG. 19, with neutralization titers added, one column per time-point. Neutralization responses were profiled for 84 Env-pseudotyped viruses, chosen before the swarm-selection algorithm existed, and tested against autologous sera from each time-point sampled.

FIG. 26 shows cumulative distribution of peak TF loss over 953 aligned Env sites. Peak TF loss is the greatest proportion of non-TF variants in any timepoint sampled. A third of sites are invariant. 36 sites with at least 80% peak TF loss (vertical line) were selected for further study.

FIG. 27 shows variant dynamics in 36 selected sites. Initially consisting of solely the TF form (dotted lines), forms emerge in the virus population, with varied dynamics across sites. Sites are numbered above each panel by alignment column and HXB2 position, e.g. “357/N279K” indicates alignment column 357, which corresponds to HXB2 279, and mutated from ancestral TF asparagine to lysine. Shades of grey indicate positive (medium grey) and negative (light grey) residue charges and “O” indicates a potentially gylcosylated asparagine (lighter grey). A dash (grey) indicates an insertion or deletion (indel) and the caret (̂) symbol in site numbers denotes an insertion at the C-terminal end of the given HXB2 position. For clarity, rare variants are not shown.

FIG. 28 shows variant dynamics in 36 selected sites, with confidence intervals. This is the same information presented in FIG. 27, with 95% confidence intervals on variant frequencies (shaded regions), estimated from the number of sequences sampled by the binomial distribution.

FIG. 29 shows the progression of TF loss in 36 CH505 Env sites. Symbol height indicates amino acid frequency per sample. To emphasize TF loss, frequencies of the TF form below the first row are blank. Each row corresponds to one timepoint sampled for the three-year interval studied, weeks 0-160 (w000 through w160). Colors correspond to FIG. 27, and indels appear as grey boxes. Dots after HXB2 numbers indicate C-terminal insertions. Site order follows FIG. 27.

FIG. 30 shows clone selection algorithm. Provided a sequence alignment and list of sites selected for representation in the swarm set, a greedy, deterministic approach identifies viable clones and tabulates variants among selected sites. This table of variant counts is used to track which mutations remain to be included in the swarm set. Variants that only appear once are ignored. Selection among multiple clones is resolved by a series of criteria, first to minimize distance (number of mutations, gaps included) to the TF form among selected sites, then the full-length clone, and finally to minimize average distance to clones in the swarm set. The selected clone is included in the swarm set, counts in the table of needed variants are set to zero, and iteration continues. This produces a swarm of clones that represents the variant diversity. Stacked boxes signify iteration.

FIG. 31 shows selected swarm clones are distinct from randomly selected swarms. Redundancy (number of duplicated concatamers) and clustering coefficient from concatamer Hamming distances are lower for the selected set of 57 clones (red) than 500 sets of 57 clones, randomly selected from the non-redundant set of 263 viable full-length clones. Redundancy values have jitter added for less overplotting.

FIGS. 32A-32C show swarm variant frequency from 57 concatamers over 36 selected sites. (FIG. 32A) TF amino acids. (FIG. 32B) Variant frequencies among 56 non-TF concatamers. Symbol height is proportional to amino acid frequency per sample. To emphasize TF loss, frequencies of the TF form are blank. (FIG. 32C) Combined frequencies of TF and non-TF variants. Indels appear as grey boxes. Colors correspond to FIG. 27. Dots after site numbers indicate C-terminal insertions.

FIG. 33 shows a table of CH505 Env sites with at least 80% peak TF loss. For the “Rank” column, sites were ranked by earliest to lose TF majority, then by increasing TF area. “ALN” refers to position in CH505 alignment. The “Week” column refers to timepoint at which this site first exceeds 50% TF loss. “TF area” refers to the cumultative TF loss, i.e. area under TF frequency as a function of time (dotted lines in FIG. 27). For entry “412/N332O” in the “Name column” (corresponding to Rank 3), asparagine is followed by a potential glycosylation motif Nx[ST], where x is not a proline. The caret symbol (̂) indicates an insertion at the C-terminal position of the HXB2 site.

FIG. 34 shows a table of concatamers from a swarm of 57 Env clones that represent selected sites. The sequences associated with the Genbank accession numbers KC and KM are incorporated by reference.

FIG. 35 shows the identification of sites by TF loss.

FIG. 36 shows the selection of clones with representative diversity.

FIG. 37 shows loss of ancestral transmitted-founder (TF) amino acids in Envs from CH505.

FIG. 38 shows variant frequency across 35 sites selected from CH505 Env gp160 stratified by time.

FIG. 39 shows the number of sites varying with TF loss cutoff for CH0505 gp160 (n=398 clones) and for CH0848 (n=1184 clones).

FIG. 40 shows the number of clones at minimum variant count. MVC=1 excludes singletons, i.e. mutations or indels only seen once.

FIG. 41 shows concatamers from a swarm of 54 env clones that represent selected sites.

FIG. 42 shows swarm variant frequency over 35 selected sites. The top row shows TF amino acids. The middle row shows variant frequencies among non-TF concatamers. Symbol height is proportional to amino acid frequency per sample. The bottom row shows combined frequencies of TF and non-TF variants. Indels appear as grey boxes.

FIG. 43 shows concatamers from a swarm of 90 env clones that represent selected sites.

FIG. 44 shows a plot of the sites above cutoff versus the non-UA cutoff

FIG. 45 shows variant frequency across 15 VH sites stratified by time.

FIG. 46 shows CH103 clonal family with time of appearance and VHDJH mutations. Maximum likelihood phylogram showing the CH103 lineage with the inferred intermediates (circles, I1-4, I7 and I8), and percentage mutated VH sites and timing indicated. Mutation frequency is 4-17%.

FIG. 47 shows CH103 clonal family binding affinity maturation. Binding affinities (Kd, nM) of antibodies to autologous subtype C CH505 (C.CH505; left box) and heterologous B.63521 (right box) were measured by surface plasmon reasonance.

FIG. 48 shows the development of neutralization breadth in the CH103 clonal lineage. The phylogenetic CH103 clonal lineage tree showing the IC50 (mg ml21) of neutralization of the autologous transmitted/founder (C.CH505), heterologous tier clades A (A.Q842) and B (B.BG1168) viruses as indicated. There is increasing neutralization potency and breadth (TZM-bl assay).

FIG. 49 shows the steps of a B-cell-lineage—based approach to vaccine design. Step 1 is to isolate VH and VL chain members from the peripheral blood or tissues of patients containing BnAbs and to express these native Ig chain pairs as whole antibodies. Step 2 is to infer intermediate ancestor antibodies (IAs, labeled 1, 2 and 3) and the unmutated ancestor antibody (UA). Step 3 requires producing the unmutated and intermediate ancestors as recombinant mAbs and using structure-based alterations in the antigen (changes in Env constructs predicted to enhance binding to the unmutated or intermediate ancestors) or deriving altered antigens using a suitably designed selection strategy. Vaccine administration might prime with the antigen that binds the unmutated ancestor most tightly, and this is then followed by sequential boosts with antigens optimized for binding to each intermediate ancestor. Shown here is an actual clonal lineage of the V1/V2-directed BnAbs CH01-CH04. Targeting the unmutated ancestor with an immunogen that has enhanced binding may induce higher antibody responses. If high-affinity ligands for unmutated ancestors cannot be found, then high-affinity ligands targeting the intermediate ancestors may be equally useful for triggering a response.

FIGS. 50A-50B show a comparison of the pace of viral sequence evolution in CH505 (indicated here by the 9-digit anonymous study-participant identifier 703010505) in regions relevant to the CH103 epitope with other subjects. The regions of interest include the CH103 contacts defined by the structure in this paper, as well as VRC01 contacts and CD4bs contacts, and the V1 and V5 loops immediately adjacent to these contacts. (FIG. 50A) The distribution of sequence distances expressed as the percentage of amino acids that are different between two sequences, resulting from a pair-wise comparison of all sequences sampled in a given time point. Because these are all homogeneous (single-founder) infection cases, very few mutations appear in the CH103 relevant regions or other sites in the virus during acute infection (left hand panels). By 24 weeks after enrollment (week 30 from infection in (A) 703010505, labeled month 6 here as it is approximate), extensive mutations have begun to accrue, focused in CH103 relevant regions (top middle panel), but not in other regions of Env (bottom middle panel). Subject 703010505 has the highest ranked diversity among 15 subjects (B-Q) sampled in this time frame (p=0.067), indicating a focused selective pressure began unusually early in this subject. By 1 year (month 12 indicates samples taken between 10-14 months from enrollment, due to variation in timing of patient visits), this region has begun to evolve in many individuals, possibly due to autologous NAb responses active later in infection. (FIG. 50B) Phylogenetic trees based on concatenated CH103 relevant regions (HXB2 sites 124-127, 131, 132, 279-283, 364-371, 425-432, 455-465, 471-477) were created with PhyML3.0, using HIVw, a within-subject HIV protein substitution model, which was selected to be the optimum model for these sequences using ProtTest. Indels were treated as an additional character state, rather than as missing information. In this view, the extensive evolution away from the T/F virus by month 6, shown in gold, is particularly striking. Distances between sequences sampled in 703010505 (A) at month 6 and the T/F ancestral state were significantly greater than the sequences in the next most variable individual (L) designated by the 9-digit identifier 704010042 (Wilcoxon rank sum, p=0.0003: CH505, median=0.064, range=0.019-0.13, N=25, and 704010042, median=0.0271, range=0.009-0.056, N=26).

### DETAILED DESCRIPTION OF THE INVENTION

The development of a safe, highly efficacious prophylactic HIV-1 vaccine is of paramount importance for the control and prevention of HIV-1 infection. A major goal of HIV-1 vaccine development is the induction of broadly neutralizing antibodies (bnAbs) (Immunol. Rev. 254: 225-244, 2013). BnAbs are protective in rhesus macaques against SHIV challenge, but as yet, are not induced by current vaccines.

For the past 25 years, the HIV vaccine development field has used single or prime boost heterologous Envs as immunogens, but to date has not found a regimen to induce high levels of bnAbs.

Recently, a new paradigm for design of strategies for induction of broadly neutralizing antibodies was introduced, that of B cell lineage immunogen design (Nature Biotech. 30: 423, 2012) in which the induction of bnAb lineages is recreated. It was recently demonstrated the power of mapping the co-evolution of bnAbs and founder virus for elucidating the Env evolution pathways that lead to bnAb induction (Nature 496: 469, 2013). From this type of work has come the hypothesis that bnAb induction will require a selection of antigens to recreate the “swarms” of sequentially evolved viruses that occur in the setting of bnAb generation in vivo in HIV infection (Nature 496: 469, 2013).

A critical question is why the CH505 immunogens are better than other immunogens. This rationale comes from three recent observations. First, a series of immunizations of single putatively “optimized” or “native” trimers when used as an immunogen have not induced bnAbs as single immunogens. Second, in all the chronically infected individuals who do develop bnAbs, they develop them in plasma after ˜2 years. When these individuals have been studied at the time soon after transmission, they do not make bnAbs immediately. Third, now that individual's virus and bnAb co-evolution has been mapped from the time of transmission to the development of bnAbs, the identification of the specific Envs that lead to bnAb development have been identified-thus taking the guess work out of envelope choice.

Two other considerations are important. The first is that for the CH103 bnAb CD4 binding site lineage, the VH4-59 and Vλ3-1 genes are common as are the VDJ, VJ recombinations of the lineage (Liao, Nature 496: 469, 2013). In addition, the bnAb sites are so unusual, the same VH and VL usage has been found to be recurring in multiple individuals. Thus, it can be expected that the CH505 Envs induce CD4 binding site antibodies in many different individuals.

Finally, regarding the choice of gp120 vs. gp160, for the genetic immunization, gp160 would not normally even be considered for use. However, in acute infection, gp41 non-neutralizing antibodies are dominant and overwhelm gp120 responses (Tomaras, G et al. J. Virol. 82: 12449, 2008; Liao, H X et al. JEM 208: 2237, 2011). Recently, it has been found that the HVTN 505 DNA prime, rAd5 vaccine trial that utilized gp140 as an immunogen, also had the dominant response of non-neutralizing gp41 antibodies. Thus, the early-on use of gp160 vs gp120 for gp41 dominance will be explored.

In certain aspects the invention provides a strategy for induction of bnAbs is to select and develop immunogens and combinations designed to recreate the antigenic evolution of Envs that occur when bnAbs do develop in the context of infection.

That broadly neutralizing antibodies (bnAbs) occur in nearly all sera from chronically infected HIV-1 subjects suggests anyone can develop some bnAb response if exposed to immunogens via vaccination. Working back from mature bnAbs through intermediates enabled understanding their development from the unmutated ancestor, and showed that antigenic diversity preceded the development of population breadth. See Liao et al. (2013) Nature 496, 469-476. In this study, an individual “CH505” was followed from HIV-1 transmission to development of broadly neutralizing antibodies. This individual developed antibodies targeted to CD4 binding site on gp120. In this individual the virus was sequenced over time, and broadly neutralizing antibody clonal lineage (“CH103”) was isolated by antigen-specific B cell sorts, memory B cell culture, and amplified by VH/VL next generation pyrosequencing. The CH103 lineage began by binding the T/F virus, autologous neutralization evolved through somatic mutation and affinity maturation, escape from neutralization drove rapid (clearly by 20 weeks) accumulation of variation in the epitope, antibody breadth followed this viral diversification.

Further analysis of envelopes and antibodies from the CH505 individual indicated that a non-CH103 Lineage (DH235) participates in driving CH103-BnAb induction. See Gao et al. (2014) Cell 158:481-491. For example V1 loop, V5 loop and CD4 binding site loop mutations escape from CH103 and are driven by CH103 lineage. Loop D mutations enhanced neutralization by CH103 lineage and are driven by another lineage. Transmitted/founder Env, or another early envelope for example W004.26, triggers naïve B cell with CH103 Unmutated Common Ancestor (UCA) which develop in to intermediate antibodies. Transmitted/founder Env, or another early envelope for example W004.26, also triggers non-CH103 autologous neutralizing Abs that drive loop D mutations in Env that have enhanced binding to intermediate and mature CH103 antibodies and drive remainder of the lineage. In certain embodiments, the inventive composition and methods also comprise loop D mutant envelopes (e.g. but not limited to M10, M11, M19, M20, M21, M5, M6, M7, M8, M9) as immunogens. In certain embodiments, the D-loop mutants are included in an inventive composition used to induce an immune response in a subject. In certain embodiments, the D-loop mutants are included in a composition used as a prime.

The invention provides various methods to choose a subset of viral variants, including but not limited to envelopes, to investigate the role of antigenic diversity in serial samples. In other aspects, the invention provides compositions comprising viral variants, for example but not limited to envelopes, selected based on various criteria as described herein to be used as immunogens. In some embodiments, the immunogens are selected based on the envelope binding to the UCA, and/or intermediate antibodies. In some embodiments the immunogens are selected based on their chronological appearance and/or sequence diversity during infection.

In other aspects, the invention provides immunization strategies using the selections of immunogens to induce cross-reactive neutralizing antibodies. In certain aspects, the immunization strategies as described herein are referred to as “swarm” immunizations to reflect that multiple envelopes are used to induce immune responses. The multiple envelopes in a swarm could be combined in various immunization protocols of priming and boosting.

In certain embodiments the invention provides that sites losing the ancestral, transmitted-founder (T/F) state are most likely under positive selection. From acute, homogenous infections with 3-5 years of follow-up, identified herein are sites of interest among plasma single genome analysis (SGA) Envs by comparing the proportion of sequences per time-point in the T/F state with a threshold, typically 5%. Sites with T/F frequencies below threshold are putative escapes. Clones with representative escape mutations were selected. Where more information was available, such as tree-corrected neutralization signatures and antibody contacts from co-crystal structure, additional sites of interest were considered.

Co-evolution of a broadly neutralizing HIV-1 antibody (CH103) and founder virus was previously reported in African donor (CH505). See Liao et al. (2013) Nature 496, 469-476. In CH505, which had an early antibody that bound autologous T/F virus, 398 envs from 14 time-points over three years (median per sample: 25, range: 18-53) were studied. 36 sites with T/F frequencies under 20% were found in any sample. Neutralization and structure data identified 28 and 22 interesting sites, respectively. Together, six gp41 and 53 gp120 sites were identified, plus six V1 or V5 insertions not in HXB2.

The invention provides an approach to select reagents for neutralization assays and subsequently investigate affinity maturation, autologous neutralization, and the transition to heterologous neutralization and breadth. Given the sustained coevolution of immunity and escape this antigen selection based on antibody and antigen coevolution has specific implications for selection of immunogens for vaccine design.

In one embodiment, 54 envelopes were selected that represent the selected sites. In another embodiment, 27 envelopes were selected that represent the selected sites. These sets of envelopes represent antigenic diversity by deliberate inclusion of polymorphisms that result from immune selection by neutralizing antibodies, and had a lower clustering coefficient and greater diversity in selected sites than sets sampled randomly. These selections represent various levels of antigenic diversity in the HIV-1 envelope. In some embodiments the selections are based on the genetic diversity of longitudinally sampled SGA envelopes. In some embodiments the selections are based on antigenic and or neutralization diversity. In some embodiments and are based on the genetic diversity of longitudinally sampled SGA envelopes, and correlated with other factors such as antigenic/neutralization diversity, and antibody coevolution.

Sequences/Clones

Described herein are nucleic and amino acids sequences of HIV-1 envelopes. In certain embodiments, the described HIV-1 envelope sequences are gp160s. In certain embodiments, the described HIV-1 envelope sequences are gp120s. Other sequences, for example but not limited to gp145s, gp140s, both cleaved and uncleaved, gp150s, gp41s, which are readily derived from the nucleic acid and amino acid gp160 sequences. In certain embodiments the nucleic acid sequences are codon optimized for optimal expression in a host cell, for example a mammalian cell, a rBCG cell or any other suitable expression system. Described herein are nucleic and amino acids sequences of HIV-1 envelopes. In certain embodiments, the described HIV-1 envelope sequences are gp160s. In certain embodiments, the described HIV-1 envelope sequences are gp120s. Other sequences, for example but not limited to gp140s, both cleaved and uncleaved, gp140 Envs with the deletion of the cleavage (C) site, fusion (F) and immunodominant (I) region in gp41—named as gp140ΔCFI, gp140 Envs with the deletion of only the cleavage (C) site and fusion (F) domain—named as gp140ΔCF, gp140 Envs with the deletion of only the cleavage (C)—named gp140ΔC (See e.g. Liao et al. Virology 2006, 353, 268-282), gp145s, gp150s, gp41s, which are readily derived from the nucleic acid and amino acid gp160 sequences. In certain embodiments the nucleic acid sequences are codon optimized for optimal expression in a host cell, for example a mammalian cell, a rBCG cell or any other suitable expression system.

In certain embodiments, the envelope design in accordance with the present invention involves deletion of residues (e.g., 5-11, 5, 6, 7, 8, 9, 10, or 11 amino acids) at the N-terminus. For delta N-terminal design, amino acid residues ranging from 4 residues or even fewer to 14 residues or even more are deleted. These residues are between the maturation (signal peptide, usually ending with CX, X can be any amino acid) and “VPVXXXX . . . ”. In case of CH505 T/F Env as an example, 8 amino acids (italicized and underlined in the below sequence) were deleted: MRVMGIQRNYPQWWIWSMLGFWMLMICNGMWVTVYYGVPVWKEAKTTLFCASDA KAYEKEVHNVWATHACVPTDPNPQE . . . (rest of envelope sequence is indicated as “ . . . ”). In other embodiments, the delta N-design described for CH505 T/F envelope can be used to make delta N-designs of other CH505 envelopes. In certain embodiments, the invention relates generally to an immunogen, gp160, gp120 or gp140, without an N-terminal Herpes Simplex gD tag substituted for amino acids of the N-terminus of gp120, with an HIV leader sequence (or other leader sequence), and without the original about 4 to about 25, for example 4, 5, 6, 7, 8, 9, 10, 11, 12, 13, 14, 15, 16, 17, 18, 19, 20, 21, 22, 23, 24, 25 amino acids of the N-terminus of the envelope (e.g. gp120). See WO2013/006688, e.g. at pages 10-12, the contents of which publication is hereby incorporated by reference in its entirety.

The general strategy of deletion of N-terminal amino acids of envelopes results in proteins, for example gp120s, expressed in mammalian cells that are primarily monomeric, as opposed to dimeric, and, therefore, solves the production and scalability problem of commercial gp120 Env vaccine production. In other embodiments, the amino acid deletions at the N-terminus result in increased immunogenicity of the envelopes.

In certain embodiments, the invention provides envelope sequences, amino acid sequences and the corresponding nucleic acids, and in which the V3 loop is substituted with the following V3 loop sequence TRPNNNTRKSIRIGPGQTFY ATGDIIGNIRQAH (SEQ ID NO: 150). This substitution of the V3 loop reduced product cleavage and improves protein yield during recombinant protein production in CHO cells.

In certain embodiments, the CH505 envelopes will have added certain amino acids to enhance binding of various broad neutralizing antibodies. Such modifications could include but not limited to, mutations at W680G or modification of glycan sites for enhanced neutralization.

In certain aspects, the invention provides composition and methods which use a selection of sequential CH505 Envs, as gp120s, gp 140s cleaved and uncleaved, gp145s, gp150s and gp160s, as proteins, DNAs, RNAs, or any combination thereof, administered as primes and boosts to elicit immune response. Sequential CH505 Envs as proteins would be co-administered with nucleic acid vectors containing Envs to amplify antibody induction. In certain embodiments, the compositions and methods include any immunogenic HIV-1 sequences to give the best coverage for T cell help and cytotoxic T cell induction. In certain embodiments, the compositions and methods include mosaic and/or consensus HIV-1 genes to give the best coverage for T cell help and cytotoxic T cell induction. In certain embodiments, the compositions and methods include mosaic group M and/or consensus genes to give the best coverage for T cell help and cytotoxic T cell induction. In some embodiments, the mosaic genes are any suitable gene from the HIV-1 genome. In some embodiments, the mosaic genes are Env genes, Gag genes, Pol genes, Nef genes, or any combination thereof. See e.g. U.S. Pat. No. 7,951,377. In some embodiments the mosaic genes are bivalent mosaics. In some embodiments the mosaic genes are trivalent. In some embodiments, the mosaic genes are administered in a suitable vector with each immunization with Env gene inserts in a suitable vector and/or as a protein. In some embodiments, the mosaic genes, for example as bivalent mosaic Gag group M consensus genes, are administered in a suitable vector, for example but not limited to HSV2, would be administered with each immunization with Env gene inserts in a suitable vector, for example but not limited to HSV-2.

In certain aspects the invention provides compositions and methods of Env genetic immunization either alone or with Env proteins to recreate the swarms of evolved viruses that have led to bnAb induction. Nucleotide-based vaccines offer a flexible vector format to immunize against virtually any protein antigen. Currently, two types of genetic vaccination are available for testing—DNAs and mRNAs.

In certain aspects the invention contemplates using immunogenic compositions wherein immunogens are delivered as DNA. See Graham B S, Enama M E, Nason M C, Gordon I J, Peel S A, et al. (2013) DNA Vaccine Delivered by a Needle-Free Injection Device Improves Potency of Priming for Antibody and CD8+ T-Cell Responses after rAd5 Boost in a Randomized Clinical Trial. PLoS ONE 8(4): e59340, page 9. Various technologies for delivery of nucleic acids, as DNA and/or RNA, so as to elicit immune response, both T-cell and humoral responses, are known in the art and are under developments. In certain embodiments, DNA can be delivered as naked DNA. In certain embodiments, DNA is formulated for delivery by a gene gun. In certain embodiments, DNA is administered by electroporation, or by a needle-free injection technologies, for example but not limited to Biojector® device. In certain embodiments, the DNA is inserted in vectors. The DNA is delivered using a suitable vector for expression in mammalian cells. In certain embodiments the nucleic acids encoding the envelopes are optimized for expression. In certain embodiments DNA is optimized, e.g. codon optimized, for expression. In certain embodiments the nucleic acids are optimized for expression in vectors and/or in mammalian cells. In non-limiting embodiments these are bacterially derived vectors, adenovirus based vectors, rAdenovirus (e.g. Barouch D H, et al. Nature Med. 16: 319-23, 2010), recombinant mycobacteria (e.g. rBCG or M smegmatis) (Yu, J S et al. Clinical Vaccine Immunol. 14: 886-093, 2007; ibid 13: 1204-11, 2006), and recombinant vaccinia type of vectors (Santra S. Nature Med. 16: 324-8, 2010), for example but not limited to ALVAC, replicating (Kibler K V et al., PLoS One 6: e25674, 2011 Nov. 9.) and non-replicating (Perreau M et al. J. virology 85: 9854-62, 2011) NYVAC, modified vaccinia Ankara (MVA)), adeno-associated virus, Venezuelan equine encephalitis (VEE) replicons, Herpes Simplex Virus vectors, and other suitable vectors.

In certain aspects the invention contemplates using immunogenic compositions wherein immunogens are delivered as DNA or RNA in suitable formulations. Various technologies which contemplate using DNA or RNA, or may use complexes of nucleic acid molecules and other entities to be used in immunization. In certain embodiments, DNA or RNA is administered as nanoparticles consisting of low dose antigen-encoding DNA formulated with a block copolymer (amphiphilic block copolymer 704). See Cany et al., Journal of Hepatology 2011 vol. 54 j 115-121; Arnaoty et al., Chapter 17 in Yves Bigot (ed.), Mobile Genetic Elements: Protocols and Genomic Applications, Methods in Molecular Biology, vol. 859, pp 293-305 (2012); Arnaoty et al. (2013) Mol Genet Genomics. 2013 August; 288(7-8):347-63. Nanocarrier technologies called Nanotaxi® for immunogenic macromolecules (DNA, RNA, Protein) delivery are under development. See for example technologies developed by incellart.

In certain aspects the invention contemplates using immunogenic compositions wherein immunogens are delivered as recombinant proteins. Various methods for production and purification of recombinant proteins suitable for use in immunization are known in the art. In certain embodiments recombinant proteins are produced in CHO cells.

The immunogenic envelopes can also be administered as a protein boost in combination with a variety of nucleic acid envelope primes (e.g., HIV -1 Envs delivered as DNA expressed in viral or bacterial vectors).

Dosing of proteins and nucleic acids can be readily determined by a skilled artisan. A single dose of nucleic acid can range from a few nanograms (ng) to a few micrograms GO or milligram of a single immunogenic nucleic acid. Recombinant protein dose can range from a few μg micrograms to a few hundred micrograms, or milligrams of a single immunogenic polypeptide.

Administration: The compositions can be formulated with appropriate carriers using known techniques to yield compositions suitable for various routes of administration. In certain embodiments the compositions are delivered via intramascular (IM), via subcutaneous, via intravenous, via nasal, via mucosal routes, or any other suitable route of immunization.

The compositions can be formulated with appropriate carriers and adjuvants using techniques to yield compositions suitable for immunization. The compositions can include an adjuvant, such as, for example but not limited to, alum, poly IC, MF-59 or other squalene-based adjuvant, ASOIB, or other liposomal based adjuvant suitable for protein or nucleic acid immunization. In certain embodiments, the adjuvant is GSK AS01E adjuvant containing MPL and QS21. This adjuvant has been shown by GSK to be as potent as the similar adjuvant AS01B but to be less reactogenic using HBsAg as vaccine antigen [Leroux-Roels et al., IABS Conference, April 2013, 9]. In certain embodiments, TLR agonists are used as adjuvants. In other embodiment, adjuvants which break immune tolerance are included in the immunogenic compositions.

In certain embodiments, the compositions and methods comprise any suitable agent or immune modulation which could modulate mechanisms of host immune tolerance and release of the induced antibodies. In non-limiting embodiments modulation includes PD-1 blockade; T regulatory cell depletion; CD40L hyperstimulation; soluble antigen administration, wherein the soluble antigen is designed such that the soluble agent eliminates B cells targeting dominant epitopes, or a combination thereof. In certain embodiments, an immunomodulatory agent is administered in at time and in an amount sufficient for transient modulation of the subject's immune response so as to induce an immune response which comprises broad neutralizing antibodies against HIV-1 envelope. Non-limiting examples of such agents is any one of the agents described herein: e.g. chloroquine (CQ), PTP1B Inhibitor—CAS 765317-72-4—Calbiochem or MSI 1436 clodronate or any other bisphosphonate; a Foxol inhibitor, e.g. 344355|Foxo1 Inhibitor, AS1842856—Calbiochem; Gleevac, anti-CD25 antibody, anti-CCR4 Ab, an agent which binds to a B cell receptor for a dominant HIV-1 envelope epitope, or any combination thereof. In certain embodiments, the methods comprise administering a second immunomodulatory agent, wherein the second and first immunomodulatory agents are different.

There are various host mechanisms that control bNAbs. For example highly somatically mutated antibodies become autoreactive and/or less fit (Immunity 8: 751, 1998; PloS Comp. Biol. 6 e1000800, 2010; J. Thoret. Biol. 164:37, 1993); Polyreactive/autoreactive naïve B cell receptors (unmutated common ancestors of clonal lineages) can lead to deletion of Ab precursors (Nature 373: 252, 1995; PNAS 107: 181, 2010; J. Immunol. 187: 3785, 2011); Abs with long HCDR3 can be limited by tolerance deletion (JI 162: 6060, 1999; JCI 108: 879, 2001). BnAb knock-in mouse models are providing insights into the various mechanisms of tolerance control of MPER BnAb induction (deletion, anergy, receptor editing). Other variations of tolerance control likely will be operative in limiting BnAbs with long HCDR3s, high levels of somatic hypermutations.

The invention is described in the following non-limiting examples.

### EXAMPLES

### Example 1

HIV-1 sequences, including envelopes, and antibodies from HIV-1 infected individual CH505 were isolated as described in Liao et al. (2013) Nature 496, 469-476 including supplementary materials; See also Gao et al. (2014) Cell 158:481-491.

Recombinant HIV-1 Proteins

HIV-1 Env genes for subtype B, 63521, subtype C, 1086, and subtype CRF_01, 427299, as well as subtype C, CH505 autologous transmitted/founder Env were obtained from acutely infected HIV-1 subjects by single genome amplification, codon-optimized by using the codon usage of highly expressed human housekeeping genes, de novo synthesized (GeneScript) as gp140 or gp120 (AE.427299) and cloned into a mammalian expression plasmid pcDNA3.1/hygromycin (Invitrogen). Recombinant Env glycoproteins were produced in 293F cells cultured in serum-free medium and transfected with the HIV-1 gp140- or gp120-expressing pcDNA3.1 plasmids, purified from the supernatants of transfected 293F cells by using Galanthus nivalis lectin-agarose (Vector Labs) column chromatography, and stored at −80° C. Select Env proteins made as CH505 transmitted/founder Env were further purified by superose 6 column chromatography to trimeric forms, and used in binding assays that showed similar results as with the lectin-purified oligomers.

ELISA

Binding of patient plasma antibodies and CH103, and DH235(CH235), See Gao et al. (2014) Cell 158:481-491, clonal lineage antibodies to autologous and heterologous HIV-1 Env proteins was measured by ELISA as described previously. Plasma samples in serial threefold dilutions starting at 1:30 to 1:521,4470 or purified monoclonal antibodies in serial threefold dilutions starting at 100 μg ml-1 to 0.000 μg ml-1 diluted in PBS were assayed for binding to autologous and heterologous HIV-1 Env proteins. Binding of biotin-labelled CH103 at the subsaturating concentration was assayed for cross-competition by unlabeled HIV-1 antibodies and soluble CD4-Ig in serial fourfold dilutions starting at 10 μg ml-1. The half-maximal effective concentration (EC50) of plasma samples and monoclonal antibodies to HIV-1 Env proteins were determined and expressed as either the reciprocal dilution of the plasma samples or concentration of monoclonal antibodies.

Surface Plasmon Resonance Affinity and Kinetics Measurements

Binding Kd and rate constant (association rate (Ka)) measurements of monoclonal antibodies and all candidate UCAs to the autologous Env C. CH05 gp140 and/or the heterologous Env B.63521 gp120 are carried out on BIAcore 3000 instruments as described previously. Anti-human IgG Fc antibody (Sigma Chemicals) is immobilized on a CM5 sensor chip to about 15,000 response units and each antibody is captured to about 50-200 response units on three individual flow cells for replicate analysis, in addition to having one flow cell captured with the control Synagis (anti-RSV) monoclonal antibody on the same sensor chip. Double referencing for each monoclonal antibody—HIV-1 Env binding interactions is used to subtract nonspecific binding and signal drift of the Env proteins to the control surface and blank buffer flow, respectively. Antibody capture level on the sensor surface is optimized for each monoclonal antibody to minimize rebinding and any associated avidity effects. C.CH505 Env gp140 protein is injected at concentrations ranging from 2 to 25 μg ml-1, and B.63521 gp120 was injected at 50-400 μg ml-1 for UCAs and early intermediates IA8 and IA4, 10-100 μg ml-1 for intermediate IA3, and 1-25 μg ml-1 for the distal and mature monoclonal antibodies. All curve-fitting analyses are performed using global fit of to the 1:1 Langmuir model and are representative of at least three measurements. All data analysis was performed using the BIAevaluation 4.1 analysis software (GE Healthcare).

Neutralization Assays

Neutralizing antibody assays in TZM-bl cells are performed as described previously. Neutralizing activity of plasma samples in eight serial threefold dilutions starting at 1:20 dilution and for recombinant monoclonal antibodies in eight serial threefold dilutions starting at 50 μg ml-1 are tested against autologous and herologous HIV-1 Env-pseudotyped viruses in TZM-bl-based neutralization assays using the methods known in the art. Neutralization breadth of CH103 is determined using a panel of 196 of geographically and genetically diverse Env-pseudoviruses representing the major circulated genetic subtypes and circulating recombinant forms. HIV-1 subtype robustness is derived from the analysis of HIV-1 clades over time. The data are calculated as a reduction in luminescence units compared with control wells, and reported as IC50 in either reciprocal dilution for plasma samples or in micrograms per microlitre for monoclonal antibodies.

The GenBank accession numbers for 292 CH505 Env proteins are KC247375-KC247667, and accessions for 459 V_{H}DJ_{H} and 174 V_{L}J_{L} sequences of antibody members in the CH103 clonal lineage are KC575845-KC576303 and KC576304-KC576477, respectively.

### Example 2

Binding of sequential envelopes to CH103 CD4 binding site bnAb lineage members. The binding assay was an ELISA with the envelope protein bound to the well surface of a 96 well plate, and the antibody in questions incubated with the envelope bound to the plate. After washing, an enzyme-labeled anti-human IgG antibody was added and after incubation, washed away. The intensity of binding was determined by the intensity of enzyme-activated color in the well.

TABLE 1
ELISA binding, log-transformed area under the curve (AUC) values for a realization that
embodies 54 Env-derived gp120 antigens, assayed against members of the CH103 bnAb lineage
from universal ancestor (UCA), through intermediate ancestors (IA8-IA1) to the mature
bnAb. Values of 0 indicate no binding. Only 27 of the 54 antigens in this particular
embodiment were assayed for binding. The TF antigen was derived from Env w004.3.

| Antigen | UCA | IA8 | IA7 | IA6 | IA4 | IA3 | CH105 | IA2 | CH104 | IA1 | CH106 | CH103 |
|---|---|---|---|---|---|---|---|---|---|---|---|---|
| TF | 3.5 | 5.5 | 9.2 | 9.1 | 10.1 | 11 | 11.2 | 10.8 | 10.4 | 10.4 | 11.3 | 12.6 |
| w020.15 | 1.6 | 4.2 | 8.2 | 7.8 | 9.1 | 10.2 | 10.8 | 10.5 | 10.5 | 9.9 | 10.5 | 11.8 |
| w030.13 | 0.3 | 2 | 4.7 | 6.5 | 7.4 | 9 | 10.5 | 11.4 | 11.3 | 10.5 | 11.9 | 12.9 |
| w020.25 | 0.8 | 2.4 | 6.4 | 6 | 7.3 | 8.6 | 8.2 | 9 | 8.3 | 8.6 | 9.4 | 10.3 |
| w004.54 | 0 | 0.5 | 2.3 | 2.9 | 2.8 | 5.1 | 8.3 | 6.8 | 8.1 | 6.2 | 8.1 | 9.2 |
| w020.11 | 0 | 0.9 | 0.1 | 0.8 | 0.8 | 0.8 | 3.6 | 2.6 | 2.2 | 1.8 | 5.4 | 9.6 |
| w078.15 | 0 | 0 | 0.7 | 1 | 1.3 | 3 | 10.1 | 11.5 | 10.8 | 10.9 | 11 | 10.7 |
| w053.22 | 0 | 0 | 0 | 0 | 0.2 | 1.1 | 9 | 9.3 | 9.9 | 8.8 | 9.8 | 11.6 |
| w136.B23 | 0 | 0 | 0 | 0 | 0 | 0 | 13.7 | 14.3 | 14.2 | 14.4 | 13.3 | 11.8 |
| w053.31 | 0 | 0 | 0 | 0 | 0 | 0 | 13.5 | 13.3 | 13.7 | 13.4 | 13.4 | 13.6 |
| w136.B2 | 0 | 0 | 0 | 0 | 0 | 0 | 12.4 | 13.1 | 13.2 | 13.2 | 12.7 | 10.8 |
| w100.A13 | 0 | 0 | 0 | 0 | 0 | 0 | 11.4 | 12.5 | 12.9 | 12.6 | 12 | 12.9 |
| w100.B4 | 0 | 0 | 0 | 0 | 0 | 0 | 11.9 | 13.4 | 13.1 | 13.7 | 12.6 | 9.7 |
| w160.T4 | 0 | 0 | 0 | 0 | 0 | 0 | 12.2 | 13.4 | 12.8 | 13.6 | 12.3 | 9 |
| w030.21 | 0 | 0 | 0 | 0 | 0 | 0 | 10.6 | 11.5 | 11.3 | 11.8 | 10.9 | 12.2 |
| w053.15 | 0 | 0 | 0 | 0 | 0 | 0 | 9.4 | 9.5 | 10.4 | 9.2 | 10.1 | 11.2 |
| w078.17 | 0 | 0 | 0 | 0 | 0 | 0 | 9.5 | 9.9 | 10 | 9 | 8.7 | 11.3 |
| w136.B10 | 0 | 0 | 0 | 0 | 0 | 0 | 8.5 | 9.7 | 8.9 | 9.2 | 9 | 11.5 |
| w053.29 | 0 | 0 | 0 | 0 | 0 | 0 | 8.5 | 9.2 | 9.8 | 8.8 | 9.7 | 10.2 |
| w078.33 | 0 | 0 | 0 | 0 | 0 | 0 | 8.9 | 9 | 9 | 8.2 | 9.5 | 11.1 |
| w136.B5 | 0 | 0 | 0 | 0 | 0 | 0 | 10.5 | 9.9 | 10.6 | 9.5 | 10.9 | 4.3 |
| w030.36 | 0 | 0 | 0 | 0 | 0 | 0 | 7.5 | 7.3 | 7.8 | 6.7 | 8.5 | 9.1 |
| w030.17 | 0 | 0 | 0 | 0 | 0 | 0 | 6.7 | 7 | 7 | 5.8 | 8.2 | 9.9 |
| w078.9 | 0 | 0 | 0 | 0 | 0 | 0 | 6.6 | 7.2 | 6.9 | 6.3 | 8.3 | 8.9 |
| w030.20 | 0 | 0 | 0 | 0 | 0 | 0 | 7.1 | 6.3 | 7.5 | 5.7 | 7.3 | 9.5 |
| w100.B2 | 0 | 0 | 0 | 0 | 0 | 0 | 7.5 | 7.5 | 6.1 | 7.3 | 7.8 | 3.2 |
| w078.6 | 0 | 0 | 0 | 0 | 0 | 0 | 3.8 | 4.6 | 4.5 | 3 | 6.7 | 9.9 |

### Example 3

Combinations of antigens derived from CH505 envelope sequences for swarm immunizations

Provided herein are non-limiting examples of combinations of antigens derived from CH505 envelope sequences for a swarm immunization. Without limitations, these selected combinations comprise envelopes which provide representation of the sequence and antigenic diversity of the HIV-1 envelope variants which lead to the induction and maturation of the CH103 and CH235 antibody lineages.

The selection includes priming with a virus which binds to the UCA, for example a T/F virus or another early (e.g. but not limited to week 004.3, or 004.26) virus envelope. In certain embodiments the prime could include D-loop variants. In certain embodiments the boost could include D-loop variants. In certain embodiments, these D-loop variants are envelope escape mutants not recognized by the UCA. Non-limiting examples of such D-loop variants are envelopes designated as M10, M11, M19, M20, M21, M5, M6, M7, M8, M9, M14 (TF_M14), M24 (TF_24), M15, M16, M17, M18, M22, M23, M24, M25, M26. See Gao et al. (2014) Cell 158:481-491.

Non-limiting embodiments of envelopes selected for swarm vaccination are shown as the selections described below. A skilled artisan would appreciate that a vaccination protocol can include a sequential immunization starting with the “prime” envelope(s) and followed by sequential boosts, which include individual envelopes or combination of envelopes. In another vaccination protocol, the sequential immunization starts with the “prime” envelope(s) and is followed with boosts of cumulative prime and/or boost envelopes. In certain embodiments, the sequential immunization starts with the “prime” envelope(s) and is followed by boost(s) with all or various combinations of the envelopes in the selection. In certain embodiments, the prime does not include T/F sequence (W000.TF). In certain embodiments, the prime includes w004.03 envelope. In certain embodiments, the prime includes w004.26 envelope. In certain embodiment the prime includes M11. In certain embodiments the prime includes M5. In certain embodiments, the immunization methods do not include immunization with HIV-1 envelope T/F. In certain embodiments, the immunization methods do not include a schedule of four valent immunization with HIV-1 envelopes T/F, w053.16, w078.33, and w100.B6.

In certain embodiments, there is some variance in the immunization regimen; in some embodiments, the selection of HIV-1 envelopes may be grouped in various combinations of primes and boosts, either as nucleic acids, proteins, or combinations thereof.

In certain embodiments the immunization includes a prime administered as DNA, and MVA boosts. See Goepfert, et al. 2014; “Specificity and 6-Month Durability of Immune Responses Induced by DNA and Recombinant Modified Vaccinia Ankara Vaccines Expressing HIV-1 Virus-Like Particles” J Infect Dis. 2014 Feb. 9. [Epub ahead of print].

HIV-1 Envelope selection A (54 envelopes):
w000.TF
w004.31, w004.54
w007.8, w007.21, w007.25, w007.34
w008.20
w009.19
w010.7
w020.15, w020.11, w020.24, w020.25
w022.6, w022.5, w022.9, w022.22
w030.20, w030.17, w030.21, w030.36, w030.26, w030.13, w030.32
w053.15, w053.29, w053.22, w053.8, w053.31, w053.9
w078.6, w078.36, w078.9, w078.26, w078.29, w078.30, w078.33, w078.17, w078.15, w078.27
w100.T3, w100.B10, w100.B2, w100.B4, w100.A11, w100.A13
w136.B10, w136.B5, w136.B2, w136.B23
w160.C1, w160.T3, w160.T4

HIV-1 Envelope selection B (27 envelopes): The bolded envelopes from selection A above:
w000.TF, w004.54, w020.15, w020.11, w020.25, w030.20, w030.17, w030.21, w030.36, w030.13, w053.15, w053.29, w053.22, w053.31, w078.6, w078.9, w078.33, w078.17, w078.15, w100.B2, w100.B4, w100.A13, w136.B10, w136.B5, w136.B2, w136.B23, w160.T4.

In certain embodiments the selections above could include additional envelopes from later time points. In certain embodiments, the selections above could include a D-loop mutant, or a combination thereof.

The selections of CH505-Envs were down-selected from a series of 400 CH505 Envs isolated by single-genome amplification followed for 3 years after acute infection, based on experimental data. The enhanced neutralization breadth that developed in the CD4-binding site (bs) CH103 antibody lineage that arose in subject CH505 developed in conjunction with epitope diversification in the CH505's viral quasispecies. It was observed that at 6 months post-infection in there was more diversification in the CD4bs epitope region in this donor than sixteen other acutely infected donors. Population breadth did not arise in the CH103 antibody lineage until the epitope began to diversify. A hypothesis is that the CH103 linage drove viral escape, but then the antibody adapted to the relatively resistant viral variants. As this series of events was repeated, the emerging antibodies evolved to tolerate greater levels of diversity in relevant sites, and began to be able to recognize and neutralize diverse heterologous forms for the virus and manifest population breadth. In certain embodiments, 54 envs are selected from CH505 sequences to reflect diverse variants for making Env pseudoviruses, with the goal of recapitulating CH505 HIV-1 antigenic diversity over time, making sure selected site (i.e. those sites reflecting major antigenic shifts) diversity was represented.

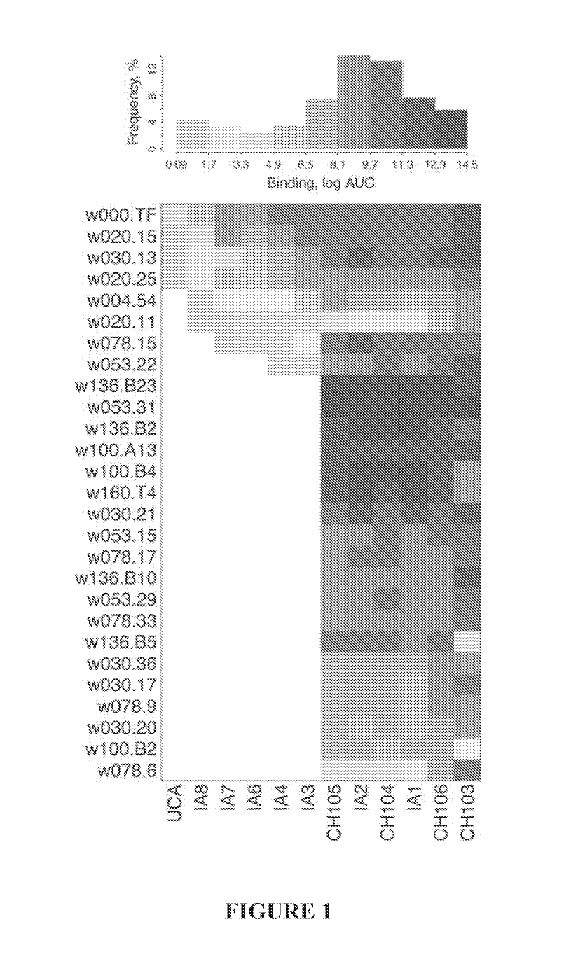

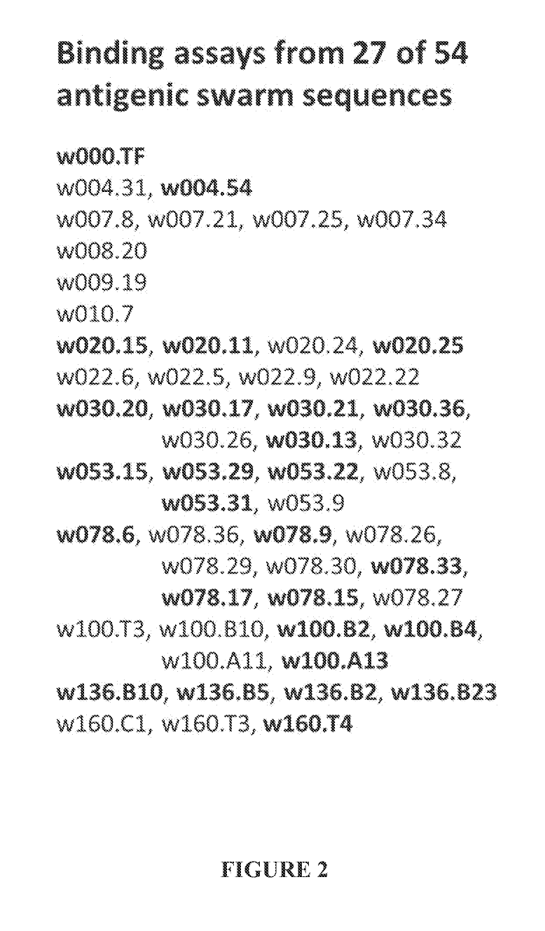

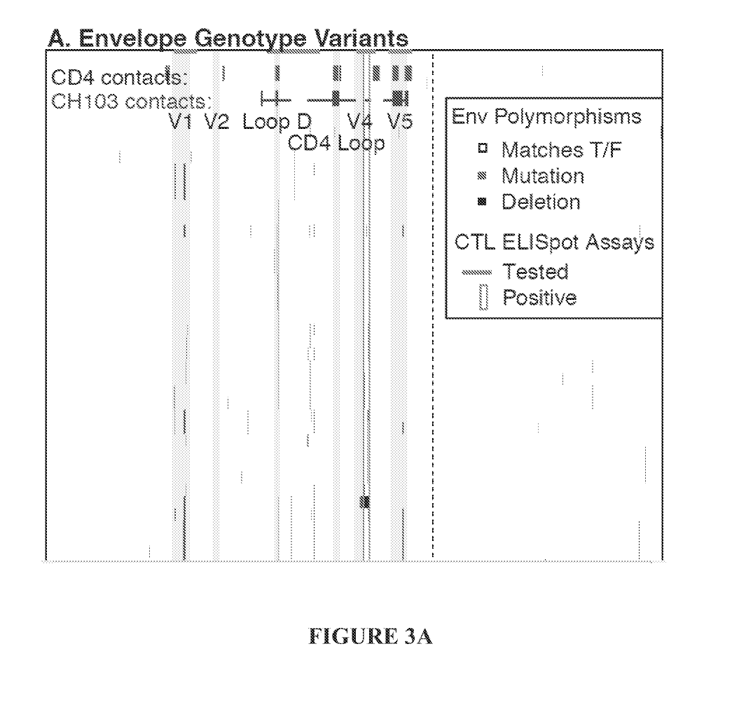

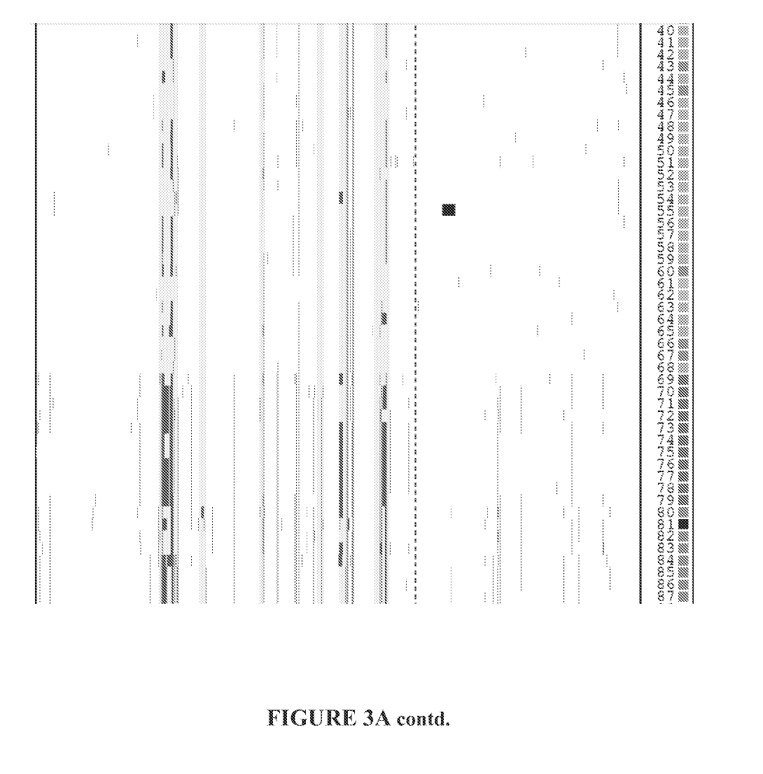

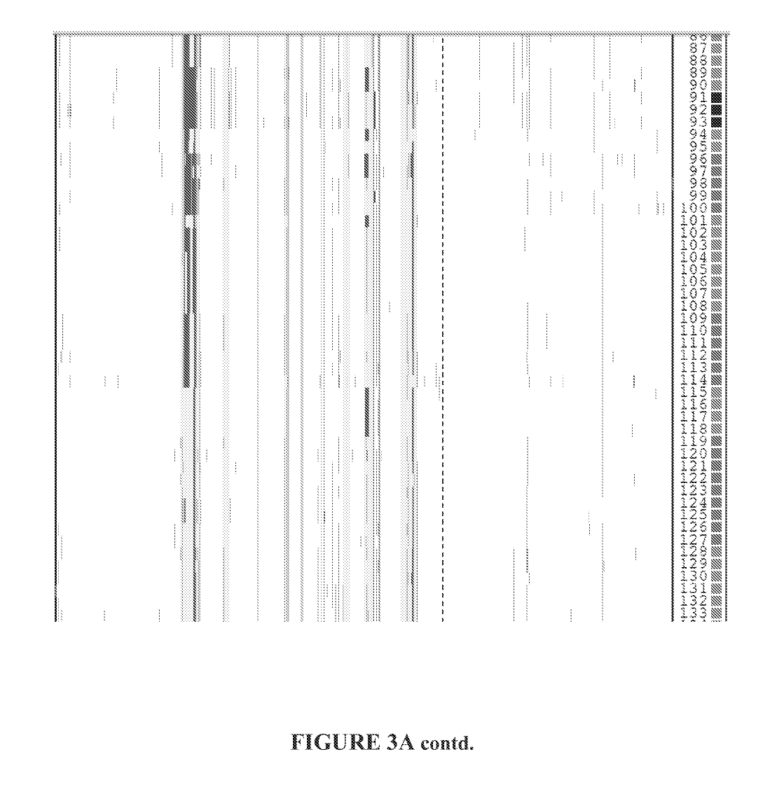

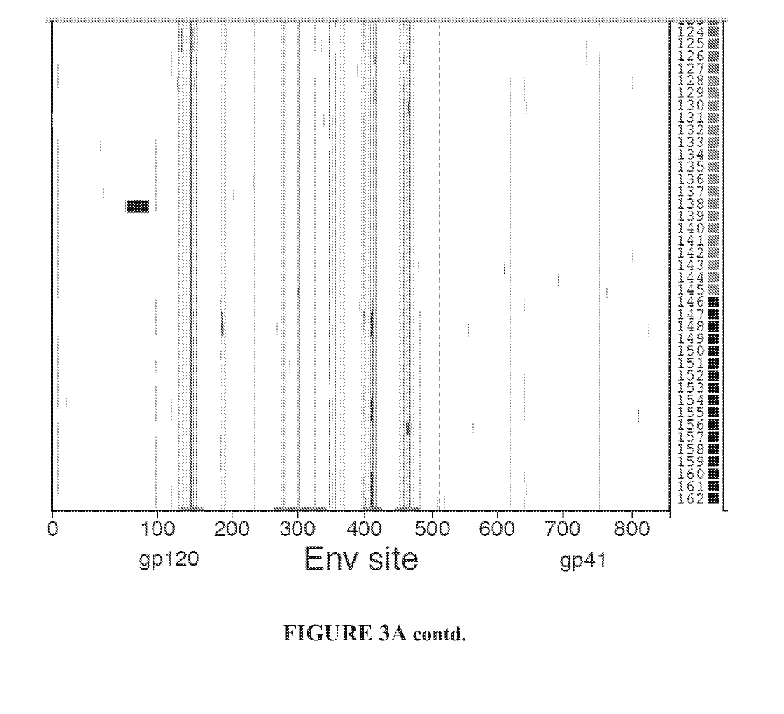

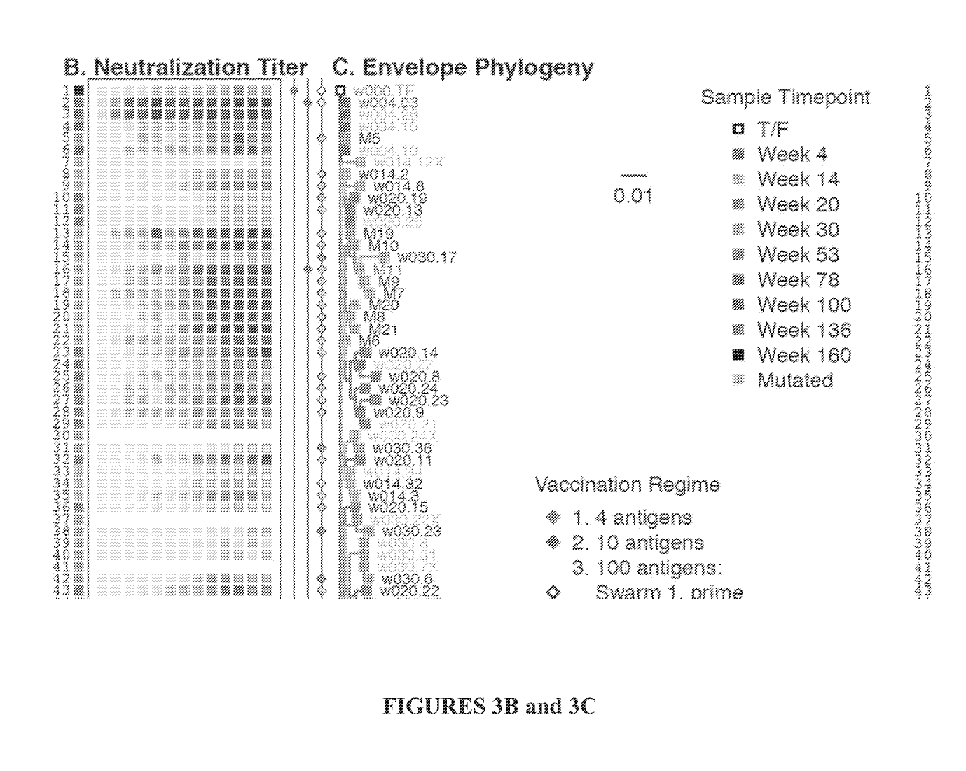

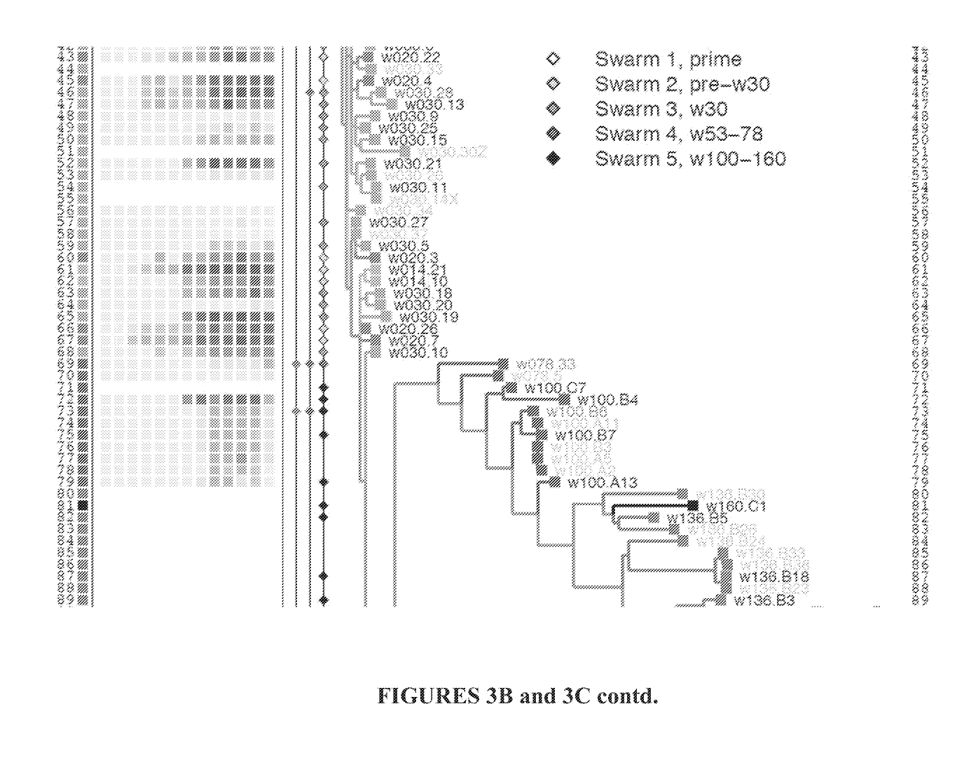

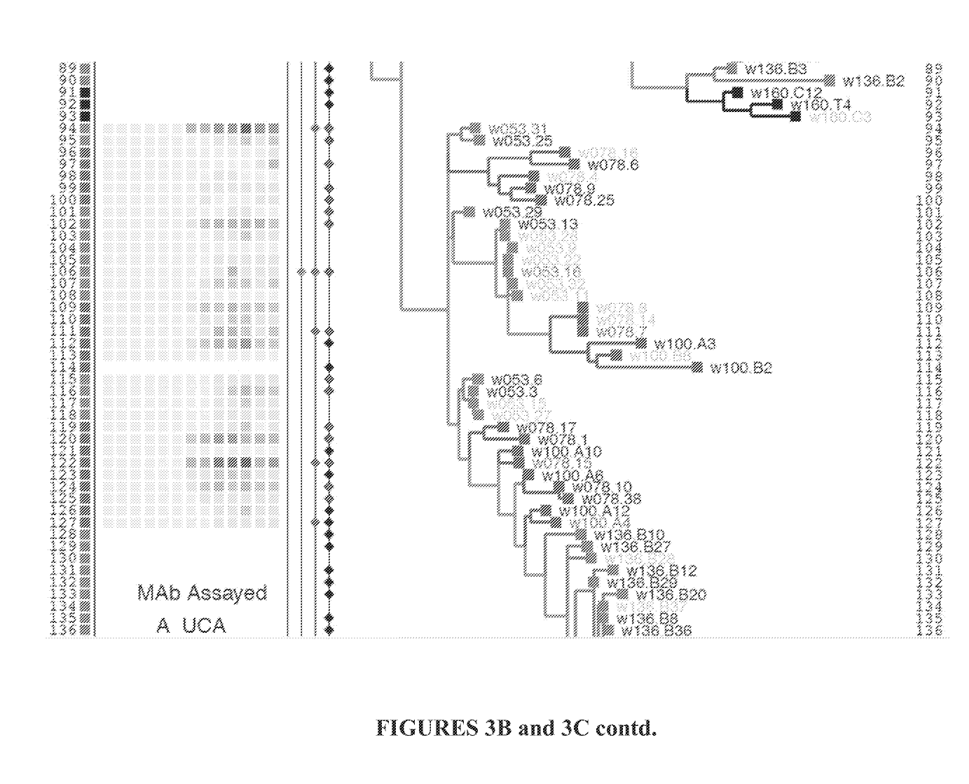

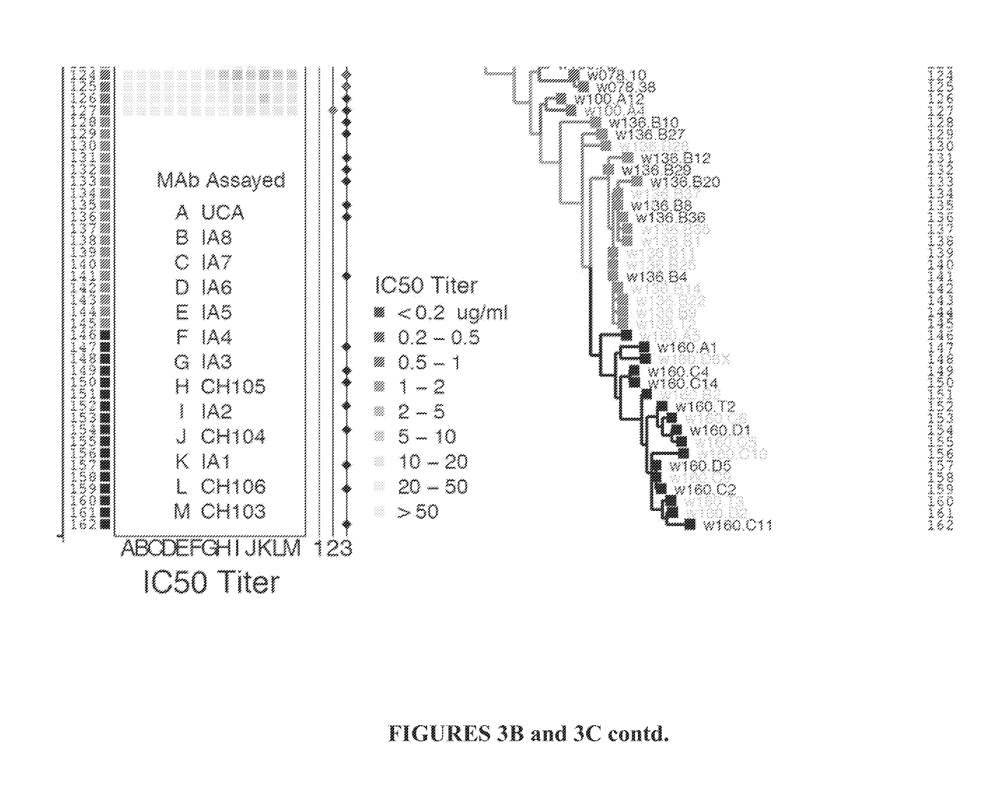

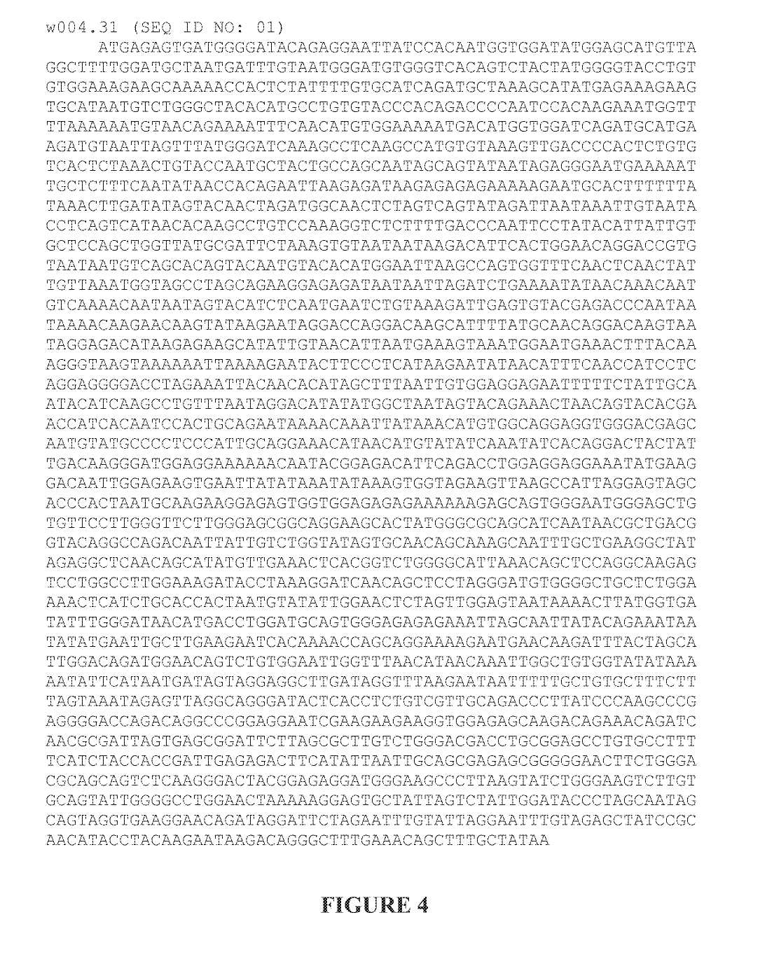

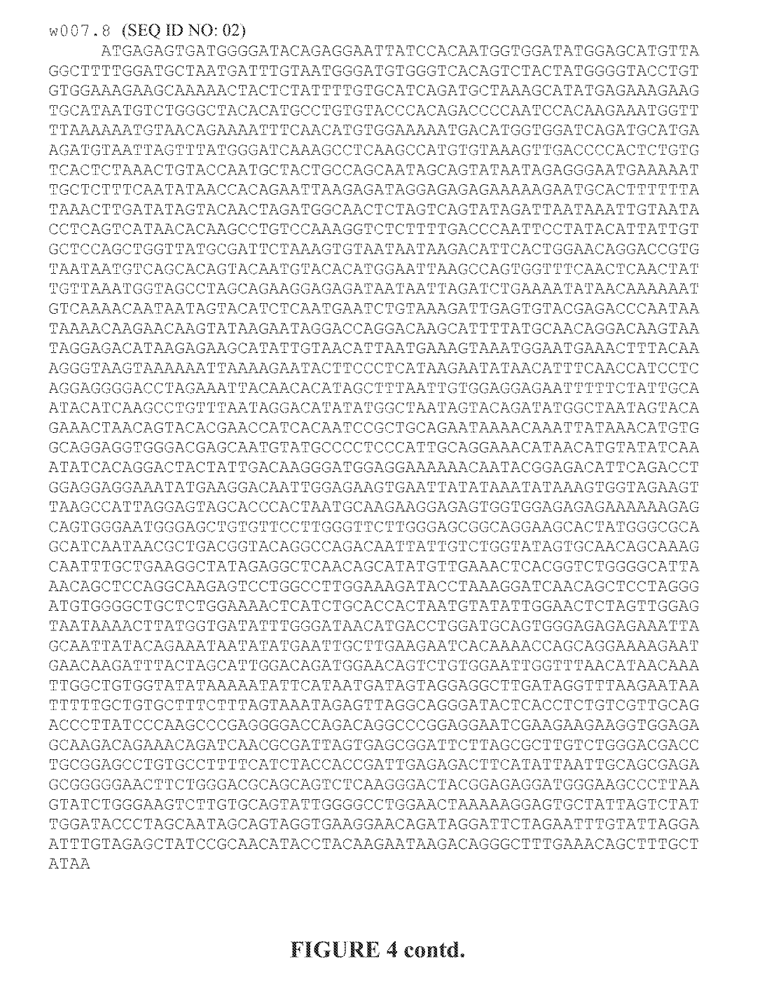

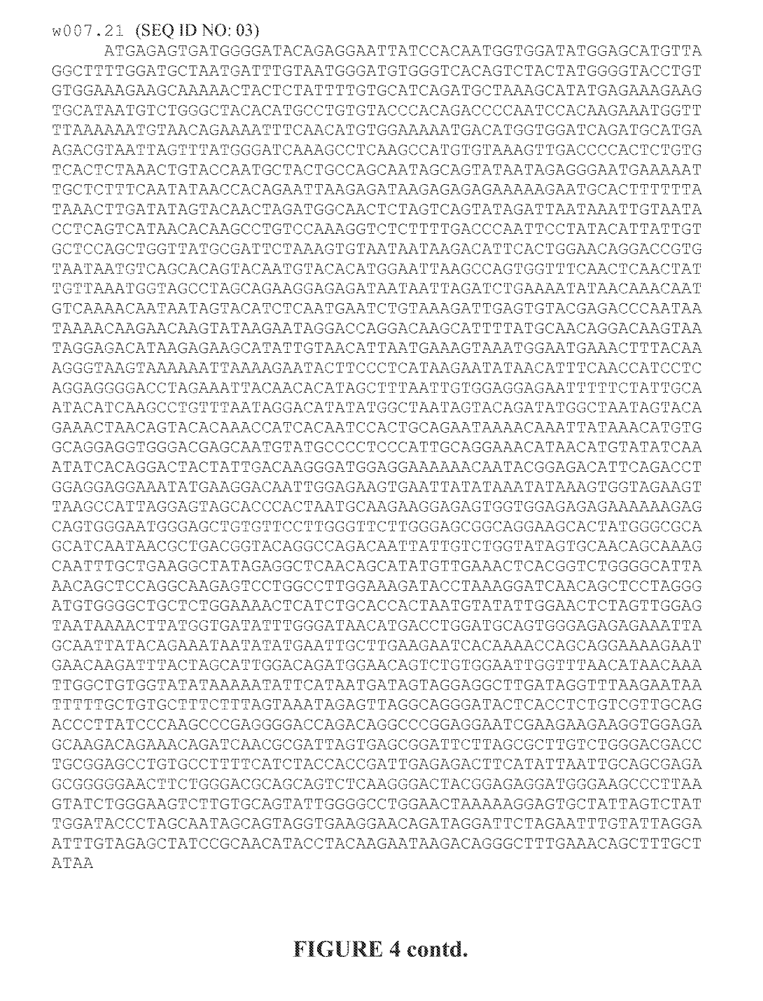

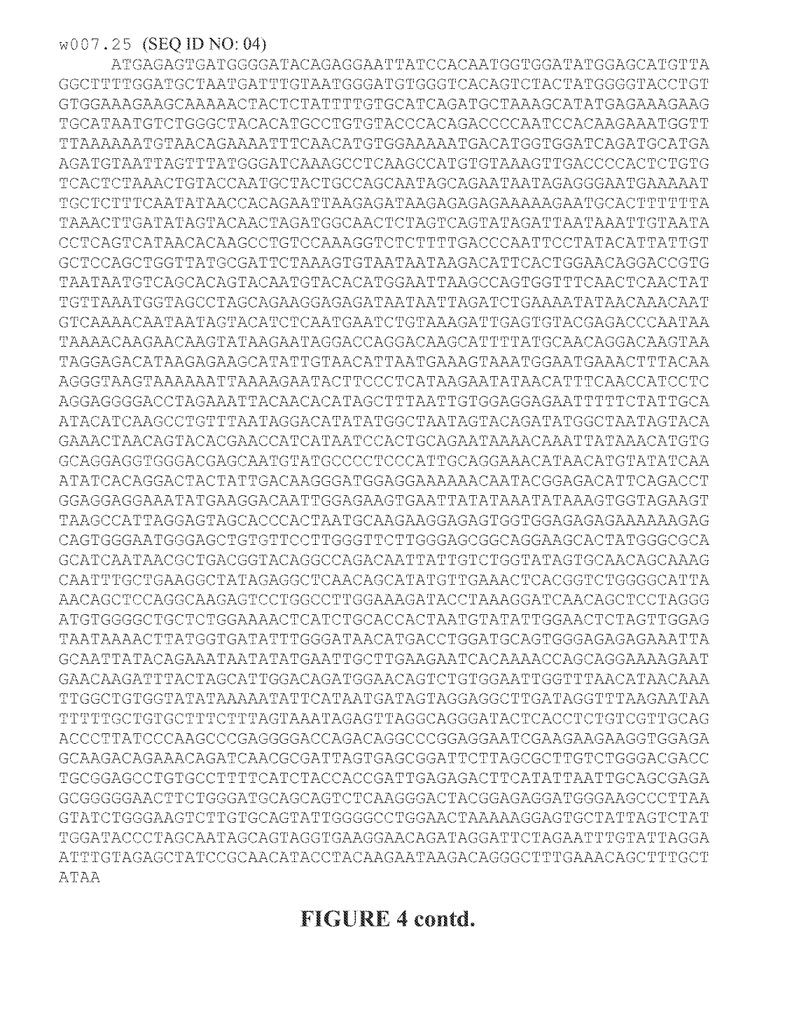

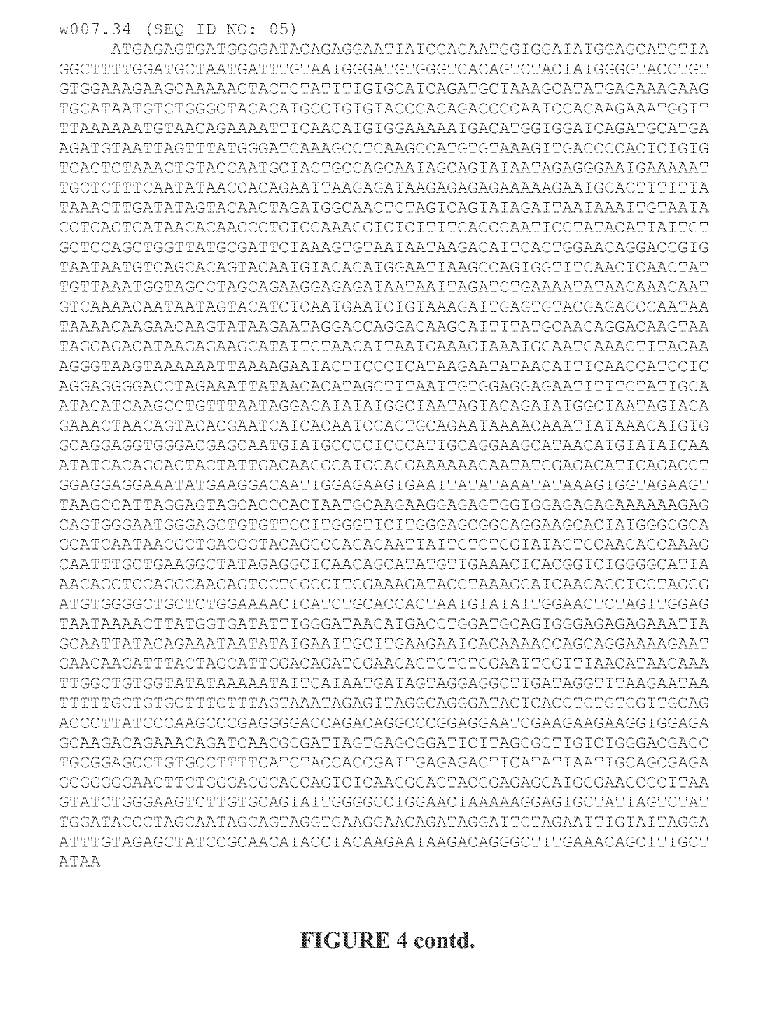

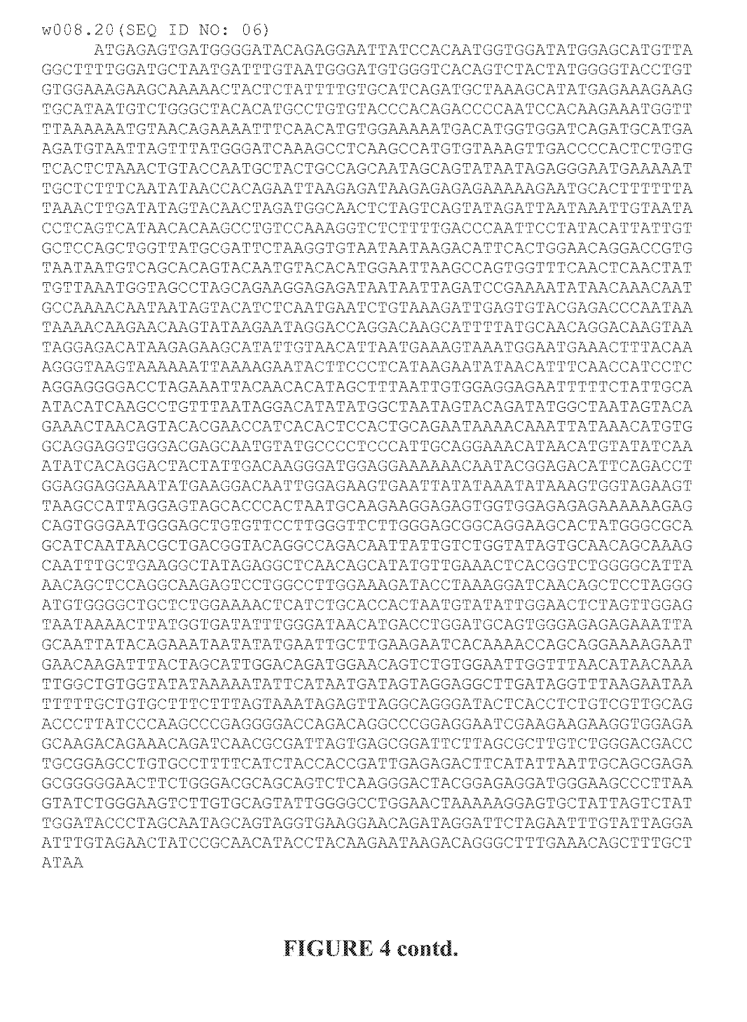

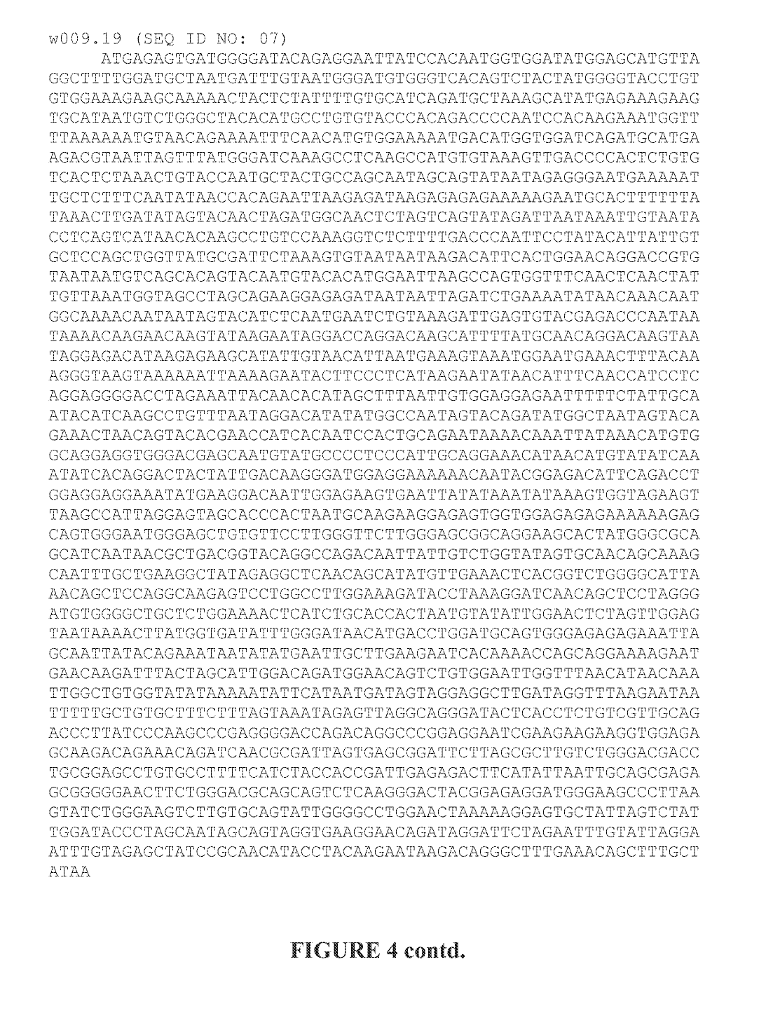

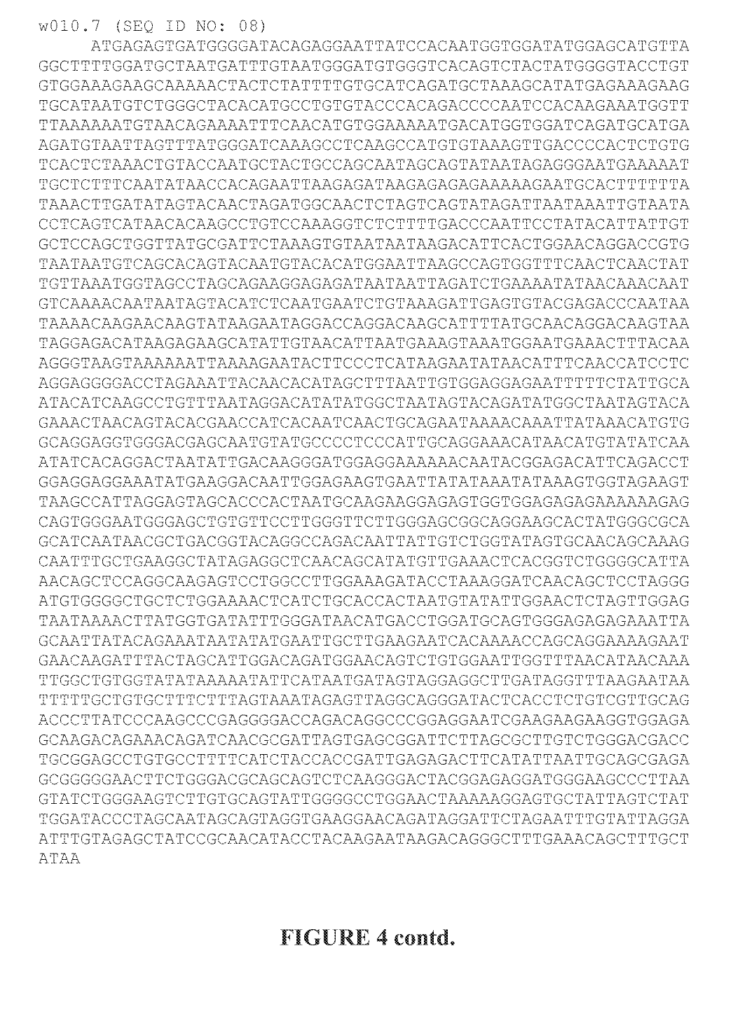

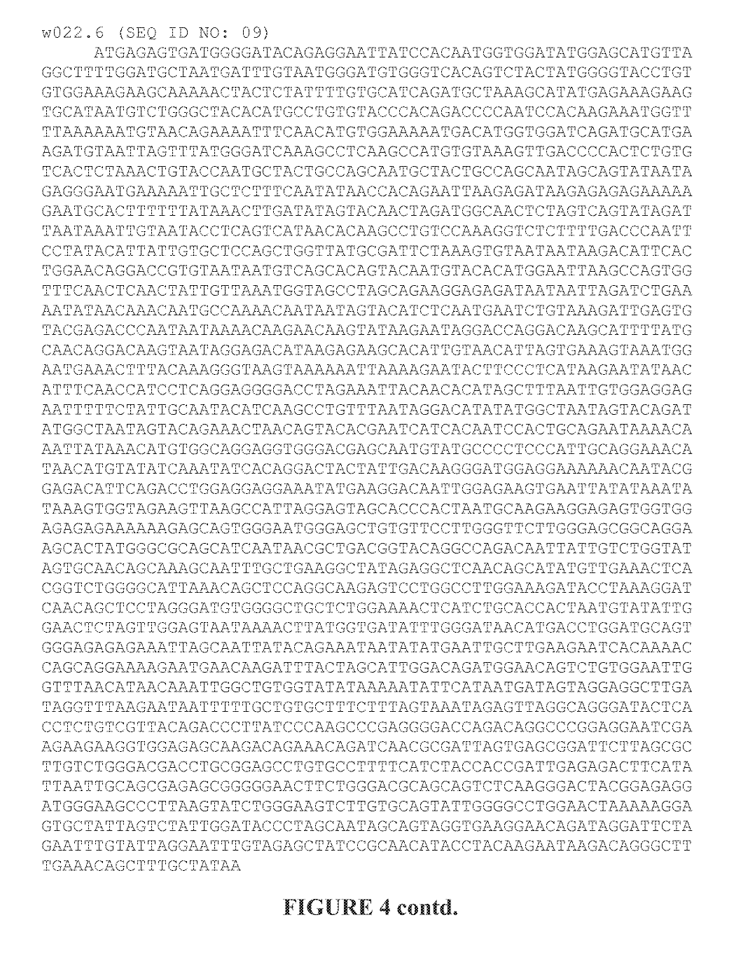

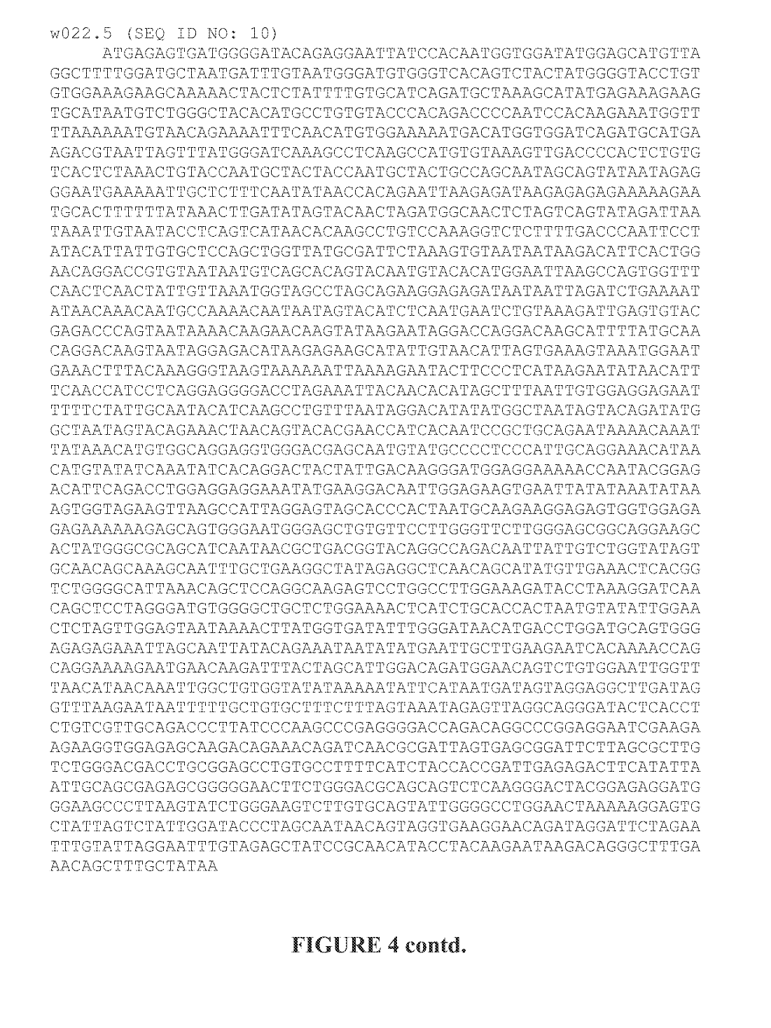

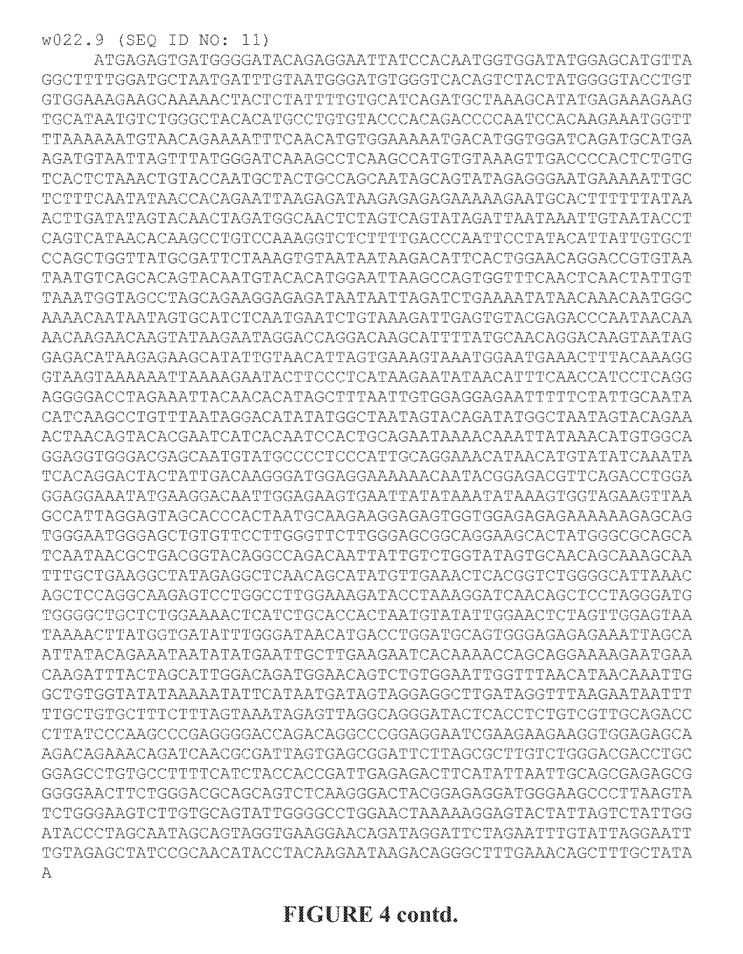

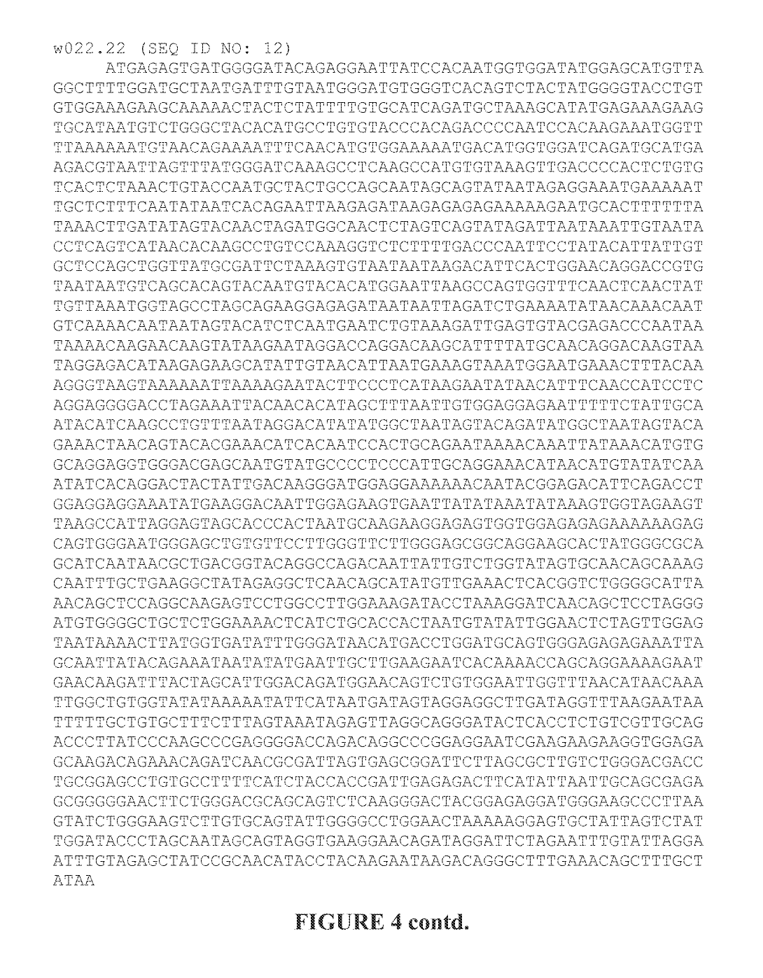

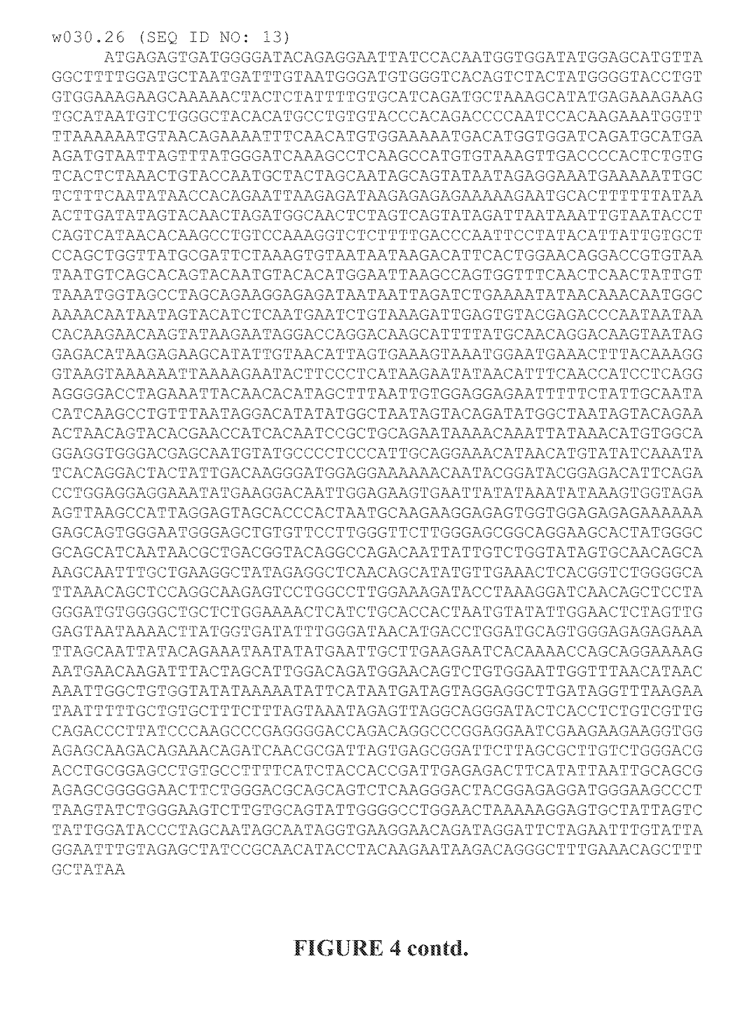

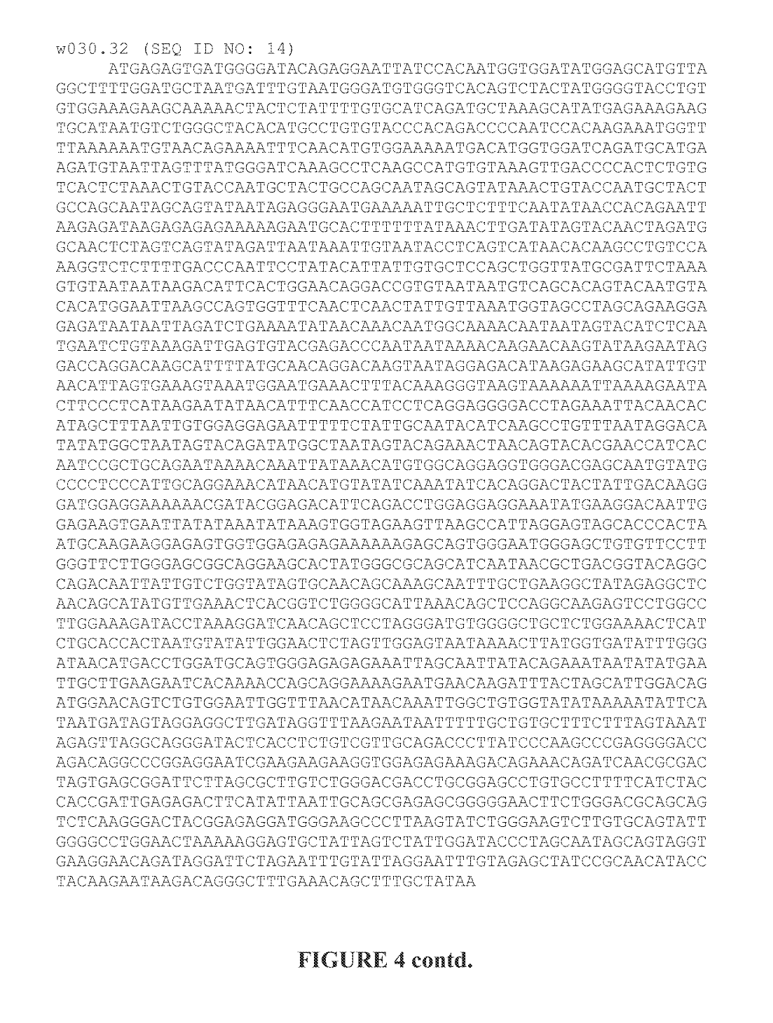

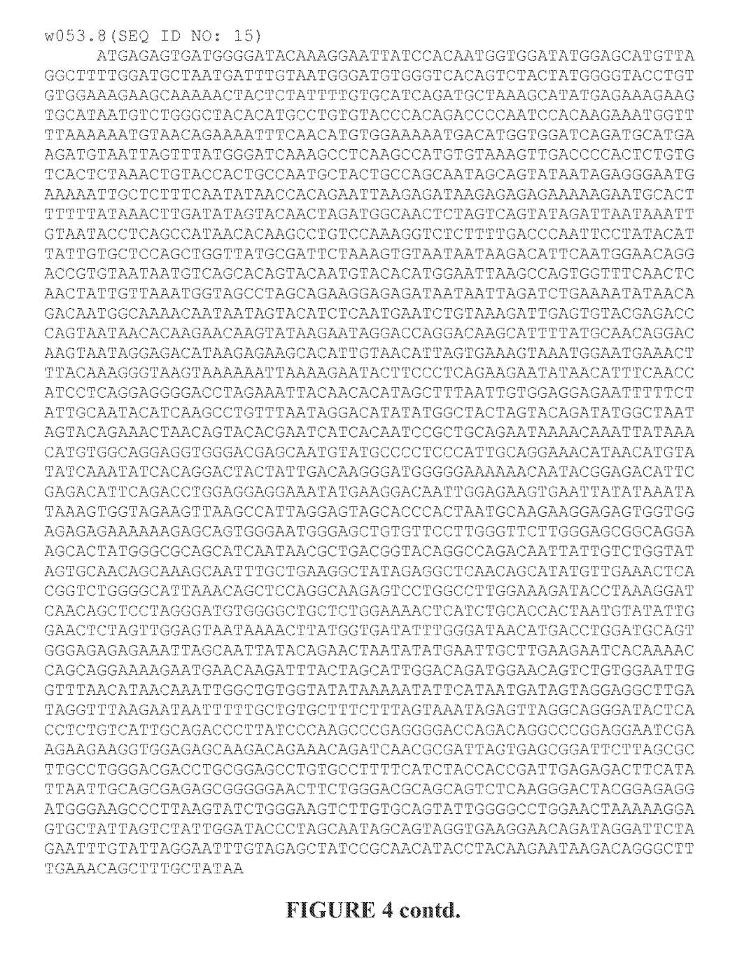

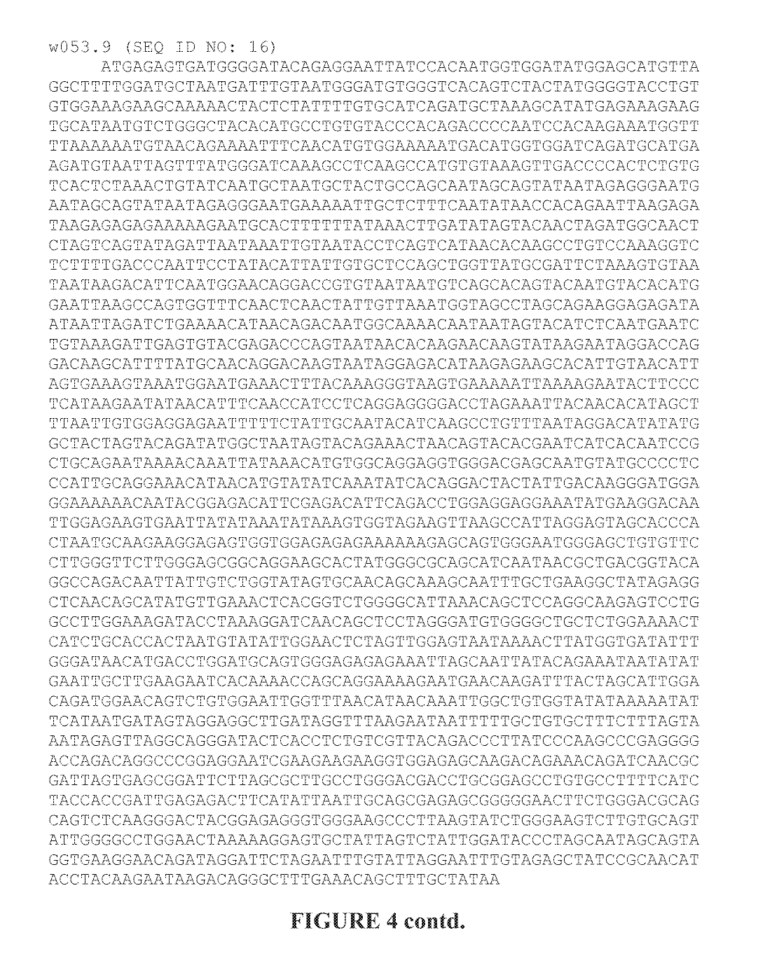

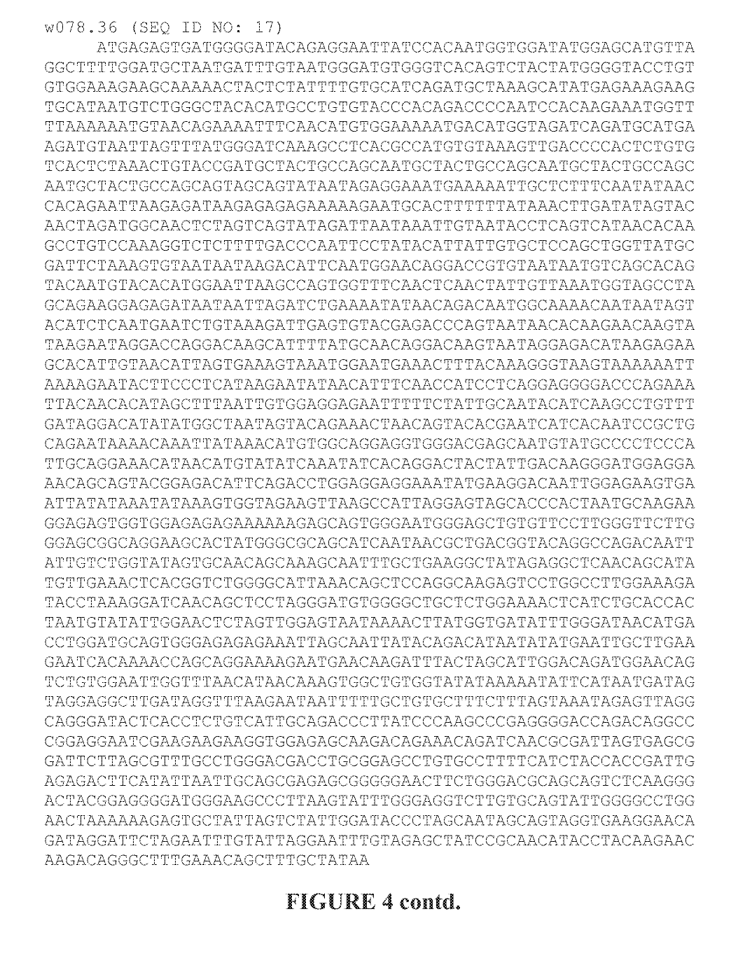

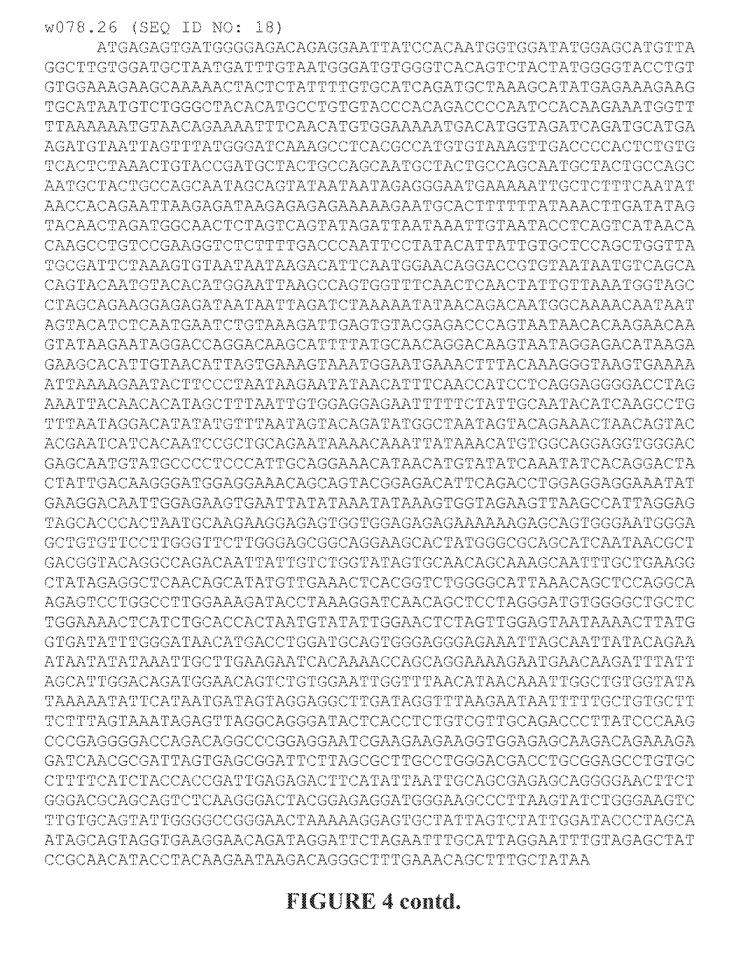

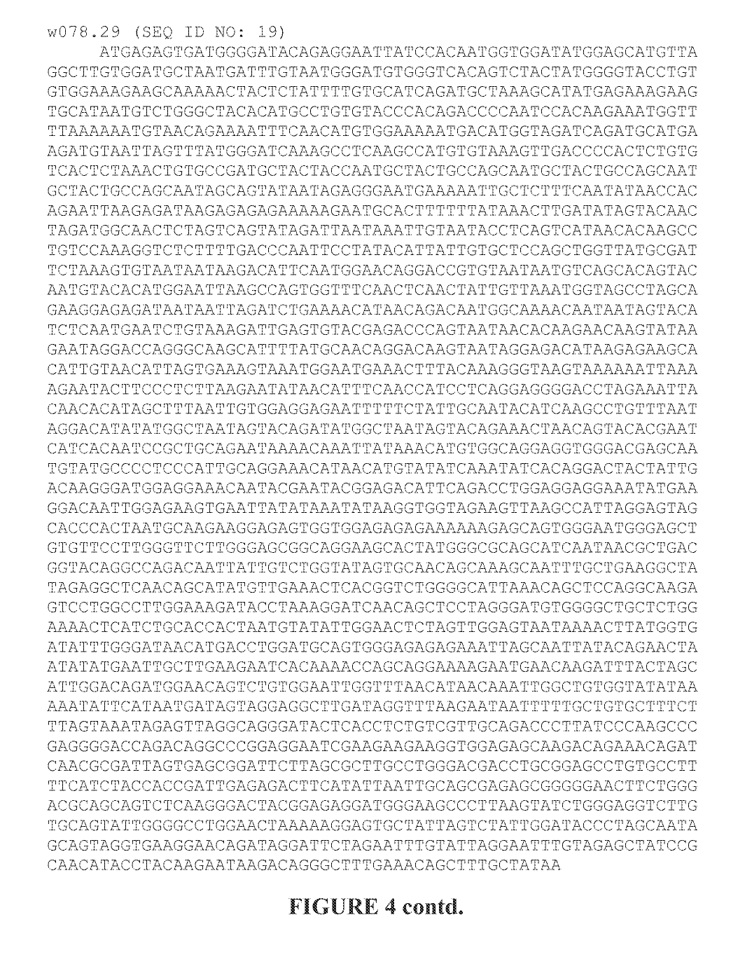

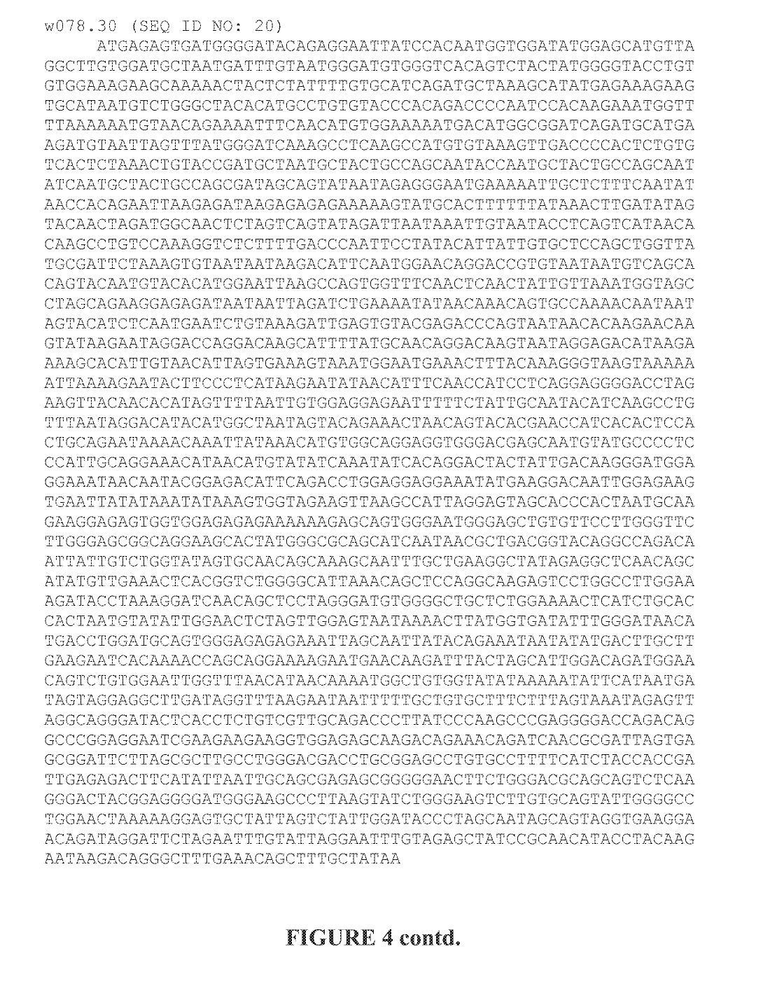

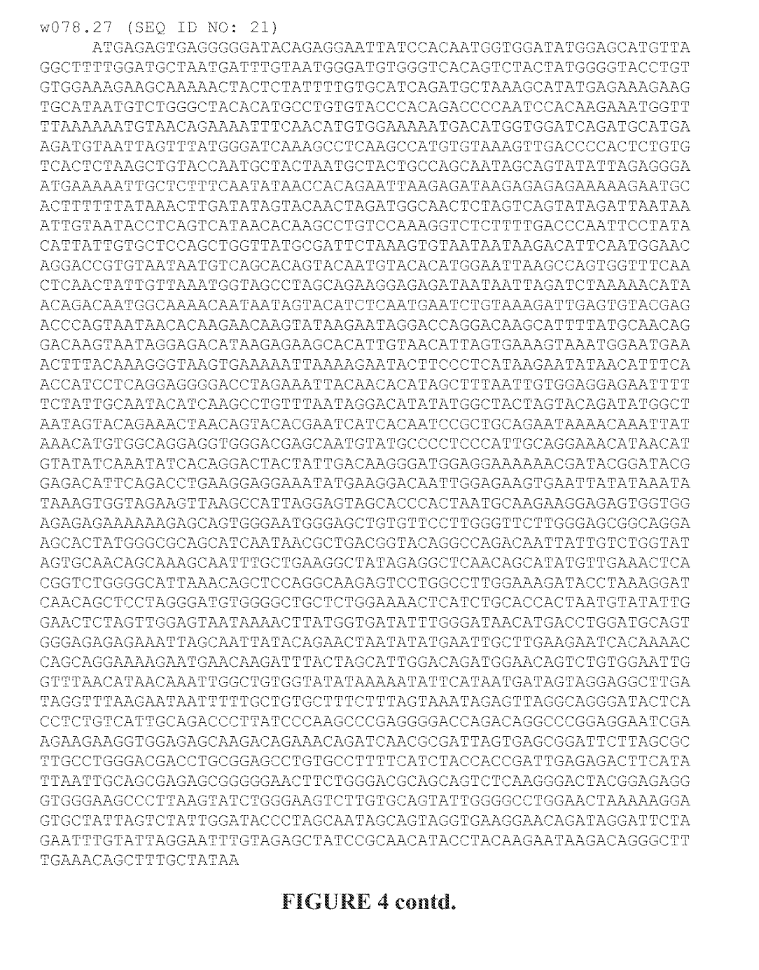

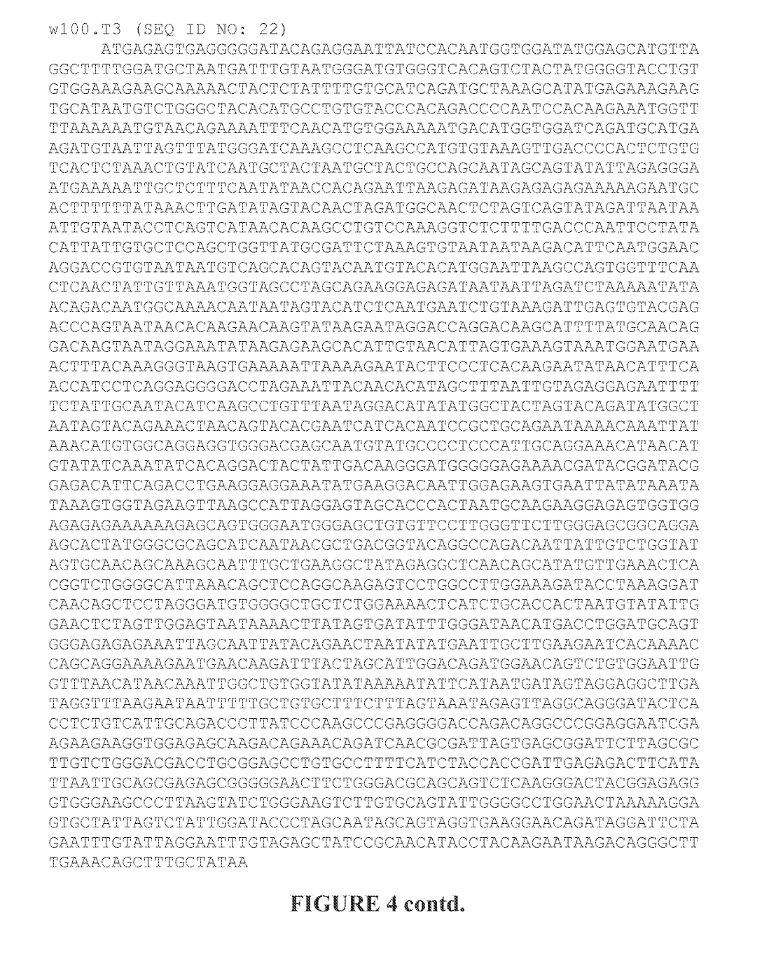

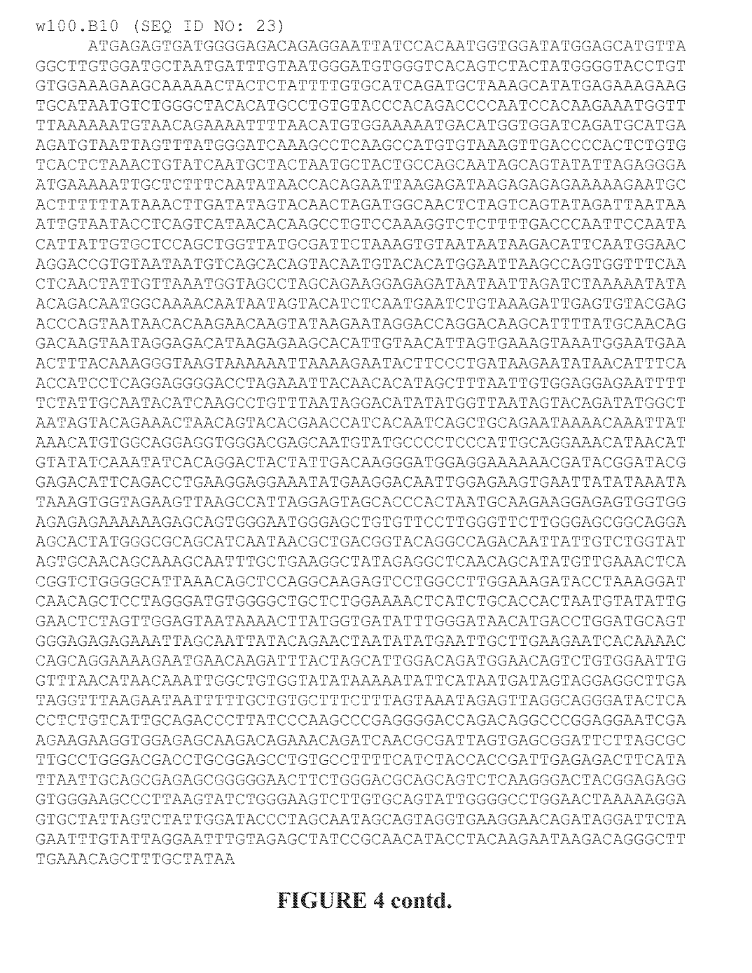

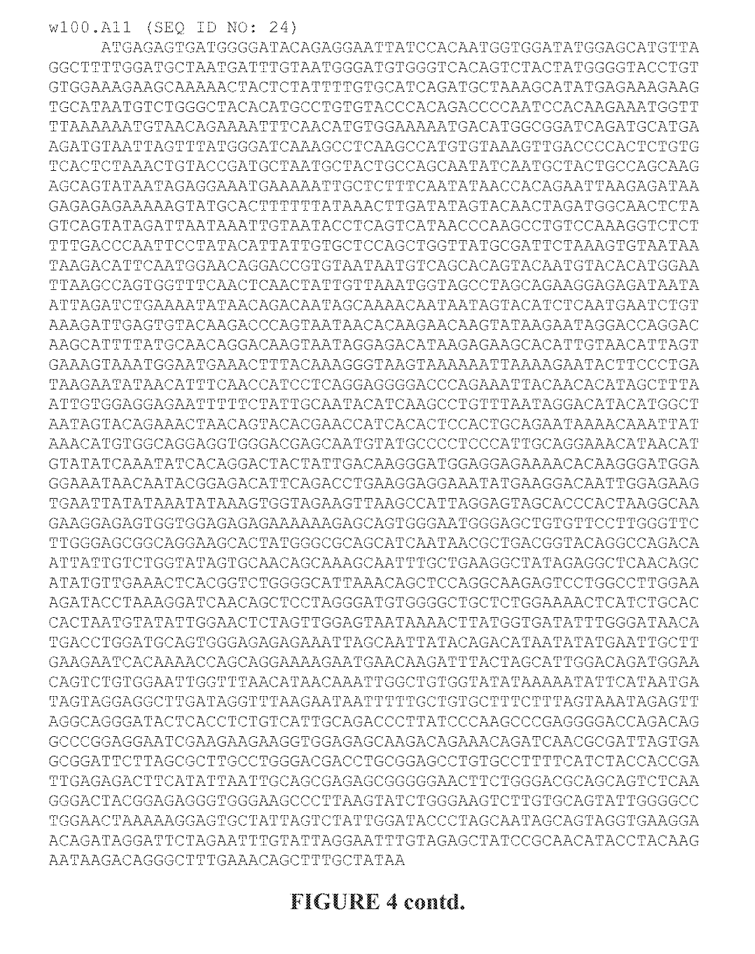

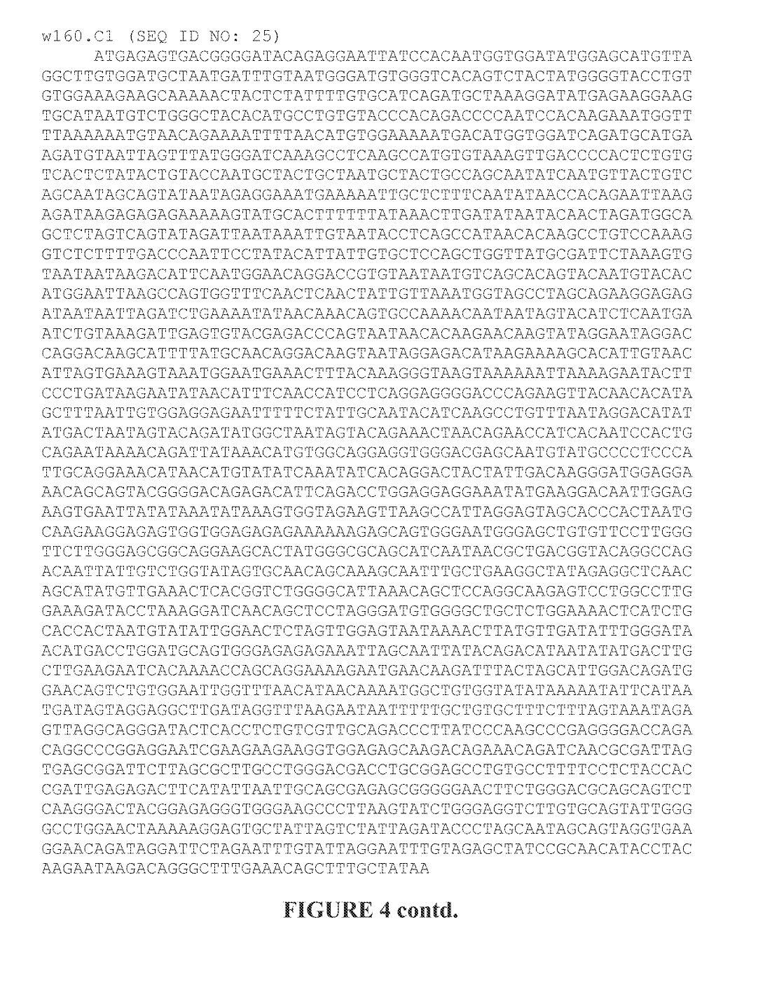

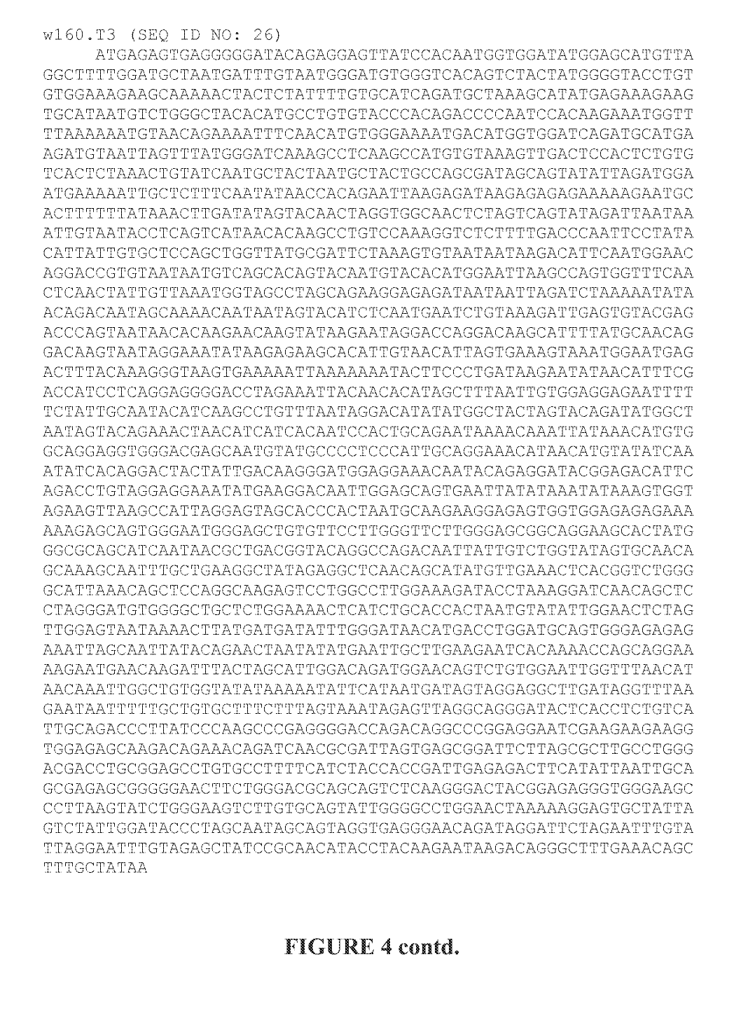

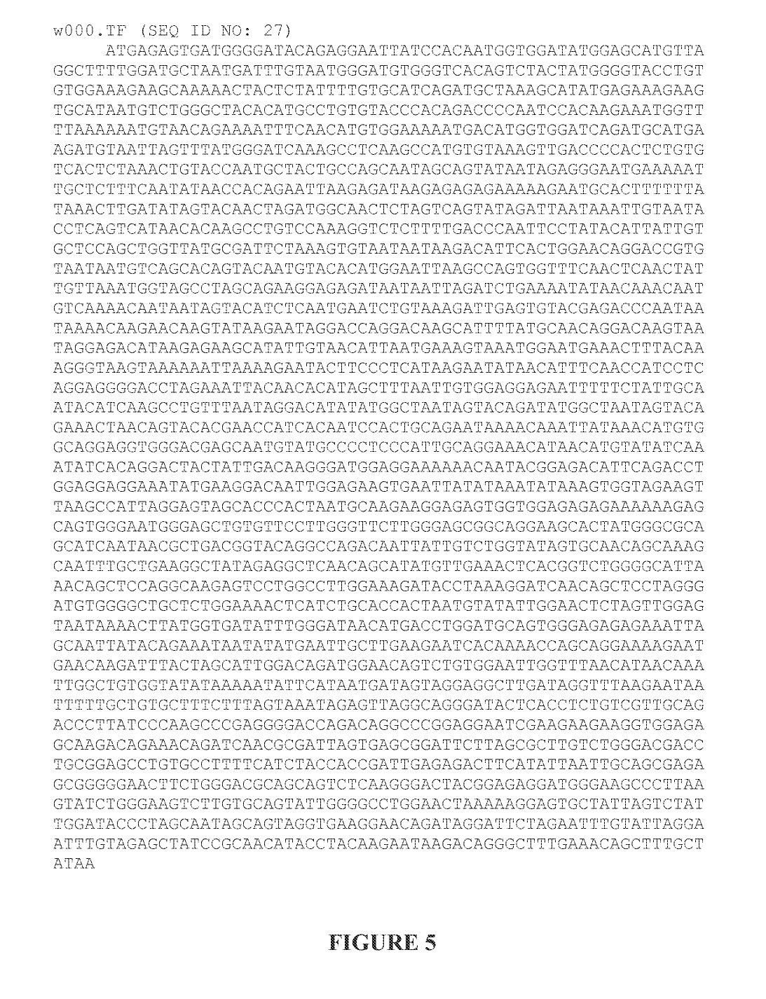

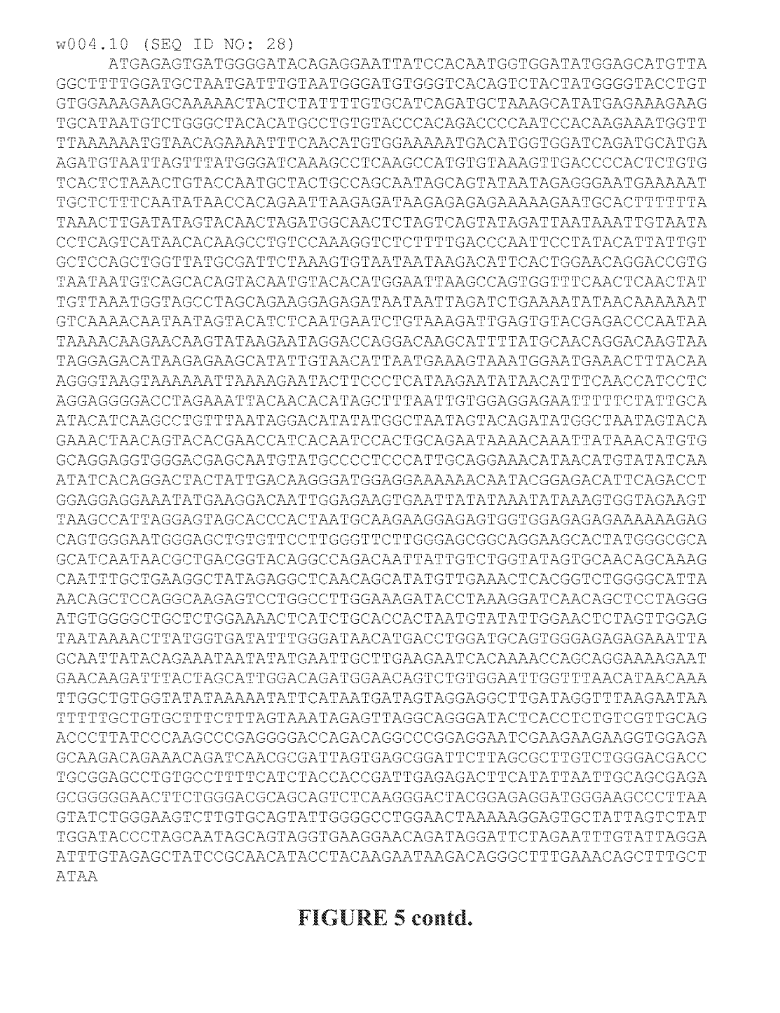

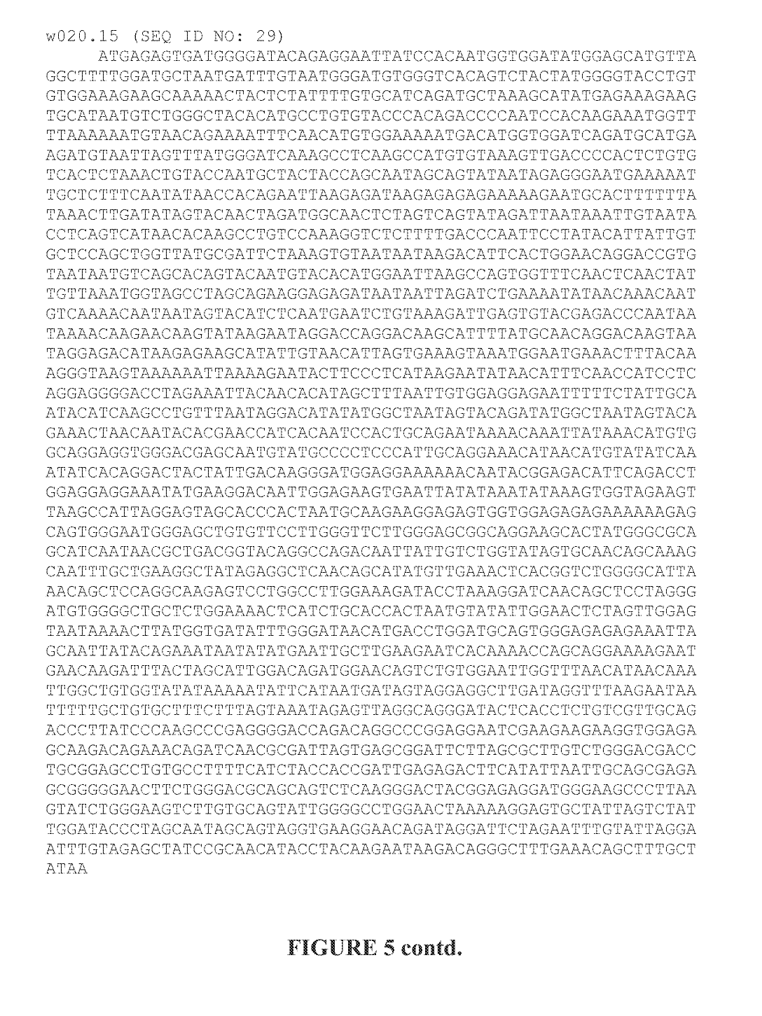

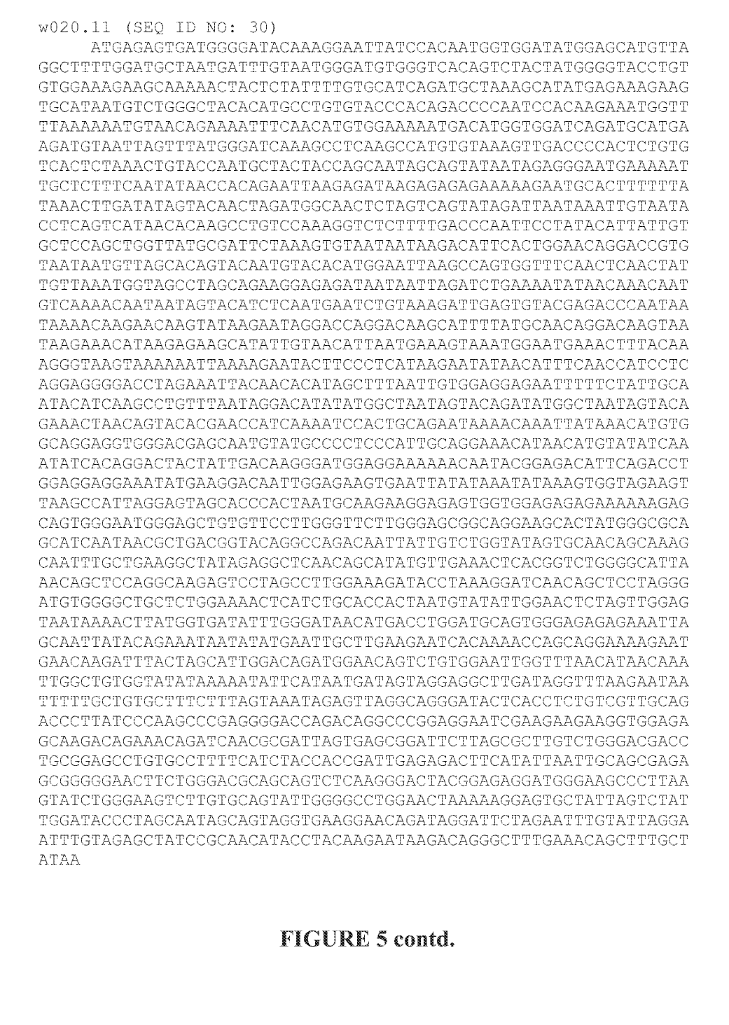

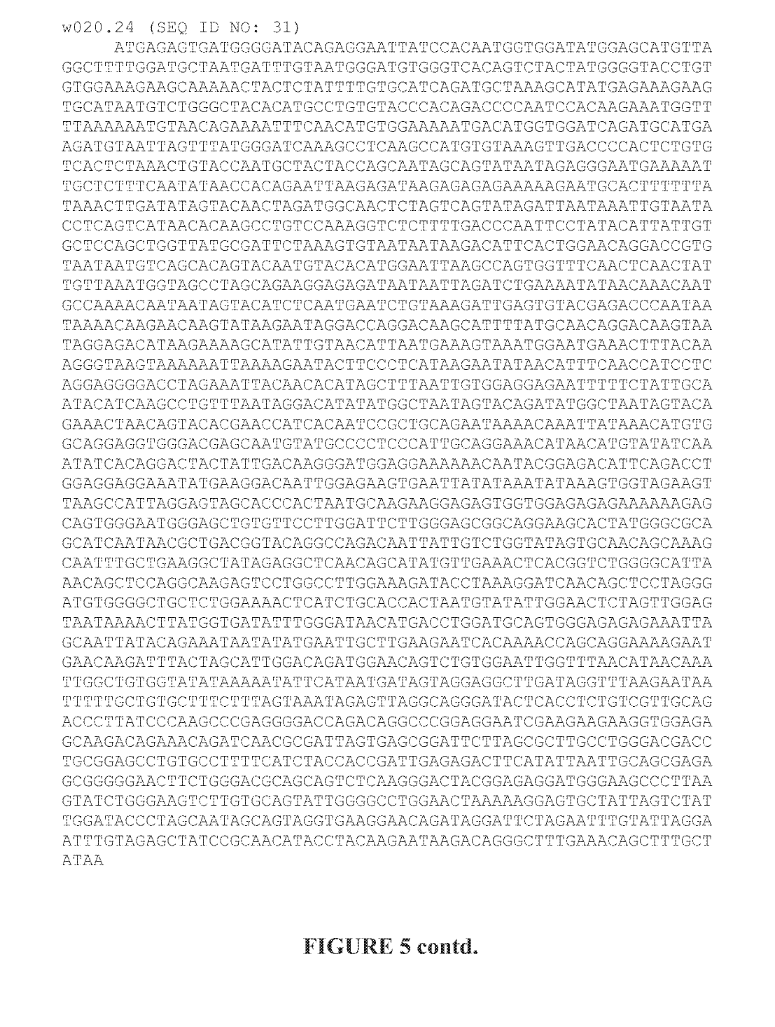

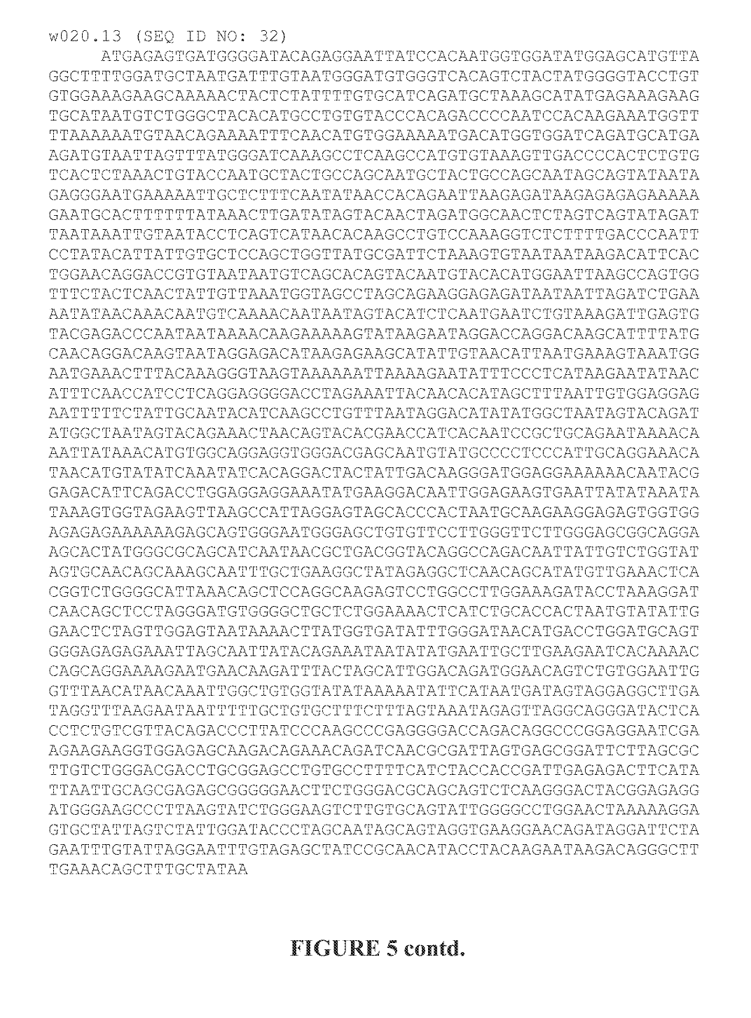

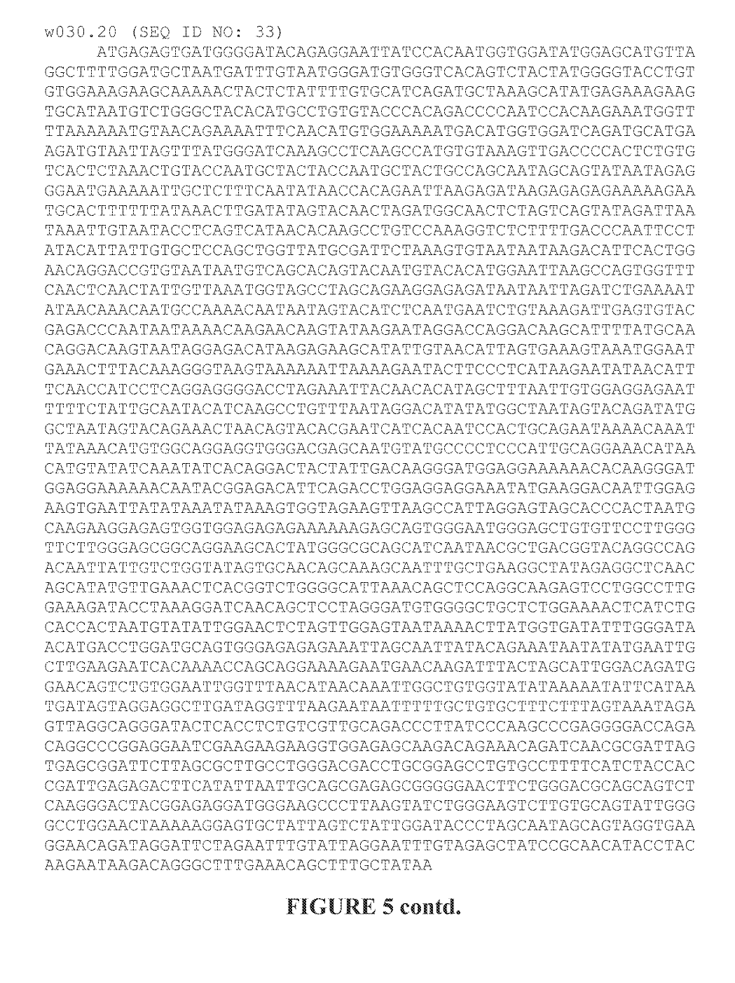

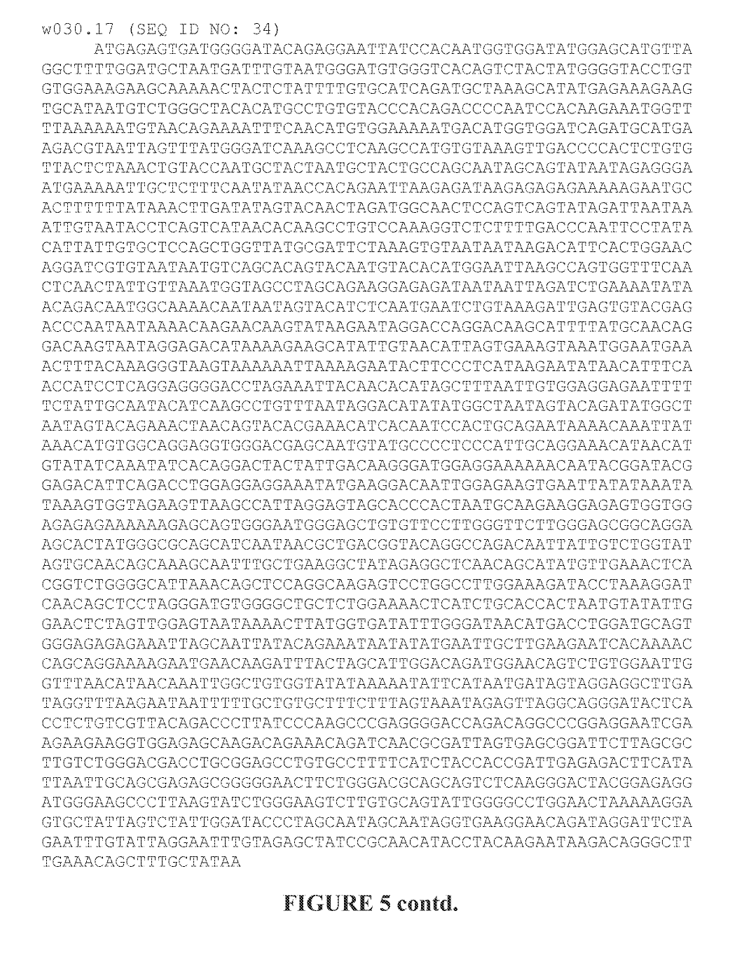

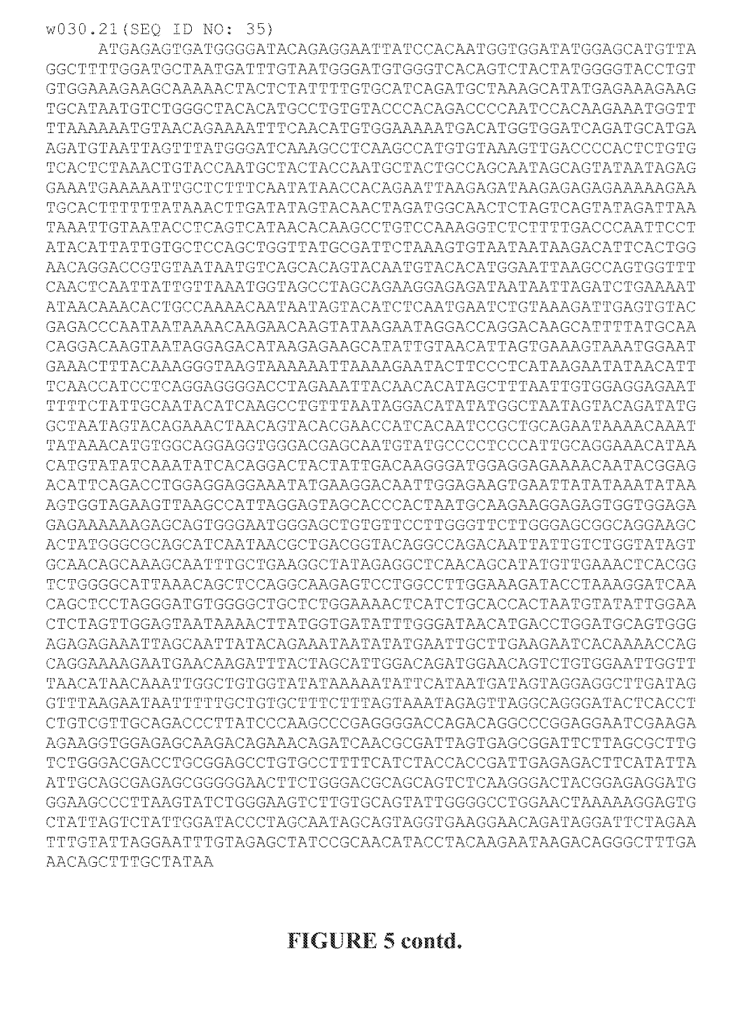

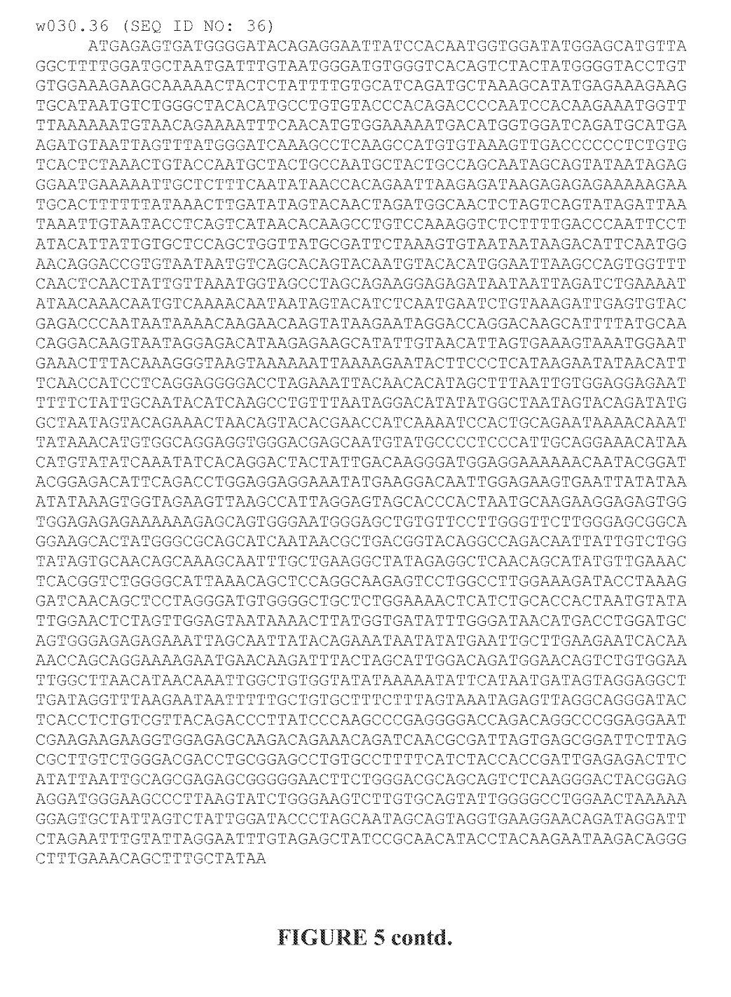

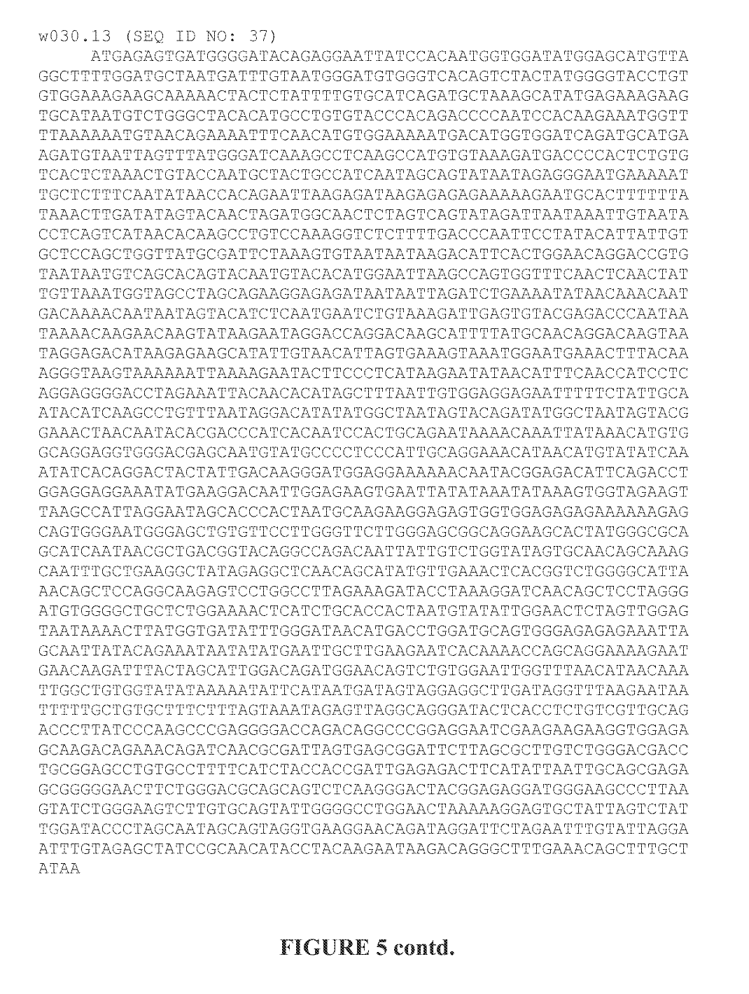

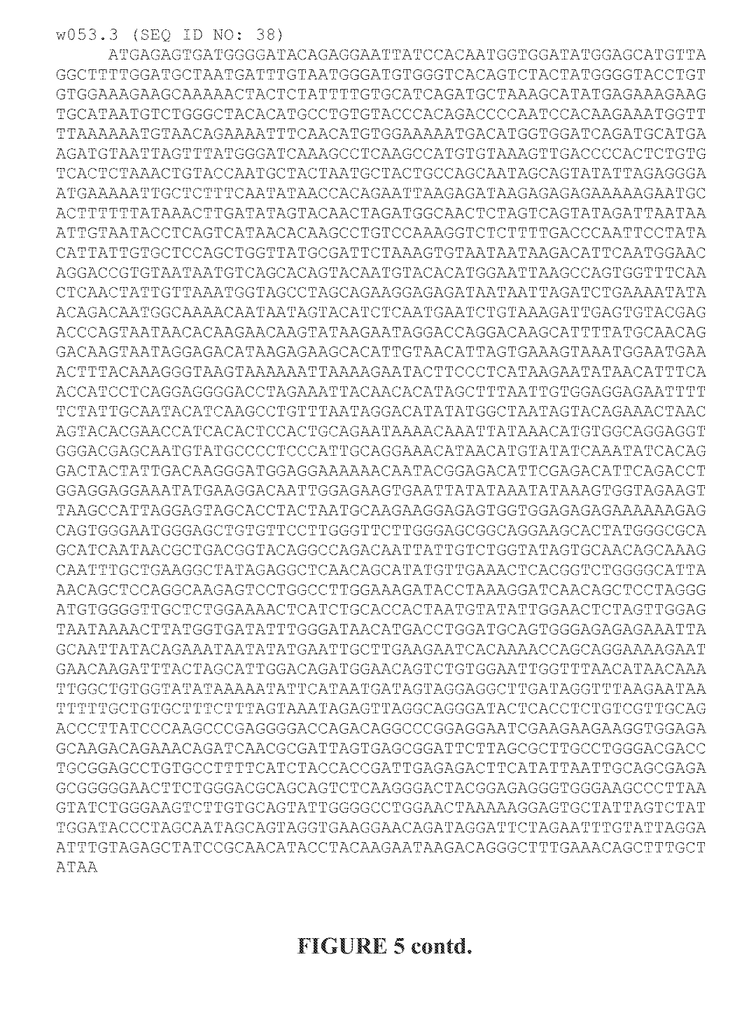

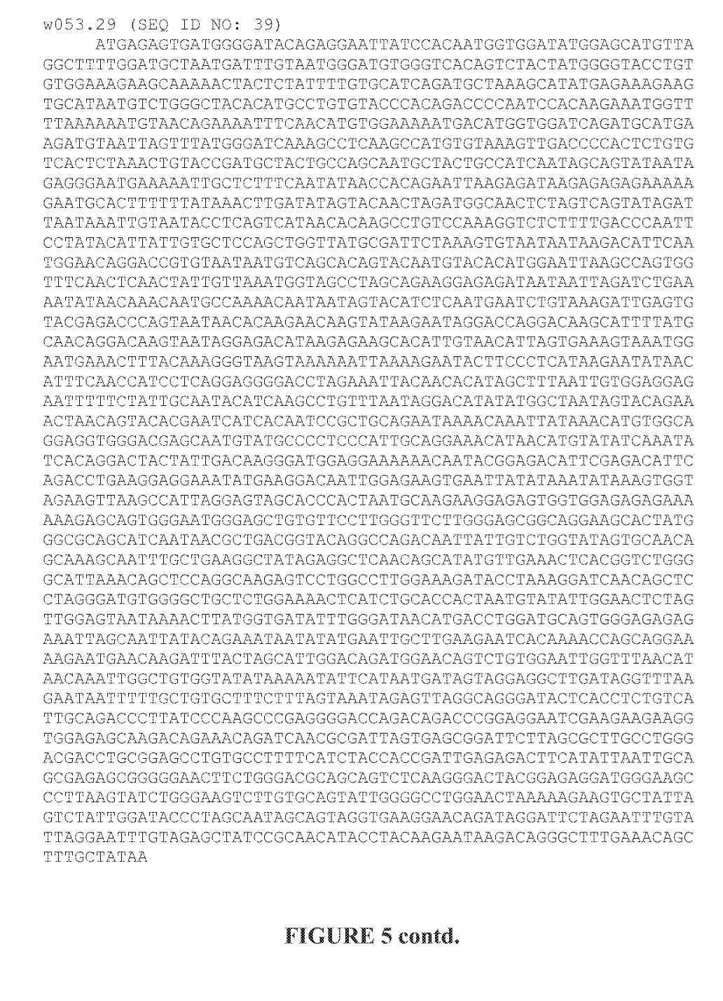

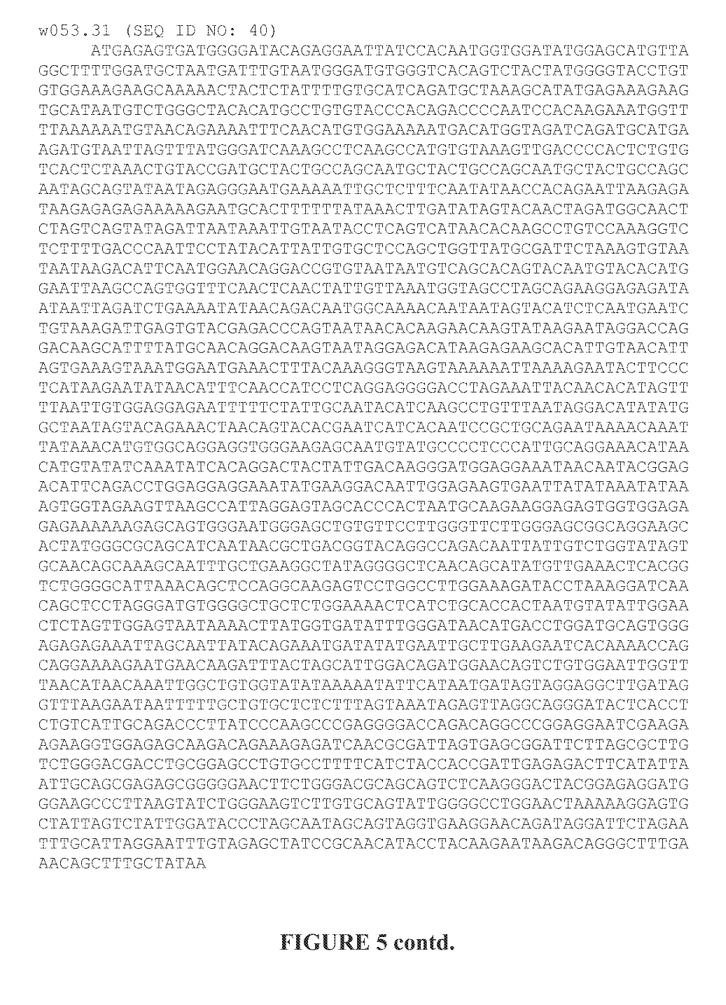

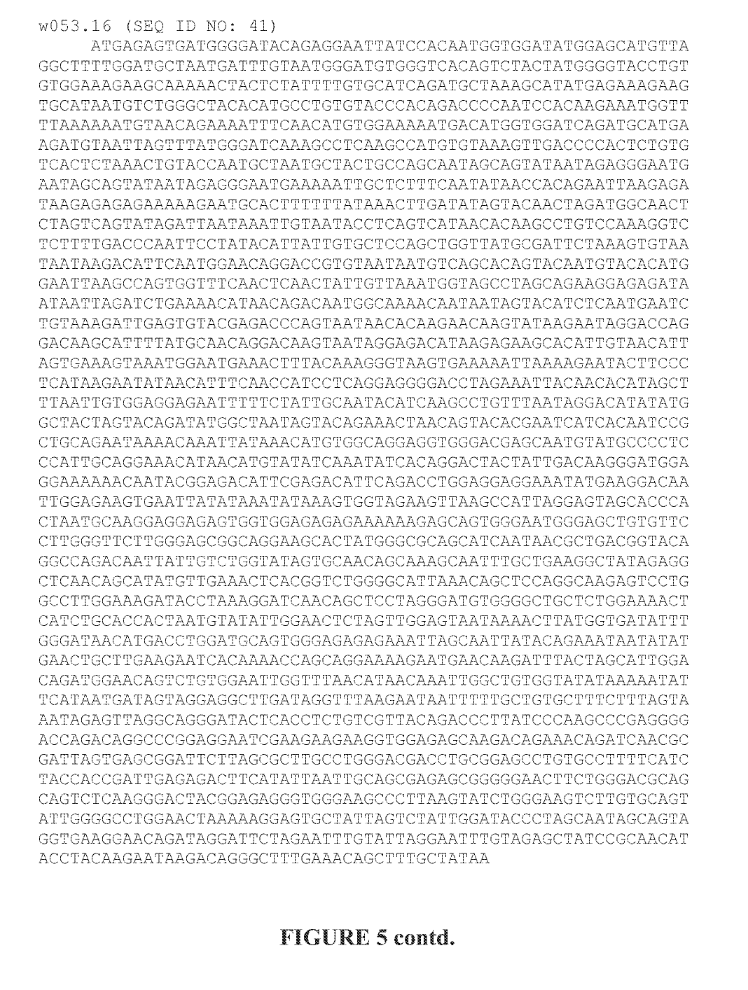

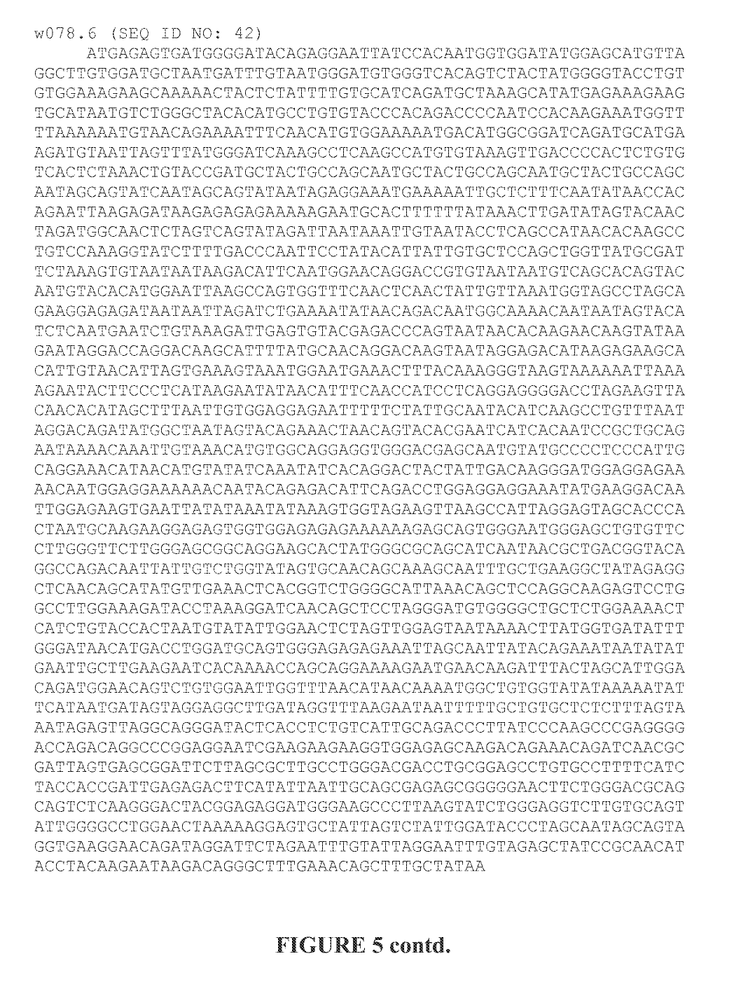

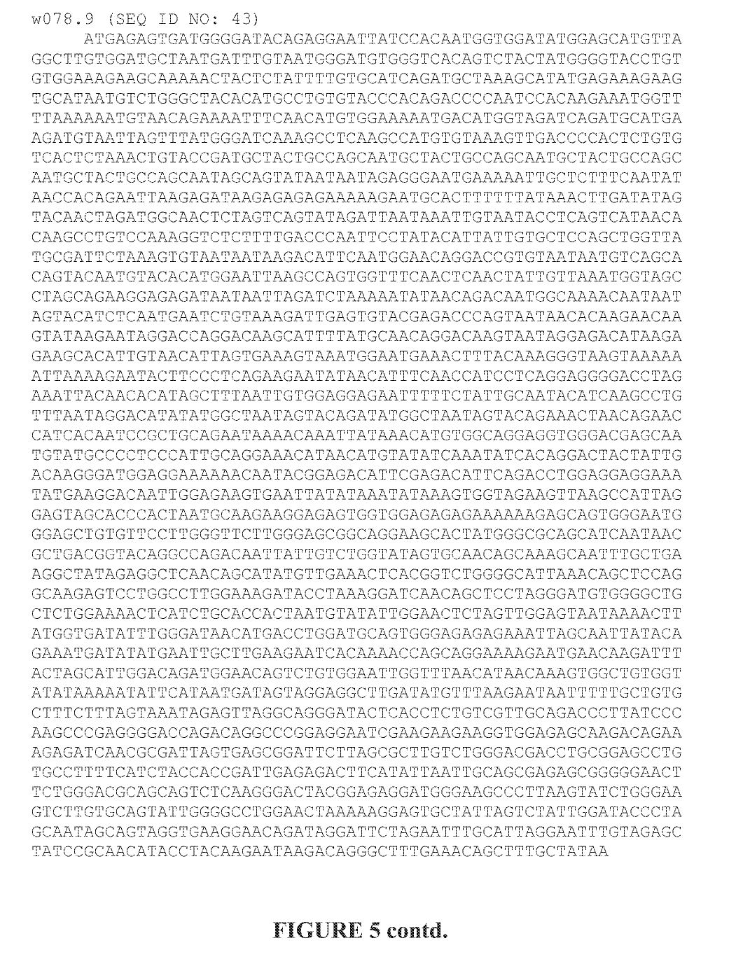

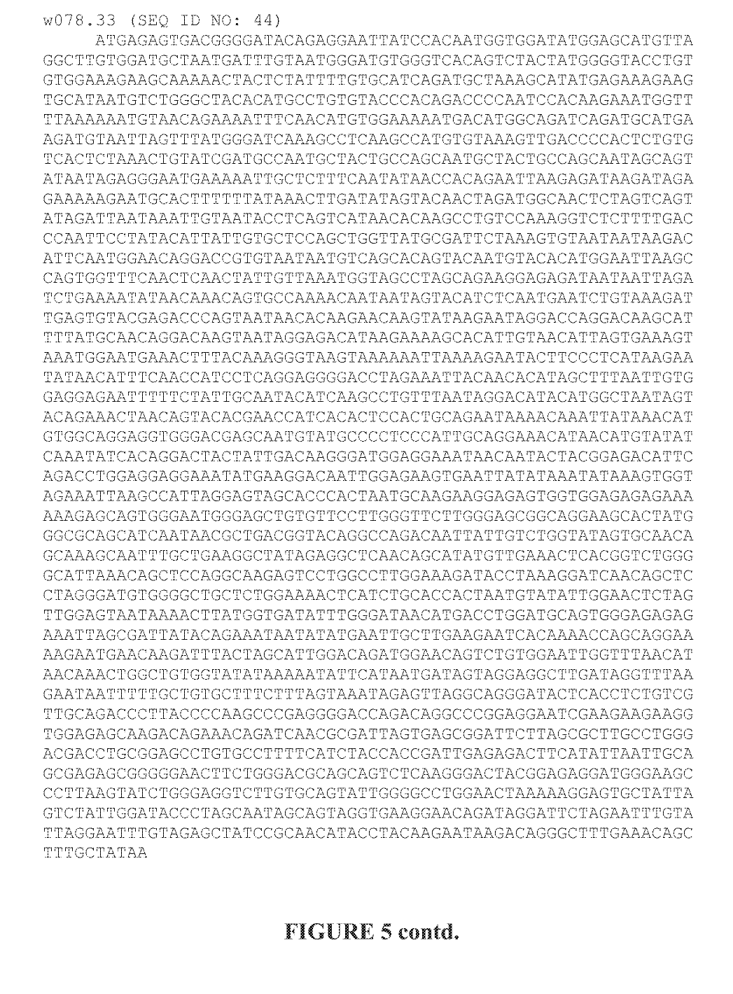

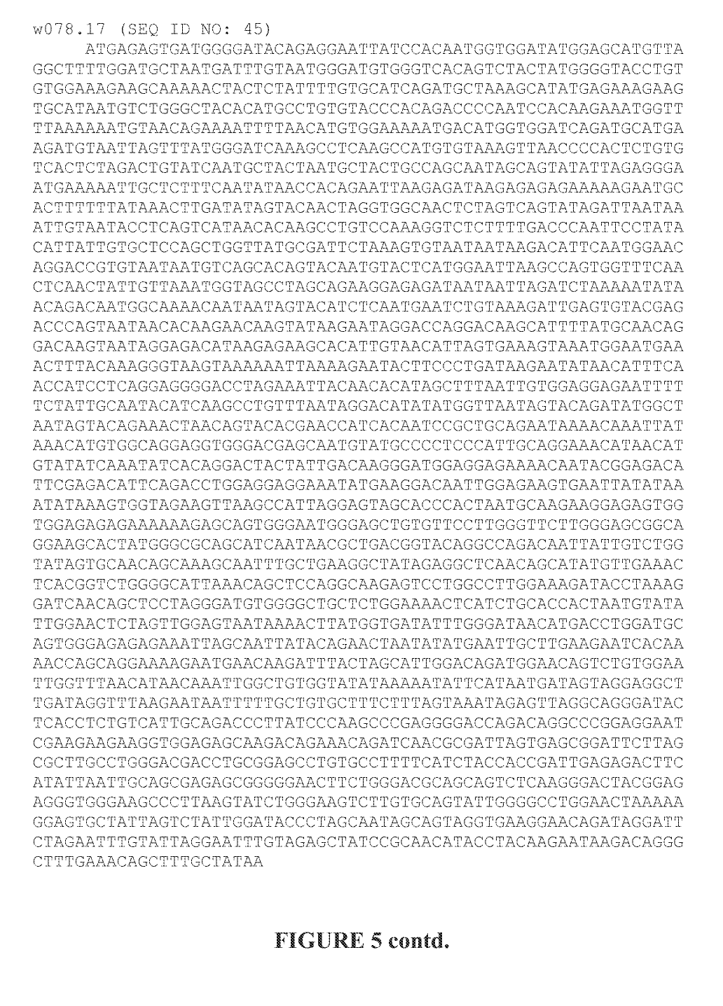

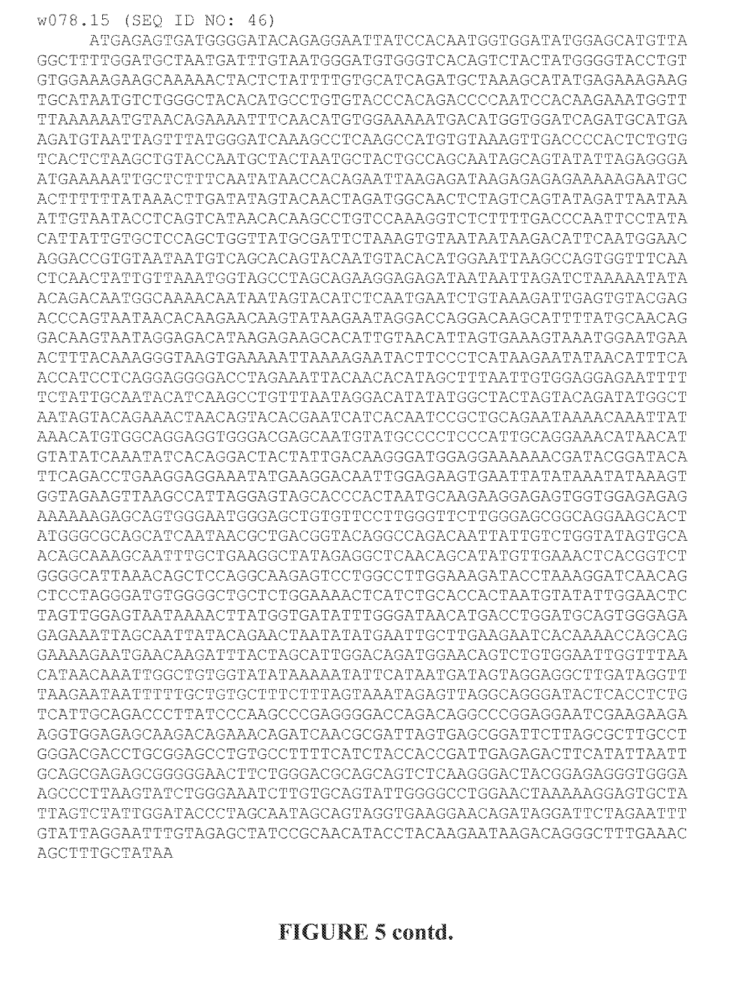

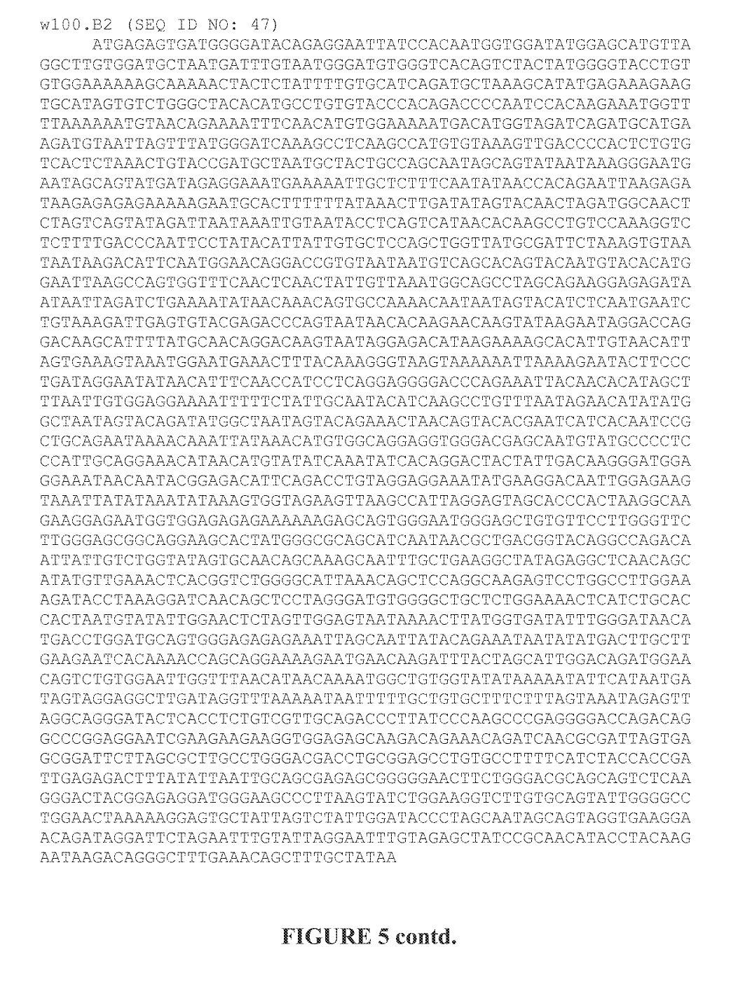

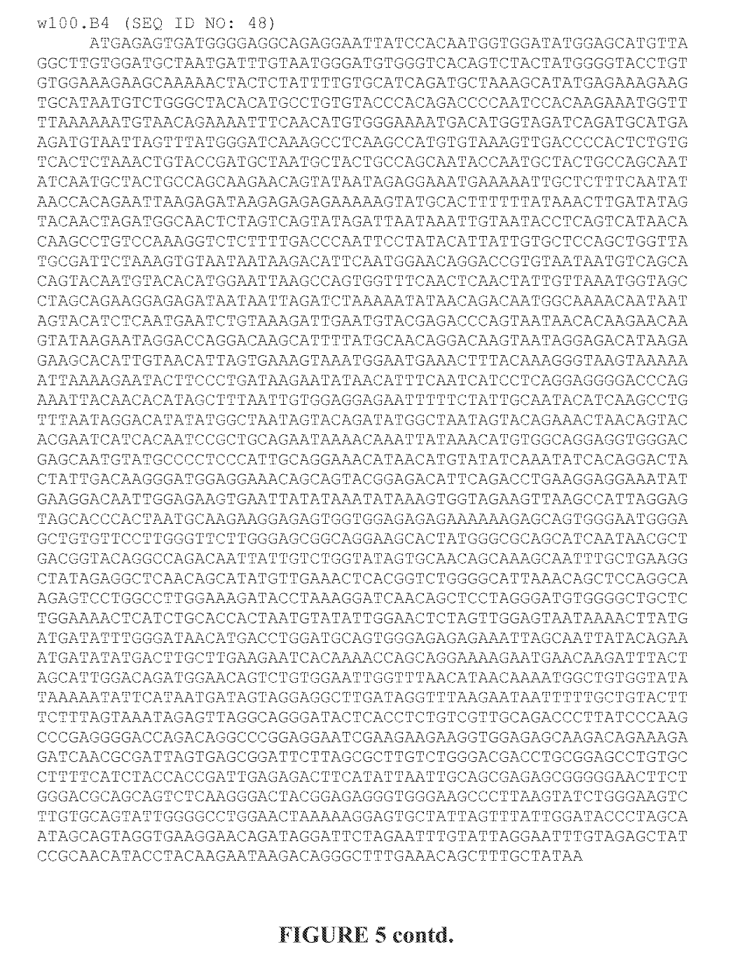

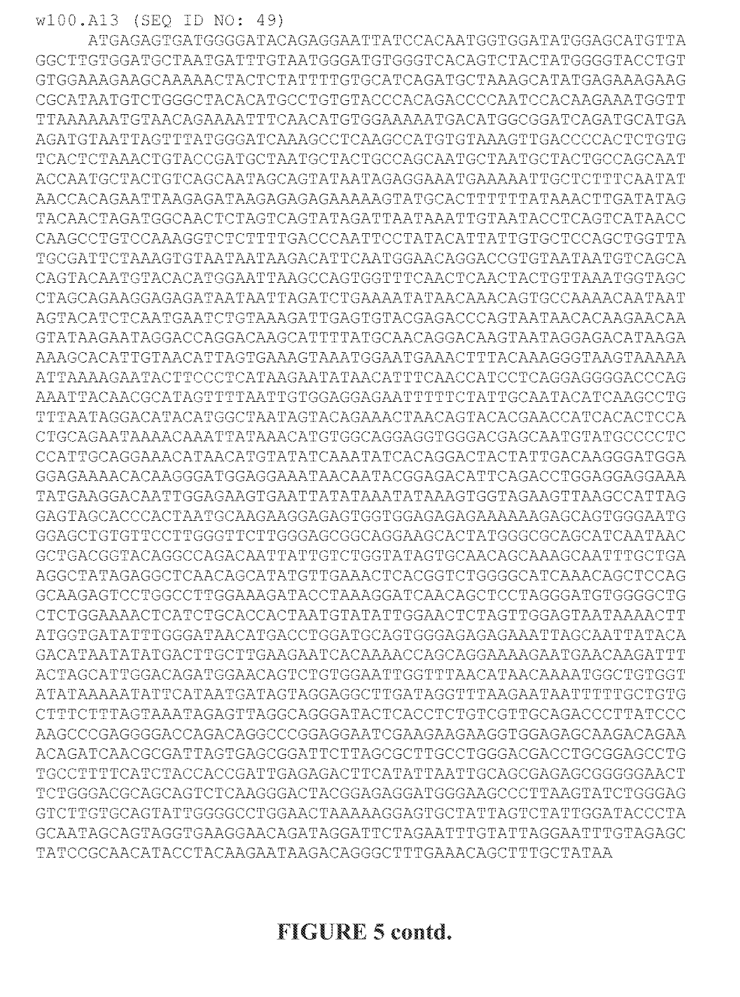

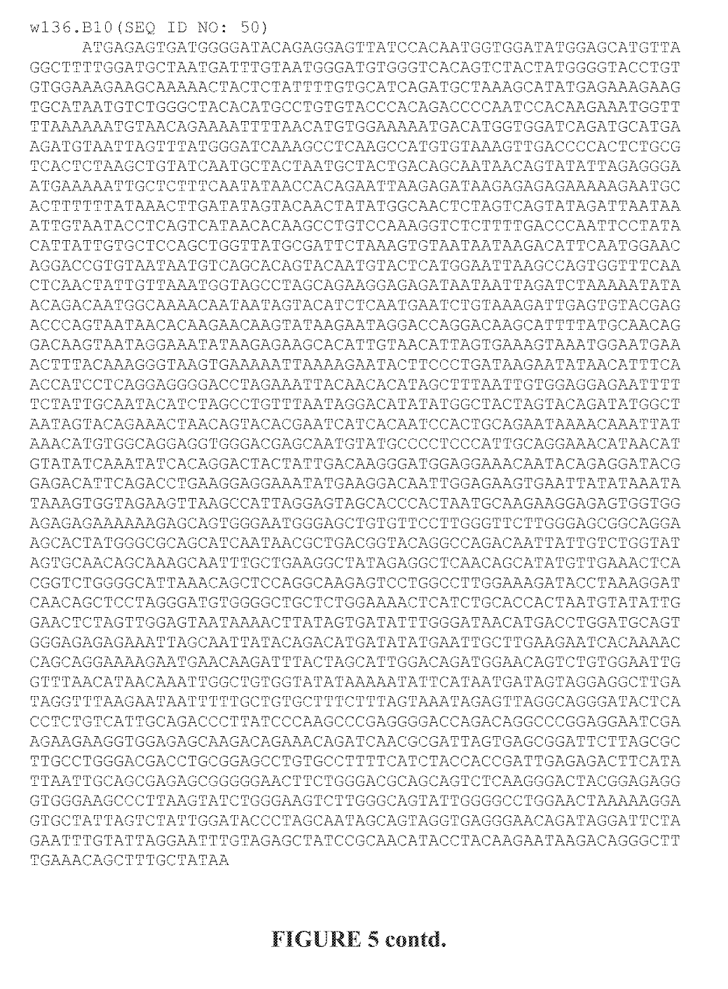

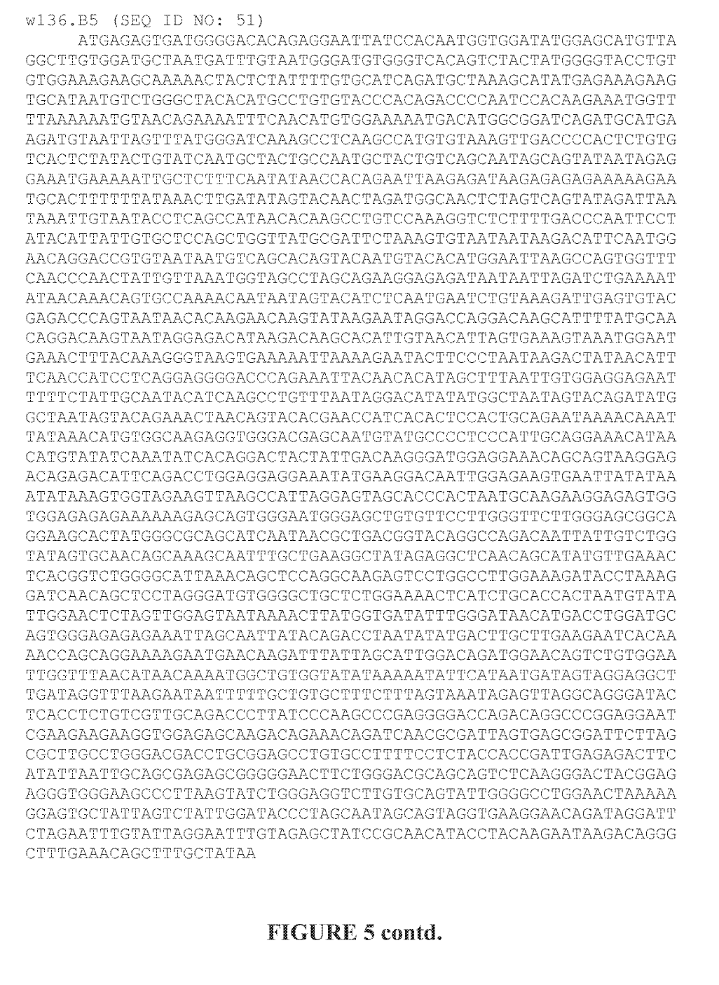

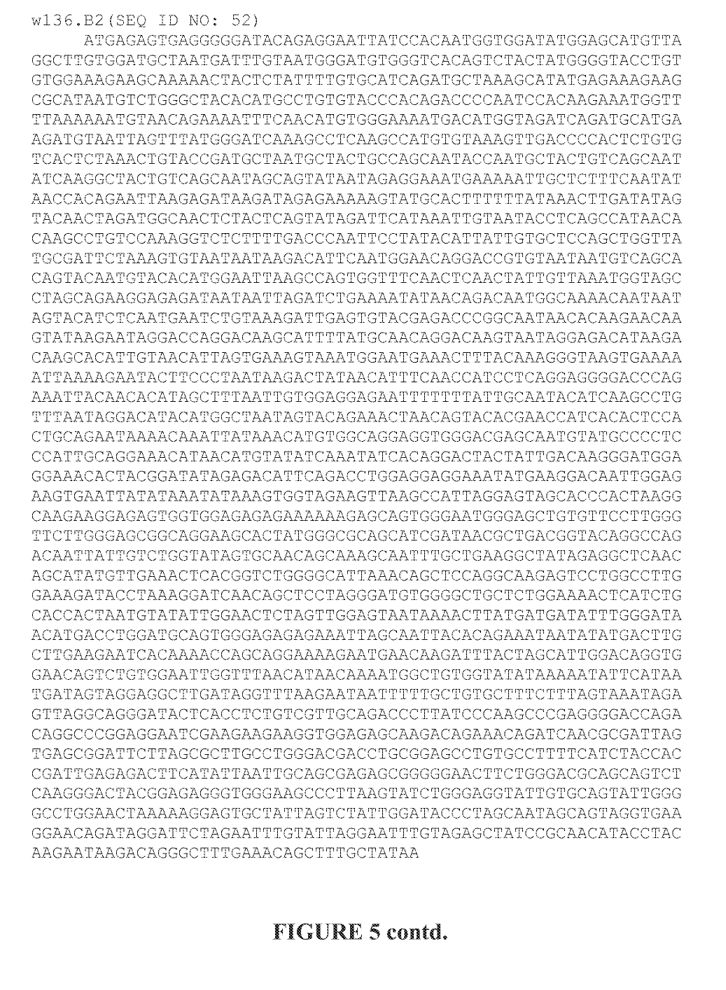

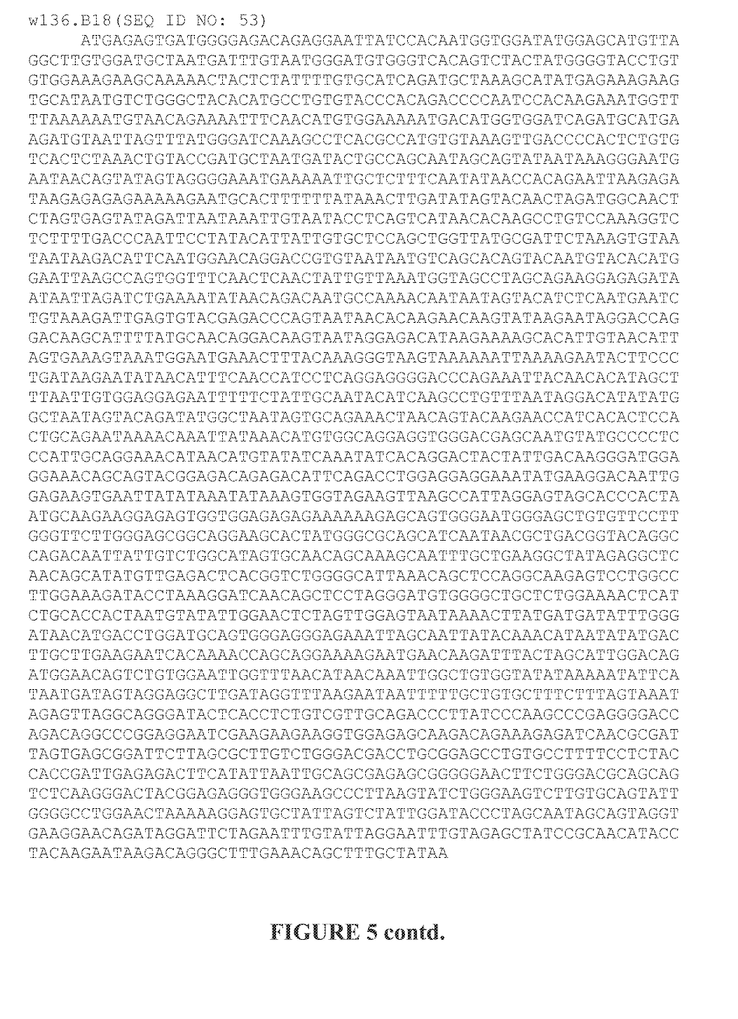

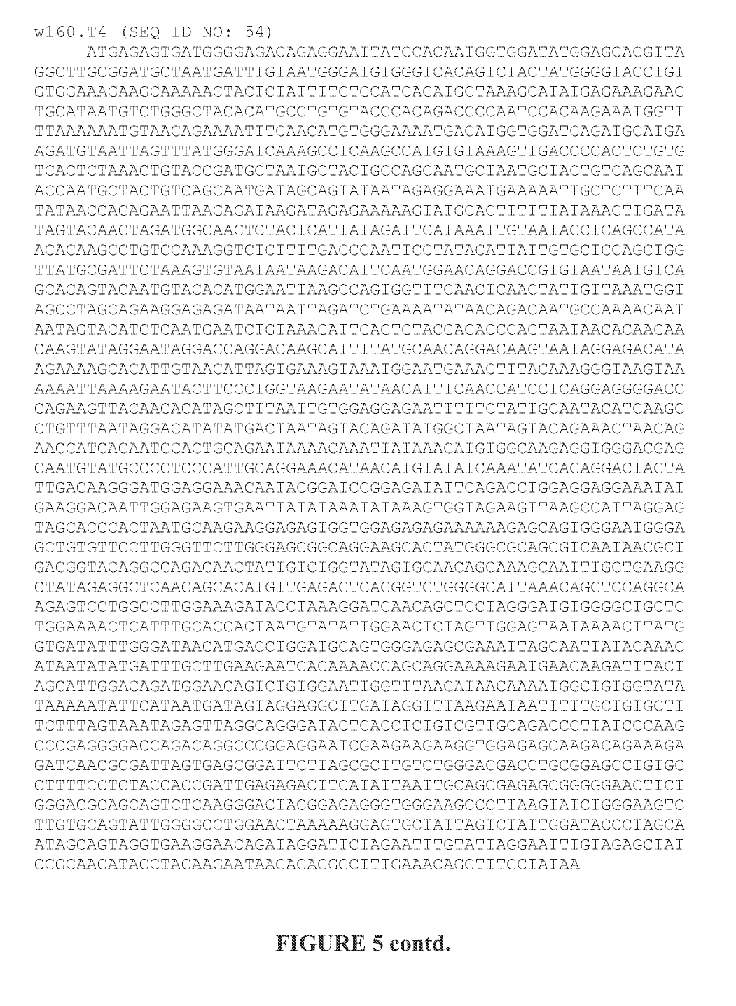

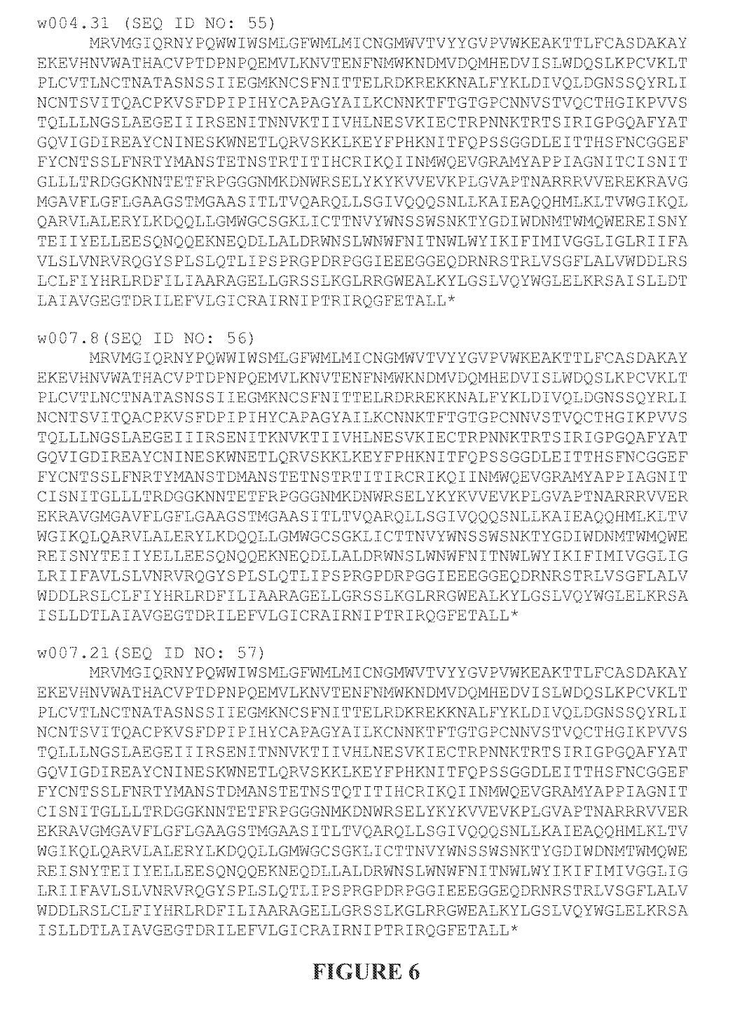

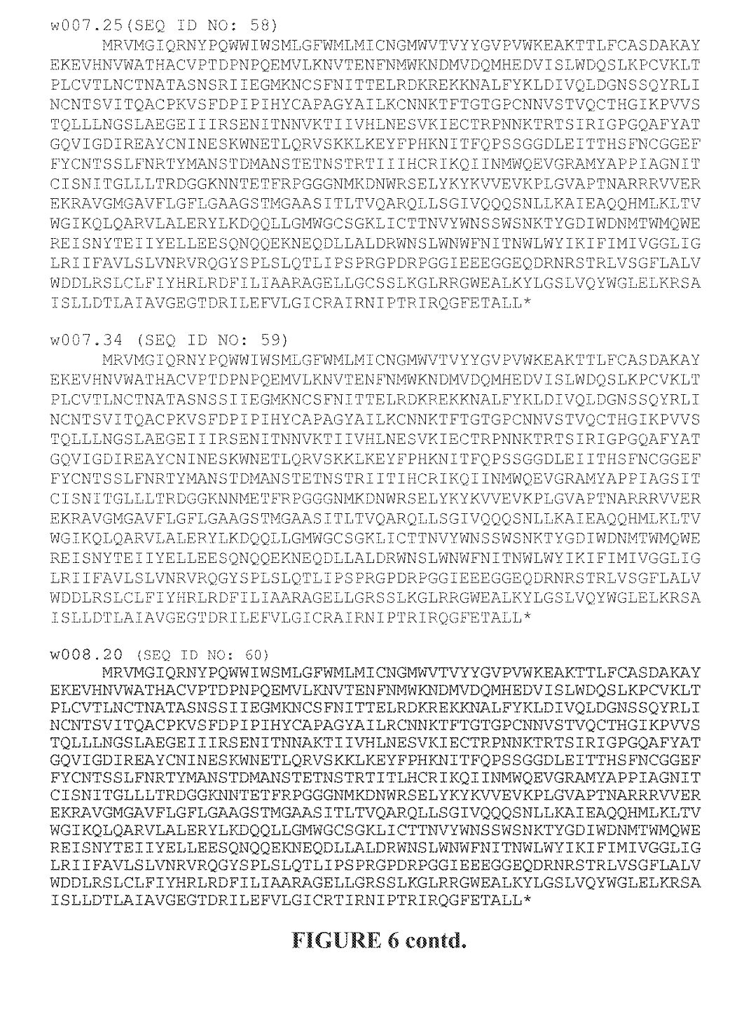

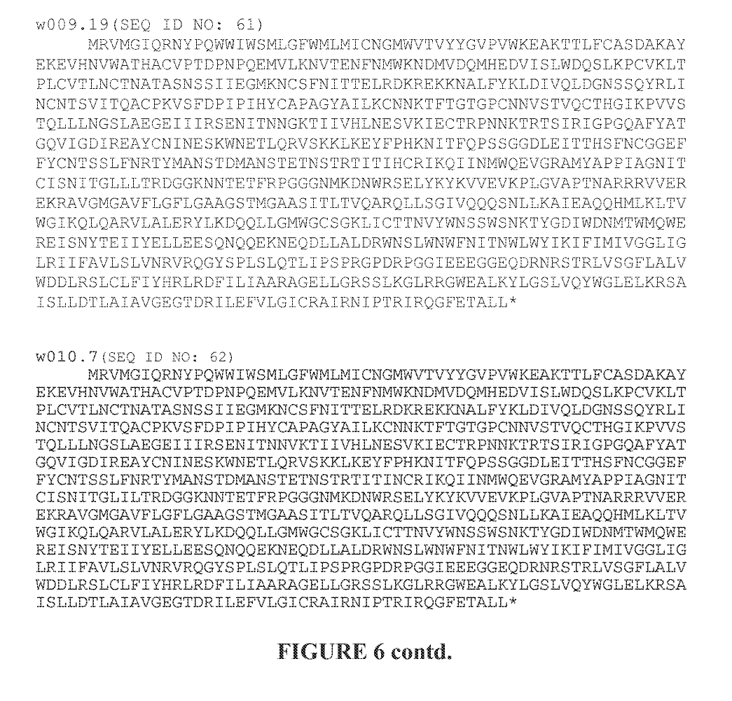

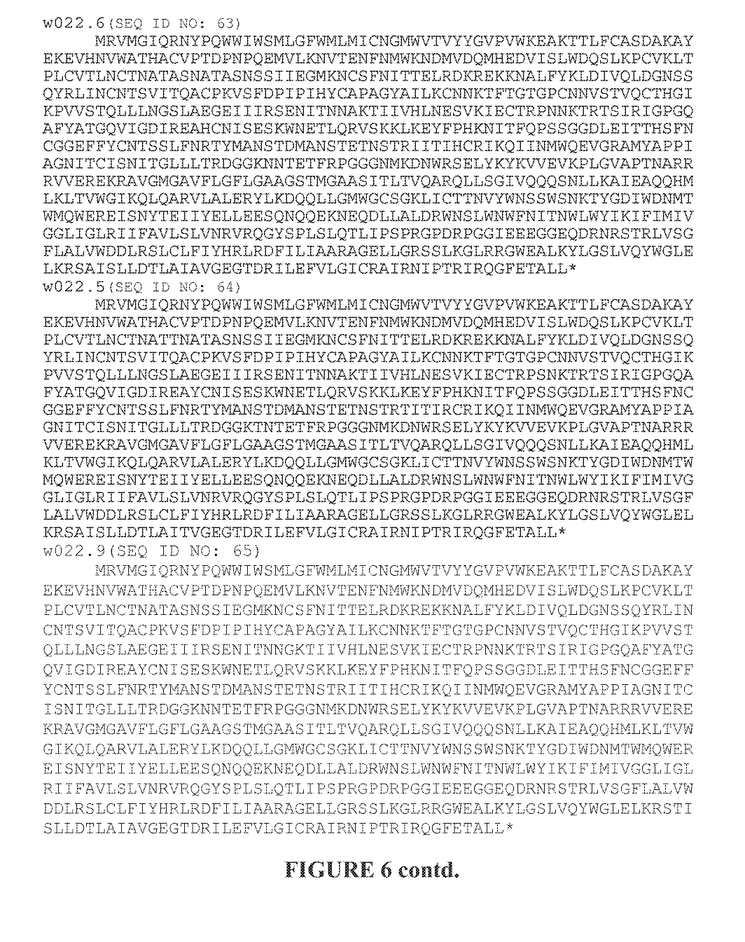

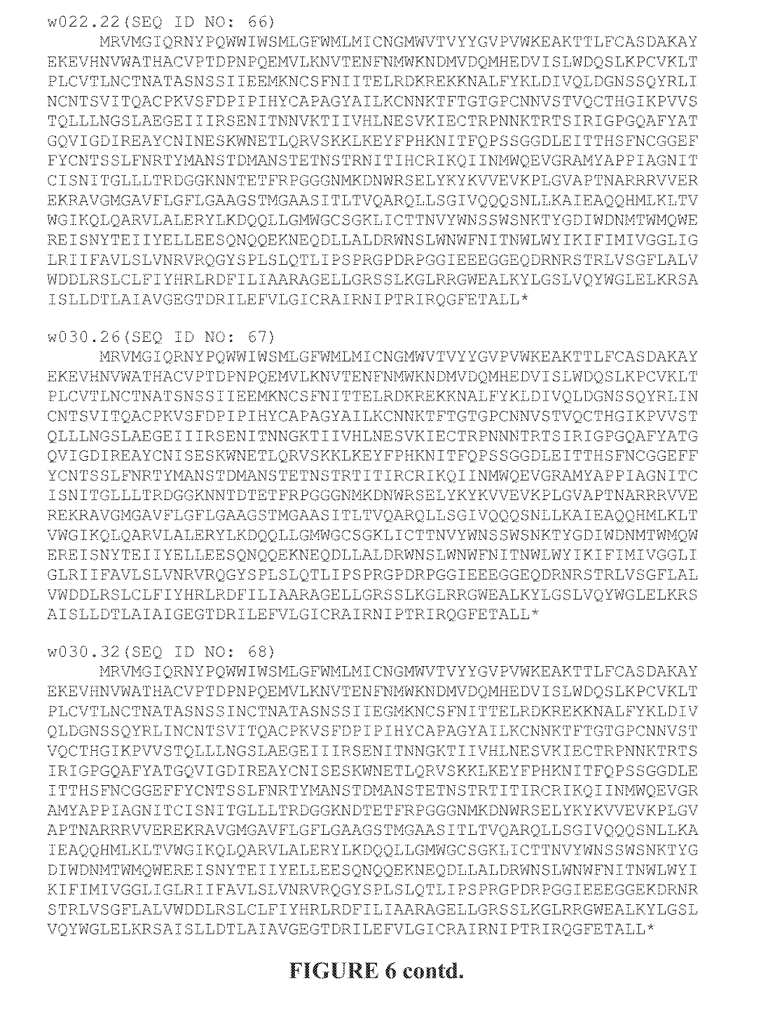

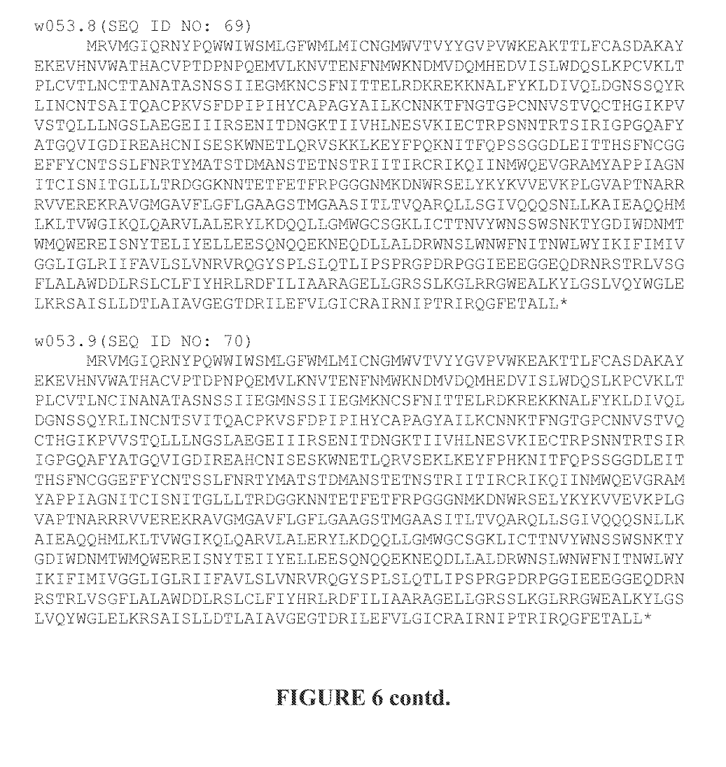

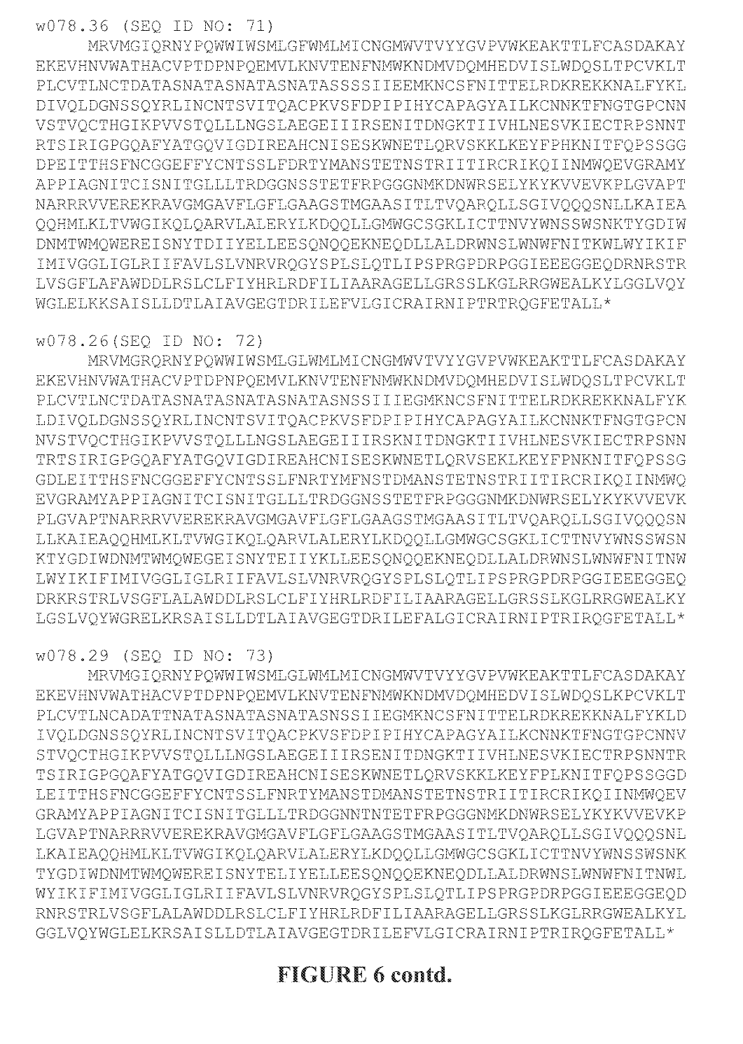

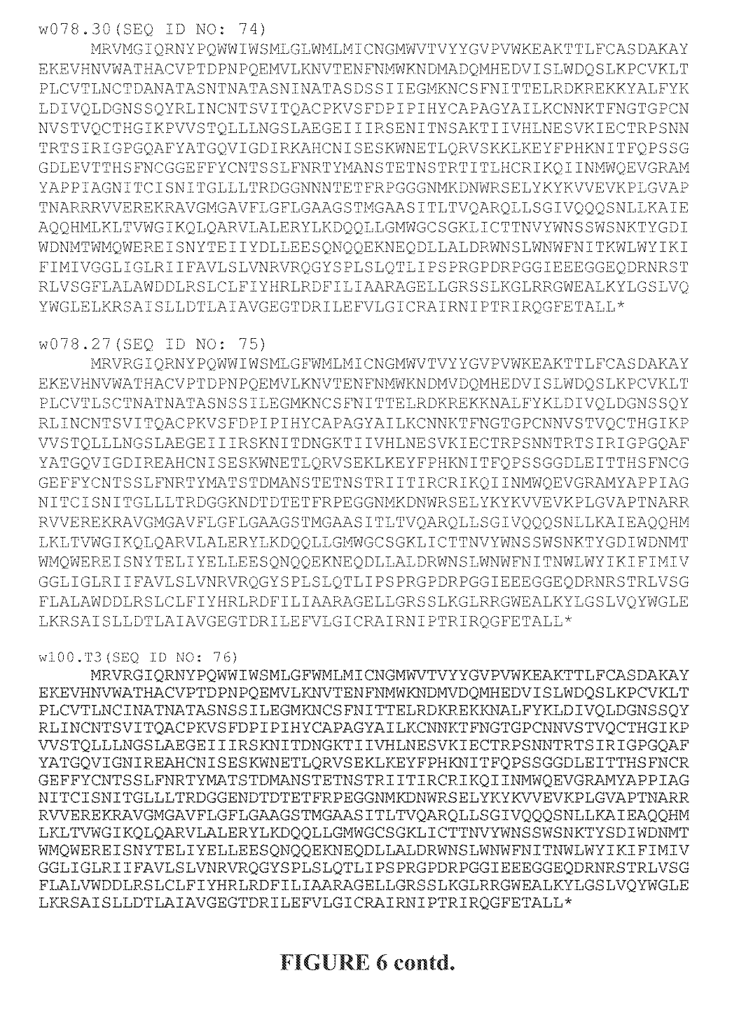

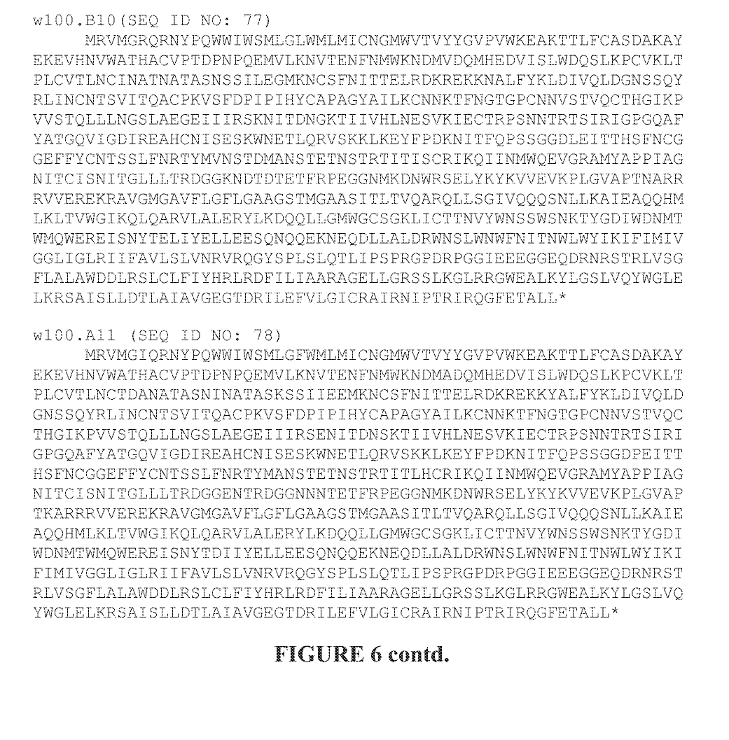

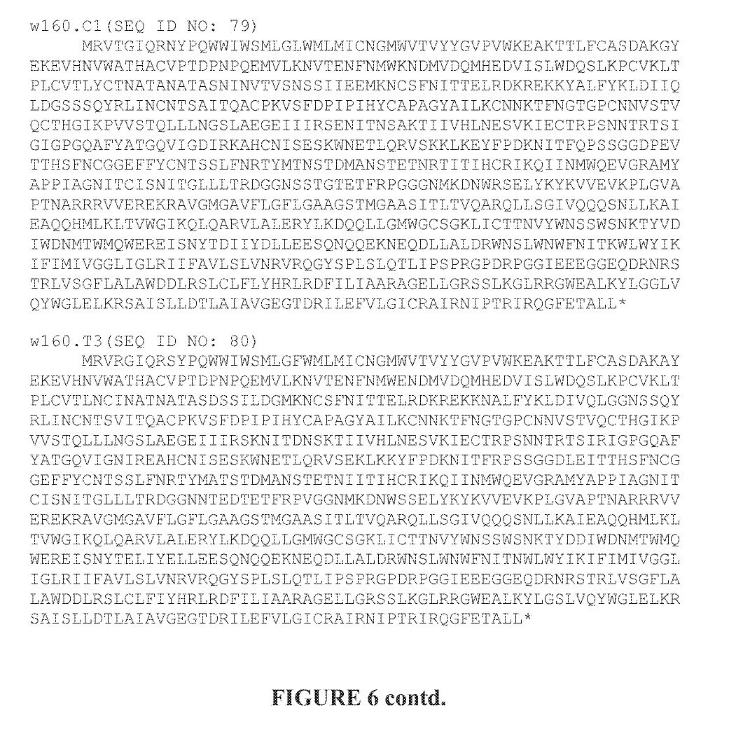

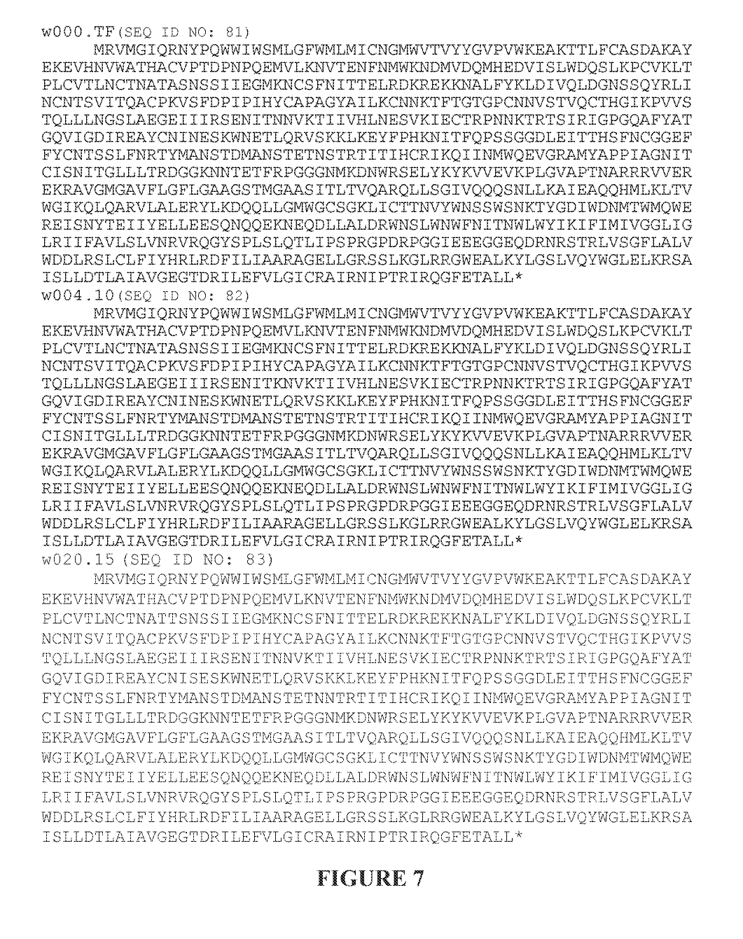

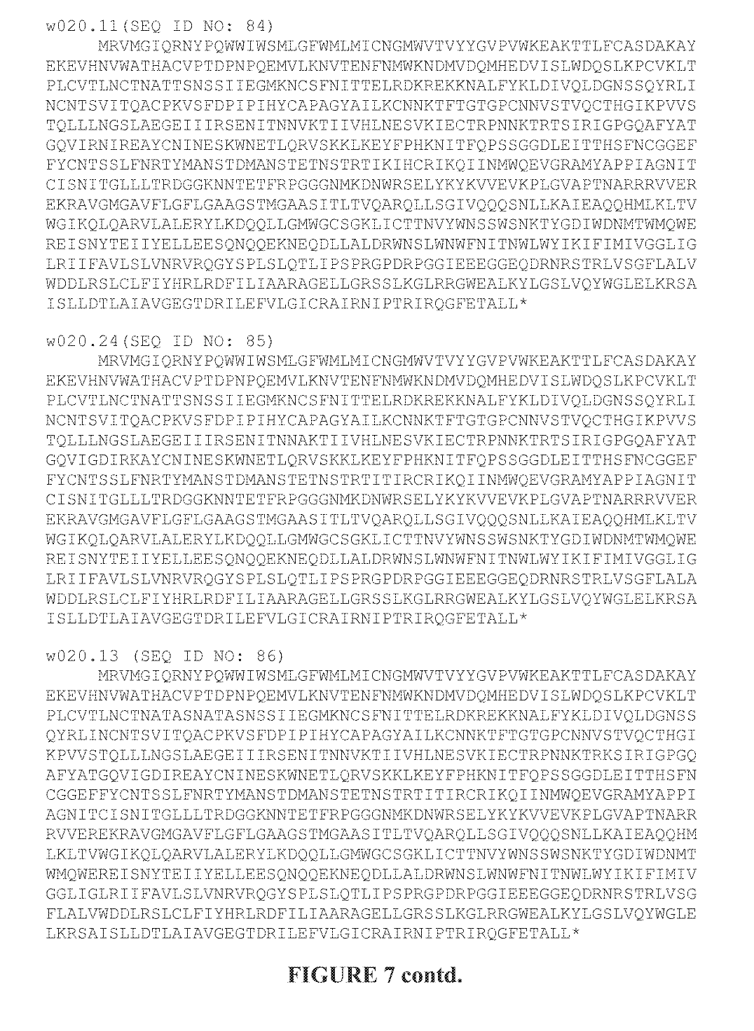

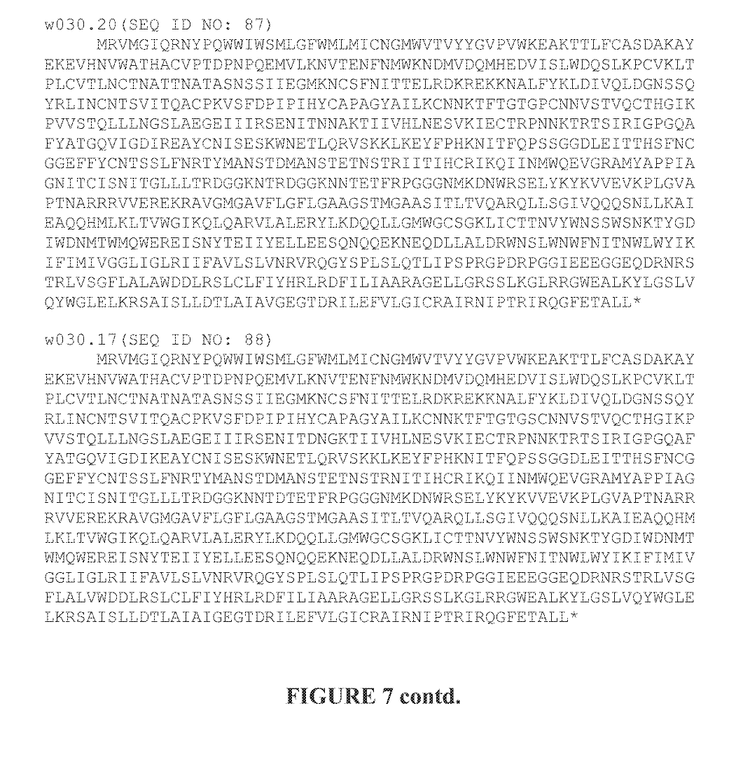

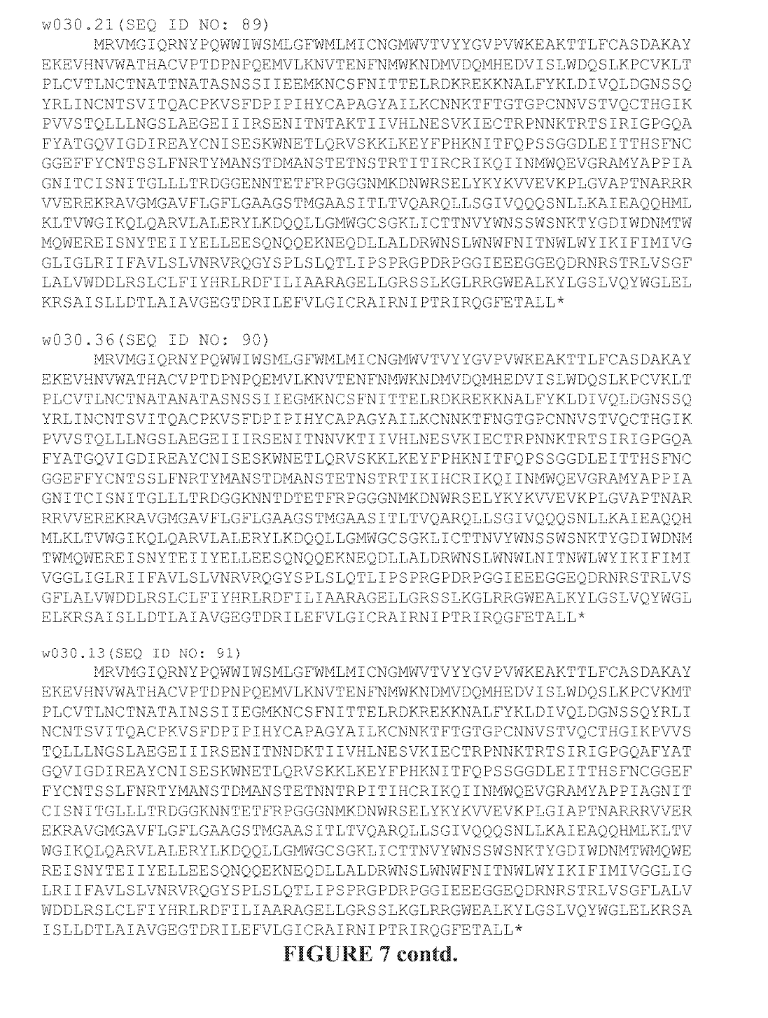

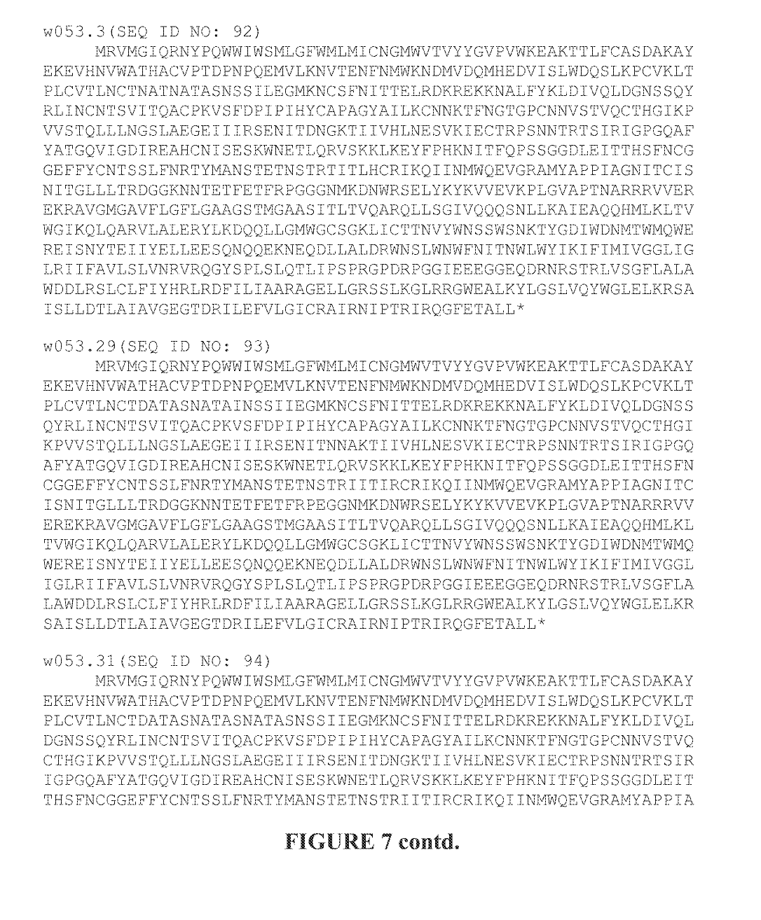

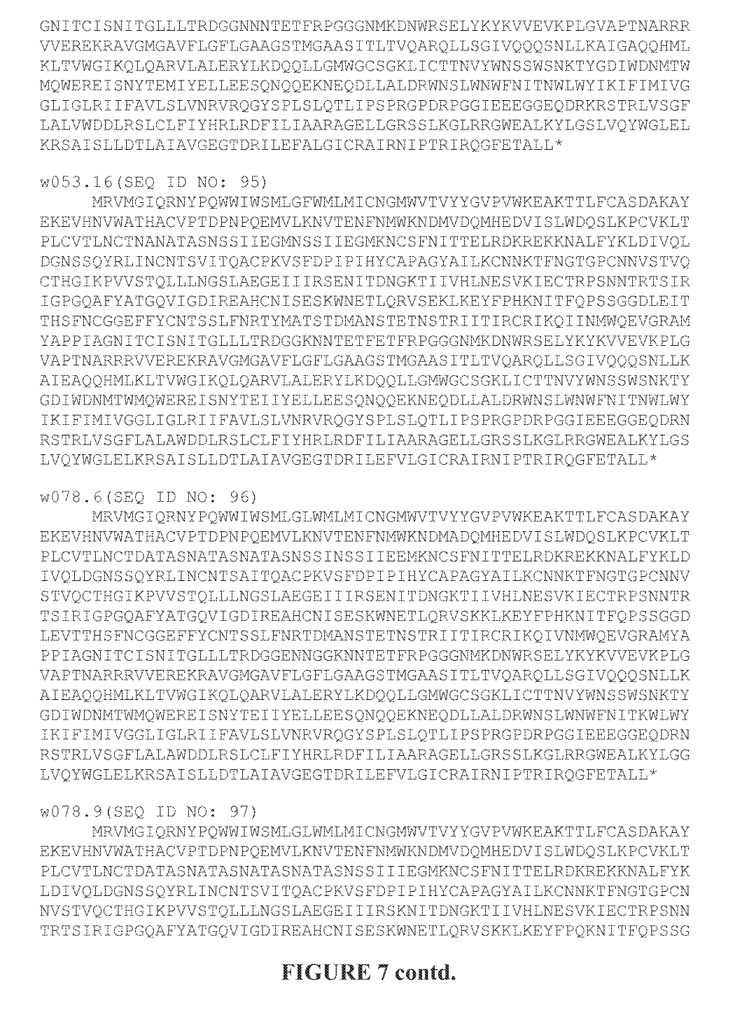

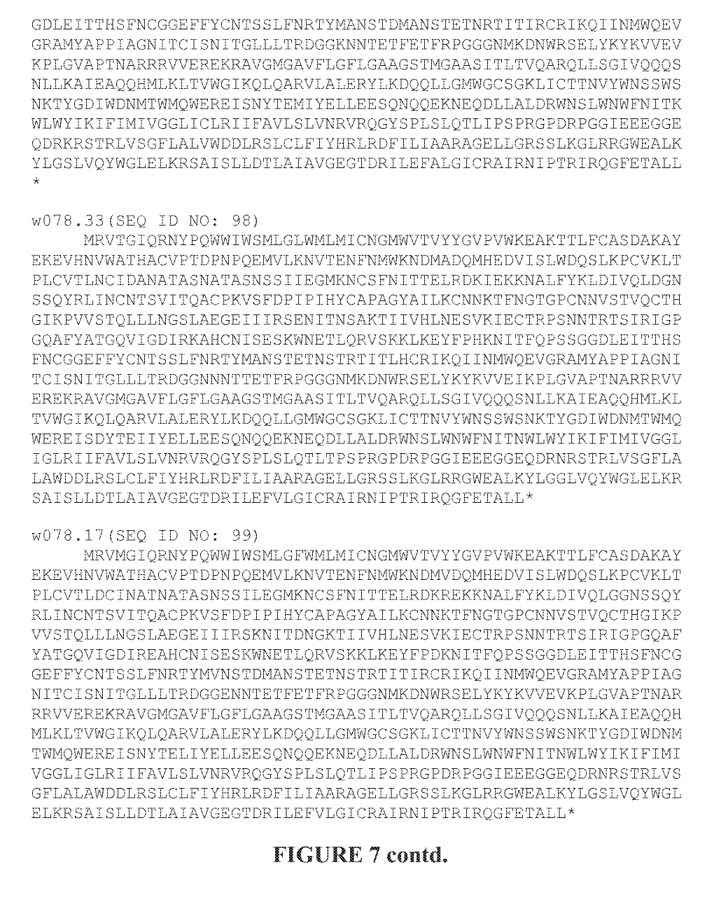

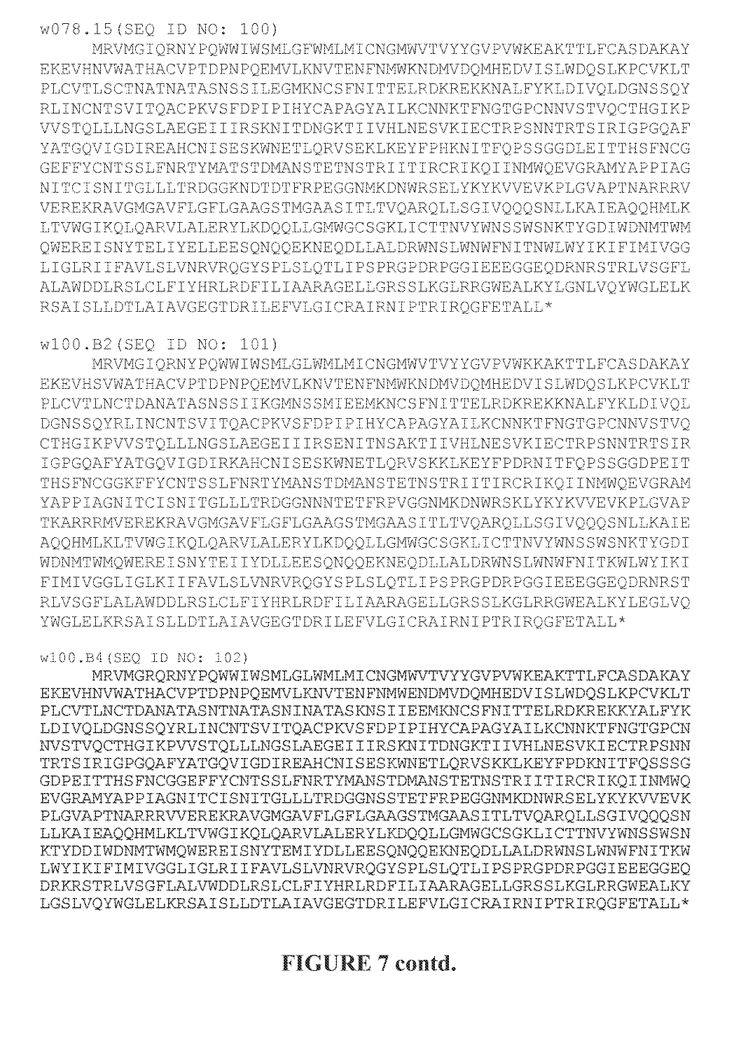

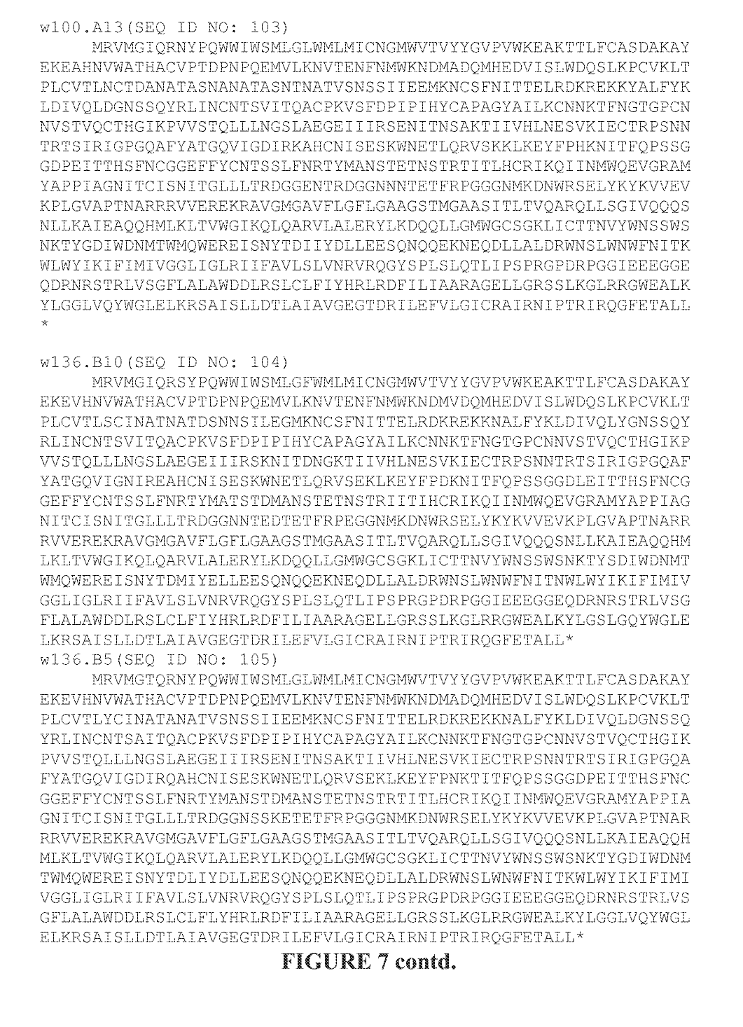

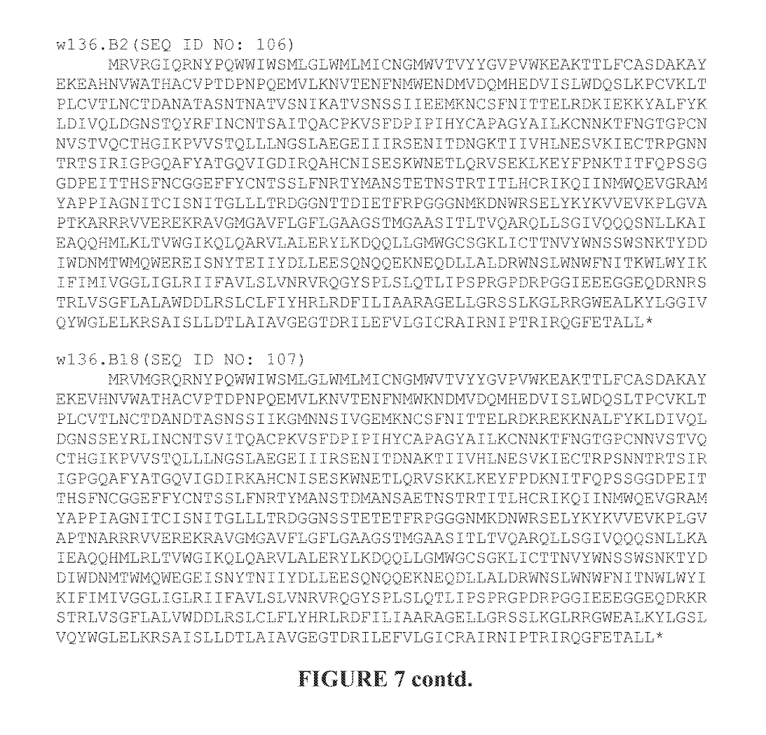

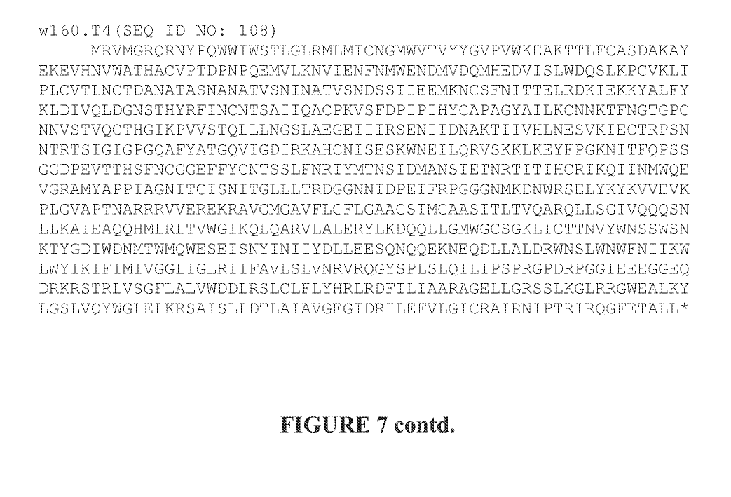

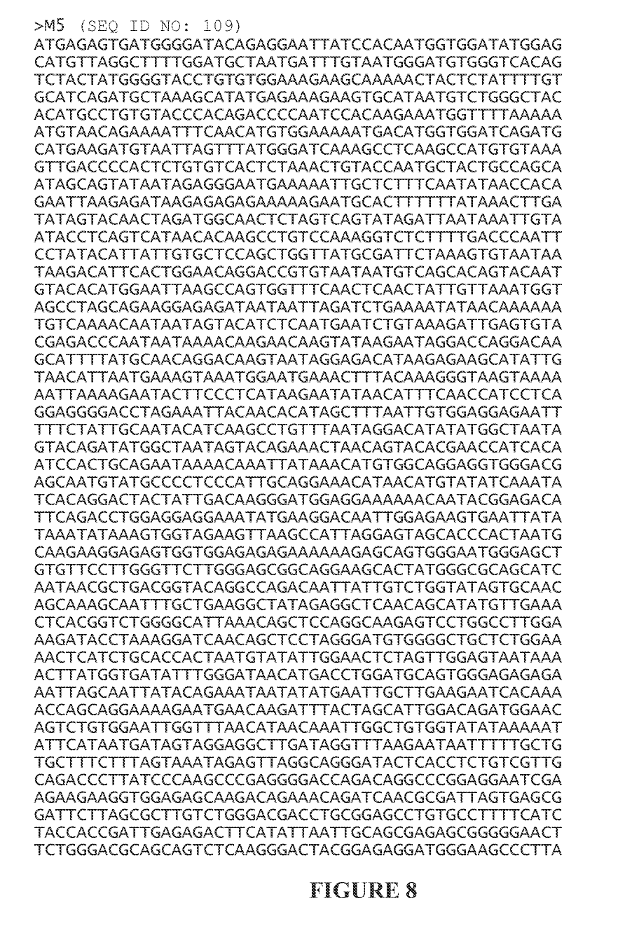

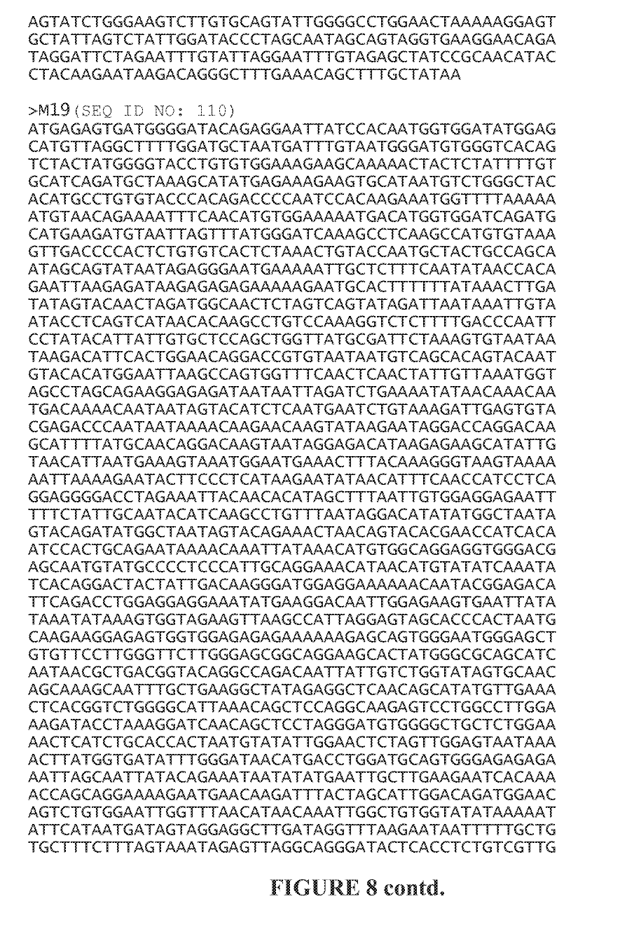

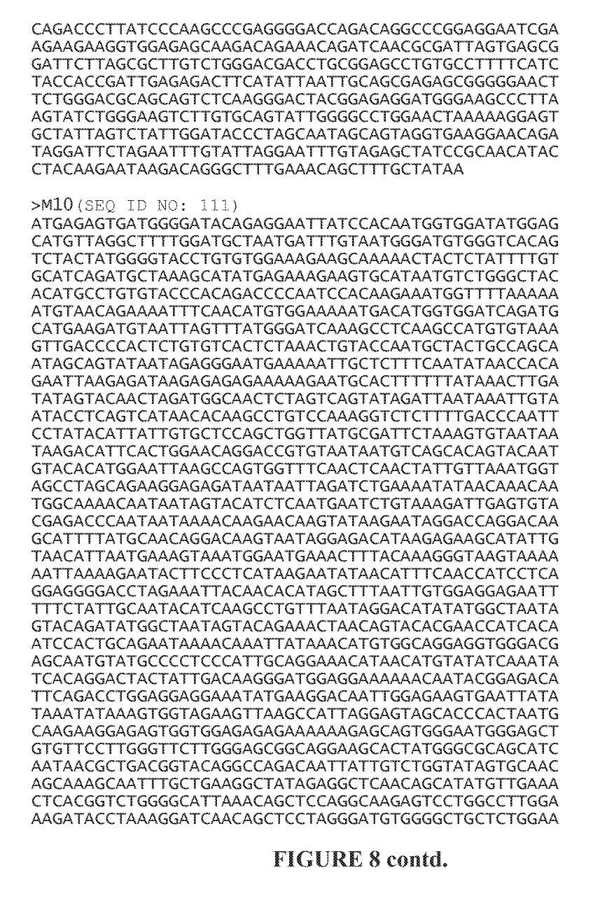

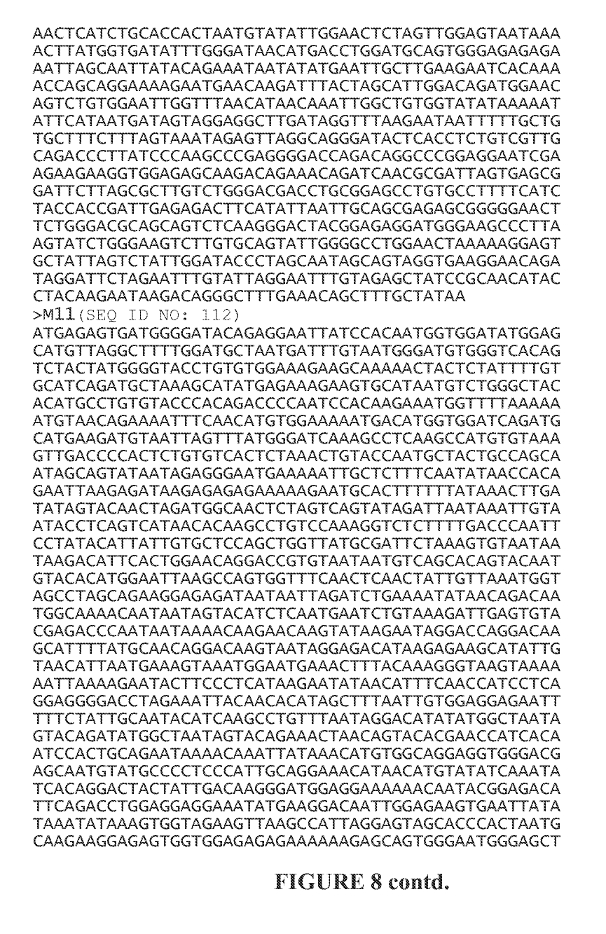

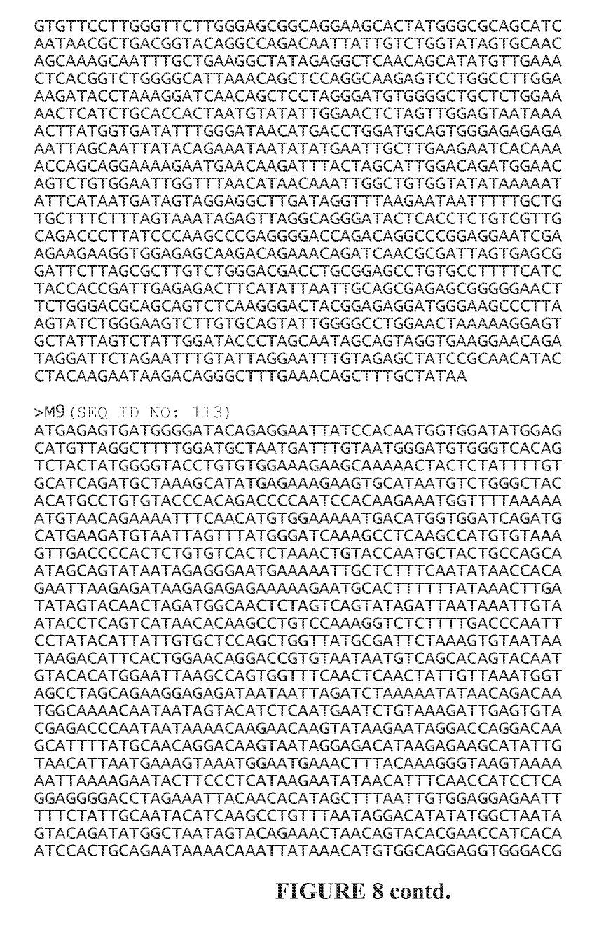

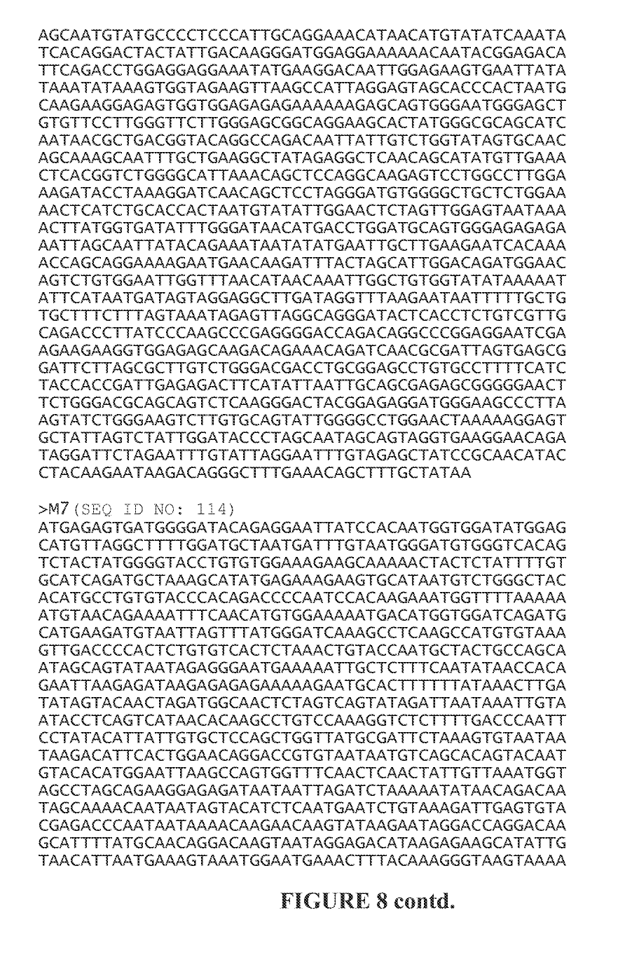

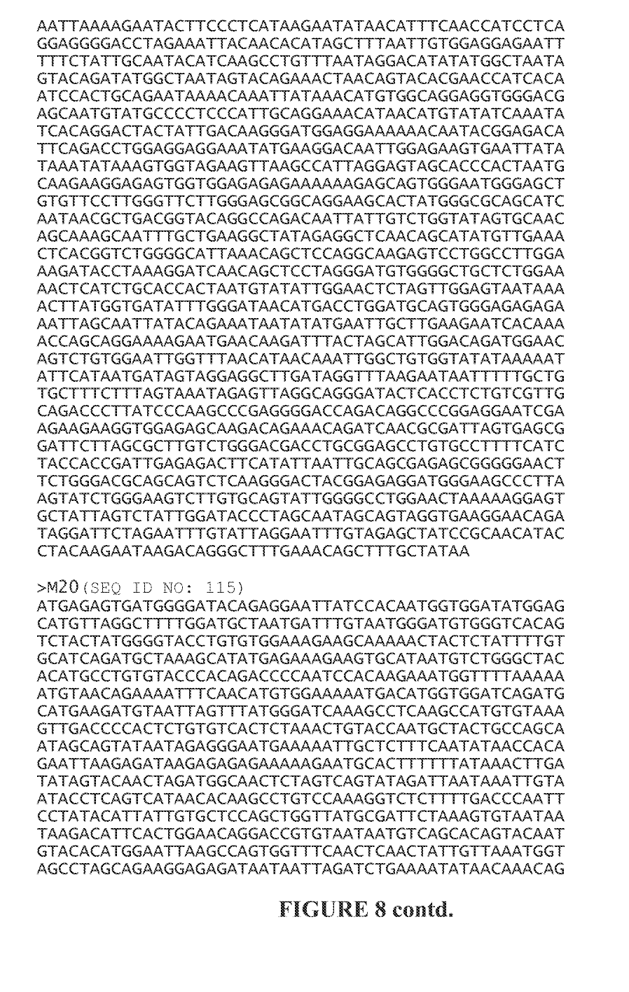

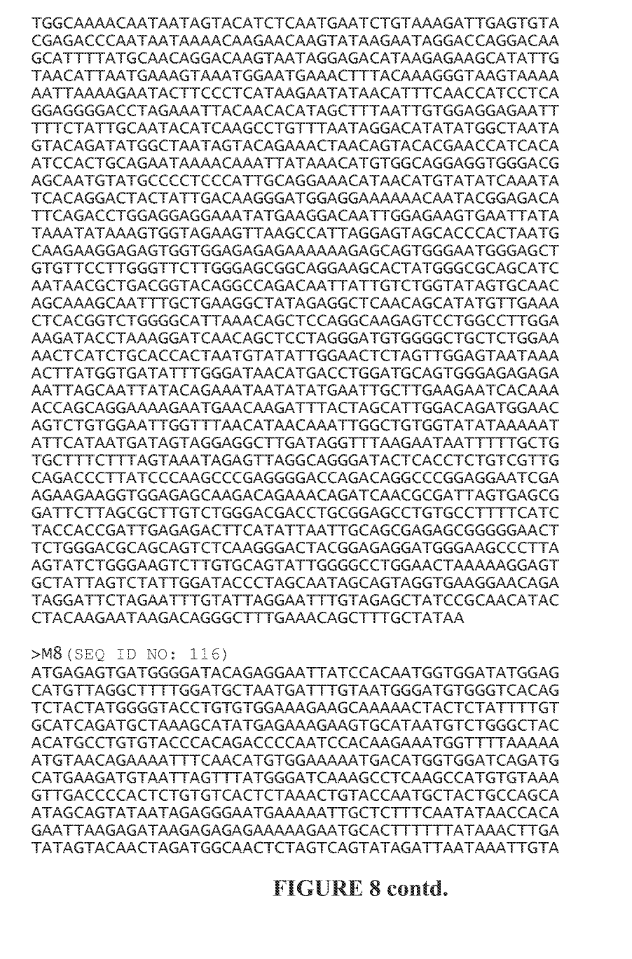

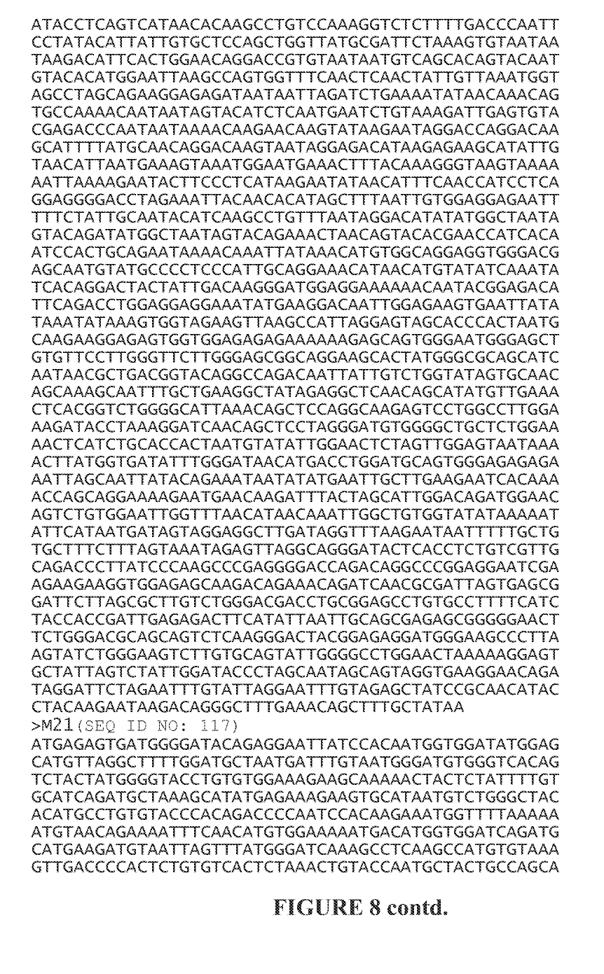

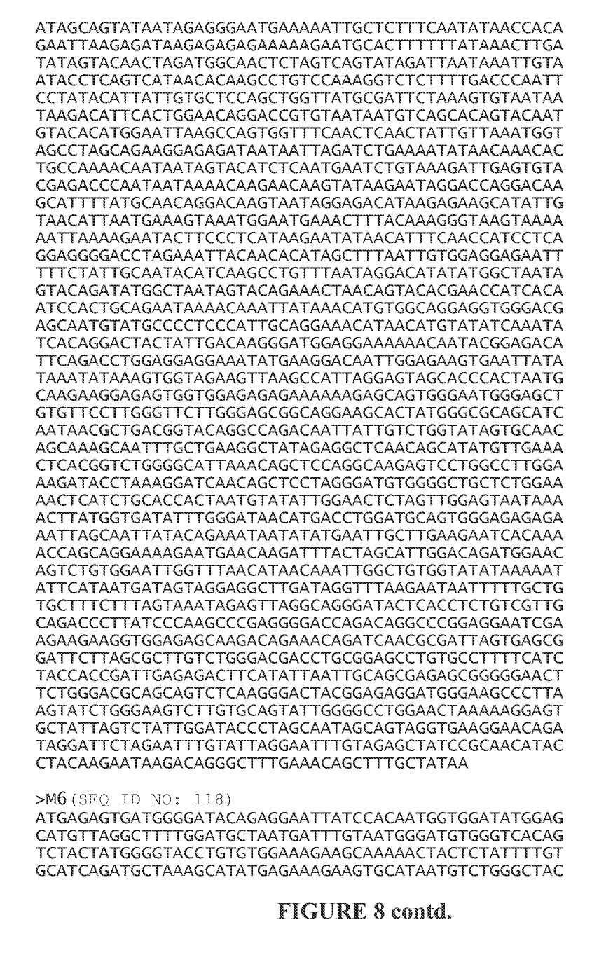

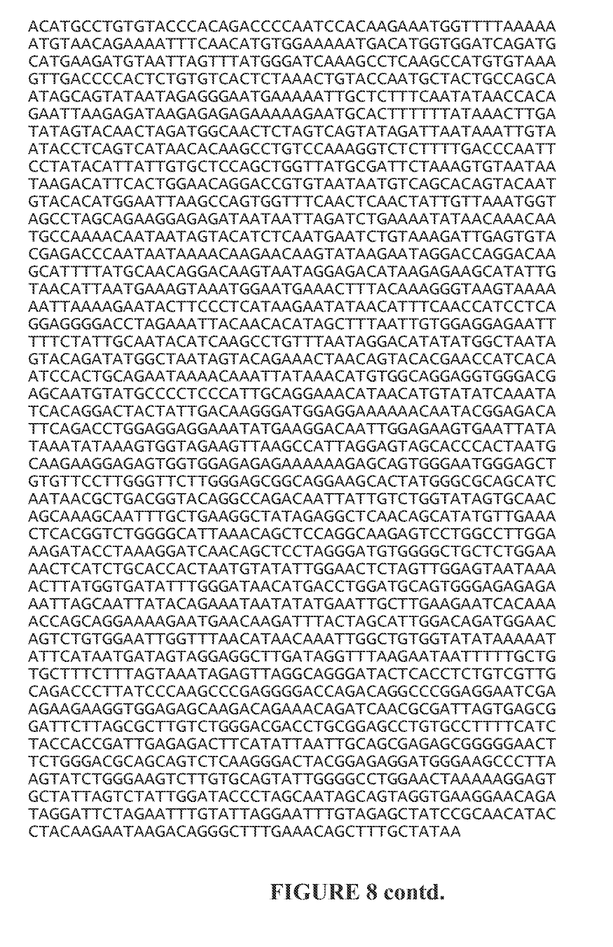

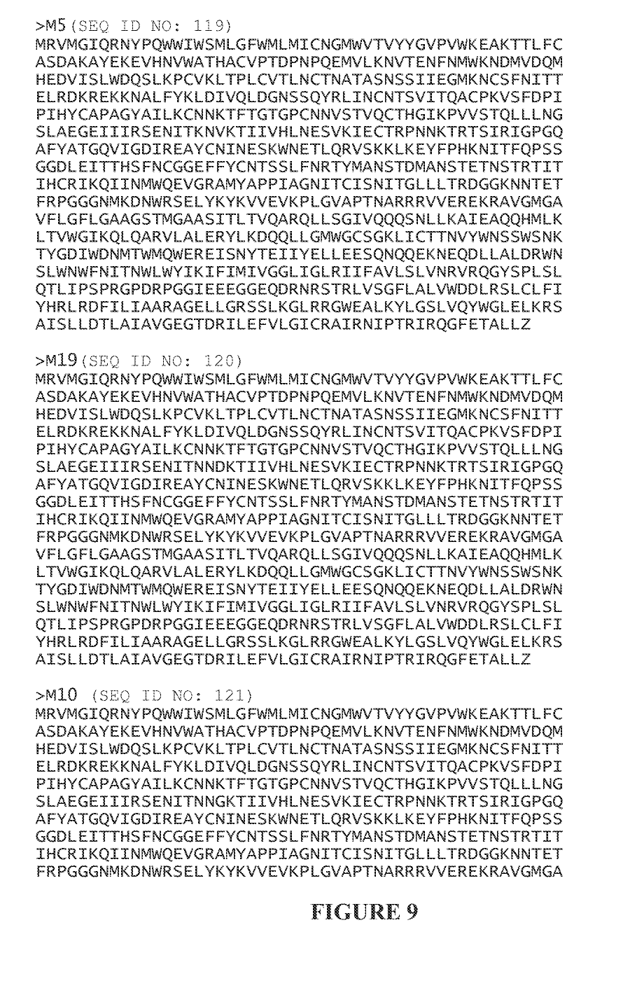

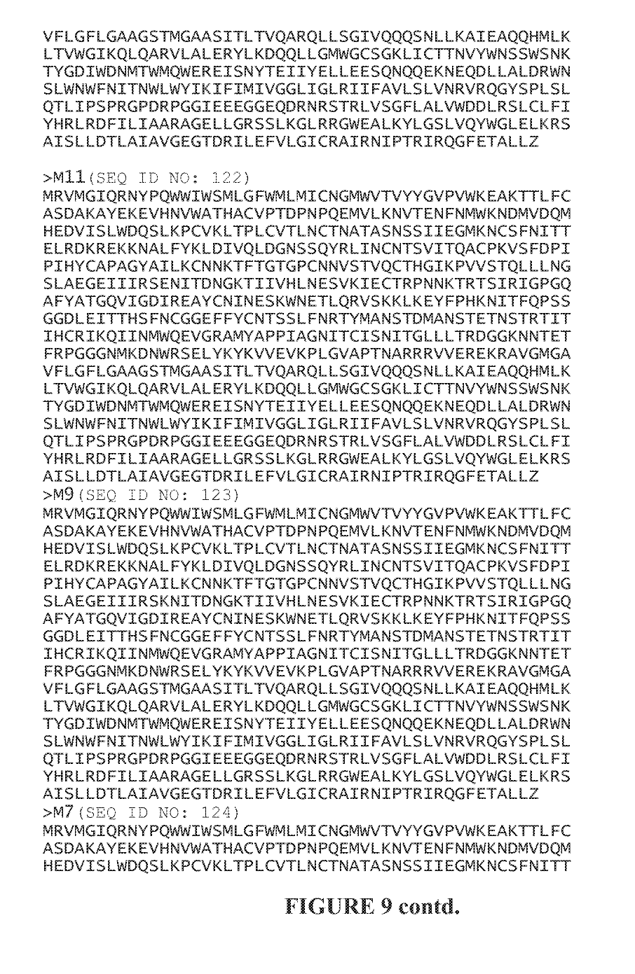

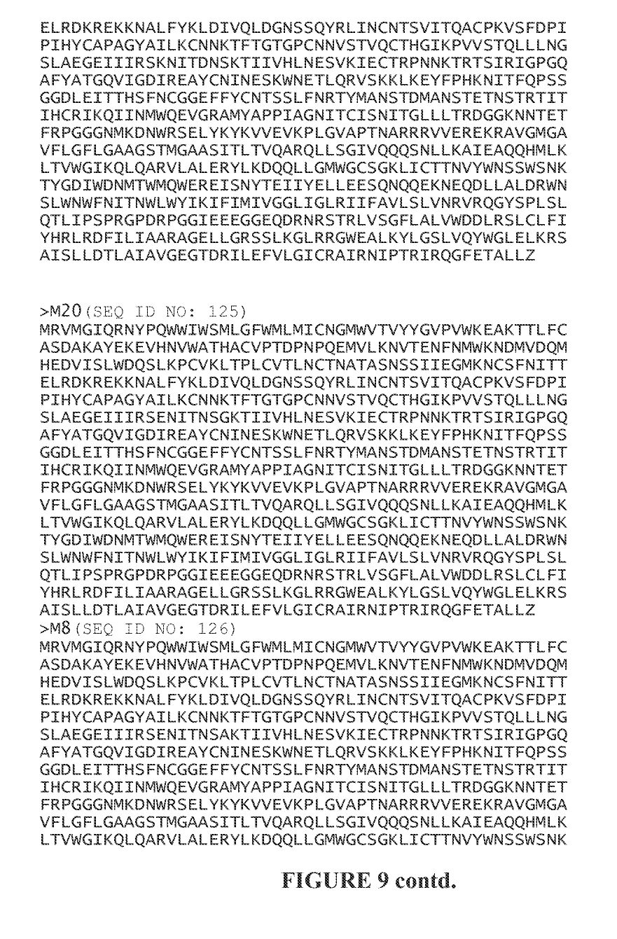

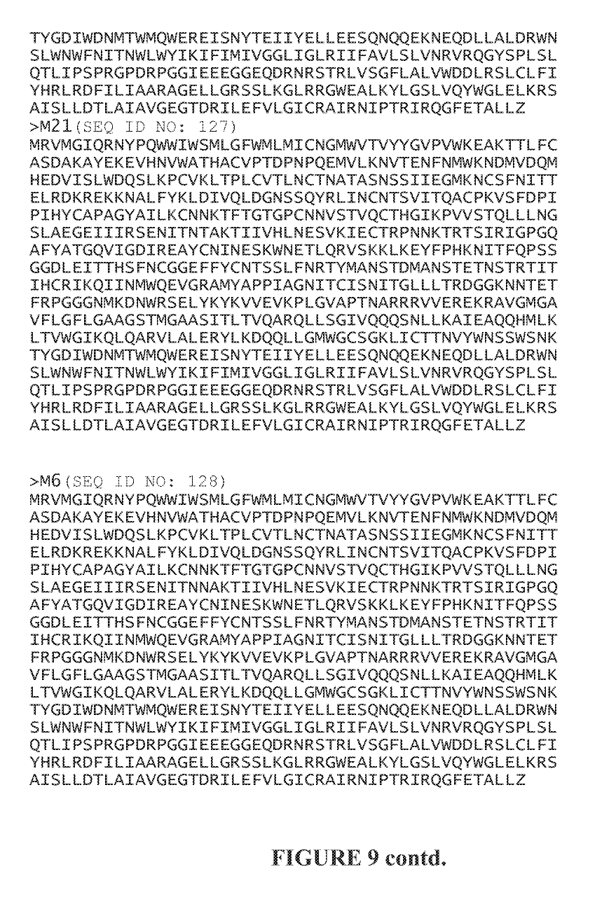

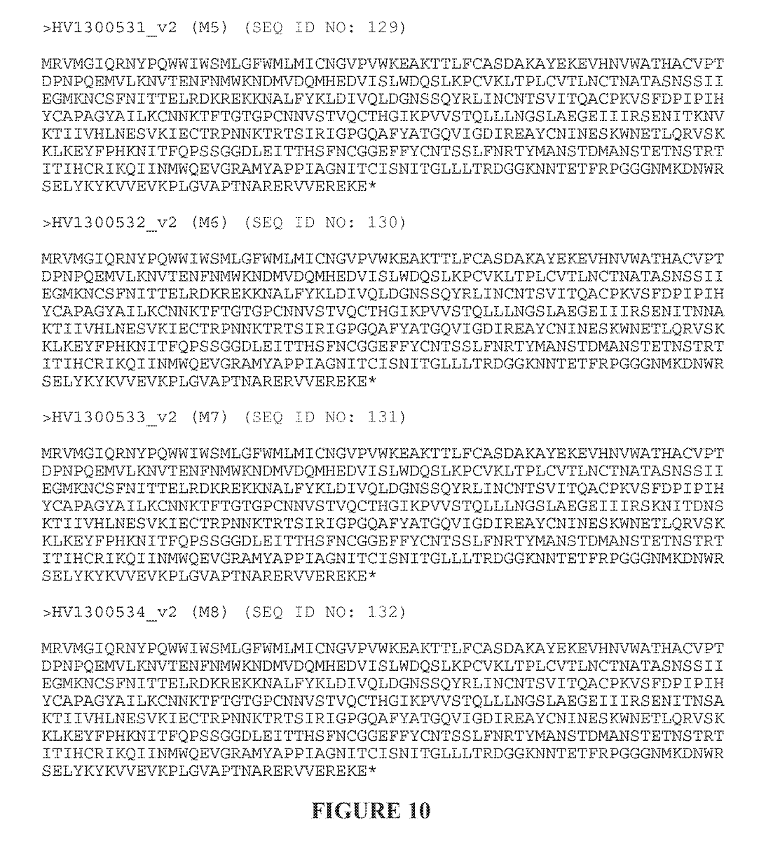

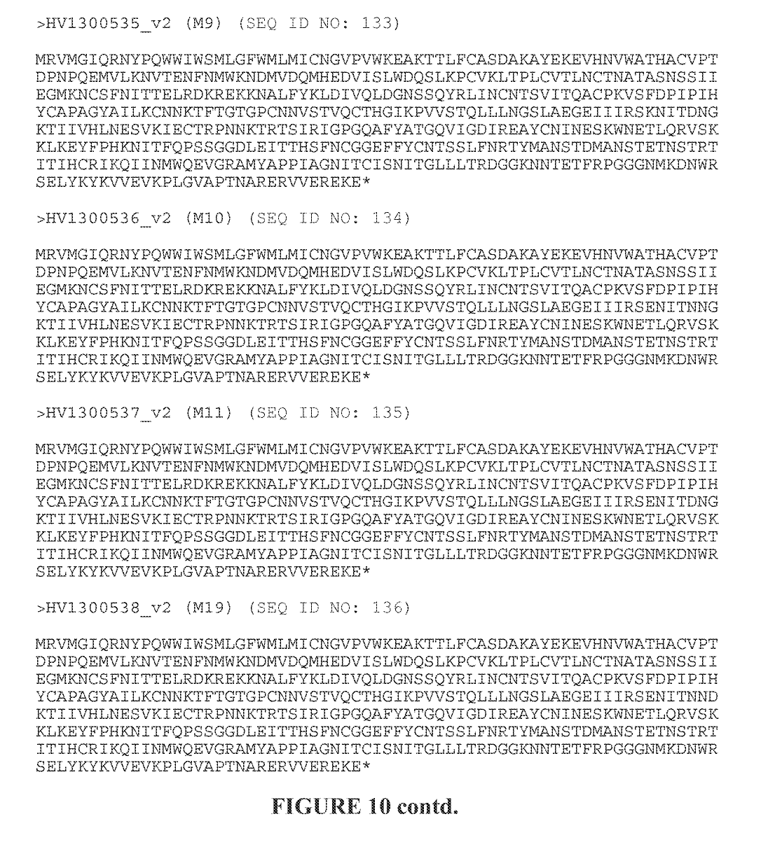

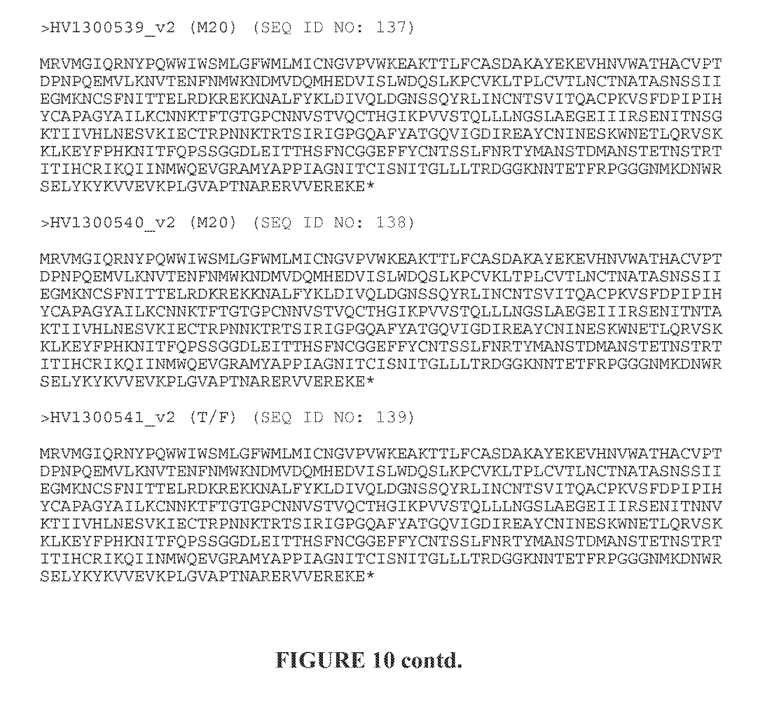

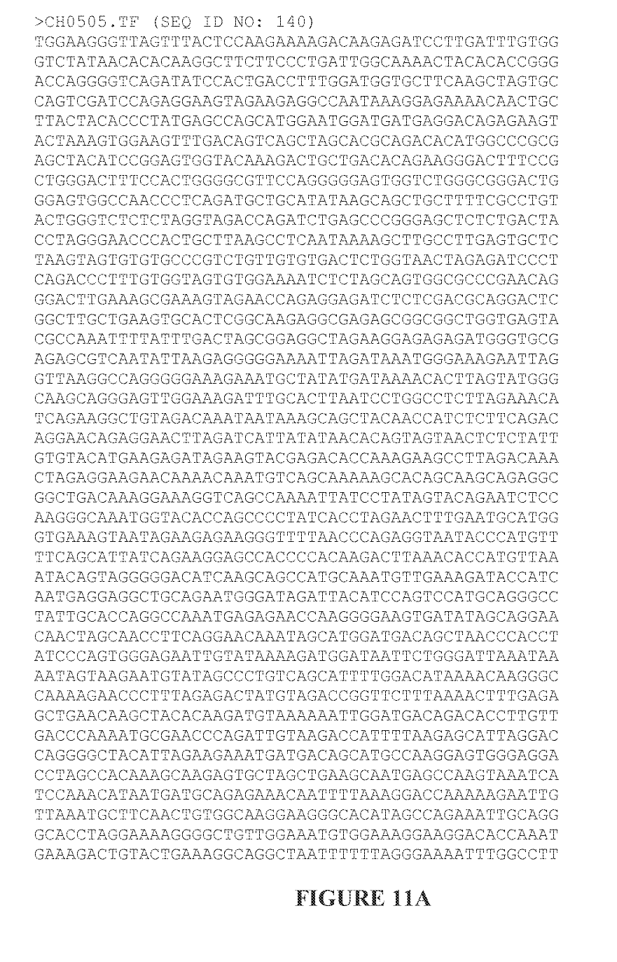

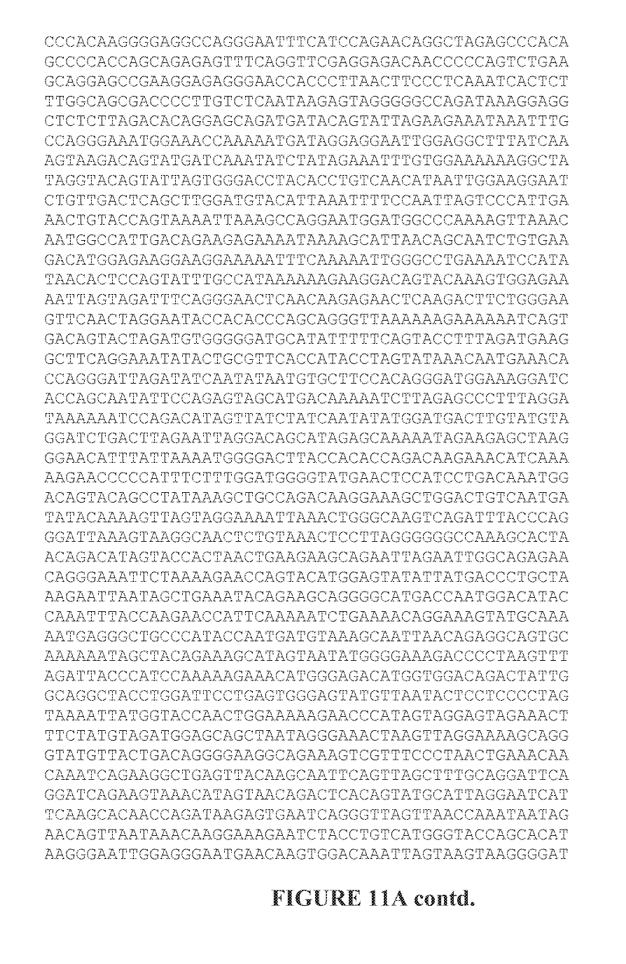

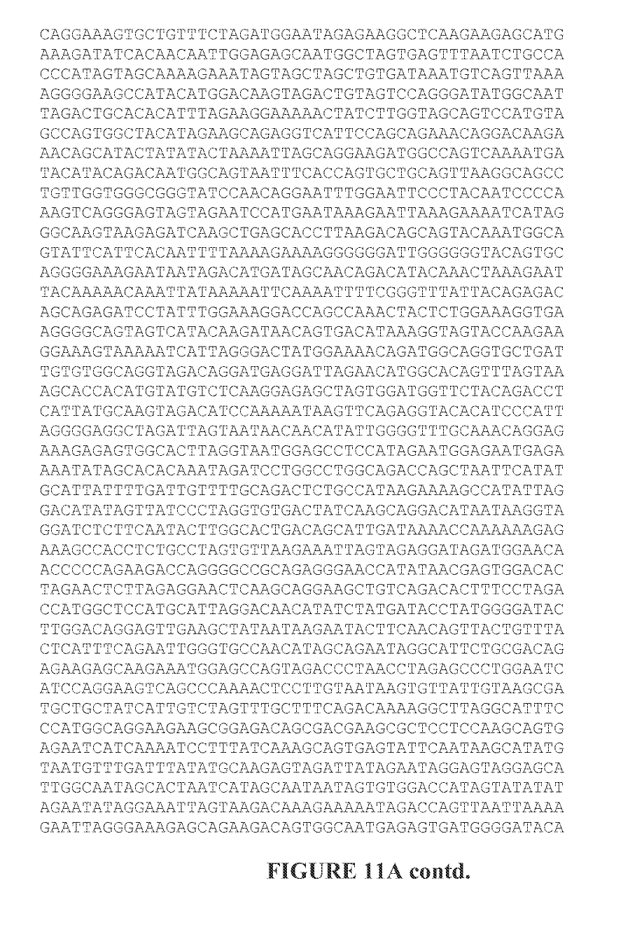

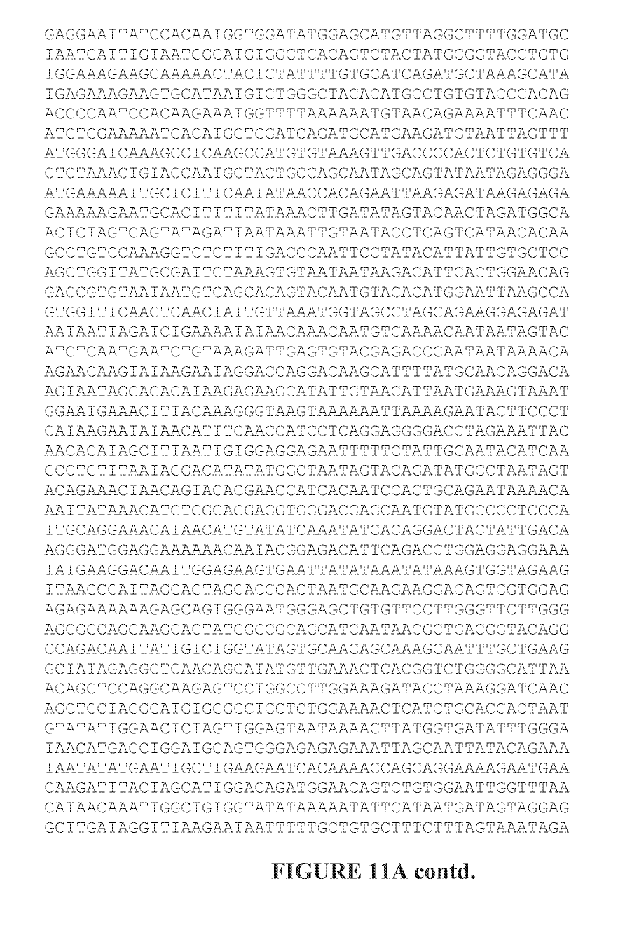

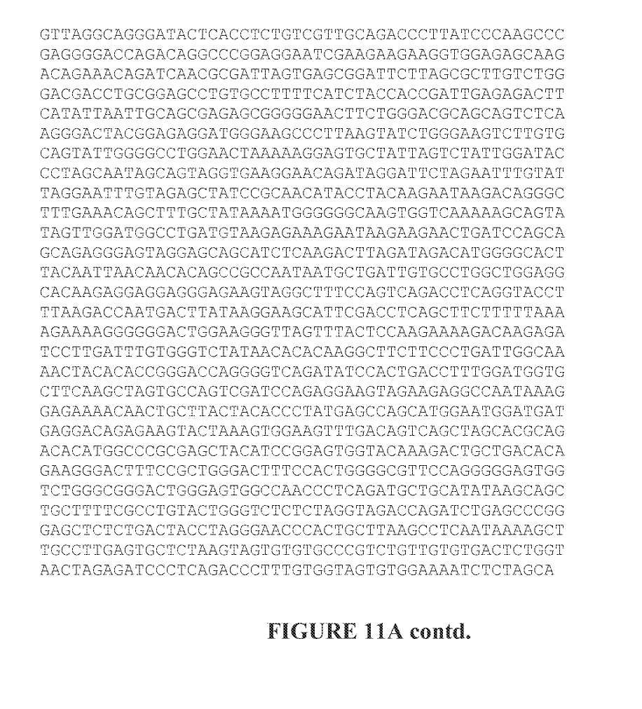

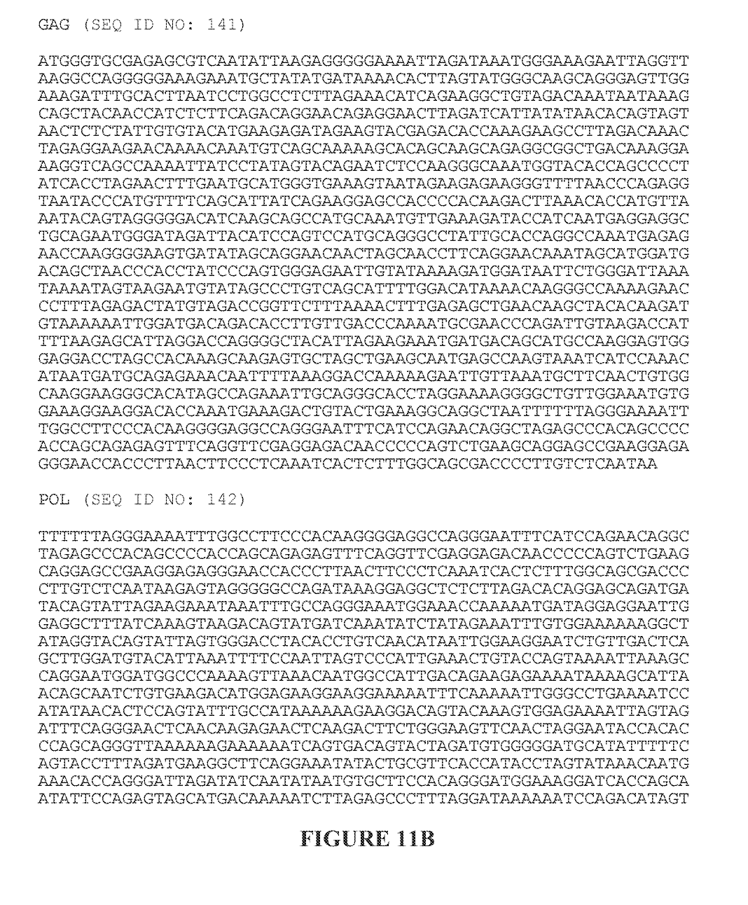

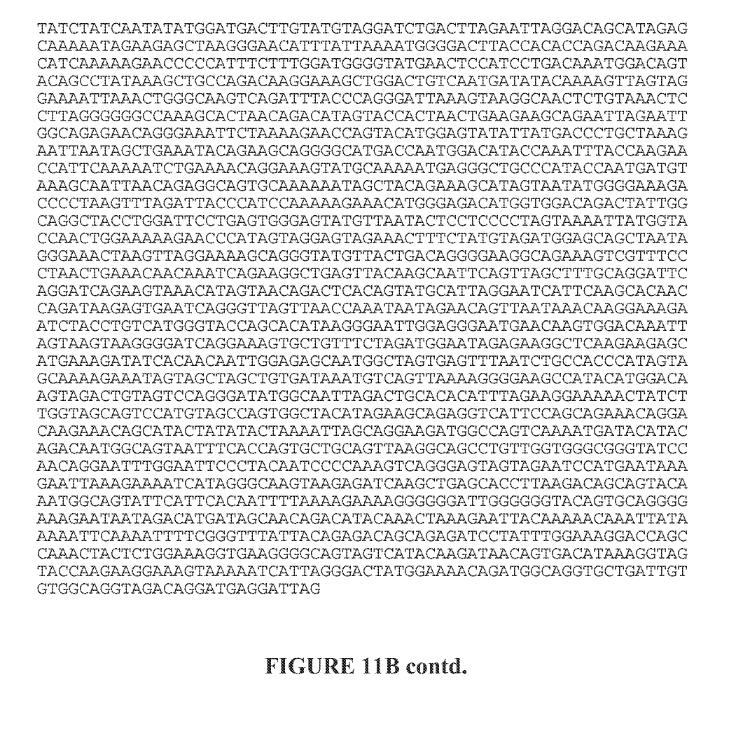

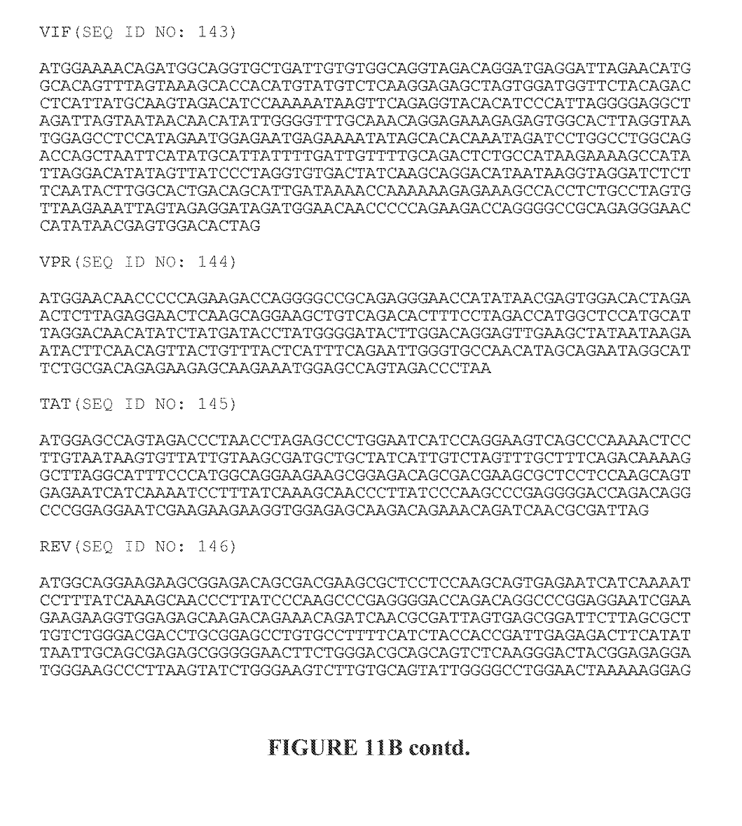

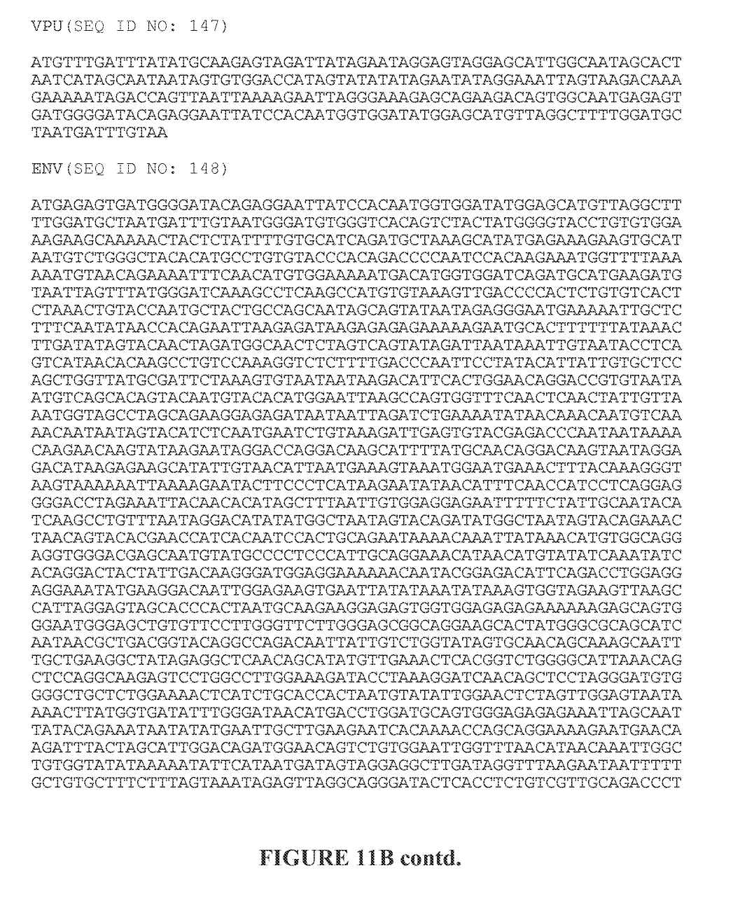

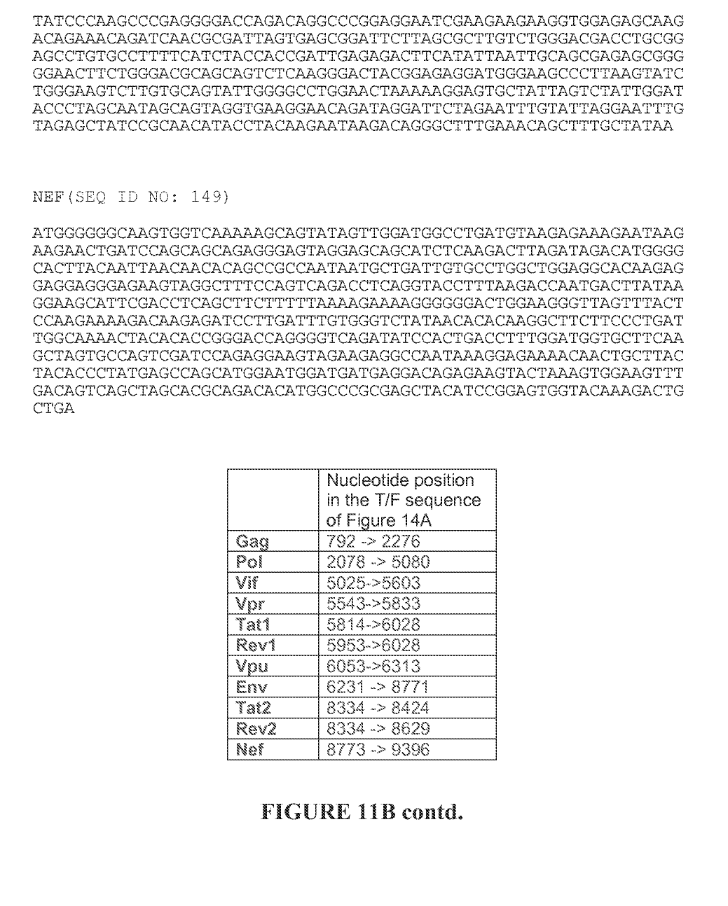

Specifically, for CH505 the virus and envelope evolution were mapped, and the CH103 CD4 binding-site bnAb evolution. In addition, 135 CH505 varied envelope pseudotyped viruses were made and tested them for neutralization sensitivity by members of the CH103 bnAb lineage (e.g, FIGS. 3). From this large dataset, in one embodiment, Env variants were chosen for immunization based on sequence diversity, and antigenic diversity, for example binding to antibodies in the CH103 lineage (FIGS. 1 and 2, Table 1).

In certain embodiments, the envelopes are selected based on Env mutants with sites under diversifying selection, in which the transmitted/founder (T/F) Env form vanished below 20% in any sample, i.e. escape variants; signature sites based on autologous neutralization data, i.e. Envs with statistically supported signatures for escape from members of the CH103 bnAb lineage; and sites with mutations at the contact sites of the CH103 antibody and HIV Env. In this manner, a sequential swarm of Envs was selected for immunization to represent the progression of virus escape mutants that evolved during bnAb induction and increasing neutralization breadth in the CH505 donor.

In certain embodiments, additional sequences are selected to contain five additional specific amino acid signatures of resistance that were identified at the global population level. These sequences contain statistically defined resistance signatures, which are common at the population level and enriched among heterologous viruses that CH103 fails to neutralize. When they were introduced into the TF sequence, they were experimentally shown to confer partial resistance to antibodies in the CH103 lineage. Following the reasoning that serial viral escape and antibody adaptation to escape is what ultimate selects for neutralizing antibodies that exhibit breadth and potency against diverse variants, in certain embodiments, inclusion of these variants in a vaccine may extend the breadth of vaccine-elicited antibodies even beyond that of the CH103 lineage. Thus the overarching goal will be to trigger a CH103-like lineage first using the CH505TF modified M11, that is well recognized by early CH103 ancestral states, then vaccinating with antigenic variants, to allow the antibody lineage to adapt through somatic mutation to accommodate the natural variants that arose in CH505. In certain embodiments, vaccination regimens include a total of 27 sequences (Selection B) that capture the antigenic diversity of CH505. In another embodiment, additional antigenic diversity is added (Selection A), to enable the induction of antibodies by vaccination that may have even greater breadth than those antibodies isolated from CH505.

In some embodiments, the CH505 sequences that represent the accumulation of viral sequence and antigenic diversity in the CD4bs epitope of CH103 in subject CH505 are represented by selection A, or selection B.

M11 is a mutant generated to include two mutations in the loop D (N279D+V281G relative to the TF sequence) that enhanced binding to the CH103 lineage . These were early escape mutations for another CD4bs autologous neutralizing antibody lineage, but might have served to promote early expansion of the CH103 lineage.

In certain embodiments, the two CH103 resistance signature-mutation sequences added to the antigenic swarm are: M14 (TF with S364P), and M24 (TF with S375H+T202K+L520F+G459E). They confer partial resistance to the TF with respect to the CH103 lineage. In certain embodiments, these D-loop mutants are administered in the boost.

### Example 4

### Immunization Protocols in Subjects with Swarms of HIV-1 Envelopes

Immunization protocols contemplated by the invention include envelopes sequences as described herein including but not limited to nucleic acids and/or amino acid sequences of gp160s, gp150s, gp145, cleaved and uncleaved gp140s, gp120s, gp41s, N-terminal deletion variants as described herein, cleavage resistant variants as described herein, or codon optimized sequences thereof. A skilled artisan can readily modify the gp160 and gp120 sequences described herein to obtain these envelope variants. The swarm immunization protocols can be administered in any subject, for example monkeys, mice, guinea pigs, or human subjects.

In non-limiting embodiments, the immunization includes a nucleic acid which is administered as DNA, for example in a modified vaccinia vector (MVA). In non-limiting embodiments, the nucleic acids encode gp160 envelopes. In other embodiments, the nucleic acids encode gp120 envelopes. In other embodiments, the boost comprises a recombinant gp120 envelope. The vaccination protocols include envelopes formulated in a suitable carrier and/or adjuvant, for example but not limited to alum. In certain embodiments the immunizations include a prime, as a nucleic acid or a recombinant protein, followed by a boost, as a nucleic acid or a recombinant protein. A skilled artisan can readily determine the number of boosts and intervals between boosts.

In certain embodiments an immunization protocol could prime with a bivalent or trivalent Gag mosaic (Gag1 and Gag 2, Gag 1, Gag 2 and Gag3) in a suitable vector.

### Example 5

### Env Mixtures of the CH505 Virus are Expected to Induce the Beginning of CD4 Binding Site BnAb Lineages

In one immunization regimen, the prime is M6, M5, M11 then groups of envelopes from the selection of 54 envelopes are added either sequentially or additively.

### Example 6

One of the major obstacles to developing an efficacious preventive HIV-1 vaccine is the challenge of inducing broadly neutralizing antibodies (bnAbs) against the virus. There are several reasons why eliciting bnAbs has been challenging and these include the conformational structure of the viral envelope, molecular mimicry of host antigens by conserved epitopes which may lead to the suppression of potentially useful antibody responses, and the high level of somatic mutations in the variable domains and the requirement for complex maturation pathways [1-3]. It has been shown that up to 25% of HIV-1—infected individuals develop bnAbs that are detected 2-4 years after infection. To date, all bnAbs have one or more of these unusual antibody traits: high levels of somatic mutation, autoreactivity with host antigens, and long heavy chain third complementarity determining regions (HCDR3s)—all traits that are controlled or modified by host immunoregulatory mechanisms. Thus, the hypothesis has been put forth that typical vaccinations of single primes and boosts will not suffice to be able to induce bnAbs; rather, it will take sequential immunizations with Env immunogens, perhaps over a prolonged period of time, to mimic bnAb induction in chronically infected individuals [4].

A process to circumvent host immunoregulatory mechanisms involved in control of bnAbs is termed B cell lineage immunogen design, wherein sequential Env immunogens are chosen that have high affinities for the B cell receptors of the unmutated common ancestor (UCA) or germline gene of the bnAb clonal lineage [4]. Envs for immunization can either be picked randomly for binding or selected, as described herein, from the evolutionary pathways of Envs that actually give rise to bnAbs in vivo. Liao and colleagues recently described the co-evolution of HIV-1 and a CD4 binding site bnAb from the time of seroconversion to the development of plasma bnAb induction, thereby presenting an opportunity to map out the pathways that lead to generation of this type of CD4 binding site bnAb [5]. They showed that the single transmitted/founder virus was able to bind to the bnAb UCA, and identified a series of evolved envelope proteins of the founder virus that were likely stimulators of the bnAb lineage. Thus, this work presents an opportunity to vaccinate with naturally-derived viral envelopes that could drive the desired B-cell responses and induce the development of broad and potent neutralizing antibodies. While the human antibody repertoire is diverse, it has been found that only a few types of B cell lineages can lead to bnAb development, and that these lineages are similar across a number of individuals [6,7]. Thus, it is feasible that use of Envs from one individual will generalize to others.

In certain embodiments the invention provides methods for selecting the Env immunogens, among multitude of diverse viruses that induced a CD4 binding site bnAb clonal lineage in an HIV-infected individual, by making sequential recombinant Envs from that individual and using these Envs for vaccination. The B-cell lineage vaccine strategy thus includes designing immunogens based on unmutated ancestors as well as intermediate ancestors of known bnAb lineages. A candidate vaccine could use transmitted/founder virus envelopes to, at first, stimulate the beginning stages of a bnAb lineage, and subsequently boost with evolved Env variants to recapitulate the high level of somatic mutation needed for affinity maturation and bnAb activity. The goal of such a strategy is to selectively drive desired bnAb pathways.

Broadly neutralizing antibodies likely will not be induced by a single Env, and even a mixture of polyvalent random Envs (e.g. HVTN 505) is unlikely to induce bnAbs. Rather, immunogens must be designed to trigger the UCAs of bnAb lineages to undergo initial bnAb lineage maturation, and then use sequential immunogens to fully expand the desired lineages. The proposed trial will represent the first of many experimental clinical trials testing this concept in order to develop the optimal set of immunogens to drive multiple specificities of bnAbs. The HVTN will be at the cutting edge of this effort.

The concept is applicable to driving CD4 binding site lineage in multiple individuals due to the convergence of a few bnAb motifs among individuals. The adjuvant will be the GSK AS01E adjuvant containing MPL and QS21. Other suitable adjuvants can be used. This adjuvant has been shown by GSK to be as potent as the similar adjuvant AS01B but to be less reactogenic using HBsAg as vaccine antigen [Leroux-Roels et al., IABS Conference, April 2013, [9].

1. Mascola J R, Haynes B F. HIV-1 neutralizing antibodies: understanding nature's pathways. Immunol Rev 2013; 254:225-44.

2. Verkoczy L, Kelsoe G, Moody M A, Haynes B F. Role of immune mechanisms in induction of HIV-1 broadly neutralizing antibodies. Curr Opin Immunol 2011; 23:383-90.

3. Verkoczy L, Chen Y, Zhang J, Bouton-Verville H, Newman A, Lockwood B, Scearce R M, Montefiori D C, Dennison S M, Xia S M, Hwang K K, Liao H X, Alam S M, Haynes B F. Induction of HIV-1 broad neutralizing antibodies in 2F5 knock-in mice: selection against membrane proximal external region-associated autoreactivity limits T-dependent responses. J Immunol 2013; 191:2538-50.

4. Haynes B F, Kelsoe G, Harrison S C, Kepler T B. B-cell-lineage immunogen design in vaccine development with HIV-1 as a case study. Nat Biotechnol 2012; 30:423-33.

5. Liao H X, Lynch R, Zhou T, Gao F, Alam S M, Boyd S D, Fire A Z, Roskin K M, Schramm C A, Zhang Z, Zhu J, Shapiro L, Mullikin J C, Gnanakaran S, Hraber P, Wiehe K, Kelsoe G, Yang G, Xia S M, Montefiori D C, Parks R, Lloyd K E, Scearce R M, Soderberg K A, Cohen M, Kamanga G, Louder M K, Tran L M, Chen Y, Cai F, Chen S, Moquin S, Du X, Joyce M G, Srivatsan S, Zhang B, Zheng A, Shaw G M, Hahn B H, Kepler T B, Korber B T, Kwong P D, Mascola J R, Haynes B F. Co-evolution of a broadly neutralizing HIV-1 antibody and founder virus. Nature 2013; 496:469-76.

6. Morris L, Chen X, Alam M, Tomaras G, Zhang R, Marshall D J, Chen B, Parks R, Foulger A, Jaeger F, Donathan M, Bilska M, Grey E S, Abdool Karim S S, Kepler T B, Whitesides J, Montefiori D, Moody M A, Liao H X, Haynes B F. Isolation of a human anti-HIV gp41 membrane proximal region neutralizing antibody by antigen-specific single B cell sorting. PLoS One 2011;6:e23532.

7. Zhou T, Zhu J, Wu X, Moquin S, Zhang B, Acharya P, Georgiev I S, Altae-Tran H R, Chuang G Y, Joyce M G, Do K Y, Longo N S, Louder M K, Luongo T, McKee K, Schramm C A, Skinner J, Yang Y, Yang Z, Zhang Z, Zheng A, Bonsignori M, Haynes B F, Scheid J F, Nussenzweig M C, Simek M, Burton D R, Koff W C, Mullikin J C, Connors M, Shapiro L, Nabel G J, Mascola J R, Kwong P D. Multidonor analysis reveals structural elements, genetic determinants, and maturation pathway for HIV-1 neutralization by VRC01-class antibodies. Immunity 2013; 39:245-58.

8. Lynch R M, Tran L, Louder M K, Schmidt S D, Cohen M, Dersimonian R, Euler Z, Grey E S, Abdool K S, Kirchherr J, Montefiori D C, Sibeko S, Soderberg K, Tomaras G, Yang Z Y, Nabel G J, Schuitemaker H, Morris L, Haynes B F, Mascola J R. The Development of CD4 Binding Site Antibodies During HIV-1 Infection. J Virol 2012; 86:7588-95.

9. Leroux-Roels I, Koutsoukos M, Clement F, Steyaert S, Janssens M, Bourguignon P, Cohen K, Altfeld M, Vandepapeliere P, Pedneault L, McNally L, Leroux-Roels G, Voss G. Strong and persistent CD4+ T-cell response in healthy adults immunized with a candidate HIV-1 vaccine containing gp120, Nef and Tat antigens formulated in three Adjuvant Systems. Vaccine 2010; 28:7016-24.

### Example 7

### Longitudinal Antigenic Sequences and Sites (LASS): Computational Methods to Characterize Positively Selected Sites and Select Variant Sets for Reagent Design from Serial Samples

Abstract

One strategy for studying broadly neutralizing antibody (bnAb) development is to characterize the coevolution of virus and B-cell clonal lineages during affinity maturation and the development of neutralization breadth. Such longitudinal bnAb studies involve sequencing hundreds to thousands of Envelope (Env) variants from one donor. It is feasible, however to construct Envs reagents for protein expression and detailed analysis for only a fraction of these. Presented herein is a method to select a subset of variants that represents the gradual acquisition of selected mutations from among longitudinal sequences. It uses loss of the transmitted/founder (TF) virus, or the consensus of the first time point in the case of subjects that are enrolled during chronic infection, to identify sites that under strong positive selective pressure. Visualization tools have been developed to readily track mutations in these sites over time. An algorithm then is used to select Envs that represent the gradual acquisition of all recurrent mutations in the selected sites, sampling them in the context that they first appear in the subject. A detailed example of a retrospective application of this method to a subject, CH505, who has already been extensively studied, is provided to enable the assessment of how the method performed. Using 398 single-genome amplification (SGA)-derived Envs that spanned three years of infection, the algorithm identified 35 sites under putative immune selection. Encouragingly, these sites corresponded to verified immune targets: a T cell epitope, and epitopes recognized by neutralizing antibodies isolated from CH505: the CD4bs and the V3 loop. Thus, in this case patterns of mutations identified to be under selection were directly indicative of the antibody specificities of the subject. The algorithm identified 54 Envs that represent all recurrent mutations in selected sites. The Envs were well dispersed throughout the phylogeny, and represented the development of binding and neutralization in a set of 135 previously handpicked Envs. The algorithm chooses sequence sets with more recurrent mutations and less redundancy than would be chosen randomly or by hand. Thus, the algorithm objectively provides a minimal, manageable number of Envs to represent diversity in natural infection, to help study virus-antibody coevolution. This minimal representation of antigenic diversity is called an “antigen swarm.” Initially, this was developed as a strategy to explore mutational patterns and for reagent design. However, given the emergence of new vaccine technologies that may enable the use of high valency antigen cocktails, this approach could also be used to design a vaccine that mimics viral evolution in an individual who made potent bnAb responses.

Genetic sequencing of samples collected over time gives a dynamic view of how viruses evade host immunity while maintaining replication fitness. HIV-1 is a chronic infection, and persists as a diverse and evolving swarm of viral variants within an infected individual. HIV has a high mutation rate, and viral fitness is achieved by selection in an ever-changing immunological landscape within the host. Identifying mutations essential for immune escape, and simultaneously, those that may be important eliciting subsequent immune responses, can be biologically and computationally challenging. Given current state-of-the-art experimental practice, far more viral sequences can be obtained from a subject who is studied over time than can be cloned into constructs suitable for testing and analysis, necessitating down-selection for reagent design. Historically, this has typically been performed by inspection, often by picking some designated number of variants, (based on resources that can be applied to the problem), that represent different time points and different clades within a phylogenetic tree. Such strategies may miss the most relevant mutations, and may have large genetic distance between key variants. A computational strategy has been developed, working only from initial viral sequence data, to identify and visualize viral protein evolutionary “hot spots” and then to select compact virus sets that carry all candidate immune escape mutations. By inference, these sites might also be key in eliciting the ever-broadening immune response. This method uses the loss of the TF form of the virus as a measure of positive selection driven by immune response. An algorithm then chooses sequence variants that represent all recurrent amino acid mutations at each of the selected sites. By capturing each selected mutation as it first arises in the context of earlier and less divergent viruses, the algorithm captures the observed gradual accumulation of mutations as they arise. Such epitope diversification in vivo is associated with the development of a broadening immune response. Adjusting parameters fine-tunes how many sequences result. An advantage of the method is that to minimize costs, no more sequences are chosen than are necessary to represent the composite of variants detected. Use on a well-characterized set of Env sequences from a bnAb individual confirmed that the selected sites were concentrated in antibody contact areas, and that selected sequences represented diverse antigenic phenotypes. Such a compact set of variants is referred to as an antigen swarm, and suggest potential use of antigen swarms for reagent design, to characterize the evolving antibody responses, as well as for an antigen swarm vaccine.

Introduction

It is not yet known how to stimulate protective immunity against HIV-1 with broadly cross-reactive neutralizing antibodies (nAbs) via vaccination, and neutralizing antibody induction remains a central focus of HIV vaccine field. Neutralizing antibodies are immune correlates of protection in all antiviral vaccines licensed to date [1, 2], and administration of neutralizing antibodies can confer protection in SHIV challenge models in rhesus macaques [3, 4]. During the natural course of HIV infection, a single transmitted-founder (TF) virion typically establishes infection, and the virus population grows exponentially, with random mutations that initially follow a Poisson distribution of intersequence distances [5, 6]. The viral load eventually declines and resolves to a quasistationary set-point [7], influenced by both host and viral factors [8]. HIV is maintained as a continuously evolving population throughout chronic infection [9], with diversification driven by adaptive immune responses, including both antibodies [10-18] and T cells [19-21]. Mutations that facilitate immune evasion are positively selected and become more common, while mutations that result in a relative fitness disadvantage do not persist. Neutral mutations may also drift to higher frequency, with rates that depend on the effective population size [22].

Previous studies have revealed that viral diversification precedes the acquisition of breadth, which suggests antigenic diversity may be necessary for acquisition of bnAb breadth in vivo [15, 18], and also that several antibody lineages can concurrently impact selection on the same epitope region [16]. While essentially all HIV-1 infected individuals can elicit antibodies with some cross-reactive neutralization responses during the chronic phase of infection, and neutralization responses vary over a continuous spectrum across individuals [17]. Plasma samples from individuals with the most potent and broad antibody neutralization are frequently singled out for detailed study [23-26]. Such study includes isolation of monoclonal antibodies and investigations of both viral and B cell lineages to understand the immunological processes underlying elicitation of effective immune responses and inform strategistudyes for vaccine design [15, 16, 18, 27-29]. In general, autologous, strain-specific nAbs begin to develop in the initial months after infection, and rapidly select for viral escape variants [11, 14]. High titers of more broadly neutralizing antibodies develop in a subset of cases, but only after years of infection, and perhaps more in cases with persistently high levels of viral replication [30, 31].

Among the subjects with broad cross-reactivity characterized to date, the contemporary co-circulating autologous virus has escaped from an otherwise broadly-reactive neutralizing antibody response [32]. Antibodies that recapitulate much of the potency and breadth of polyclonal sera have been cloned from subjects with high bnAb titers [cite]. The developmental pathway of B cell immunoglobulin genes from early to later infection is an active research frontier, now only beginning to be understood [cite]. It remains unknown what properties of evolving viral Env proteins stimulate or facilitate the important transition from autologous to heterologous reactivity. Understanding these events should ultimately enable new strategies for immunogen design to elicit potent, broadly cross-reactive nAbs.

A continuing research priority has been to characterize virus co-evolution with antibodies in individuals who develop the greatest potency and breadth of neutralization [15, 16, 18, 29, 33, 34]. Working back from mature bnAb clonal lineages, through ancestral intermediates, ultimately to the unmutated germline precursor, has begun to help understand this process of bnAb development [15, 18, 27, 28, 33, 35-37]. To explore antibody/viral co-evolution, mutational patterns that are selected over time in both the antibody population, as it undergoes affinity maturation, and the virus population, as it evolves to evade the ongoing immune responses, are defined by sequencing and sequence analysis of serially obtained, or longitudinal, samples [15, 18].

Described herein is a new approach to such longitudinal sequence analysis, which involves two steps. The first part of our bioinformatics approach allows one to define and visualize sites that are under positive selective pressure in the viral population. Defining the sites under selective pressure can help infer the antibody specificities that are active in the plasma, and to identify key mutations for characterization during experimental follow-up studies. The second part of the approach is a computational method to down-select sequences objectively from a very large sequence sample, yielding a representative subset of viral variants. The resulting set of sequences is an “antigenic swarm,” which captures mutations in the sites that are under the most potent selective pressure as they first emerge in the evolving HIV-1 quasispecies [15, 18, 29]. The size of the sequence subset involves a trade-off between the cost of including more variants and the degree of selection to be represented. Our approach involves two parameters that can be adjusted to balance these two factors, explore the data, and choose the most representative set given experimental feasibility and sample-size limitations. This process has been worked through retrospectively in individual CH505, where extensive information regarding antibody interactions and targeted Env epitopes [15, 16] is available, to determine how well the relevant diversity is captured by our informatics method in this case. This approach can be used to select Envs (or similarly diversifying variants) as reagents, e.g. for Env production for synthesis and use in binding assays, or to generate pseudoviruses for use in neutralization assays. In turn, the resulting reagents can be used to study relationships between viral phenotype and genotype, and to investigate in better detail how neutralizing antibody responses develop by affinity maturation.

Resulting sets of antigens provide a useful baseline for basic research and may also inform immunogen design. A working hypothesis to explain the observation that bnAbs tend to arise late infection, after antigenic diversification has arisen in the subject, is that serial immune escape in vivo drives antibody lineages to adapt to the emerging viral variants, eventually enabling recognition of the diverse forms of the targeted epitope found in the circulating population. Thus, a polyvalent vaccine that represents Env diversity may be one strategy for inducing antibodies with greater breadth than single, invariant clonal antigens. Related work in vaccine design against HIV-1 has suggested that Env variants sampled during development of heterologous neutralization breadth could be administered as immunogens [38, 39]. Described herein is a a method for Env selection to ensure comprehensive representation of antigenic diversity.

Results

A process for antigenic swarm selection has been implemented, which consists of two phases. The first phase identified protein sites most likely to be under positive (diversifying) selection, by considering the extent to which the TF amino acid is “lost” at any one time-point during the longitudinal sampling period. This yielded a list of sites of interest, from which all amino acid mutations that arose over time were tabulated. The second phase selected sequences that represented the mutational variants among sites selected in the first phase. The two phases of analysis, and parameters that influenced the number of sites and sequences thereby obtained, are detailed below.

It is worth noting that the single-genome amplification (SGA) sequences analyzed here were obtained by limiting-dilution PCR, which provides genetic linkage across all of the env gp160, without recombination artifacts, and limited nucleotide substitution errors in cDNA synthesis [40]. Unlike Sanger sequencing from bulk PCR or large numbers of fragmentary high-throughput reads from unlinked templates, SGA sequences provide high-quality sequence data ideally suited to understand how viruses adapt to host immune responses over time [5, 14, 21, 40, 41]

Site Selection

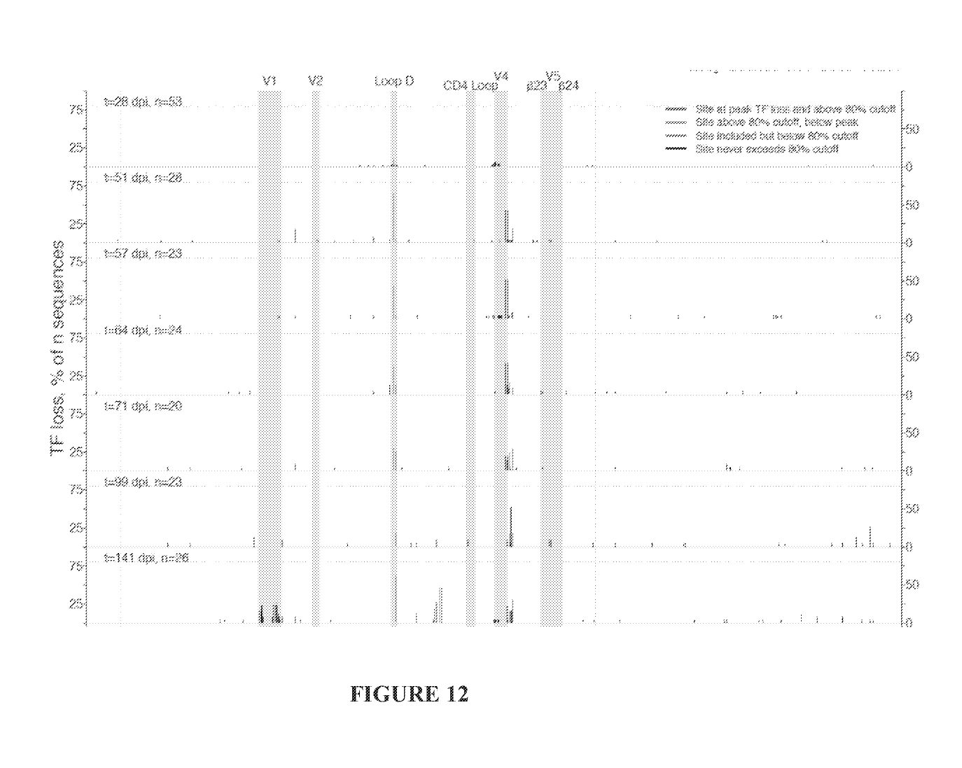

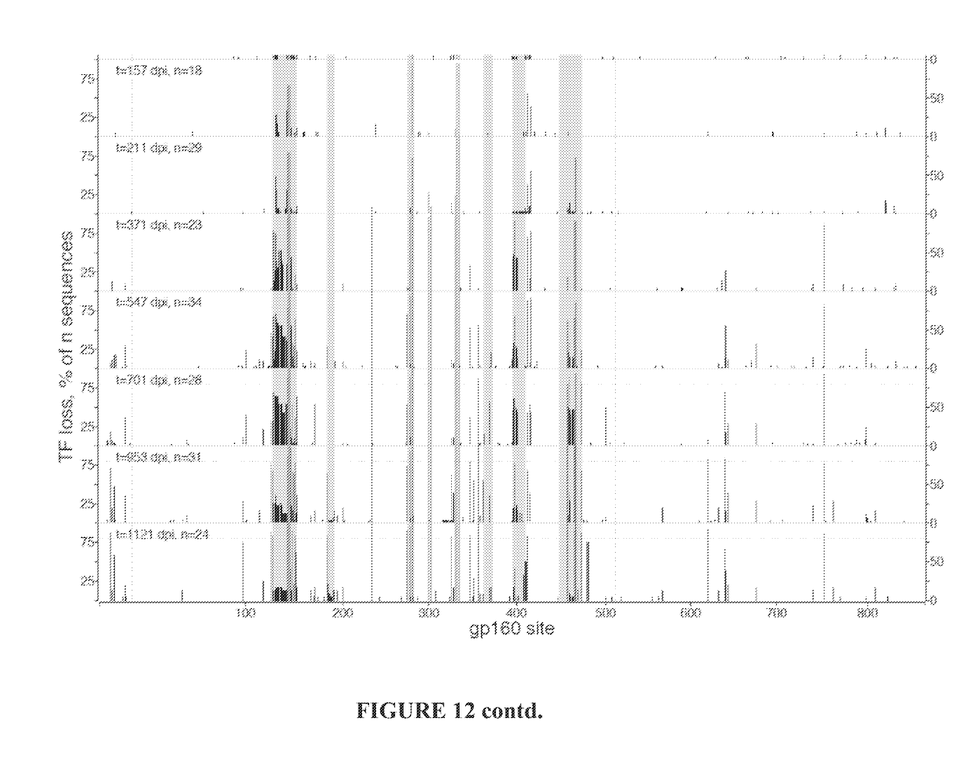

TF loss varied across sites. Building upon recent studies of antibody/Env coevolution in the study subject CH505 [15, 16], first the strategy was applied to this subject to determine how well the method performed in a case where key epitopes have been defined and well characterized. 398 sequences from 14 time-points sampled over three years were aligned across 953 sites in the Env protein. FIG. 12 depicts TF loss per site for each time-point sampled, from week 4 through week 160 post-infection. Clearly, most sites show little or no TF loss. Sites with high levels of TF loss are candidates for key escapes due to immune selection. Because an insertion or deletion was counted relative to the TF virus as a change, rather than a missing datum, the hypervariable regions of V1, V2, V4, and V5 also showed TF loss, largely due to length variation. TF loss was used to list sites where frequency of the TF form fell below a fixed cutoff percentage. The cutoff is a parameter that can be adjusted as needed, as described below.

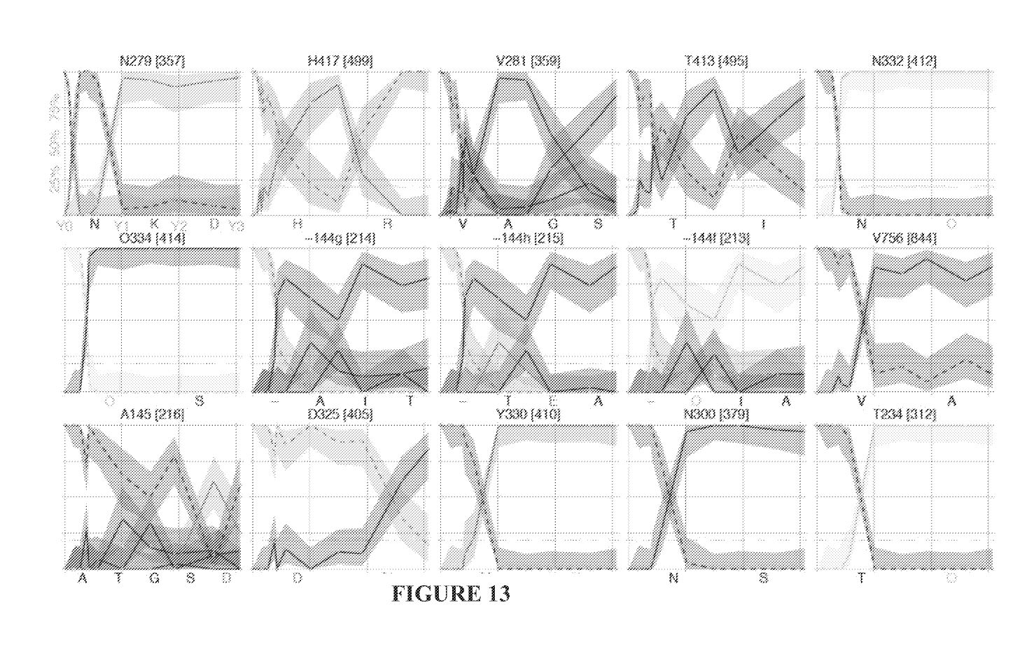

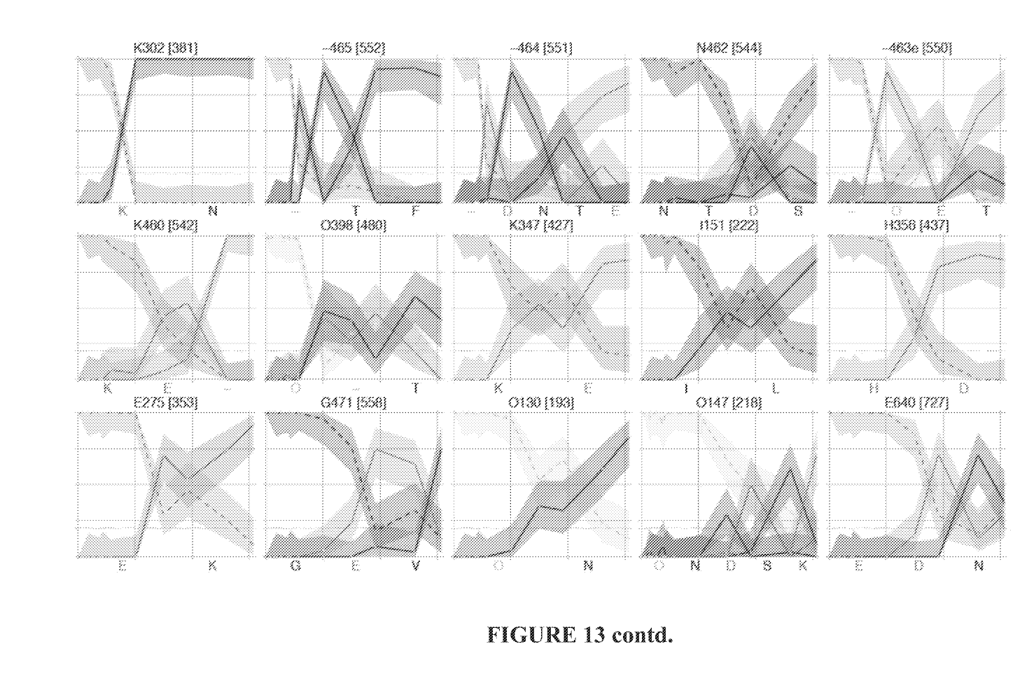

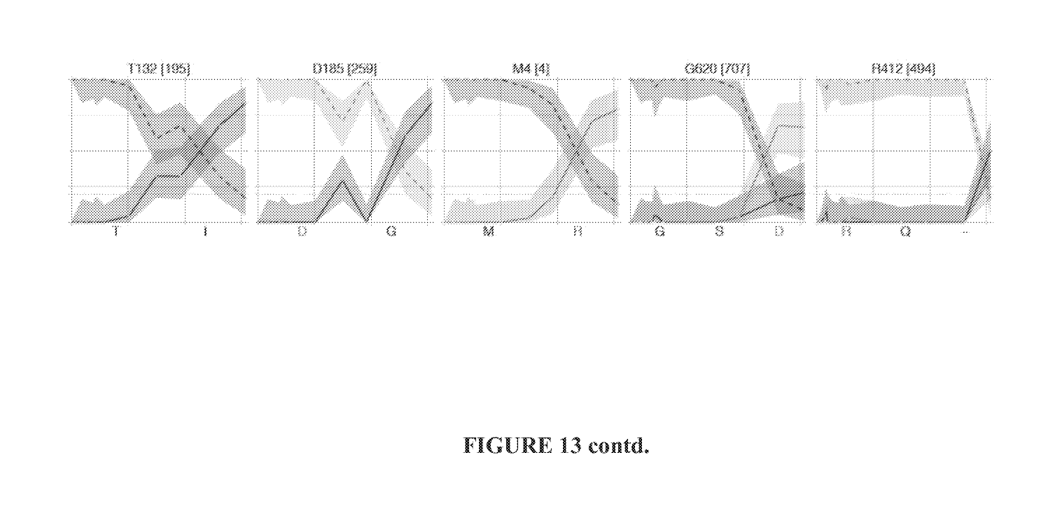

Initially dominated by the TF form, mutational variants developed over time, and displayed a variety of dynamics among sites with high TF loss. FIG. 13 depicts evolution of variant frequencies in subject CH505. All 35 sites shown in this figure had over 80% TF loss in at least one time point. The rate of TF loss was lower in some sites than in others. Such slow transitions could reflect the evolving immune response and newly arising selective pressure. In qualitative terms, there were four dynamic categories, designated i-iv. First (i), some sites showed replacement of the TF form with another single mutational variant, whether fast or slow, e.g. the V3 glycan shift at sites 332 (top-right panel “N332” in FIGS. 13) and 334. Next (ii), in some sites, TF replacements were followed by secondary mutations. For example, site 279, located in Loop D, was initially an asparagine, but a transient lysine mutation yielded to an aspartic acid after transient reversion to the TF asparagine (FIG. 13, top-left). Third (iii), some sites reverted to the TF form after high TF loss. For example, site 417, initially histidine, is predominantly an arginine from about six months to nearly two years after infection, but then reverts to the ancestral histidine. Finally (iv), some sites exhibited sustained polymorphisms. These were particularly common in hypervariable loops, where distinct subpopulations carried divergent forms. Simple shifts, like (i) above, were the most common form of TF loss. Serial mutations, like (ii) above, were also common and could be the direct result of serial escape, due to new pressures imposed by adaptation of the evolving antibody response to an initial escape mutation, driving continued selection. Alternatively, serial replacements could result from complex interactions with multiple antibodies in a polyclonal response [16], or pressures resulting from balancing fitness costs and/or compensatory mutations in a changing evolutionary milieu. Transient losses reverting to the TF form were rare, and different underlying reasons for this pattern could be at play, such as a fitness cost for a mutation that was carried along with a neighboring mutation, or a changing immunological environment in the host, which could transiently favor a mutation with a modest fitness cost [14, 42-44]. Sampling twenty or more sequences per time-point (median 25, range 18-53) across 14 time-points gives a sample size sufficient to detect uncommon variants; 95% confidence intervals on variant frequencies show similar dynamics with sampling uncertainty considered (FIG. 13).

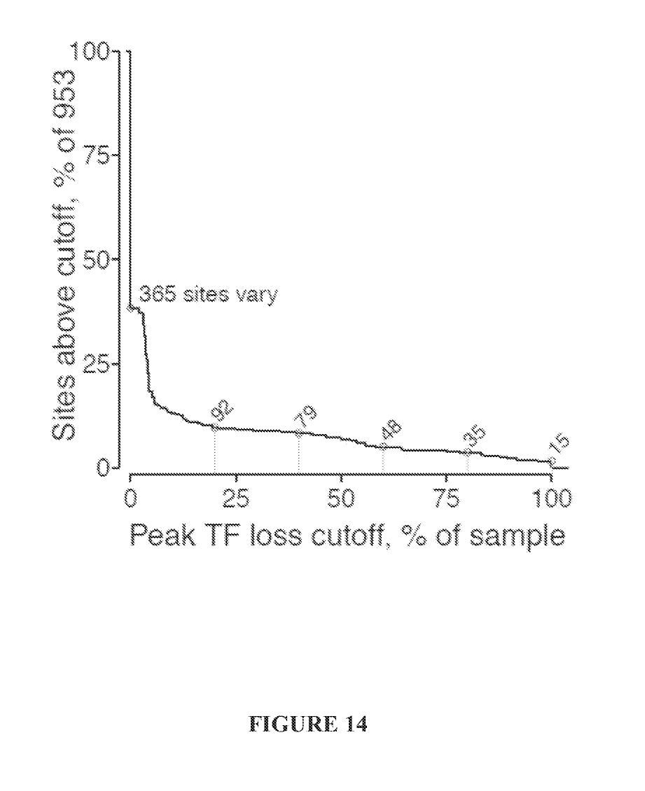

Peak TF loss identified selected sites. The “peak” TF loss per site was defined as the highest TF loss in that site over all time-points sampled, and it was used to select candidates for sites under immune selection. In all, 15 sites completely lost the TF form during the three-year sampling period, while the other sites never reached 100% TF loss. The cumulative distribution of peak TF loss per site, depicted in FIG. 14, indicated that one-third of sites were strictly invariant and 64 sites lost over 50% TF. From this distribution, 35 sites with at least 80% peak TF loss for were selected further study and Env selection. The choice of an 80% cutoff might have been different for other data (addressed below) or for use in different contexts. Increasing the TF loss cutoff decreases the number of sites selected, and working with other cutoff values produces more or less selected sites for subsequent investigation, to be chosen in light of available resources.

Selected sites were consistent with antibody-driven selection. As illustrated in FIG. 13, the time at which each selected site started to emerge in the sampled virus population varied from one site to another. The cumulative amount of TF loss also varied, and was zero in sites that never changed. Cumulative TF loss had a simple geometric interpretation as the area above the dashed TF line in the plots of frequency over time that appeared in FIG. 13. Its calculation weighed TF loss for two consecutive samples by the amount of time elapsed between when the two samples were drawn. Cumulative TF loss was lower in sites that reverted to the TF form than in sites that quickly mutated away from the TF and never reverted. Sites were sorted by these two criteria; time to initial TF loss and cumulative TF loss, to obtain an informative representation of the accumulation of mutations among selected sites.

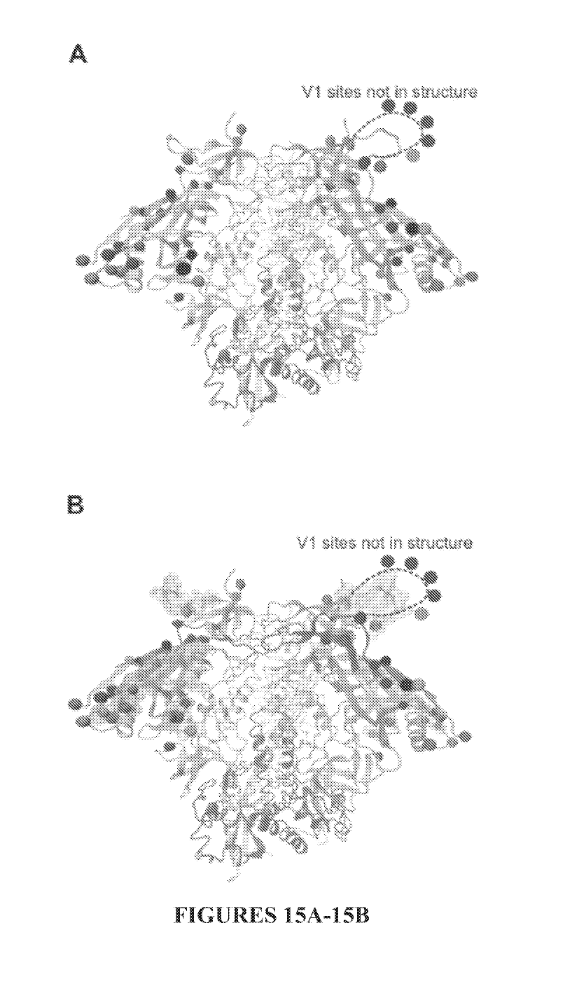

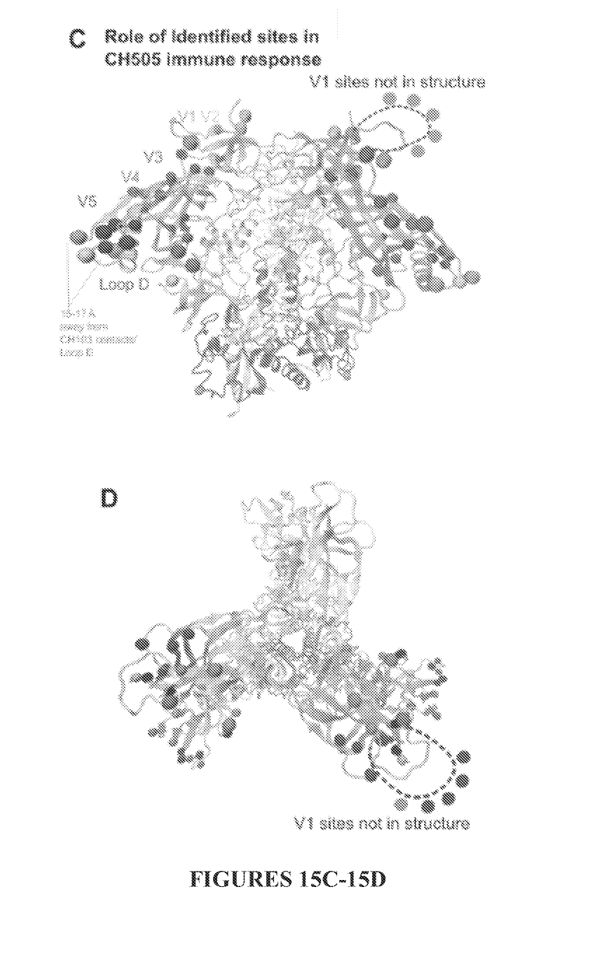

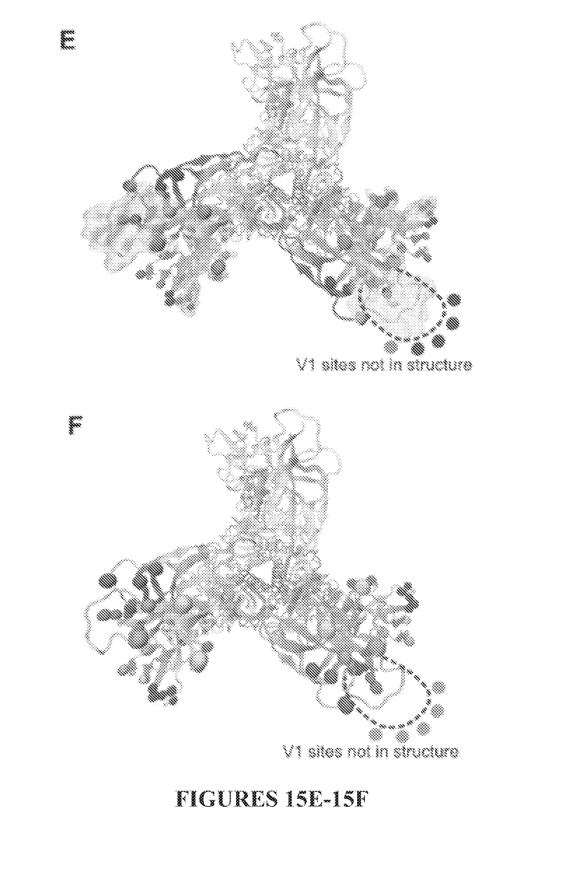

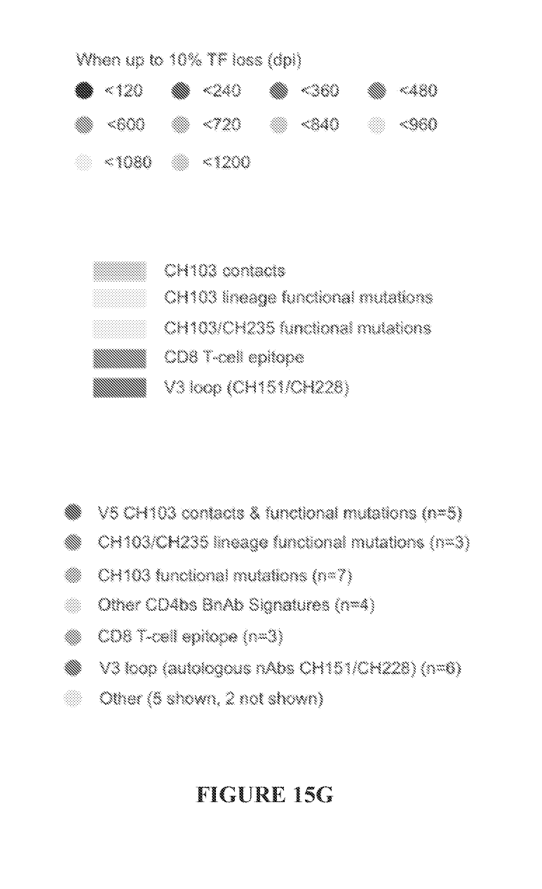

Table 2 lists the 35 selected sites with 80% TF loss, ranked by the earliest time at which any non-TF variant exceeded 10%, with ties resolved by cumulative TF loss sorted in descending order. Most (91%) of the selected sites occurred in gp120. In the context of the Env trimer structure, the selected sites formed localized clusters on the outer domain of gp120 (FIG. 15). The clustered patches of selected sites on gp120 corresponded to the three known immunological pressure regions in subject CH505. The first cluster of three selected mutations (412, 413, 417) was in a CTL epitope that was recognized early in infection in CH505, and so conferred CTL escape [16]. The second cluster of six selected sites (300, 302, 325, 330, 332, 334) was located within the V3 loop, or in the glycosylation site at its base. Two autologous neutralizing anti-V3 antibodies, DH151 and DH228 were isolated from subject CH505. Thus, some of these sites may be relevant to this lineage [45].

TABLE 2
Selected sites. CH505 Env sites with at least 80% TF loss in at least one time-point. The symbol
color in the left-most column indicates the appearance of each selected site in FIG. 15.

| | Peak | When | | Immune | |
|---|---|---|---|---|---|
| Site | loss | up | Rank | pressure | Notes |
| 4 | 87.5 | d701 | 33 | na | Signal peptide |
| 130 | 87.5 | d547 | 28 | CD4bs | PNG site at base of V1, near VRC01 contact [47] |
| 132 | 83.3 | d547 | 31 | CD4bs | V1 indels cause CH103 resistance [16] |
| 144f | 100 | d141 | 9 | CD4bs | V1 indels cause CH103 resistance [16] |
| 144g | 100 | d141 | 7 | CD4bs | V1 indels cause CH103 resistance [16] |
| 144h | 100 | d141 | 8 | CD4bs | V1 indels cause CH103 resistance [16] |
| 145 | 96.8 | d141 | 11 | CD4bs | V1 indels cause CH103 resistance [16] |
| 147 | 91.7 | d547 | 29 | CD4bs | V1 indels cause CH103 resistance [16] |
| 151 | 83.3 | d371 | 24 | CD4bs | V1 indels cause CH103 resistance [16] |
| 185 | 83.3 | d547 | 32 | CD4bs | Signature site for CD4bs bnAb b12 [51] |
| 234 | 100 | d211 | 15 | CD4bs | Signature site for CD4bs bnAbs VRC01 & NIH45-56 [51] |
| 275 | 91.7 | d547 | 26 | CD4bs | Loop D, CH103 contact, CH235 resistance [15, 16] |
| 279 | 95.8 | d28 | 1 | CD4bs | Loop D, CH235 resistance, CH103 sensitivity [15, 16] |
| 281 | 100 | d64 | 3 | CD4bs | Loop D, CH235 resistance, CH103 sensitivity [15, 16] |
| 300 | 100 | d211 | 14 | V3 loop | V3 autologous nAb in CH505 [45] |
| 302 | 100 | d211 | 16 | V3 loop | V3 autologous nAb in CH505 [45] |
| 325 | 83.3 | d141 | 12 | V3 loop | V3 autologous nAb in CH505 [45] |
| 330 | 100 | d157 | 13 | V3 loop | V3 autologous nAb in CH505 [45] |
| 332 | 100 | d141 | 5 | V3 loop | V3 autologous nAb in CH505 [45] |
| 334 | 100 | d141 | 6 | V3 loop | V3 autologous nAb in CH505 [45] |
| 347 | 83.3 | d371 | 23 | CD4bs | 15-17 Angstroms from CH103 contacts |
| 356 | 100 | d547 | 25 | CD4bs | Adjacent to CD4bs bnAb 12A12 signature [51] |
| 398 | 91.3 | d371 | 22 | CD4bs | 15-17 Angstroms from CH103 contacts |
| 412 | 83.3 | d121 | 35 | CTL epitope | CTL epitope V4 loop [16] |
| 413 | 88.2 | d64 | 4 | CTL epitope | CTL epitope V4 loop [16] |
| 417 | 91.2 | d51 | 2 | CTL/CD4bs | CTL epitope V4 loop/CD4bs bnAb b12 contact [16] |
| 460 | 100 | d371 | 21 | CD4bs | V5, CH103 contact region, resistance [15, 16] |
| 462 | 89.3 | d211 | 19 | CD4bs | V5, CH103 contact region, resistance [15, 16] |
| 463e | 100 | d371 | 20 | CD4bs | V5, CH103 contact region, resistance [15, 16] |
| 464 | 100 | d211 | 18 | CD4bs | V5, CH103 contact region, resistance [15, 16] |
| 465 | 100 | d211 | 17 | CD4bs | V5, CH103 contact region, resistance [15, 16] |
| 471 | 87.5 | d547 | 27 | CD4bs | CH103 contact [16] |
| 620 | 91.7 | d953 | 34 | na | gp41 |
| 640 | 83.9 | d547 | 30 | na | gp41 |
| 756 | 92.9 | d141 | 10 | na | gp41 cytoplasmic tail |

The third cluster, in the CD4bs, is the most complex. The CD4bs is the target of both the CH103 bnAb lineage [15] and the CH235 nAb helper lineage [16] in subject CH505. Many of the 32 selected gp120 sites included structurally defined contacts for CD4 [47, 48], and several previously studied CD4bs bnAbs, including VRC01 [47, 48], NIH45-46 [49], and b12 [50]. Although the current study is retrospective, this pattern of mutations would have indicated the presence of CD4bs antibodies in the subject, as well as indicate when they were beginning to exert selective pressure, even prior to isolation of nAb lineages. As expected, CH103 contacts and resistance mutations were well represented among the selected sites [16]. Three selected sites (279, 281, 275) were localized to CH103 light-chain contacts near loop D (FIG. 15B), a region that rapidly accumulated mutations as a result of escape from the autologous CD4bs neutralizing antibody CH235; these mutations rendered the virus more susceptible to the CH103 early lineage members. Six CH103 heavy-chain contacts (FIG. 15C) in and near V5 (460, 462-465, 471) were also among the selected sites, and mutations in this region conferred CH103 resistance. V1 loop mutations also conferred CH103 resistance, and seven sites in V1 were among the 35 selected sites (132, 144f, 144g, 144h, 145, 147, and 151). Three of these were inserted together in V1 after position 144. Finally, five additional selected sites are known to be important for other CD4bs bnAb interactions, providing indirect evidence that they may be important for either CH103 or CH235. These are: 417, a contact for the CD4bs bnAb b12 [50], and 185, a V2 region signature sites for b12 [51]; 234, a signature site for CD4bs bnAbs VRC01 and NIH45-46 that is near Loop D [51]; the glycosylation site N130, adjacent to a VRC01 contact [47]; and position 356, adjacent to a 12Al2 signature [51]. The selected sites that were relevant to other antibodies noted above were identified using the Los Alamos HIV-database genome browser and CATNAP tool (hiv.lanl.gov). Thus 29 of the 35 selected sites, or 83% are related to the three epitopes that were functionally defined in this subject, despite these sites being simply and objectively identified based solely on the TF loss criterion (Table 2). Of the six sites that were not directly related, three were gp41 sites (620, 640, 756) and one was in the signal peptide (position 4). The other two (398 and 347) were clustered near position 356 in gp120, and all three were near but not in the CH103 contact region (indicated in FIG. 15C as 10-17 Angstroms away from CH103 contacts).

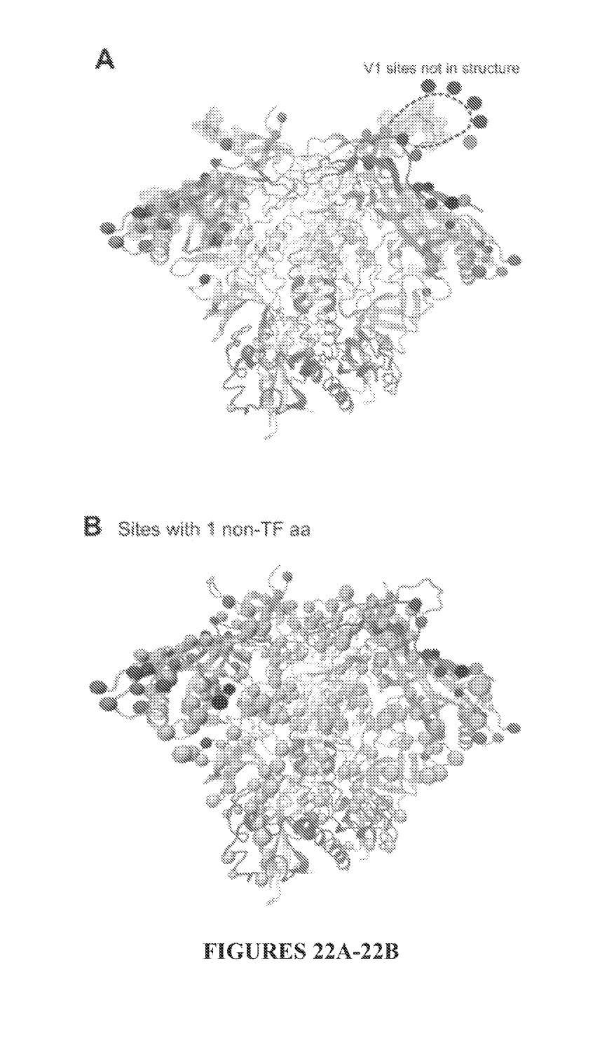

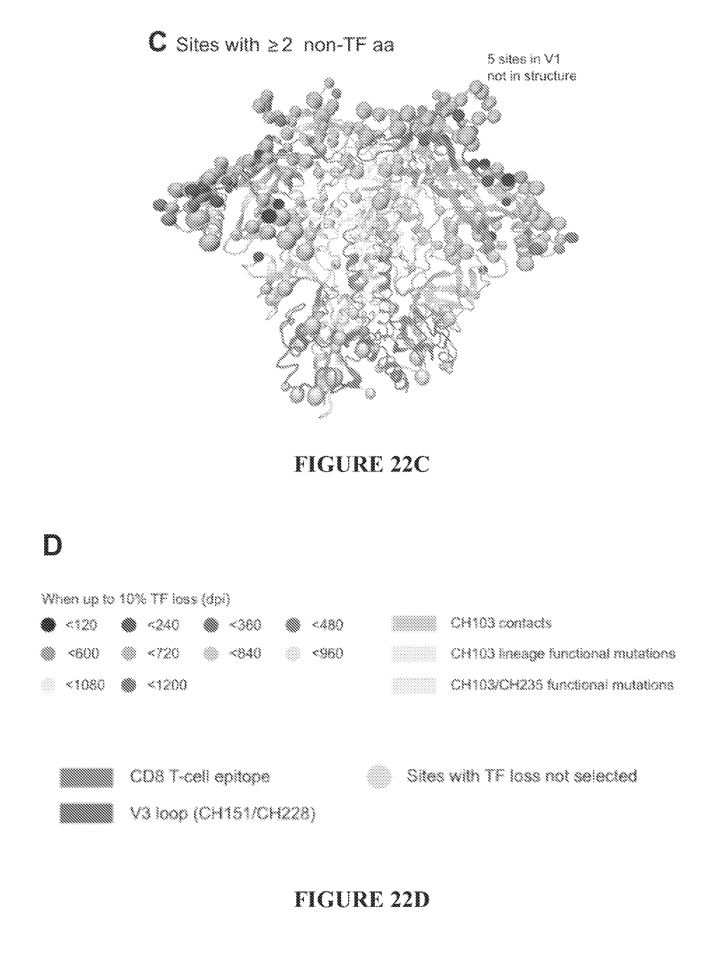

To consider what sites might be missed by the TF-loss criterion, the localization of sites that never reach 80% loss was explored. The 365 sites that varied were dispersed over the entire protein, as expected. Requiring multiple mutations among all 398 available sequences, regional patterns appeared in the spatial distribution of mutations (FIG. 22). Positions with three or four mutations began to show a clear focus towards immunologically targeted regions. This suggests that high TF loss may exclude mutations that occur in immune-targeted regions, and mutations in these sites may have phenotypic consequences for immunological sensitivity. Several localized clusters of sites were apparent, which may indicate other antibody targets (FIG. 22), whether transient, weakly selected, or just beginning to show selection at the end of the study period. However, these sites were not under the same high degree of selective pressure as sites in which the TF form was depleted. LASS allows investigators to target the most highly selected sites for further study, and adjust the threshold according to what is practical for reagent design.

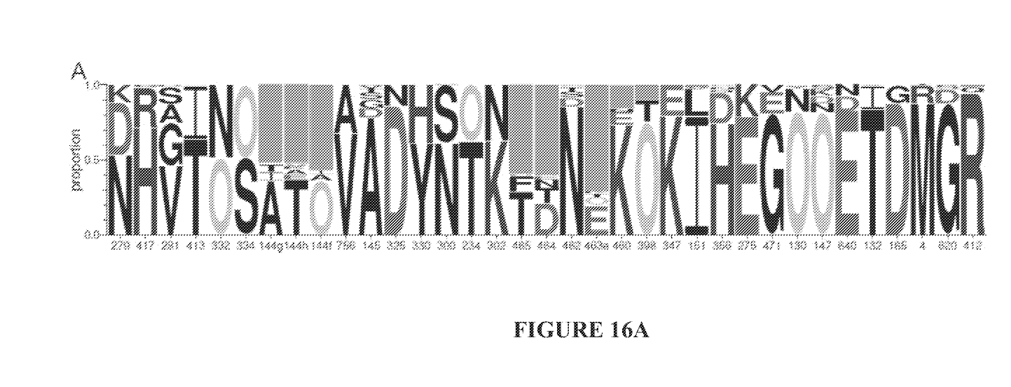

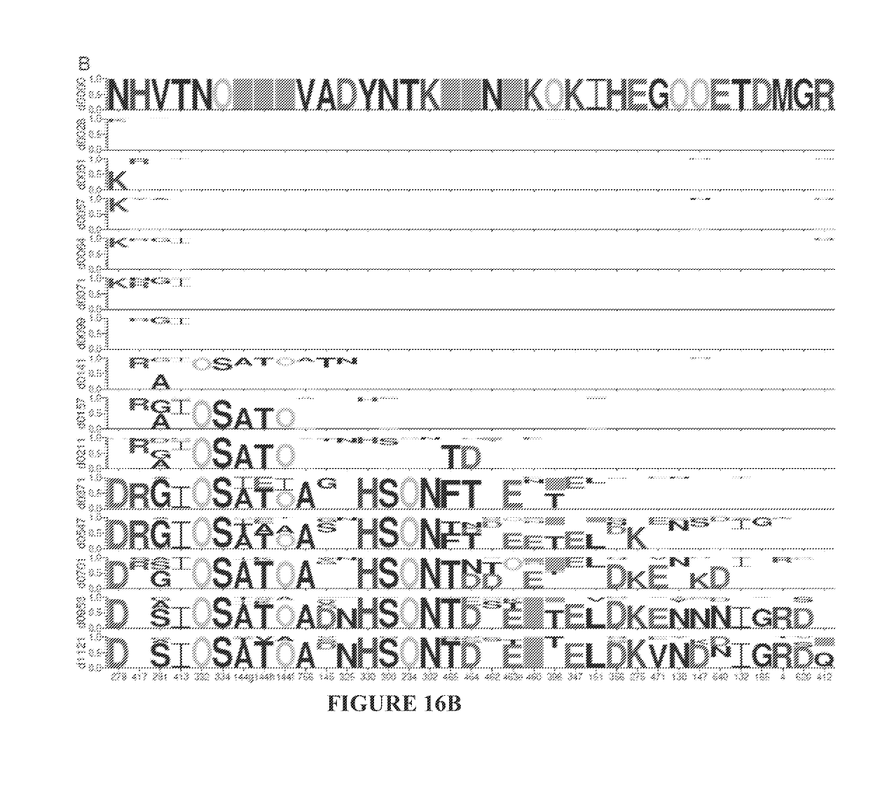

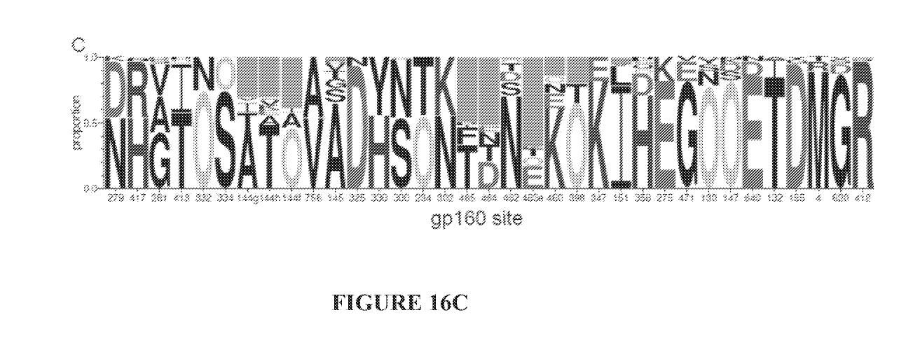

A concise representation of selected sites was to string them together to form “concatamers” of 35 amino acids. The order of sites in concatamers did not follow the primary Env sequence, but rather by when non-TF mutations first emerged and cumulative TF loss suggested a cumulative progression of mutations. Using modified sequence logos [52, 53], in which symbol height indicates frequency in a sample, shows this progression over time clearly (FIGS. 16A-16C). The top row (FIG. 16A) summarizes mutation frequencies from all 398 Envs sequenced over the first three years of infection in this individual. Below that (FIG. 16B), rows were stratified to summarize frequency in each sample, first for the TF virus (day 0), then for 14 plasma samples (day 28 through day 1121, i.e. week 4 through week 160, post-infection). To facilitate comparison, only non-TF mutations appear in these rows, TF frequencies are left blank, and alignment gaps are included as grey bars to highlight insertions and deletions.

Electrostatic charges of amino acid side chains, depicted by symbol colors (FIG. 16), changed polarity in 25% of the gp120 sites (279, 144h, 463e, 460, 347, 356, 275, 147) but not in gp41 sites. Gain or loss of N-linked glycosylation motifs appeared in 13 of the 32 (41%) gp120 sites but none of the gp41 sites. The representative sequences selected by the next stage of analysis were also depicted in this manner (FIG. 16C).

Swarm Selection

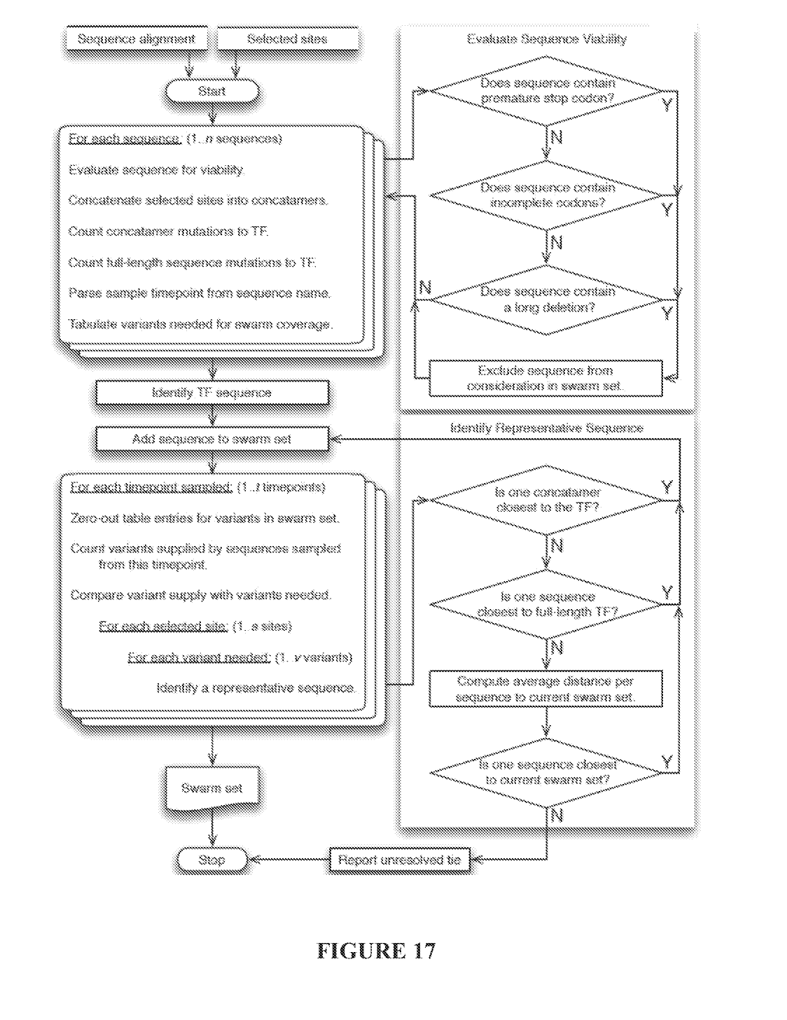

Algorithm. The swarm-selection algorithm is outlined as a flow-chart in FIG. 17. After the initial definition of sites deemed to be under selective pressure, based on the loss of the TF amino acid over time, it then consists of two passes through the sequences. The first pass (top half of FIG. 17) tabulates all mutations in the selected sites, among all available sequences. Mutations that only ever occur once in the full data set (or some other number of times, specified by the user) are omitted. This table is used on the second pass to keep track of which mutations have been included in the growing swarm. The second pass (lower half of FIG. 17) iterates over the time-points sampled, starting with the earliest time point, to include any mutations listed in the table. When a sequence is added, the table is updated to indicate the mutations that it carries have been included.

The algorithm is deterministic, meaning it will always produce the same set of sequences from a given alignment, because the algorithm does not make any random choices, and does not depend on the order in which sequences are provided in the input alignment. The algorithm was made efficient through use of vector operations and computes distance matrices only when they are needed to choose between otherwise ambiguous alternatives. Its computational complexity is expected to require no worse than a linear increase with the number of sequences in the input alignment. That is, doubling the number of input sequences should no more than double the expected run time.

Each mutation observed more than once in a selected site will ultimately be included in the antigenic swarm. The algorithm isolated mutations of interest in the least divergent sequence background possible, among available sequences sampled. It did this by progressively covering mutations that occurred in selected sites in the first time-point they appeared, and by representing them with the sequence most similar to the TF or, to resolve ties, the sequence most similar to those under consideration (lower-right box in FIG. 17).

Objective choice of representative variants among selected sites. The algorithm identified 54 Envs that covered variant diversity at the 35 sites selected by TF loss. Table 3 summarizes these as concatamers. Algorithm selection criteria had at least two clear consequences. First, the gradual accumulation of mutations found in early infection was deliberately mimicked using this strategy. Second, the appearance of each new mutation of interest is, by design, relatively isolated from other accumulating mutations emerging in the within-host virus population. Therefore, to the extent possible given sampling, each mutation in each the selected sites will be expressed in a context as close as possible to the form of the Env in which it was embedded when it first began to appear in the viral population at a high enough level to be sampled. By using the antigenic swarm to characterize variation among neutralization phenotypes in the population, if a particular mutation conferred a phenotypic change in either antigenicity or neutralization susceptibility of an isolate, then that change would be identified relative to the other mutations naturally occurring in the sampled virus population.

TABLE 3
Selected Envs. Concatamers (35 sites with at least 80% TF loss) in antigenic
swarm of 54 Envs, selected to represent polymorphisms among 398 full-
length Envs from CH505. The sequences associated with the Genbank accession
numbers KC and KM are incorporated by reference.

| Name | Accession | Concatamer |
|---|---|---|
| w000.TF | KC247556 | NHVTNO---VADYNTK--N-KOKIHEGOOETDMGR |
| w004.31 | KC247583 | .....................-............. |
| w004.54 | KC247604 | K.................................. |
| w007.8 | KM284749 | KR................................. |
| w007.21 | KM284732 | ..................................Q |
| w007.25 | KM284734 | ............................N...... |
| w007.34 | KM284744 | ...I............................... |
| w008.20 | KM284762 | ..A................................ |
| w009.19 | KM284781 | ..G................................ |
| w010.7 | KM284714 | .N................................. |
| w020.15 | KC247489 | ....OS....T........................ |
| w020.11 | KC47485 | ..........TN....................... |
| w020.24 | KC247495 | .RA......AT........................ |
| w020.25 | KC247496 | .R....ATO.......................... |
| w022.6 | KC247523 | ..AIOSATO...H...................... |
| w022.5 | KC247522 | .RA.OSATO....S..................... |
| w022.9 | KC247525 | ..GIOS.................-........... |
| w022.22 | KM284717 | ...O............................... |
| w030.20 | KC247541 | ..AIOSATOA......TNTO............... |
| w030.17 | KC247532 | D.GOOSATO.......TD................. |
| w030.21 | KC247535 | .RA.OSATO...........E.............. |
| w030.36 | KC247549 | ....OSATO.....O.TD................. |
| w030.26 | KC247539 | .RG.OS....-....NTD................. |
| w030.13 | KC247529 | ..DPOS............................. |
| w030.32 | KC247546 | .RG.OSATO.......TD-................ |
| w053.15 | KC247614 | D.G.OSATOA..HSONFT.E.-.L........... |
| w053.29 | KC247625 | .RAIOSATOA..HSONFT.E.-....E........ |
| w053.22 | KC247620 | DRGIOSIEIAG.HSONFT.E.TE............ |
| w053.8 | KC247632 | DRGIOSATOA..HSONFT.E.T..Q.......... |
| w053.31 | KC247628 | DRGIOSAT....HSON....N-............. |
| w053.9 | KC247633 | DRGIOSIEIAG.HSONFT.E.TE....N..I.... |
| w078.6 | KC247664 | DRGIOSOS.AS.HSONTN.OE-............. |
| w078.36 | KC247655 | DRGIOSTAAAS.HSON..S.O-.-....SD..... |
| w078.9 | KC247667 | DRG.OSTAA.S.HSONFT.E....QK..S...... |
| w078.26 | KC247645 | DRGIOSTAAAS.HSON..S.O.E.NK..S...... |
| w078.29 | KC247647 | DRGIOSTAAAS.HSONTN..-..-L..NS.A.... |
| w078.30 | KC247649 | ..A.OSATOA..HSON....N-......D...... |
| w078.33 | KC247652 | ..A.OSATOA..HSONT.O.N-.....N..I.T.. |
| w078.17 | KC247639 | DRG.OSATOA..HSONFT.EE..LDK.D..IG... |
| w078.15 | KC247637 | DRGIOSATOA..HSON..D..TEL.KES....... |
| w078.27 | KC247646 | DRGIOSATOA..HSONTDD..TEL.KES....R.. |
| w100.T3 | KC247401 | DRGIOSATO..NHSONTDD.ETEL.KEN..I.RS. |
| w100.B10 | KC247386 | DSG.OSATOA..HSONTDD....LDKEN..I.... |
| w100.B2 | KC247387 | .RAIOSIK.AG.HSON....N...D.V........ |
| w100.B4 | KC247389 | DRGIOSATO...HSON..S.O...DKE.K....D. |
| w100.A11 | KC247376 | D.S.OSATOA..HSONTNTOE-..D.E.KD..... |
| w100.A13 | KC247378 | ..A.OSATOAV.HSONTNTOE-.......D..... |
| w136.B10 | KC247404 | D.GIOSATOADNHSONTD.E-TELDKES.DIY.S. |
| w136.B5 | KC247429 | ..A.OSATOAV.HSONTESK-.E.O..Y.DI.... |
| w136.B2 | KC247411 | D.G.OSTVAA-.HGONIDOT--E.O.......RD. |
| w136.B23 | KC247414 | D.A.OSIK..G.HSONTEST-..VD....N...D. |
| w160.C1 | KC247465 | ..A.OSVTOAV.HSONTGST-...D..Y.D..TV. |
| w160.T3 | KC247482 | D.SIOSATOA.NHSONTD.E-TELDKVND.IGRD- |
| w160.T4 | KC247483 | D.A.OSTVA.S.HSONPD..-...G...DN..... |

Swarm variant frequencies (FIG. 16C) resembled variant frequencies sampled in the virus population (FIG. 16A), except for the deliberate inclusion of rare mutations at selected sites, which were less readily apparent in the larger population. Mutations seen only once among all of the sequences obtained were not required for inclusion, but all mutations in selected sites seen in two or more of all the sequences were represented by the 54 selected Envs. Mutations that occurred only once were not considered, as they are more likely to represent random mutations or sequencing error than mutations that occurred more frequently. The adjustment of this criterion to evaluate its effect on the number of Envs that were selected is discussed herein.

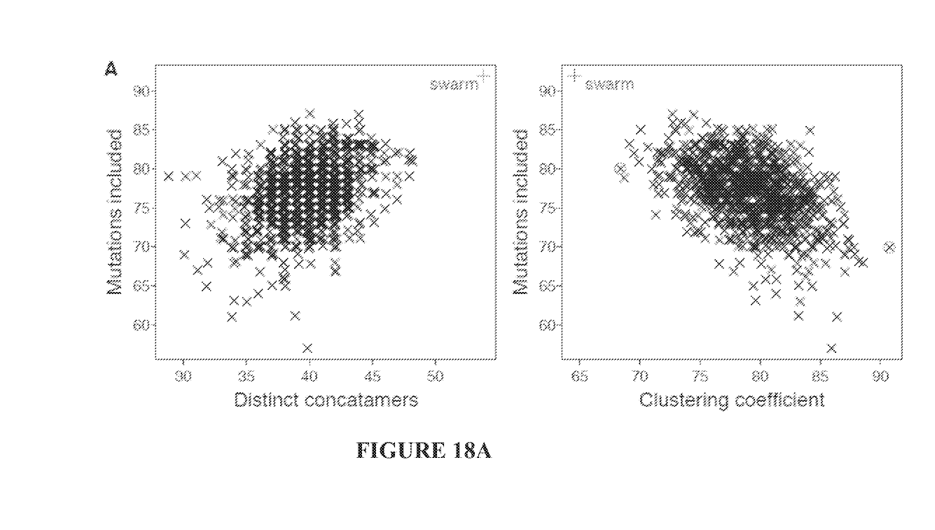

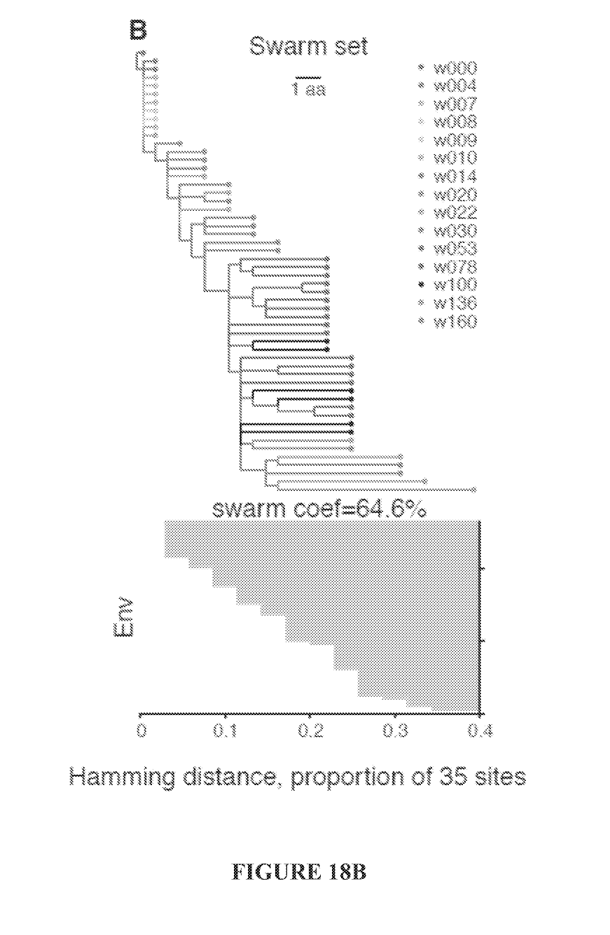

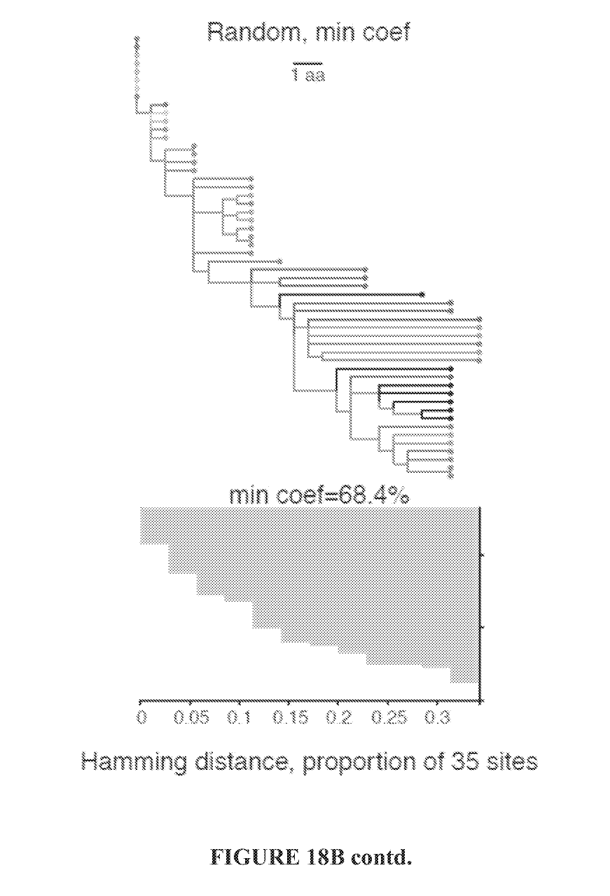

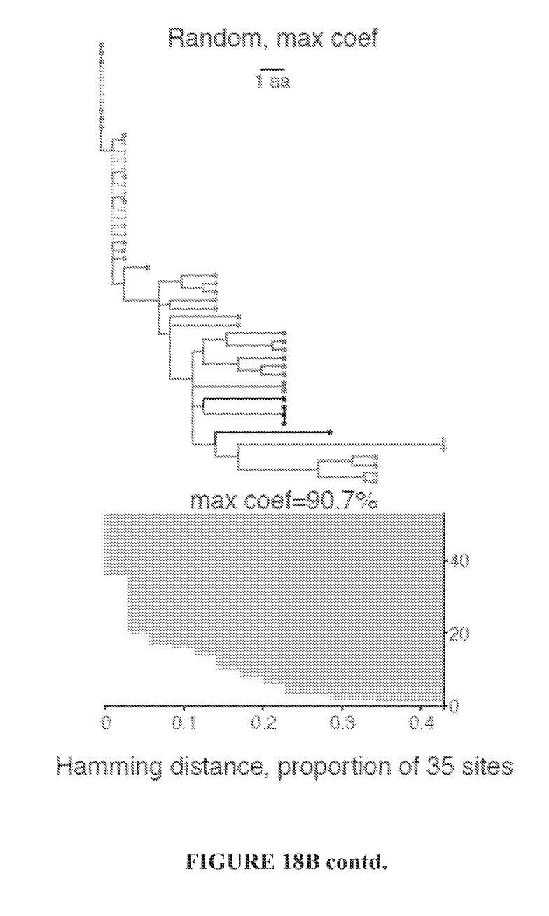

Random sequence selection. A resampling experiment was performed to evaluate the swarm-selection algorithm against a null distribution, which might be sampled by less informed methods. To eliminate multiple copies of the same Env sequence, the full-length Envs that had been normalized were randomly sampled. Removing duplicates and excluding Envs with premature stop or incomplete codons gave 260 distinct Envs, from which the same number of sequences as in the swarm set were repeated resampled, without replacement. FIG. 18 compares the null distribution from resampled results with the algorithmically chosen swarm. In our set of 54 selected Envs, no concatamers were duplicated, i.e. each Env carried a distinct combination of amino acids in the 35 positions of interest. Because the sites represented progressive adaptation of the virus in CH505, it was expected that each concatamer would have distinct phenotypic properties for sensitivity to the co-evolving antibody response, which could be identified by assaying each variant against longitudinally obtained plasmas or mAbs isolated to represent a developing lineage. (This is shown below, under “Antigenic Contexts”.) In contrast, among the randomly chosen sets of 54 Envs, redundancy among concatamers was common. Resampling 1,000 replicates gave a median of 40 distinct concatamers with 95% CI from 34 to 45 (FIG. 18A).

A comparison was made as to how many of the non-TF mutations tabulated in the first pass of the algorithm through all 398 sequences were covered. The antigen swarm set was designed to cover 92 distinct mutations that arose in the 35 selected sites. As expected, random sampling of Envs gave consistently lower coverage of the mutations needed (median 77; 95% CI: 69 to 84) than the 92 mutations that were included by the swarm-selection algorithm (FIG. 18A). This indicates that random sets of the same size do not capture all of the mutations that were considered to have the most potential relevance to antibody sensitivity.

Further, hierarchical dendrograms were computed from Hamming distance matrices for swarm and random sets, and the outcomes were summarized as clustering coefficients. The clustering coefficient, a dimensionless quantity, is the mean distance (from 0 to 1) at which sequences cluster together. It summarizes the distribution of terminal branch lengths as the expected similarity (the complement of normalized distance) among terminal branches [54]. To give an intuition for how this coefficient works, FIG. 18 also depicts the dendrogram from the swarm set, and compares it with the resampled sets that gave lowest (“min”) and highest (“max”) coefficients. The selected Envs had a lower clustering coefficient (65%) than sets of randomly selected sequences, which had a median of 79% and 95% CI: 72-80% (FIG. 18). The lower clustering coefficient indicates less hierarchical grouping structure, i.e. lower overall relatedness, among subsets of concatamers from the selected Envs than exhibited by the random sequence sets [54].

These metrics compared sequence sets from the swarm selection algorithm with null distributions that were obtained by random selection. Because the three metrics are only loosely correlated, they measure different aspects of selected sets of sequences. This appears to be the first attempt to establish criteria to quantify how well any subset of sequences from a larger related set represents diversity (distinct concatamers), polymorphisms (mutations included), and progressive divergence (clustering coefficient) in the larger set.

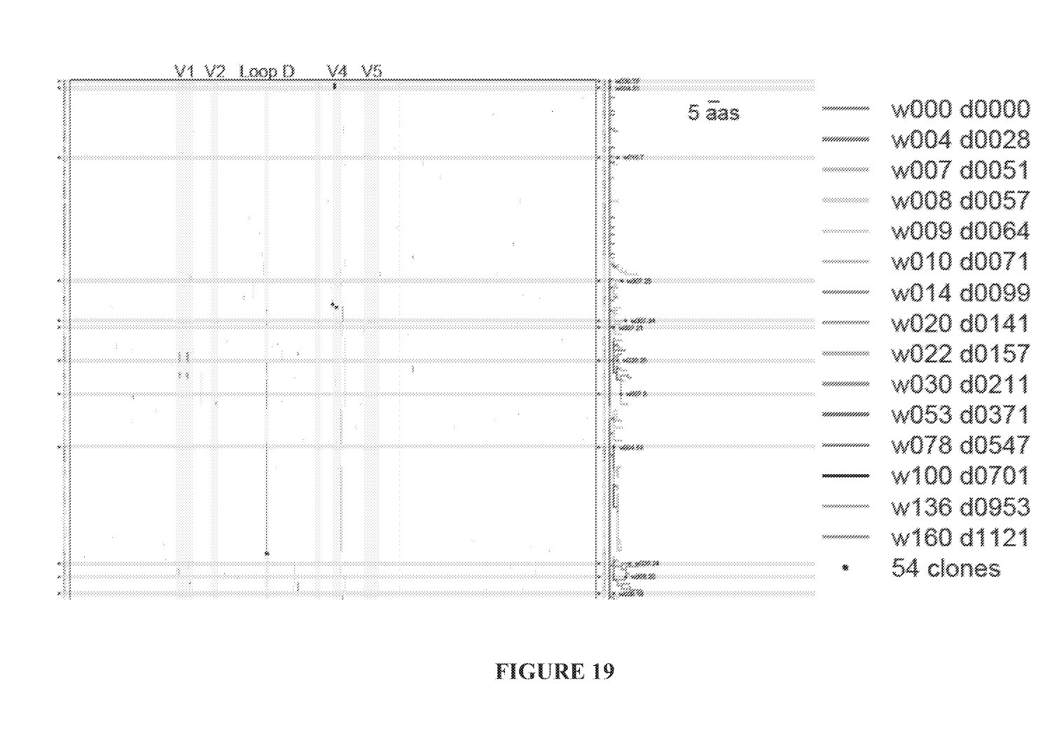

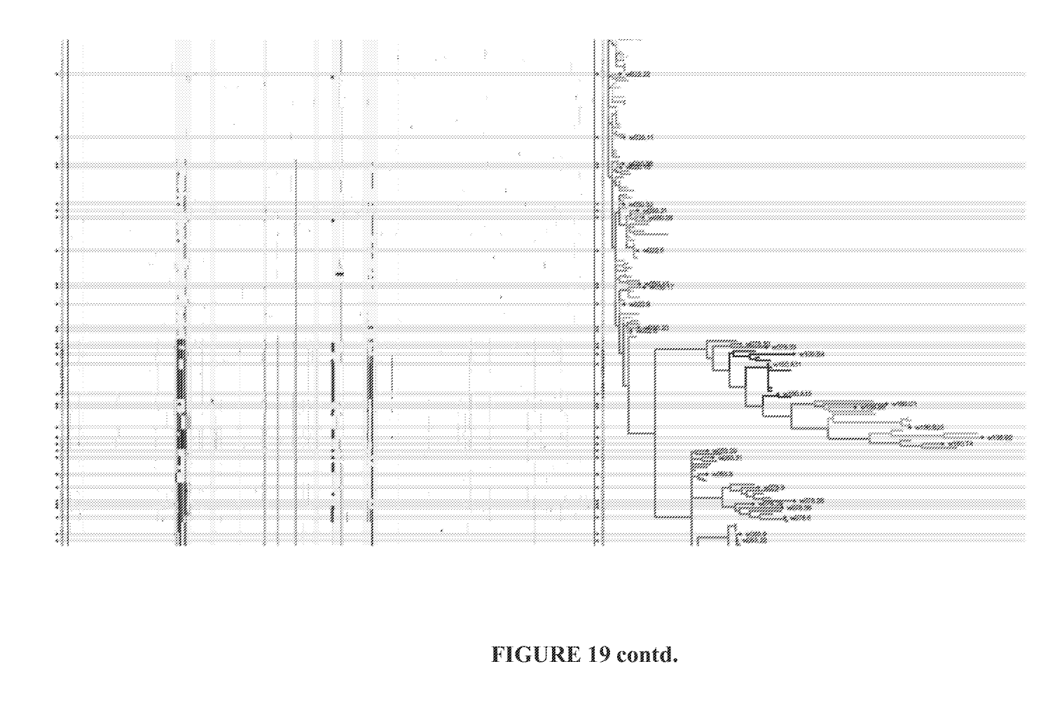

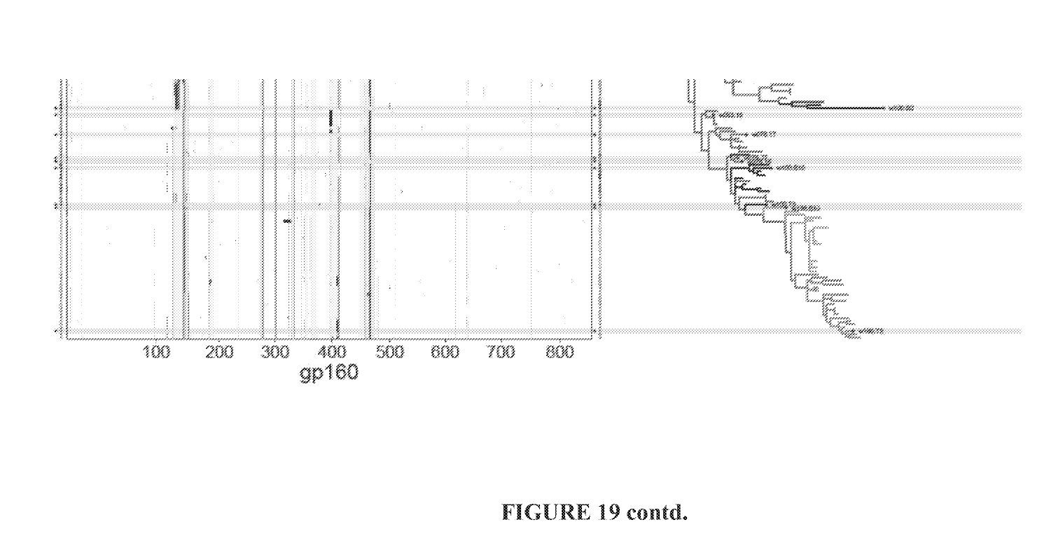

Phylogenetic and antigenic contexts. The phylogenetic context of Envs represented in the swarm set showed that selected sites persisted against the scattered background of ephemeral mutations (FIG. 19). Selected Envs were widely distributed over the tree. The earliest selected Envs (weeks 4-10) tended to carry single mutations, while some later Envs represented large clades sampled only in one or two time-points, such as the sequence w160.T3, which appears near the bottom of the tree.

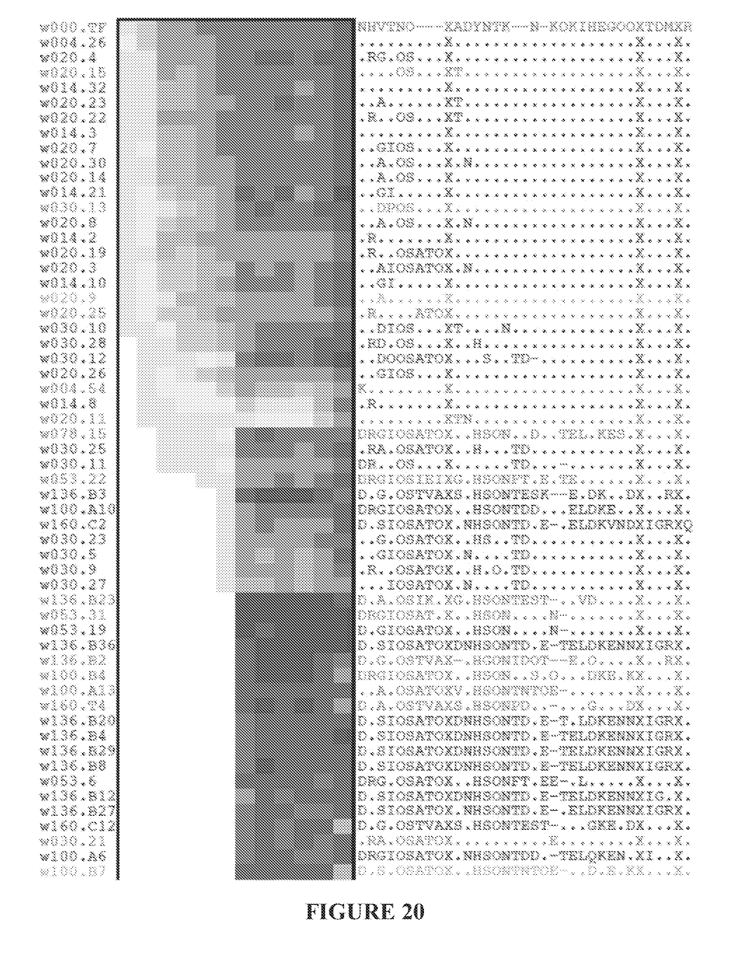

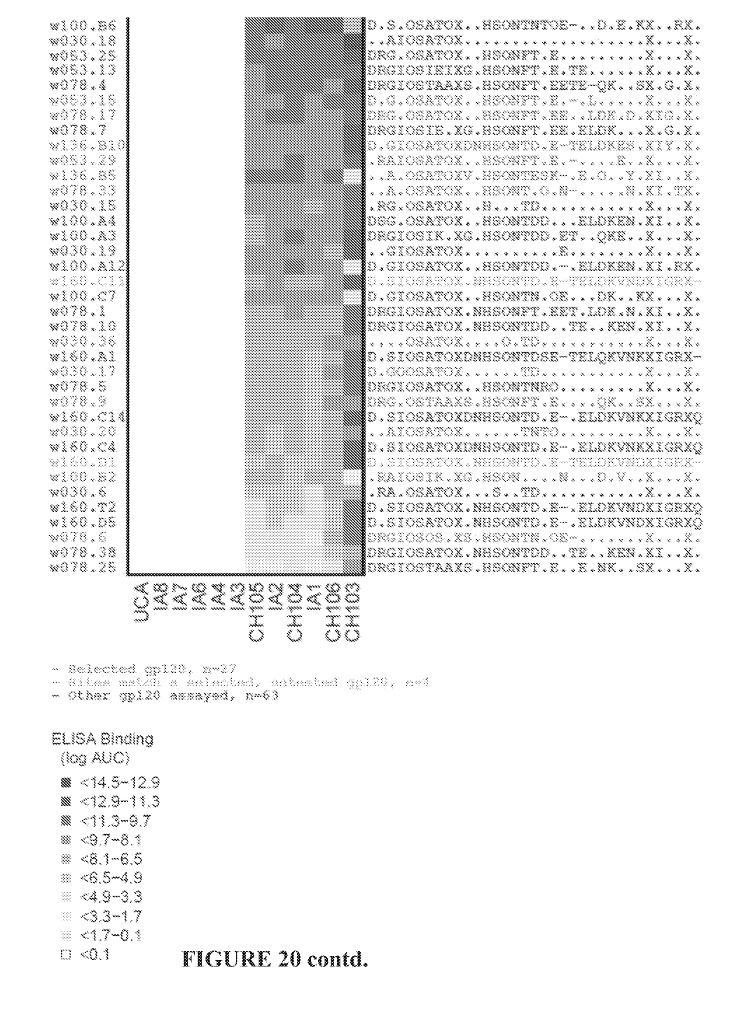

ELISA binding assays with mAbs from the CH505-derived CH103 CD4 binding site bnAb B cell lineage were available for gp120s synthesized from a subset of 27 selected Envs (FIG. 20). Binding assay results confirmed that selected viruses exhibited diverse antibody sensitivities, which increased with maturation of the bnAb lineage and generally followed the progression of mutations away from the TF virus (FIG. 20).

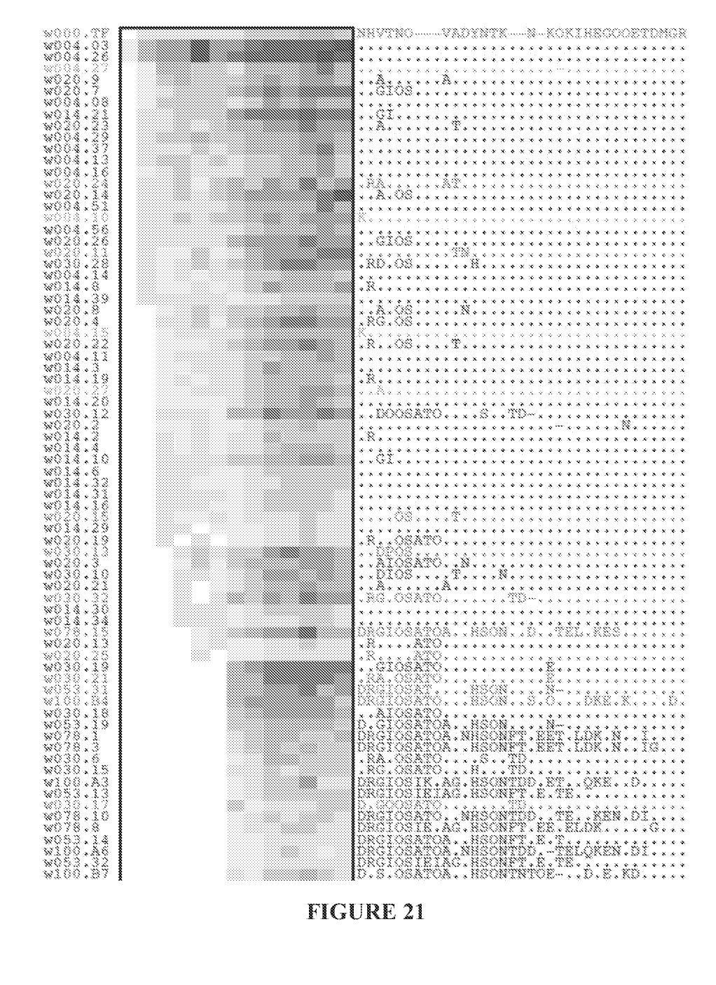

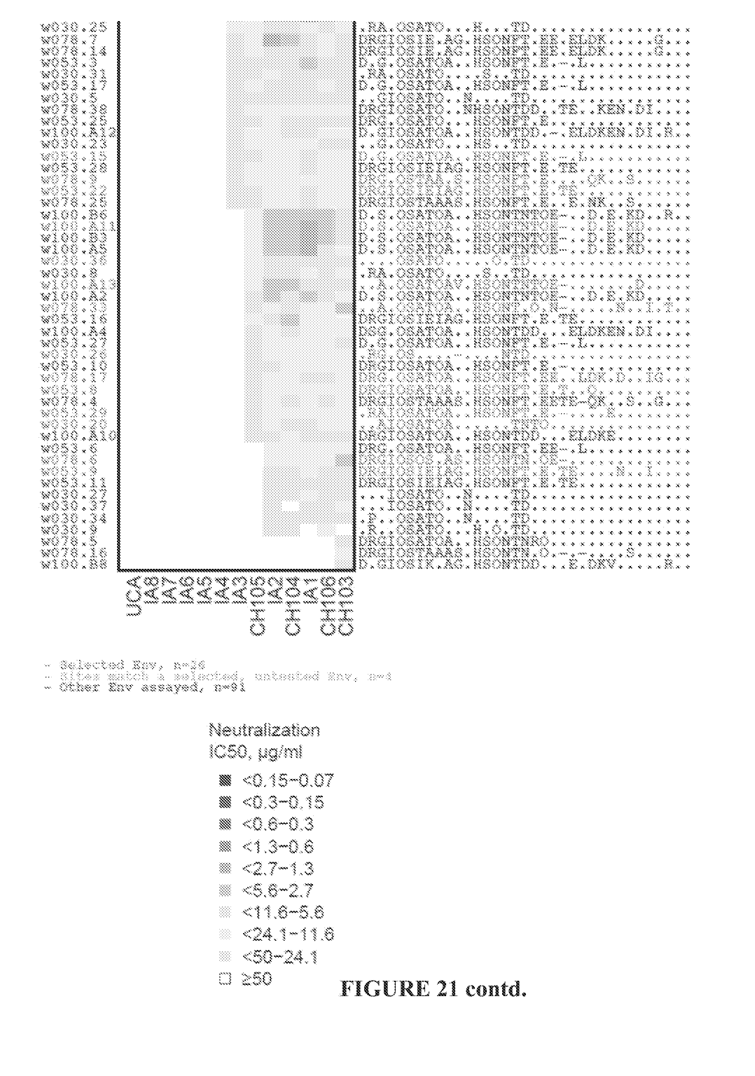

Similarly for neutralization sensitivity, 26 selected Envs were among the 121 Env-pseudotyped viruses tested for sensitivity to neutralization by mAbs of the CH103 lineage (FIG. 21). Selected Envs represent the range of sensitivities among viruses tested, reflecting the diversity of variants that developed in response to sustained selection for neutralization escape.

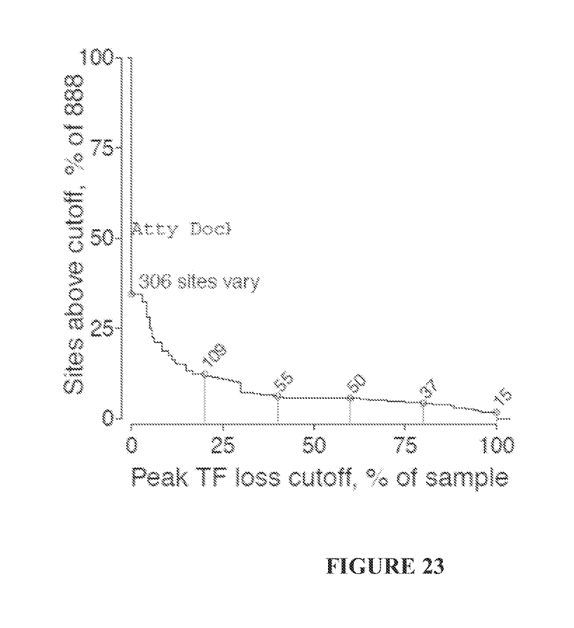

In FIG. 23, neutralization titers from all previously hand-selected viruses clearly show the development of neutralization breadth in phylogenetic context. Envs at the top of the tree are broadly susceptible to many antibodies in the CH103 lineage. Envs that evolved later appear lower in the tree. Neutralization breadth was acquired later in bnAb ontogeny, which is clear as a gradient of increasing potency from the unmutated ancestor (left) to the mature CH103 bnAb (right). By selecting Envs that represent genetic diversity sampled during bnAb development, the method selects Envs that represent relevant antigenicity over time.

Swarm Size Adjustments

A main goal of this procedure is to enable down-selection from a large set of Env sequences, an Env subset that recapitulates development of antigenic diversity in the subject, given realistic experimental and cost constraints. Amino acid sites that were most likely under strong selective pressure were identified first (an important analysis step in its own right), and then sequences were chosen to represent diversity found in those sites. Having more available sequences per time-point allows a user to choose sites with more complete TF loss. To explore how the algorithm functions when applied to larger data sets, it was applied to additional acutely infected study participants with much more extensive sampling, CH694 and CH848.

The cutoff used for loss of the TF form determined how many sites were selected. In turn, this influenced the number of sequences, here Env variants, in the antigenic swarm sets intended for synthesis for phenotypic assays (Table 4). Similarly, the minimum variant count reduced the number of sequences selected by excluding rare mutations. For example, a minimum variant count of two excluded mutations that only ever appeared once. If one did not wish to include sequences that capture each isolated mutation found in selected sites, the size of the resulting reagent set defined by the algorithm could be reduced. By exploring different parameter settings, investigators can evaluate the impact of including variants that represent increasingly rare mutations, in light of resources available for experimental reagents. Unpublished Env sequences from two additional study participants with much greater sequencing depth and more longitudinal samples than individual CH505 provided an opportunity to consider effects of varied parameters (Table 4). In these cases, increasing the TF loss cutoff to 95% or 100% was necessary to preserve a desired swarm size of 100 Envs chosen from over a thousand Env SGA sequences.

TABLE 4
Size adjustment. Number of sites and sequences selected with varied TF loss cutoff and
minimum variant counts, compared for three serially sampled, acutely infected subjects.

| | | CH505 | CH694 | CH848 |
|---|---|---|---|---|
| Time-points sampled | | 14 | 26 | 32 |
| Latest time-point, days post-infection | | 1121 | 1560 | 1720 |
| Sequences by single-genome amplification | | 398 | 1104 | 1184 |
| Median (and range) sequences per time-point | | 25 (18, 53) | 40 (30, 62) | 35 (28, 79) |
| Sites selected with TF loss cutoff 80% | | 35 | 74 | 96 |
| Envs selected with min variant count | 1 | 54 | 131 | 198 |
| Sites selected with TF loss cutoff 90% | | 23 | 64 | 85 |
| Envs selected with min variant count | 1 | 38 | 118 | 191 |
| Sites selected with TF loss cutoff 95% | | 17 | 54 | 79 |
| Envs selected with min variant count | 1 | 29 | 105 | 175 |
| | 5 | 24 | 79 | 132 |
| | 10 | 18 | 73 | 122 |
| | 15 | 18 | 65 | 119 |
| | 20 | 16 | 61 | 113 |
| Sites selected with TF loss cutoff 100% | | 15 | 36 | 65 |
| Envs selected with min variant count | 1 | 25 | 83 | 156 |
| | 5 | 20 | 63 | 121 |
| | 10 | 15 | 58 | 111 |
| | 15 | 15 | 52 | 108 |
| | 20 | 13 | 51 | 102 |

A large proportion of variants are required to represent each recurrent mutation among selected sites from hypervariable loop regions. An alternative approach may be to emphasize only those sites that can be mapped onto HXB2, and consider hypervariable regions separately. This approach assumes it is not essential to sample each particular form that appears in disordered regions, such as the hypervariable portions of V1, V2, V4, and V5. Instead, it emphasizes covering all variants among more ordered regions and picking up the linked variants in disordered regions without sampling them completely. If only distinct HXB2 positions were counted and represented, then 80% TF loss with CH848 gives 65 sites and 127 sequences against 970 sequences obtained through peak breadth at d1432. These 127 sequences capture all 209 mutations (including indels) in the 65 HXB2 positions that appear more than once. Similarly, for CH694, the algorithm chose 112 sequences from 1103 Envs to represent 181 mutations that appeared more than once over 59 sites with at least 80% TF loss.

Chronic Infection

These methods were developed initially to select sequences from longitudinal studies beginning early in infection, where the TF virus is known or reliably inferred, and the progression of escape mutations is readily apparent. This is not true for chronic infection. Still, it may be desirable to select a subset of sequences that represent diversity in chronic infection samples. To evaluate the algorithm ability to select an antigenic swarm from a chronic infection, the algorithm was applied to sequences from a study participant enrolled in chronic infection, designated CH457 [45]. 205 plasma SGA Envs from ten sample time-points were analyzed (median was 20 sequences per time-point; the distribution ranged from 12 to 35). In the chronic enrollment sample, five of twenty Envs exactly matched the within-time-point consensus. One of these (w0.e18) was used as the reference to compute variant frequencies. No variation was detected in 582 of 888 aligned sites, and an 85% cutoff identified 35 sites that were candidates for strong positive selection (FIG. 23). Nine of the 35 sites are located in gp41.

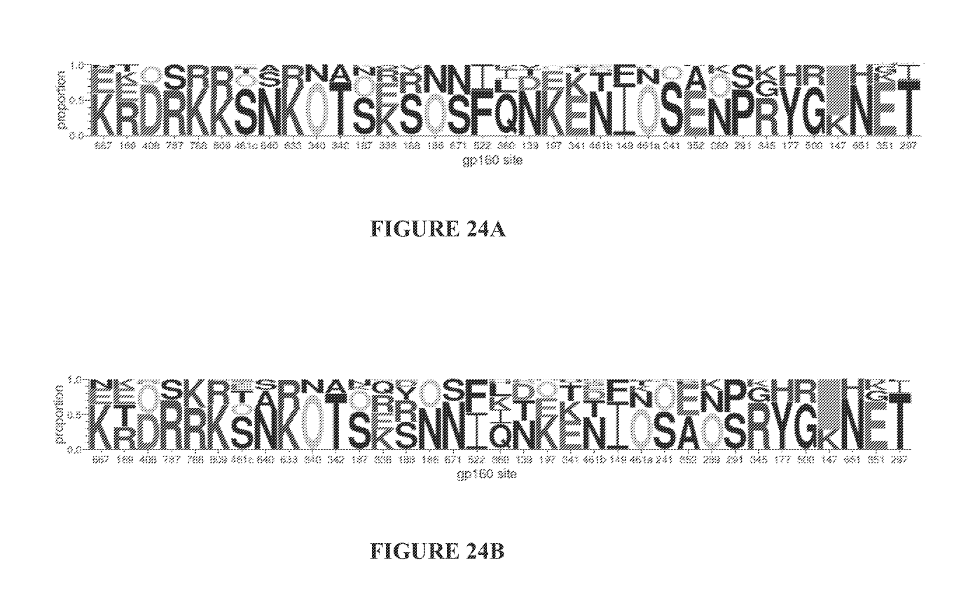

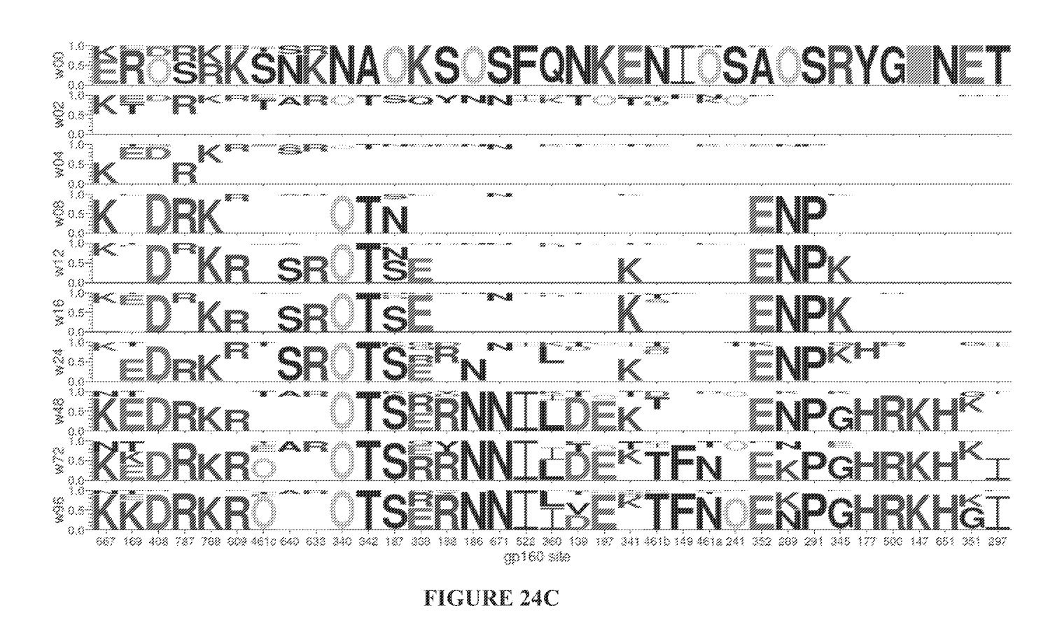

With singleton variants excluded, the algorithm selected a swarm of 44 Envs (FIGS. 24A-24C). The progressive accumulation of mutations among concatamers of selected sites is less clear in this chronically infected subject than in acute infection (cf. FIG. 16B). Furthermore, sites that appear to be under selection in the window of time studied are not clearly associated with two epitope regions, as was the case of CH505, where there was a strong imprint of CD4bs and V3 antibodies selection, and indeed antibodies with these specificities were isolated from the subject. In the case of CH457, most of the selected sites were located were not identified in the 2015 Los Alamos Database as being relevant to particularly antibodies, although two sites were in the MPER region of gp41 (667, 671) and two sites were predicted signatures of the 2F5 MPER antibody (640, 351). In addition, one site was associated with CD4bs antibodies: a changing glycosylation pattern at 461, which contacts CD4 and the CD4bs bnAbs VRC01 and NIH45-46. Two of the selected sites (651, 640) have been noted to be CD4bs antibody signatures [51]. A potent CD4bs bnAb CH27 was isolated from subject CH457, but the virus isolated from CH47 plasma had escaped by the time of enrollment, although archived provirus from CH457 cell-associated DNA was still sensitive. CH13, a weaker CD4bs nAb capable of only neutralizing Tier 1 viruses, was isolated, and may have been exerting weak selective pressure in the last weeks sampled.

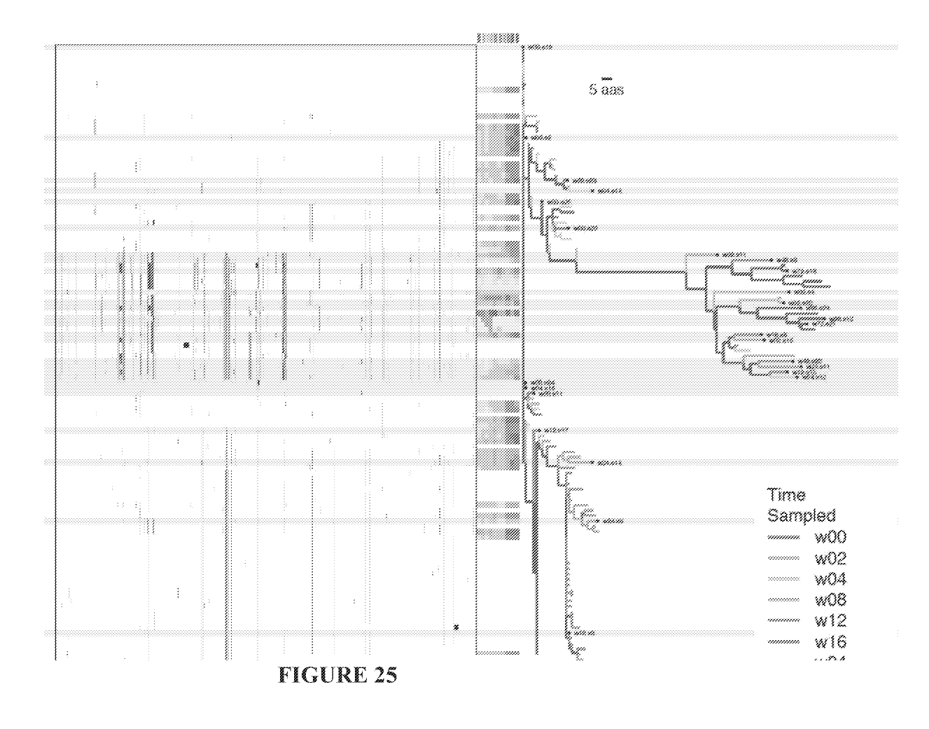

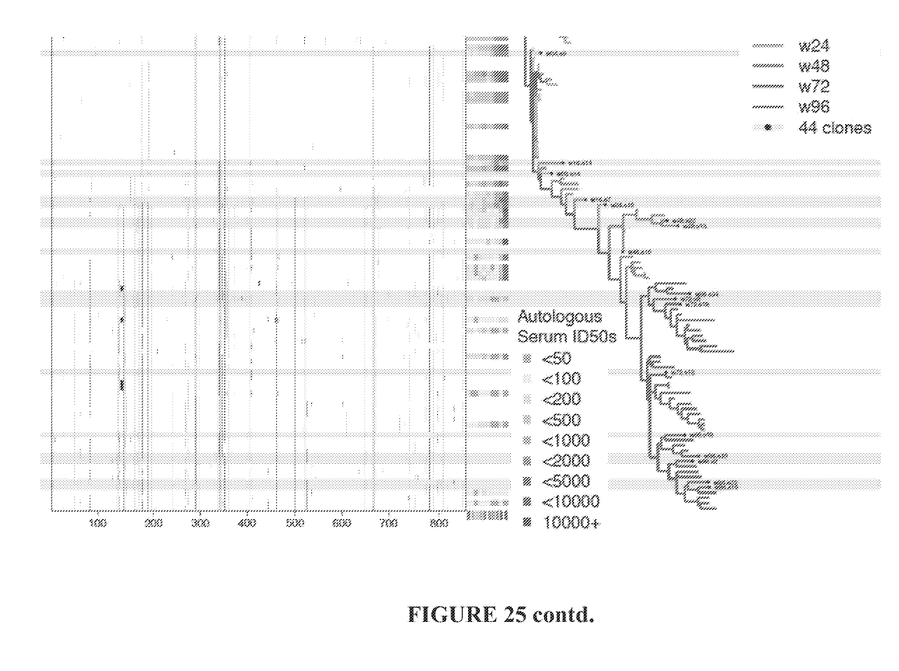

The phylogeny indicated a persistent, divergent secondary clade, represented by 24 of 205 plasma Envs (FIG. 25). This clade was not introduced by misalignment nor by simple recombination, and was also represented by cellular provirus sequences [45]. Though the divergent clade was undetected among sequences from the enrollment sample, it was represented by 14 of the 44 Envs selected (FIGS. 24A-24C). Thus, the algorithm can be used for both acute and chronic Env sequential sequence analysis and swarm design.

Discussion

The task of selecting representative variants from a larger set for follow-up studies from longitudinal samples is routine, but can be complex when choosing from hundreds to thousands of sequences. Furthermore, while methods for isolating bnAbs from HIV-1 infected subjects and vaccinees are rapidly improving, it remains a challenging task. The approach described herein suggests the task can be divided into two main parts, identification and tracking of selected sites within a subject, and identifying sequences that represent the antigenic diversification in that subject. A computational approach (LASS) to automate these tasks has been developed.

First, transmitted-founder loss is used in one or more samples in a longitudinal study as a simple way to identify sites under selective pressure. Despite the existence of a variety of methods to test for positive selection [55, 56], their utility to identify sites under positive selection in the context of within-subject viral evolution during acute infection is limited due to statistical power for inference. In contrast, the loss of the TF form at any one time-point is a simple and inclusive measure. In CH505, sites selected by this criterion were focused in regions that were highly relevant to the adaptive immune responses that were previously identified in the subject [15]. This suggests that in future studies, structural localization of selected sites could be used to raise hypotheses about specificities of bnAbs in plasma. Furthermore, the timing of TF loss identifies these important mutational events, and could help determine when antibodies exert the most selective pressure. Such information could guide the search for monoclonal antibodies in subjects with potent nAbs, by focusing on antibody specificities that recognize the epitopes under selection, and by aiding in selecting the sample used to isolate new bnAbs in a subject who was sampled over several years of follow-up.

Second, a rational, objective method is provided to guide the selection of Env sets for experimental study from large sequences sets sampled over time. LASS can select sets of sequences that represent gradual antigenic diversification induced during bnAb development, ensuring that all variants in sites identified by TF loss are represented in an Env reagent set. The method is computationally efficient, scaling linearly with the number of sequences, and minimizes redundancy, selecting only as many variants as are necessary to represent diversity in sites selected by TF loss. The algorithm starts with sequences most closely resembling the form that established the infection, and gradually increases diversity in a manner that parallels natural infection.

LASS was used to identify selected sites and representative sequence subsets in longitudinal samples from three acutely infected subjects and one subject sampled only during chronic infection. SGA sequences were analyzed from all four subjects, providing intact env gp160s with no recombination artifacts and minimal error [5, 14, 21, 40, 41]. While this provides optimal conditions, the approach could also be used in other longitudinal study designs and sequencing strategies.

In related research, sequence selection has been represented as a set-coverage problem [57], and networks of covarying sites are identified from a population-level alignment, which represents a particular clade [58], not a within subject alignment as in our case. A limitation of our approach, which will be addressed, is that sites are treated independently, while covariation between sites may influence variant suitability and TF loss. Considering covariation may potentially facilitate identification of smaller representative swarm sets. However, by progressively adopting mutations in the context of variant sequences where they first arise in the sequence sets, the swarm sets, by definition, allow the study of mutations in the context of the natural pairings as they were found in vivo. This strategy also has a potential advantage over site-specific mutagenesis, which necessarily studies mutations in isolation. A mutation observed in a later time-point and introduced into the TF, for example, may not have the same phenotypic consequences as it does in the background of the Env in which it arose, so the ability to study related natural variants isolated serially may be ultimately more informative.

Virus diversification precedes, and thus may drive, the development of neutralization breadth in HIV-1 infection [16, 18], and exposure of a neutralizing antibody lineage during affinity maturation to a gradual increase in antigen diversity could result in selection of antibodies with increased breadth. Thus, mimicking in vivo diversification has been proposed as a possible vaccination strategy [15, 18, 59-61]. With recent technological advances, it is becoming feasible to test vaccine designs that not only include 5-10 antigens, but potentially between 50-100 antigens, administered as DNA in either in series or in combination [62, 63]. As LASS uses efficient algorithm to identify candidate sets of antigens with progressively increasing diversity at important sites in polymorphic viral proteins, in could be used to aid in the design for such “antigen-swarm vaccines.” An additional potential use of the algorithm, not described here, is to analyze large antibody sequence data sets to identify, analyze selection, and select a representative subset of antibody sequences from clonal lineages of for detailed study. For example, the algorithm could be used to identify key members of antibody clonal lineages as mutations arise for HIV-antibody co-evolution studies.

In summary, computational methods have been developed to identify and track selected sites in longitudinal data, and to use these selected sites to aid in down-selecting sequence sets for reagent design, or for testing the “antigen swarm” vaccine concept. When applied to longitudinal HIV samples, a retrospective evaluation of viral sequences from the intensely studied subject CH505 showed that the LASS provided meaningful results, highlighting selected sites that were indeed under immune selective pressure, and building a non-redundant collection of sequences tailored to characterize the phenotypic consequences of those mutations. LASS may be useful in many contexts, such as assisting in bnAb isolation, as well studies of other viral infections, and studies of antibody evolution.

Methods

Site Selection

Transmitted-founder (TF) loss is the proportion of sequences sampled per time-point that have lost the ancestral TF state. This is an efficient way to select rapidly evolving sites. Here no information other than TF loss was considered, though such information could be used to select sites. This could include signature sites associated with neutralization assay outcomes and antibody contact residues from structural data, if available.

The starting point was env cDNA amplicons sequenced via single-genome amplification (SGA), also known as limiting-dilution PCR, sampled longitudinally, beginning early (3-6 weeks) after infection, with 3-5 years of clinical follow-up. Sequencing effort was intended to obtain about 20 sequences from each of 14 samples. It is common for SGA from homogeneous infections to yield multiple identical sequences, all of which were kept. A naming convention for Env sequences was used to ensure consistency and so sample time-point labels could be parsed from sequence names. To study variant dynamics, the number of elapsed days after the earliest sample from sample dates was computed, and the number of days post-infection estimated from the earliest sample was added. For homogeneous infections sampled before the onset of immune selection, a simple Poisson model of random sequence evolution provides the estimate [5, 6].

The HXB2 reference sequence was added to facilitate numbering positions, the sequences were codon-aligned, then translated, and a phylogeny was inferred. Because no algorithm aligns the HIV envelope perfectly, particularly when a translation is needed, manual alignment was started after preliminary alignment with an HIV-specific hidden Markov model. Aligning all but the hypervariable loops is trivial given such a preliminary alignment. Because hypervariable loops evolve rapidly by tandem duplications, a useful alignment criterion is self-consistency, rather than identification of homologous sites. For example, a putative N-linked glycosylation motif could be placed at either the N- or C-terminal position of an otherwise gapped region. Uniform placement of such motifs, particularly where HXB2 has no corresponding sites, facilitates analysis because the variants appear more clearly as evolutionary signal if aligned consistently.

Maximum-likelihood trees were inferred from translated amino-acid sequences with PhyML v3 and the HIVw (HIV-specific, within-host) substitution model [64-66]. The phylogeny is used to order sequences and is an organizing principle for sequence evolution from the ancestral TF virus. To identify potential N-linked glycosylation (PNG) sites, PNG sites were annotated by replacing asparagine sites that match the Nx[ST] motif to become Ox[ST]. In the PNG motif, x indicates any amino acid except proline, and the third position is either serine or threonine. For each aligned site, TF loss per time-point sequenced was computed, the maximum identified, and this peak TF loss was compared with a threshold. The threshold was adjusted and the resulting number of sites was considered. This gave a list of sites, which were considered as interesting evolutionary “hot spots” to be represented by a swarm of Envs.

Swarm Selection

Having used the TF-loss criterion to select sites from the alignment, a set of Envs was identified to represent the variants that occur at these sites. By simple combinatoric calculations, there are at least 10¹⁰⁰ distinct ways to choose k representatives from n individuals for n above 427 and k over 100. On the scale of the current example, choosing 50 representatives from 385 candidates gives over 10⁶³ distinct alternatives. To search such a vast space of possible solutions is intractable for even the fastest computers. Worse, in the regime of interest, the number of possible solutions grows exponentially with n or k, where k<<n/2.

A simple, efficient algorithm was designed and implemented to select sequences that represent variants at sites selected by TF loss. The approach is greedy, meaning it adds variants iteratively, rather than refine the entire set for potentially better solutions. Such a greedy approach is unlikely to give the best possible overall solution, but can efficiently provide reasonably good solutions, and can be refined to include other criteria as needed. It works from the same alignment used to select sites, and assumes that the TF form and sample time-points can be identified from sequence names. A virtue of the greedy approach is that it considers time of sampling, and starts with sequences most like the form that established the infection, then progressively builds diversity in a manner that follows the natural course of infection. In this way, common mutations and mutations that eventually go to fixation are sampled many times.

As outlined in FIG. 17, the swarm selection algorithm selects sets of sequence variants that recapitulate viral evolution in key residues from a table of the amino acid variants found at each selected site. This table of variant counts is used to monitor which remaining mutations need to be included in the swarm set. Variants that only ever appear once, or some other number of times specified by the minimum variant count, are disregarded. Candidates for Env selection must be functionally viable, by lacking long deletions (as specified by the operator of the algorithm) and premature stop codons or incomplete codons, which typically result from frame-shift mutations. Then, starting with the TF form, the procedure iterates chronologically over time-points sampled, and identifies an Env to represent each needed variant at each of the selected sites, should such a variant be present.

Within a time-point, the choice among multiple Envs that carry a needed variant is resolved by a series of criteria. The algorithm first tries to identify the sequence that uniquely minimizes the distance (number of mutations, including gaps) to the TF among selected sites. Then, in case of ties, a sequence is chosen that minimizes distance to the full-length TF. Finally, if ties remain, a sequence is chosen that minimizes the average distance to the current working set of sequences.

The sequence selected to represent the needed mutation is included in the swarm set, the corresponding counts in the table of needed mutations are set to zero, and iteration continues. An option exists to require that specific sequences be included, if desired. Such a sequence is added during iteration, to ensure inclusion of earlier forms that carry variants found on the specified sequence, rather than beforehand. Upon iterating over all sample time-points, selected variants, and needed sites, the swarm is complete. This approach is deterministic for a given set of sequences, though unresolved ties may exist among alternative sequences for some data. (This situation, in practice, has yet to be encountered.) Any remaining ties would indicate a need for additional selection criteria, though this outcome is yet to be encountered. An advantage of this approach is that it selects only as many sequence as are necessary to represent the mutational variants in selected sites, rather than some arbitrary number. However, the greedy approach errs towards inclusion of early point mutations that could be included with later, more divergent, viruses.

The software tools for swarm selection were written as an R package called swarmtools. Example data from CH505 and a tutorial “vignette” are included. Phylogenetic trees have been paired, the TF virus has been rooted on, ladderized, and then rendered as phylograms, together with pixel plots (derived from Highlighter plots [5]), which illustrate polymorphisms as either mutations or insertions/deletions relative to the TF sequence. These have been found to be informative representations for understanding evolution of the virus population in an acutely infected host, given the limited genetic diversity that occurs in early infections [15, 45]. Renderings such as in FIG. 19 emphasize sites with evolutionary changes that produce the branching patterns in the tree, and enable detection of recombinant clades or evolutionary associations with phenotypic assays. The code that was used to make such renderings is available in an R package called pixgram, which uses ape to draw trees [67].

### REFERENCES

1. Plotkin S A. Correlates of vaccine-induced immunity. Clin Infect Dis. 2008; 47:401-9. doi: 10.1086/589862.
2. Mascola J M, Montefiori D M. The role of antibodies in HIV vaccines. Annu Rev Immunol. 2010; 28:413-44. doi: 10.1146/annurev-immunol-030409-101256
3. Mascola J R, Lewis M G, Stiegler G, Harris D, VanCott T C, Hayes D, et al. Protection of Macaques against pathogenic simian/human immunodeficiency virus 89.6PD by passive transfer of neutralizing antibodies. J Virol. 1999; 73(5):4009-18. PubMed PMID: 10196297; PubMed Central PMCID: PMC104180.
4. Moldt B, Rakasz E G, Schultz N, Chan-Hui P Y, Swiderek K, Weisgrau K L, et al. Highly potent HIV-specific antibody neutralization in vitro translates into effective protection against mucosal SHIV challenge in vivo. Proceedings of the National Academy of Sciences of the United States of America. 2012; 109(46):18921-5. doi: 10.1073/pnas.1214785109. PubMed PMID: 23100539; PubMed Central PMCID: PMC3503218.
5. Keele B, Giorgi E, Salazar-Gonzalez J, Decker J, Pham K, Salazar M, et al. Identification and characterization of transmitted and early founder virus envelopes in primary HIV-1 infection. Proc Natl Acad Sci USA. 2008;105:7552-7.
6. Giorgi E, Funkhouser B, Athreya G, Perelson A, Korber B, Bhattacharya T. Estimating time since infection in early homogeneous HIV-1 samples using a Poisson model. BMC Bioinformatics. 2010;11:532.
7. Mellors J W, Rinaldo C R, Jr., Gupta P, White R M, Todd J A, Kingsley L A. Prognosis in HIV-1 infection predicted by the quantity of virus in plasma. Science. 1996; 272(5265):1167-70. PubMed PMID: 8638160.
8. Mackelprang R D, Carrington M, Thomas K K, Hughes J P, Baeten J M, Wald A, et al. Host genetic and viral determinants of HIV-1 RNA set point among HIV-1 seroconverters from sub-Saharan Africa. J Virol. 2015; 89(4):2104-11. doi: 10.1128/JVI.01573-14. PubMed PMID: 25473042.
9. Wolinsky S M, Korber B T, Neumann A U, Daniels M, Kunstman K J, Whetsell A J, et al. Adaptive evolution of human immunodeficiency virus-type 1 during the natural course of infection. Science. 1996; 272(5261):537-42. PubMed PMID: 8614801.
10. Weiss R, Clapham P, Weber J, Dalgleish A, Lasky L, Berman P. Variable and conserved neutralization antigens of human immunodeficiency virus. Nature. 1986; 324(6097):572-5.
11. Richman D D, Wrin T, Little S J, Petropoulos C J. Rapid evolution of the neutralizing antibody response to HIV type 1 infection. Proc Natl Acad Sci USA. 2002; 100(7):4144-9. doi: doi: 10.1073/pnas.0630530100.
12. Wei X, Decker J M, Wang S, Hui H, Kappes J C, Wu X, et al. Antibody neutralization and escape by HIV-1. Nature. 2003; 422:307-12. doi: doi:10.1038/nature01470.
13. Scheid J F, Mouquet H, Feldhahn N, Seaman M S, Velinzon K, al e. Broad diversity of neutralizing antibodies isolated from memory B cells in HIV-infected individuals. Nature. 2009; 458:636-40. doi: doi:10.1038/nature07930.
14. Bar K J, Tsao C-y, Iyer S S, Decker J M, Yang Y, Bonsignori M, et al. Early low-titer neutralizing antibodies impede HIV-1 replication and select for virus escape. PLoS Pathog. 2012; 8(5):e1002721. doi: 10.1371/journal.ppat.1002721.
15. Liao H X, Lynch R, Zhou T, Gao F, Alam S M, Boyd S D, et al. Co-evolution of a broadly neutralizing HIV-1 antibody and founder virus. Nature. 2013; 496(7446):469-76. doi: 10.1038/nature12053. PubMed PMID: 23552890; PubMed Central PMCID: PMC3637846.
16. Gao F, Bonsignori M, Liao H X, Kumar A, Xia S M, Lu X, et al. Cooperation of B cell lineages in induction of HIV-1-broadly neutralizing antibodies. Cell. 2014; 158(3):481-91. doi: 10.1016/j.ce11.2014.06.022. PubMed PMID: 25065977; PubMed Central PMCID: PMC4150607.
17. Hraber P, Seaman M S, Bailer R T, Mascola J R, Montefiori D C, Korber B T. Prevalence of broadly neutralizing antibody responses during chronic HIV-1 infection. Aids. 2014; 28(2):163-9. doi: 10.1097/QAD.0000000000000106. PubMed PMID: 24361678; PubMed Central PMCID: PMC4042313.
18. Doria-Rose N A, Schramm C A, Gorman J, Moore P L, Bhiman J N, DeKosky B J, et al. Developmental pathway for potent V1V2-directed HIV-neutralizing antibodies. Nature. 2014; 509(7498):55-62. doi: 10.1038/nature13036.
19. Goonetilleke N, Liu M, Salazar-Gonzalez J, Ferrari G, Giorgi E, Ganusov V, et al. The first T cell response to transmitted/founder virus contributes to the control of acute viremia in HIV-1 infection. J Exp Med. 2009; 206:1253-72.
20. Fischer W, Ganusov V V, Giorgi E E, Hraber P T, Keele B F, Leitner T, et al. Transmission of single HIV-1 genomes and dynamics of early immune escape revealed by ultra-deep sequencing. PLoS ONE. 2010; 5(8):e12303. doi: 10.1371/journal.pone.0012303. PubMed PMID: 20808830; PubMed Central PMCID: PMC2924888.
21. Liu M K, Hawkins N, Ritchie A J, Ganusov V V, Whale V, Brackenridge S, et al. Vertical T cell immunodominance and epitope entropy determine HIV-1 escape. The Journal of clinical investigation. 2013; 123(1):380-93. doi: 10.1172/JCI65330. PubMed PMID: 23221345; PubMed Central PMCID: PMC3533301.
22. Edwards C T, Holmes E C, Pybus O G, Wilson D J, Viscidi R P, Abrams E J, et al. Evolution of the human immunodeficiency virus envelope gene is dominated by purifying selection. Genetics. 2006; 174(3):1441-53. doi: 10.1534/genetics.105.052019. PubMed PMID: 16951087; PubMed Central PMCID: PMC1667091.
23. Walker L M, Phogat S K, Chan-Hui P Y, Wagner D, Phung P, Goss J L, et al. Broad and potent neutralizing antibodies from an African donor reveal a new HIV-1 vaccine target. Science. 2009; 326(5950):285-9. doi: 10.1126/science.1178746. PubMed PMID: 19729618; PubMed Central PMCID: PMC3335270.
24. Wu X, Yang Z Y, Li Y, Hogerkorp C M, Schief W R, Seaman M S, et al. Rational design of envelope identifies broadly neutralizing human monoclonal antibodies to HIV-1. Science. 2010; 329(5993):856-61. doi: 10.1126/science.1187659. PubMed PMID: 20616233; PubMed Central PMCID: PMC2965066.
25. Walker L M, Huber M, Doores K J, Falkowska E, Pejchal R, Julien J P, et al. Broad neutralization coverage of HIV by multiple highly potent antibodies. Nature. 2011; 477(7365):466-70. doi: 10.1038/nature10373. PubMed PMID: 21849977; PubMed Central PMCID: PMC3393110.
26. Scheid J F, Mouquet H, Ueberheide B, Diskin R, Klein F, Oliveira T Y, et al. Sequence and structural convergence of broad and potent HIV antibodies that mimic CD4 binding. Science. 2011; 333(6049):1633-7. doi: 10.1126/science.1207227. PubMed PMID: 21764753; PubMed Central PMCID: PMC3351836.
27. Kepler T B. Reconstructing a B-cell clonal lineage. I. Statistical inference of unobserved ancestors. F1000Research. 2013; 2:103. doi: 10.12688/f1000research.2-103.v1. PubMed PMID: 24555054; PubMed Central PMCID: PMC3901458.
28. Kepler T B, Munshaw S, Wiehe K, Zhang R, Yu J S, Woods C W, et al. Reconstructing a B-cell clonal lineage. II. Mutation, selection, and affinity maturation. Frontiers in immunology. 2014; 5:170. doi: 10.3389/fimmu.2014.00170. PubMed PMID: 24795717; PubMed Central PMCID: PMC4001017.
29. Wibmer C K, Bhiman J N, Grey E S, Tumba N, Abdool Karim S S, al e. Viral escape from HIV-1 neutralizing antibodies drives increased plasma neutralization breadth through sequential recognition of multiple epitopes and immunotypes. PLoS Pathog. 2013; 9(10):e1003738. doi: 10.1371/journal.ppat.1003738
30. Haynes B F, McElrath M J. Progress in HIV-1 vaccine development. Curr Opin HIV AIDS. 2013; 8(4):326-32. doi: 10:1097/COH.0b013e328361d178.
31. Haynes B F, Moody M A, Alam S M, Bonsignori M, Verkoczy L, al e. Progress in HIV-1 vaccine development. J Allergy Clin Immunol. 2014; 134:3-10. doi: 10.1016/j jaci.2014.04.025.
32. Tomaras G D, Haynes B F. HIV-1-specific antibody responses during acute and chronic HIV-1 infection. Curr Opin HIV AIDS. 2009; 4(5):373-9. doi: 10.1097/COH.0b013e32832f00c0.
33. Haynes B F, Kelsoe G, Harrison S C, Kepler T B. B-cell—lineage immunogen design in vaccine development with HIV-1 as a case study. Nature Biotechnol. 2012; 30:423-33. doi: 10.1038/nbt.2197.
34. Zhou T, Zhu J, Wu X, Moquin S, Zhang B, al e. Multidonor analysis reveals structural elements, genetic determinants, and maturation pathway for HIV-1 neutralization by VRC01-class antibodies. Immunity. 2013; 39(2):245-58. doi: 10.1016/j.immuni.2013.04.012.
35. Bonsignori M, Hwang K K, Chen X, Tsao C Y, Morris L, al e. Analysis of a clonal lineage of HIV-1 envelope V2/V3 conformational epitope-specific broadly neutralizing antibodies and their inferred unmutated common ancestors. J Virol. 2011; 85(19):9998-10009. doi: 10.1128/JVI.05045-11.
36. Kwong P D, Mascola J R. Human antibodies that neutralize HIV: identification, structures, and B cell ontogenies. Immunity. 2012; 37:412-25.
37. Mascola J M, Haynes B F. HIV-1 neutralizing antibodies: understanding nature's pathways. Immunol Rev 2013; 254:225-44.
38. Malherbe D C, Doria-Rose N A, Misher L, Beckett T, Puryear W B, al e. Sequential immunization with a subtype B HIV-1 envelope quasispecies partially mimics the in vivo development of neutralizing antibodies. J Virol. 2011; 85:5262-74. doi: 10.1128/JVI.02419-10.
39. Pissani F, Malherbe D C, Robins H, DeFilippis V R, Park B, al e. Motif-optimized sutype A HIV envelope-based DNA vaccines rapidly elicit neutralizing antibodies when delivered sequentially. Vaccine. 2012; 30:5519-26. doi: 10.1016/j.vaccine.2012.06.042.
40. Salazar-Gonzalez J F, Bailes E, Pham K T, Salazar M G, Guffey M B, Keele B F, et al. Deciphering human immunodeficiency virus type 1 transmission and early envelope diversification by single-genome amplification and sequencing. J Virol. 2008; 82(8):3952-70. doi: 10.1128/JVI.02660-07. PubMed PMID: 18256145; PubMed Central PMCID: PMC2293010.
41. Salazar-Gonzalez J, Salazar M, Keele B, Learn G, Giorgi E, Li H, et al. Genetic identity, biological phenotype, and evolutionary pathways of transmitted/founder viruses in acute and early HIV-1 infection. J Exp Med. 2009; 206:1273-89.
42. Ganusov V, Goonetilleke N, Liu M, Ferrari G, Shaw G, McMichael A, et al. Fitness costs and diversity of CTL response determine the rate of CTL escape during the acute and chronic phases of HIV infection. J Virol. 2011; 85(20):10518-28.
43. Batorsky R, Sergeev R A, Rouzine I M. The route of HIV escape from immune response targeting multiple sites is determined by the cost-benefit tradeoff of escape mutations. PLoS Comput Biol. 2014; 10(10):e1003878. doi: 10.1371/journal.pcbi.1003878.
44. Huang W, Haubold B, Hauert C, Traulsen A. Emergence of stable polymorphisms driven by evolutionary games between mutants. Nature communications. 2012; 3:919. doi: 10.1038/ncomms1930. PubMed PMID: 22735447; PubMed Central PMCID: PMC3621454.
45. Moody M A, Gao F, Gurley T C, Amos J D, Kumar A, al e. HIV neutralizing antibodies without heterologous breadth can potently neutralize autologous viruses. submitted.
46. Pancera M, Zhou T, Druz A, Georgiev I S, Soto C, Gorman J, et al. Structure and immune recognition of trimeric pre-fusion HIV-1 Env. Nature. 2014;514(7523):455-61. doi: 10.1038/nature13808. PubMed PMID: 25296255.
47. Wu X, Zhou T, Zhu J, Zhang B, Georgiev I, Wang C, et al. Focused evolution of HIV-1 neutralizing antibodies revealed by structures and deep sequencing. Science. 2011; 333(6049):1593-602. doi: 10.1126/science.1207532. PubMed PMID: 21835983; PubMed Central PMCID: PMC3516815.
48. Zhou T, Georgiev I, Wu X, Yang Z Y, Dai K, Finzi A, et al. Structural basis for broad and potent neutralization of HIV-1 by antibody VRC01. Science. 2010; 329(5993):811-7. doi: 10.1126/science.1192819. PubMed PMID: 20616231; PubMed Central PMCID: PMC2981354.
49. Diskin R, Scheid J F, Marcovecchio P M, West A P, Jr., Klein F, Gao H, et al. Increasing the potency and breadth of an HIV antibody by using structure-based rational design. Science. 2011; 334(6060):1289-93. doi: 10.1126/science.1213782. PubMed PMID: 22033520; PubMed Central PMCID: PMC3232316.
50. Zhou T, Xu L, Dey B, Hessell A J, Van Ryk D, Xiang S H, et al. Structural definition of a conserved neutralization epitope on HIV-1 gp120. Nature. 2007;445(7129):732-7. doi: 10.1038/nature05580. PubMed PMID: 17301785; PubMed Central PMCID: PMC2584968.
51. West A P, Scharf L, Horwitz J, Klein F, Nussenzweig M C, Bjorkman P J. Computational analysis of anti-HIV-1 antibody neutralization panel data to identify potential functional epitope residues. Proceedings of the National Academy of Sciences of the United States of America. 2013; 110(26):10598-603. doi: 10.1073/pnas.1309215110. PubMed PMID: 23754383; PubMed Central PMCID: PMC3696754.
52. Crooks G E, Hon G, Chandonia J M, Brenner S E. WebLogo: a sequence logo generator. Genome research. 2004; 14(6):1188-90. doi: 10.1101/gr.849004. PubMed PMID: 15173120; PubMed Central PMCID: PMC419797.
53. Schneider T D, Stephens R M. Sequence logos: a new way to display consensus sequences. Nucleic Acids Res. 1990; 18(20):6097-100. PubMed PMID: 2172928; PubMed Central PMCID: PMC332411.
54. Kaufman L, Rousseew P J. Finding groups in data: An introduction to cluster analysis. Hoboken: John Wiley and Sons; 2005.
55. Murrell B, Wertheim J O, Moola S, Weighill T, Scheffler K, al e. Detecting individual sites subject to episodic diversifying selection. PLoS Genet 2012; 8(7):e1002764. doi: 10.1371/journal.pgen.1002764.
56. Pennings P S, Kryazhimskiy S, Wakeley J. Loss and recovery of genetic diversity in adapting populations of HIV. PLoS Genet. 2014; 10(1):e1004000. doi: 10.1371/journal.pgen.1004000.
57. Maher S J, Murray J M. The unrooted set covering connected subgraph problem differentiating between HIV envelope sequences. submitted.
58. Murray J M, Moenne-Loccoz R, Velay A, Habersetzer F, Doffol M, et al. Genotype 1 hepatitis C virus envelope features that determine antiviral response assessed through optimal covariance networks. PLoS ONE. 2013; 8(6):e67254. doi: 10.1371/journal.pone.0067254.
59. Korber B, Gnanakaran S. The implications of patterns in HIV diversity for neutralizing antibody induction and susceptibility. Curr Opin HIV AIDS. 2009; 4:408-17. doi: 10.1097/COH.0b013e32832f129e.
60. Sather D N, Carbonetti S, Malherbe D, Pissani F, Stuart A B, Hessell A J, et al. Emergence of broadly neutralizing antibodies and viral co-evolution in two subjects during the early stages of infection with human immunodeficiency virus type 1. J Virol. 2014; 88(22):12968-81. doi: 10.1128/JVI.01816-14.
61. Wang S, Mata-Fink J, Kriegsman B, Hanson M, Irvine D J, Eisen H N, et al. Manipulating the selection forces during affinity maturation to generate cross-reactive HIV antibodies. Cell. 2015; 160(4):785-97. doi: 10.1016/j.ce11.2015.01.027. PubMed PMID: 25662010.
62. Mcllroy D, Barteau B, Cany J, Richard P, Gourden C, al e. DNA/Amphiphilic block co-polymer nanospheres promote low-dose DNA vaccination. Mol Ther. 2009; 17(8):1473-81. doi: 10.1038/mt.2009.84.
63. Chèvre R, Le Bihan O, Beilvert F, Chatin B, Barteau B, Mével M, et al. Amphiphilic block copolymers enhance the cellular uptake of DNA molecules through a facilitated plasma membrane transport. Nucleic Acids Res. 2011; 39(4):1610-22. doi: 10.1093/nar/gkq922.
64. Guindon S, Gascuel O. A simple, fast, and accurate algorithm to estimate large phylogenies by maximum likelihood. Syst Biol. 2003; 52(5):696-704.
65. Guindon S, Dufayard J F, Lefort V, Anisimova M, Hordijk W, Gascuel O. New algorithms and methods to estimate maximum-likelihood phylogenies: assessing the performance of PhyML 3.0. Syst Biol. 2010; 59:307-21.
66. Nickle D C, Heath L, Jensen M A, Gilbert P B, Mullins J I, Kosakovsky-Pond S L. HIV-specific probabilistic models of protein evolution. PLoS ONE. 2007; 2:e503.
67. Paradis E, Claude J, Strimmer K. APE: analyses of phylogenetics and evolution in R language. Bioinformatics. 2004; 20:289-90.

### Example 8

Example 8 describes a method for swarm immunogen selection.

Neutralization breadths are uniformly distributed across chronic sera. This suggests anyone, not only 10-20%, might develop broadly neutralizing antibodies (bnAbs) if exposed to immunogens via vaccination. Working back from mature bnAbs through intermediates has enabled understanding their development from the unmutated germ-line ancestor, and showed that viral genetic diversity preceded the development of neutralization breadth. Described herein is the selection of sets of viral variants to investigate the role of antigenic diversity in serial samples. It is hypothesized that sites losing the ancestral, transmitted-founder (TF) virus state are most likely under positive selection, not drift. From acute, homogenous infections with 3-5 years of follow-up, sites of interest among plasma SGA Envs were identified by comparing the frequency of sequences per time-point having the TF state with a threshold, typically 5%. Sites with TF frequencies below threshold are putative escapes. Additional sites of interest were considered where more information was available, i.e. tree-corrected neutralization signatures and antibody contacts determined from co-crystal structure. Progressive loss of the TF form was used to identify clones carrying representative escape mutations.

In CH505, a study participant with an early antibody that bound autologous TF virus, 398 Envs from 14 time-points over three years were studied (median per sample: 25, range: 18-53). 36 sites with TF frequencies below 20% were found in any sample. Neutralization and structure data identified 28 and 22 interesting sites, respectively. Together, this identified six gp41 and 53 gp120 sites, plus six V1 or V5 insertions not in HXB2. 100 clones that represent the sites of interest were selected. Selected clones had a lower clustering coefficient and greater diversity in selected sites than sets sampled randomly. This approach was developed to select reagents for neutralization assays, then study affinity maturation, autologous neutralization, and the transition to heterologous neutralization and breadth. Specific implications for vaccine design, given sustained coevolution of immunity and escape is described herein.

Introduction

Neutralizing antibodies are immune correlates of protection in all licensed antiviral vaccines. It is not yet known how to induce broadly cross-reactive neutralizing antibodies (bnAbs) against HIV-1 via vaccination. Variation among proteins that interact directly with antibodies provides evolutionary signal about the effects of immune selection. Motivated by previous findings that early virus diversification drives neutralization breadth in early infection with HIV-1, it was hypothesized that progressively increasing antigen diversity can induce bnAbs. Herein immunogens with progressively increasing diversity at key sites in polymorphic viral proteins are identified. A major innovation of the swarm vaccine concept is rapid turnaround from viral sequence information to immunogen candidates. Another novel aspect is its potential for general utility to promote bnAb development against highly variable viruses, bacteria, and secreted toxins.

Neutralizing antibodies (nAbs) block viruses from entering cells.

All chronic plasmas neutralize some HIV-1 Envs; half neutralize at least 50% of diverse viruses. Virus Envelope diversity and ongoing immune escape drive selection for greater breadth Divergence, bNAb breadth/potencyEnv diversity precedes Nab breadth.

Typical course in natural infection: Autologous Nabs, followed by selection for relative Env resistance, then selection of bNAbs that tolerate the Env variants.

About 80% of new infections are established by single transmitted/founder (TF) virions that diversify randomly until immune selection becomes active.

Longitudinal samples from acute infection through 3-5 years of follow-up enable following bnAb development.

Single-genome sequencing of virus envelopes yields high-quality sequence data.

Diverse swarms of viral variants that induced breadth in bnAb donor were selected.

Related work in vaccine design has suggested that Env variants sampled during development of heterologous neutralization breadth could be administered as immunogens. Diversity among such Envs are hypothesized to emulate immune selection and induce antibodies with more varied specificities than single, clonal immunogens.

In other related research, Env selection has been formally represented as a set-coverage problem. That work identifies networks of covarying sites that occur in a population-level alignment, which represents a particular clade, subtype, or (in the case of hepatitis C virus) genotype. It considers the difference between early, transmissible, transmitted-founder viruses from later, chronic viruses, and utilizes only covarying sites found to occur in the TF stage. Though the underlying vaccine concept in that line of inquiry differs from that used herein, the formalism is related to the problem approach described herein.

Sequence alone: Using evidence for selection as measured by TF loss, once sequences are obtained, a swarm vaccine can be designed.

Contacts: requires antibody/Env structure, or an analog antibody with a known structure.

Signatures: correlations of mutations with bNAb sensitivity can be identified—identify sites of interest both inside and outside of contacts.

A simple indicator of immune selection in viral proteins was developed to identify immunogens that represent diversity induced during development of broadly neutralizing antibodies. A simple, efficient algorithm was designed and implemented to select sequences that represent the accumulation of mutations involved in immune recognition for a vaccine sequence cocktail or reagent set. By factoring in time of sampling, the algorithm starts with sequences nearest the form that established the infection, and progressively builds on diversity in a manner that parallels natural infection, so common mutations and mutations that eventually go to fixation are naturally sampled many times. It is deterministic for a given input set. However, unresolved ties may exist among alternative clones for some data.

Results

Site Selection

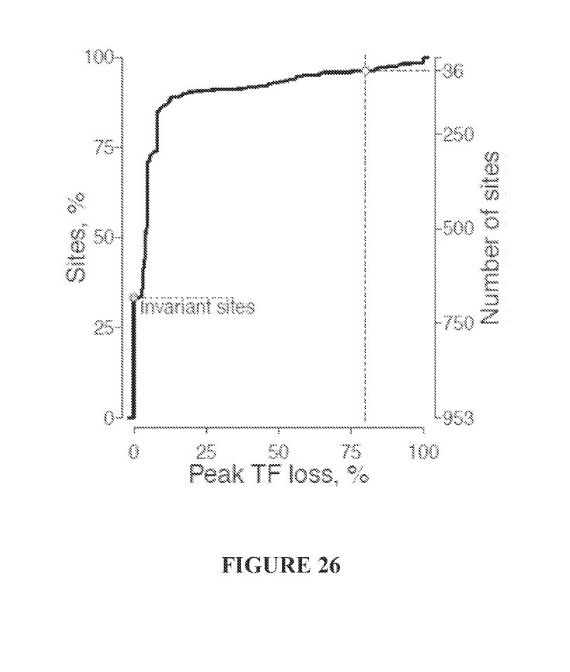

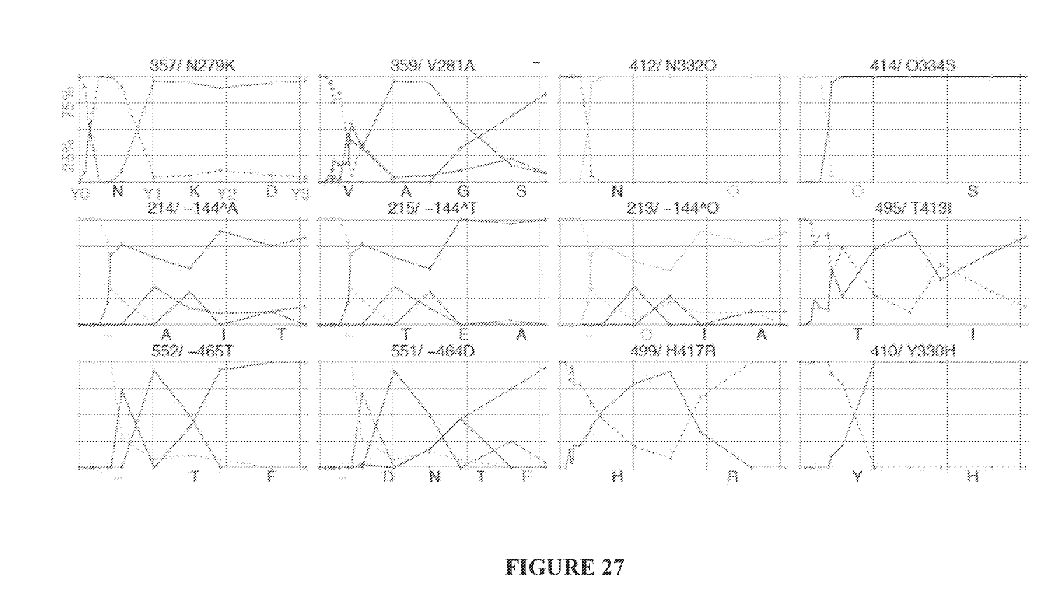

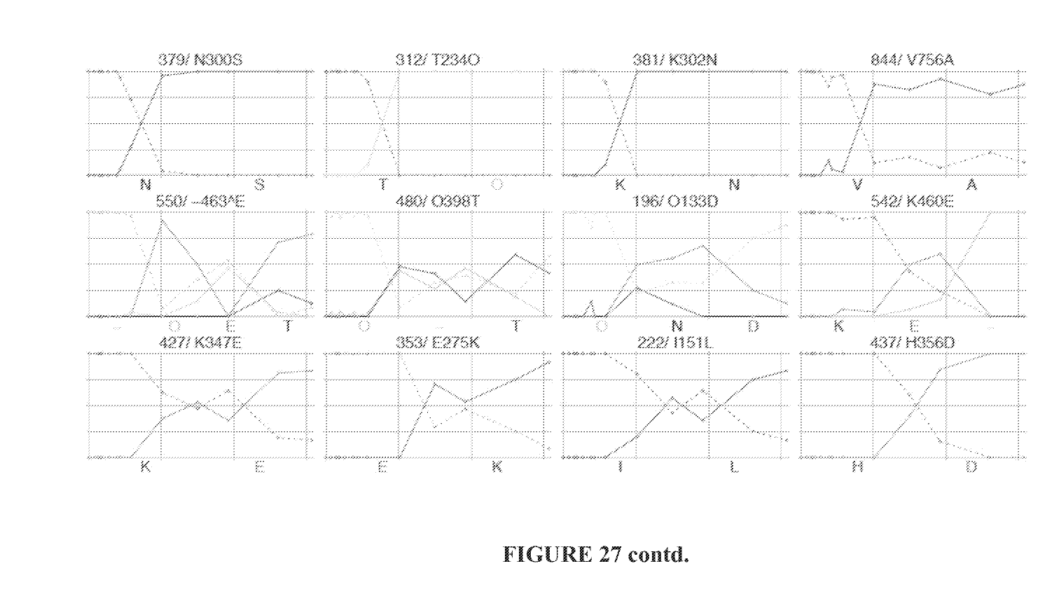

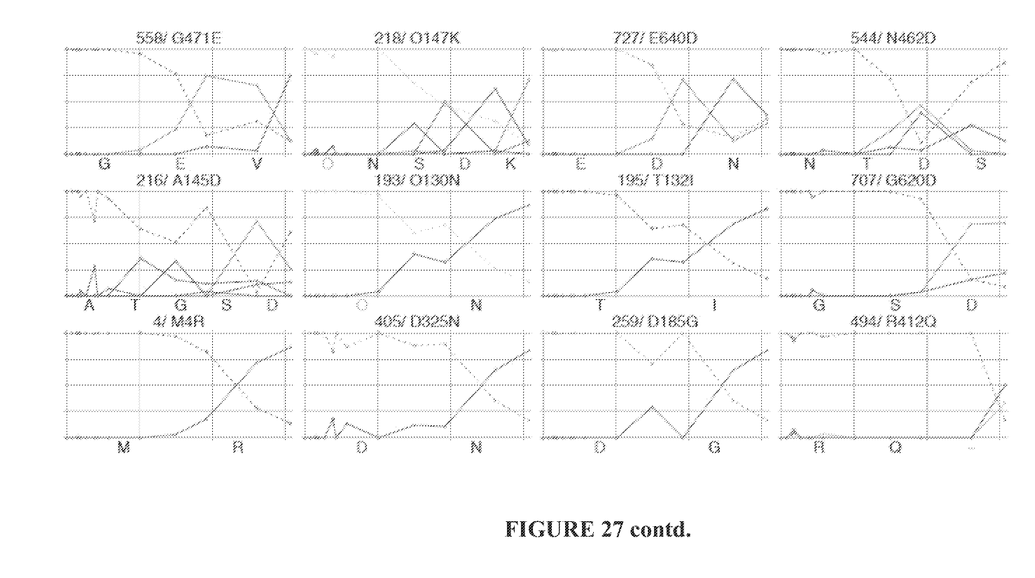

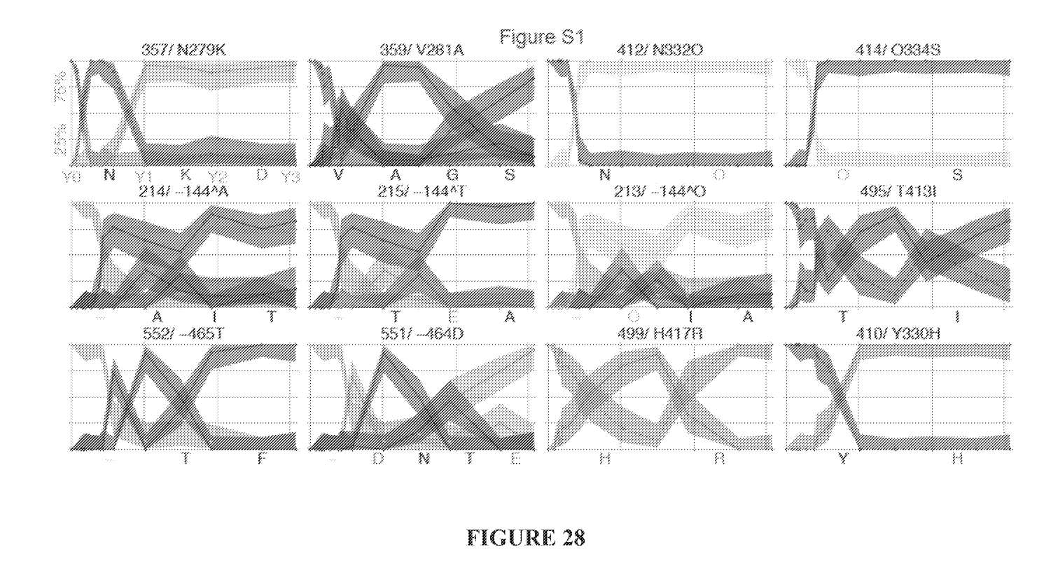

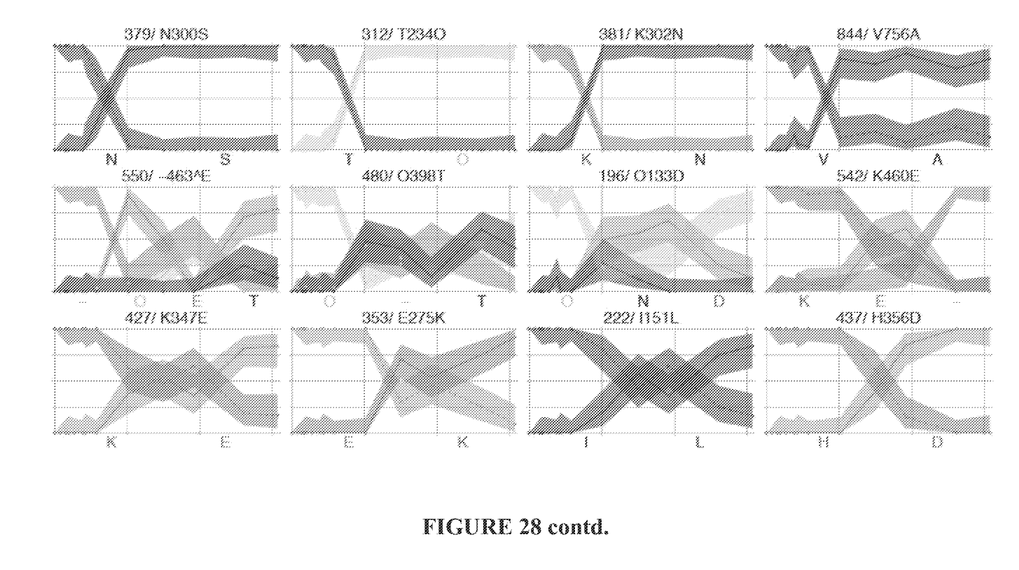

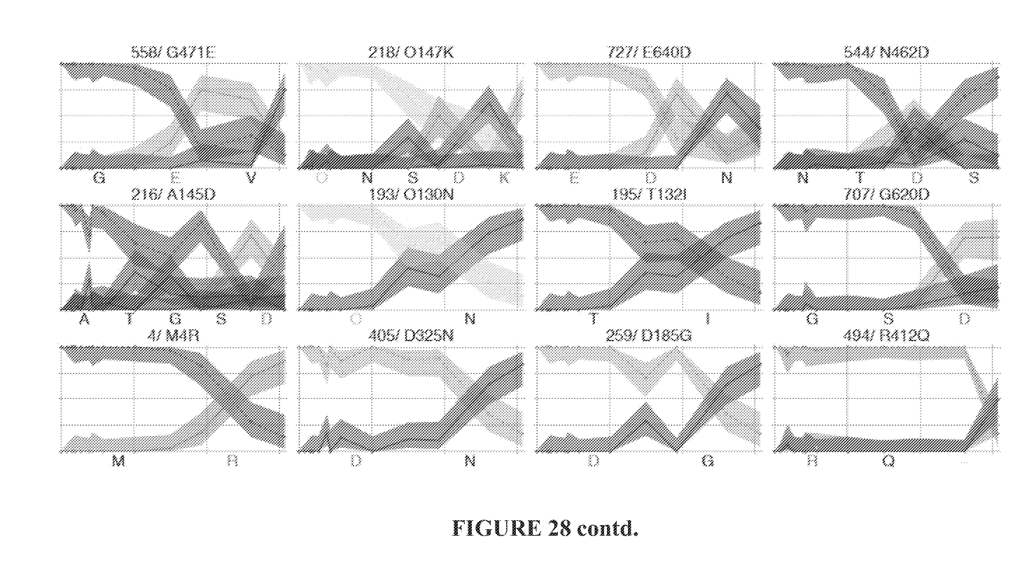

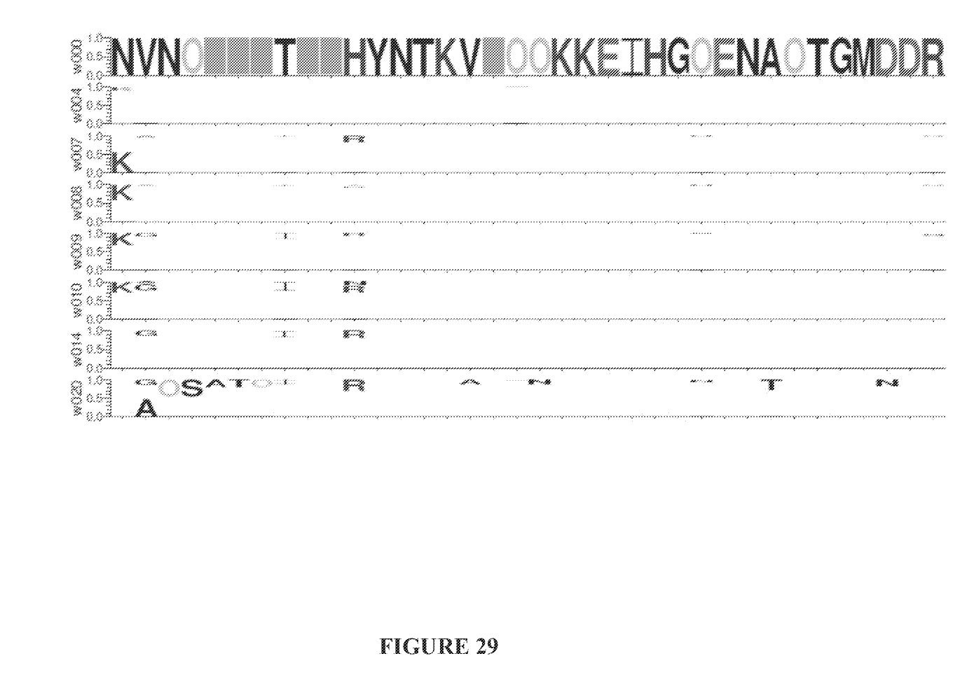

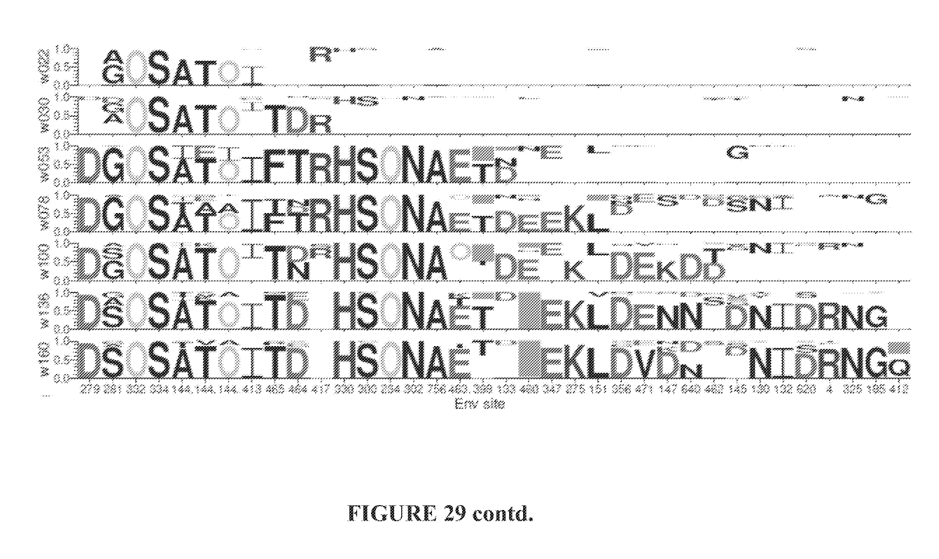

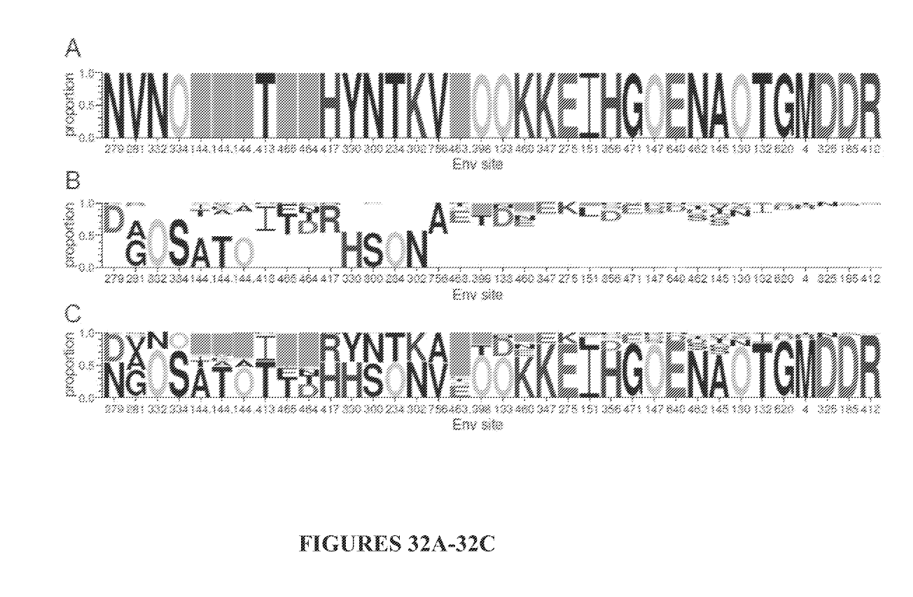

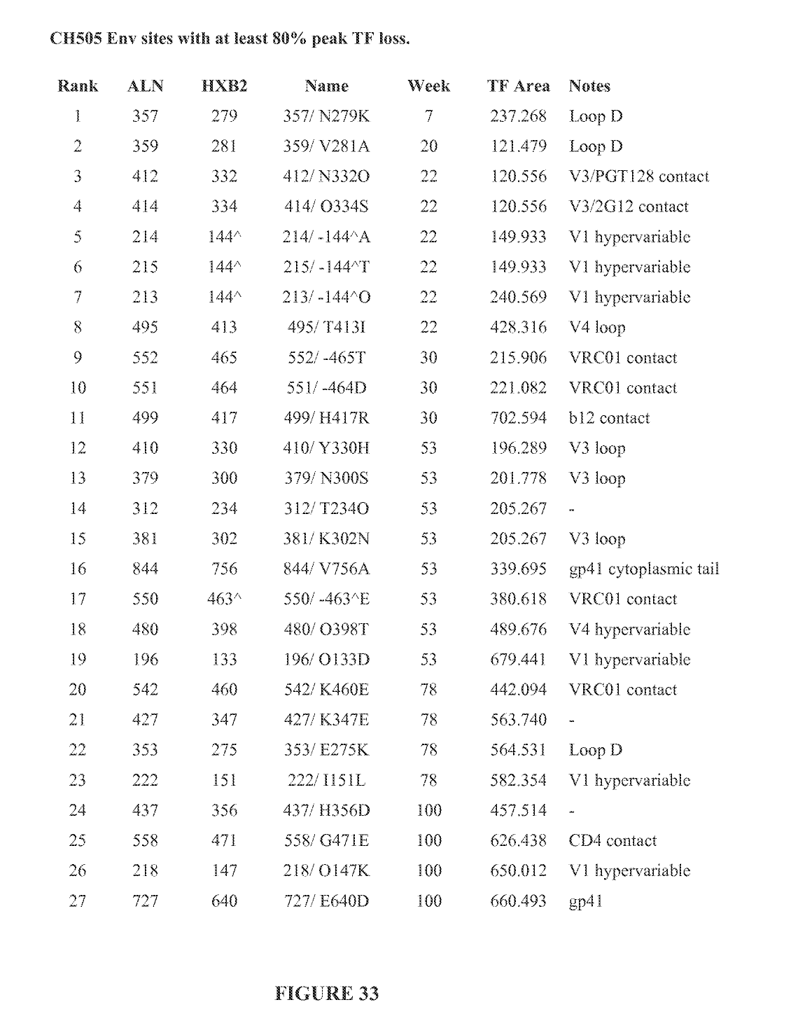

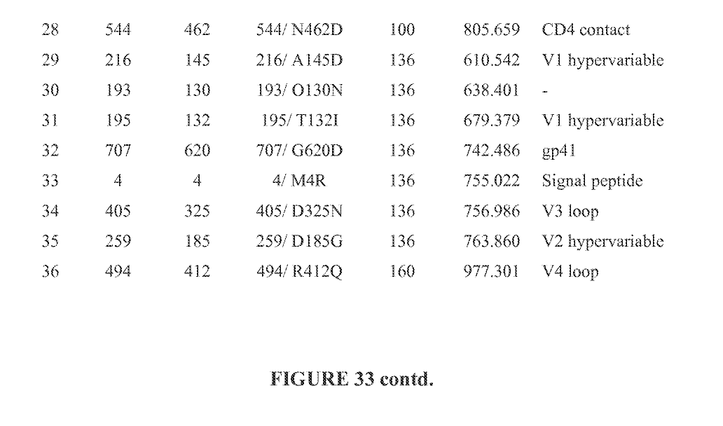

398 clones from 14 time-points over three years were aligned (median per sample: 25, range: 18-53) across 953 Env sites. TF loss per site was computed for each of 14 sample timepoints, weeks 4 through 160 (FIG. 27). Peak TF loss is the greatest TF loss per site over all timepoints sampled. The cumulative distribution of peak TF loss per site indicates a third of sites are invariant and 64 sites lose over 50% TF (FIG. 26). From this distribution, 36 sites with at least 80% peak TF loss were selected for further study (FIG. 33). These sites are putative escapes from immune selection, though their TF loss might be very slow or revert below threshold. Initially dominated by the TF form, variants emerge over time, with a variety of resulting dynamics across sites (FIG. 27). Reordering sites by when the TF first becomes minority, resolving ties with cumulative TF loss (FIG. 33), the progression of putative escapes is apparent (FIG. 29). Further, sites with 80% TF loss form localized clusters on the outer domain of gp120. The clustered patches on gp120 correspond to known antibody specificities. Two clusters are localized near the CD4 and CCRS binding sites, which correspond to the CH103 bnAb epitope (Liao H-X, Lynch R, Zhou T, Gao F, Alam S M, et al. (2013) Coevolution of a broadly neutralizing HIV-1 antibody and founder virus. Nature 496: 469-476. doi: 10.1038/nature12053). One cluster is localized to light-chain contacts and another to heavy-chain contacts. A third cluster, localized at the base of the V3 loop, corresponds to the epitope of DH151 and DH228. Three of the 36 sites appear on gp41 (620, 640, and 756; not shown).

Clone Selection

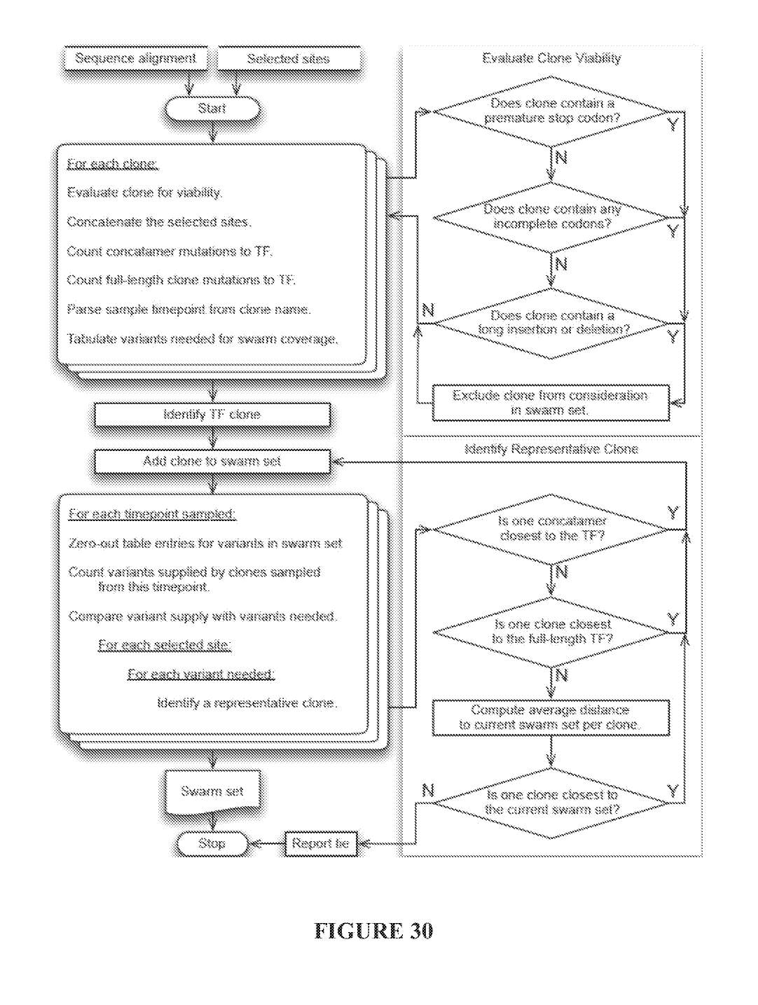

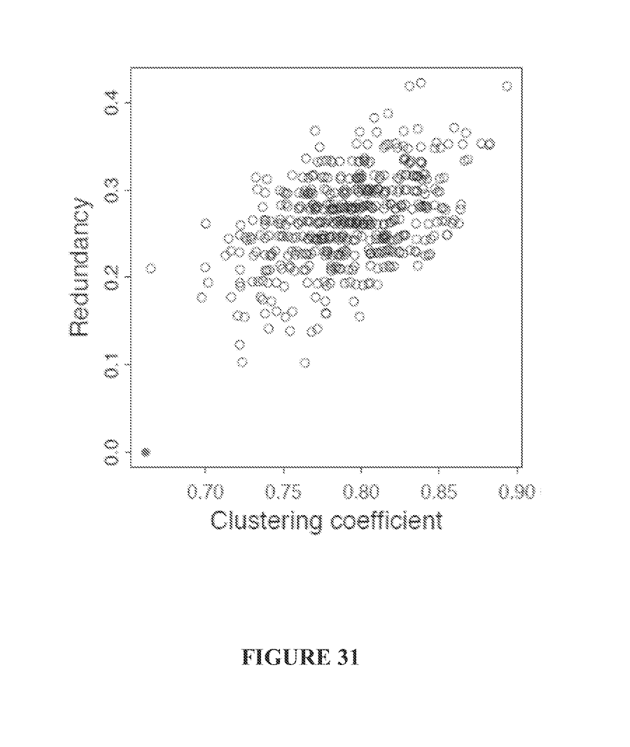

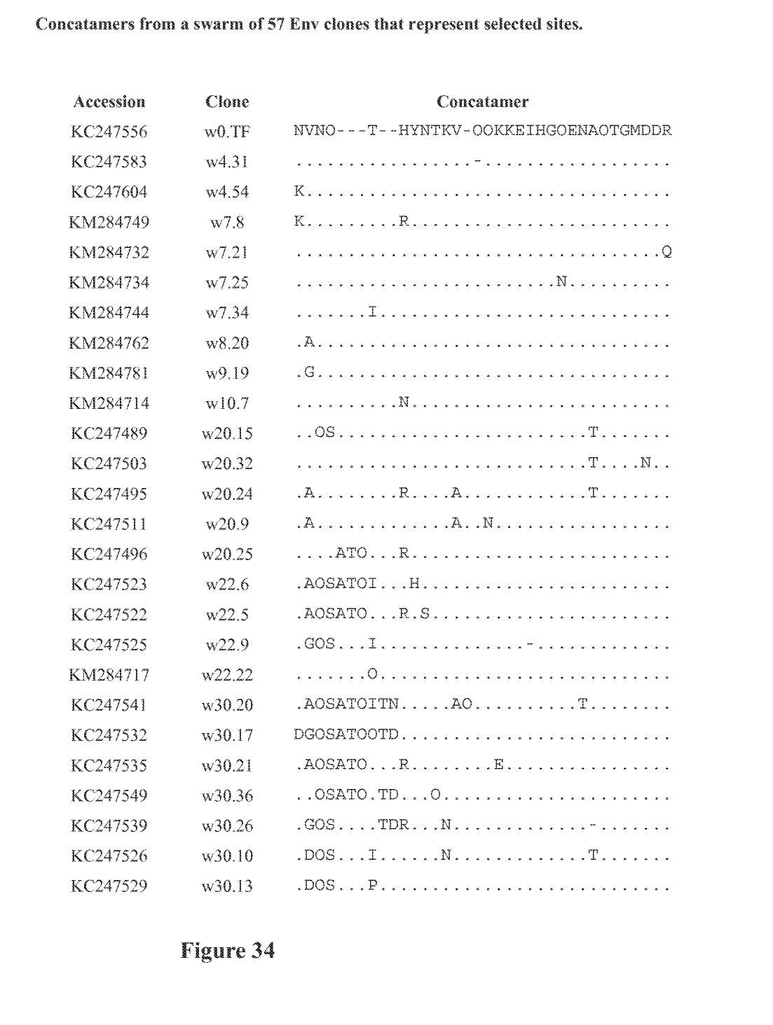

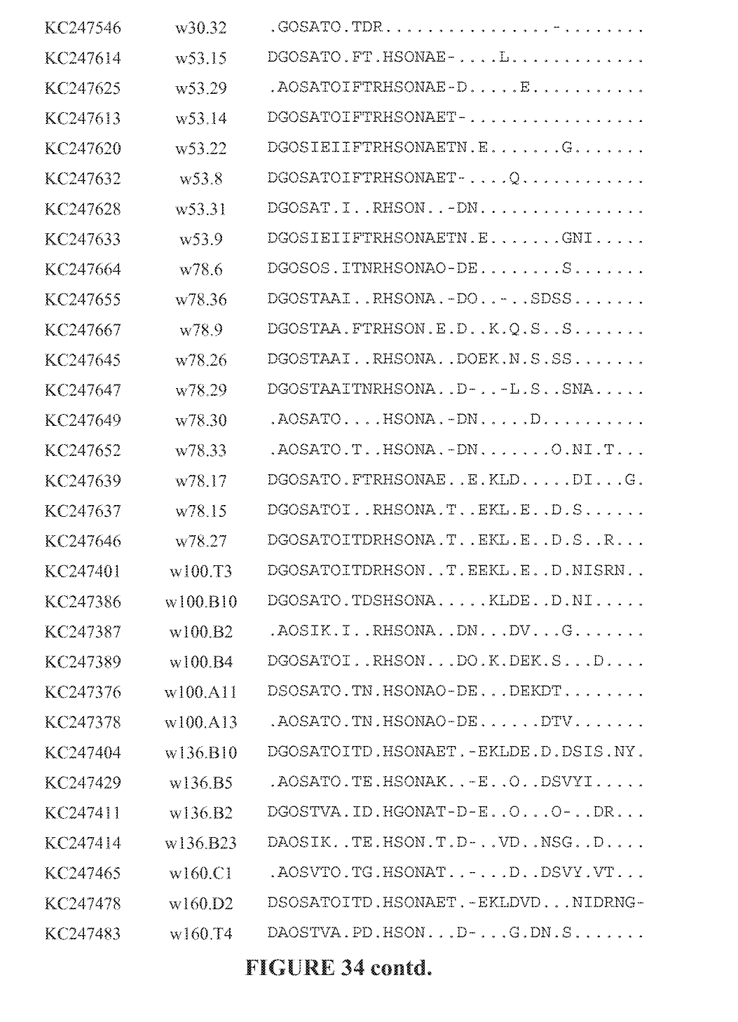

The selected sites were extracted from aligned sequences and concatenated to review Env variation among candidate clones. This representation as concatamers (sequences formed by concatenating selected sites) formed the basis for clone selection. The greedy swarm-selection algorithm (FIG. 30) identified 57 clones that cover variant diversity at 36 selected sites (FIG. 34). None of the concatamers from the selected clones are duplicates, which is unlikely to occur when choosing clones randomly (FIG. 31). The selected clones also have a lower clustering coefficient than sets of randomly selected clones (FIG. 31). The lower clustering coefficient indicates less hierarchical structure among subsets of concatamers from the selected clones (Kaufman L, Rousseew P J (2005) Finding groups in data: An introduction to cluster analysis. Hoboken: John Wiley and Sons. 342).

Discussion

Because sites are not independent, but covary, information about site covariation could facilitate smaller swarm sets that represent selected sites. Other optimization algorithms are likely to yield smaller swarms, for the small cost of more computing time.

Experimental validation as immunogens will be carried out.

A strategy to identify candidates for Env sites under immune selection from longitudinally sampled sequences was developed. In CH505, two thirds of these selected sites were ultimately related to the CH103 bNAb lineages, by either signature analysis or structural contacts proximity. Whether this information can guide selection of vaccine antigen sets that recapitulate the evolutionary pressure imposed by Env antigenic diversity on bNAb lineages is being explored. In some embodiments, gradual accumulation of epitope diversity may be key.

Methods

Site Selection

Transmitted-founder (TF) loss is the proportion of sequences sampled per time-point that have lost the ancestral TF state. This is an efficient way to select rapidly evolving sites. Herein, no other information than TF loss was considered, though such information could be used to select sites. This could include signature sites associated with neutralization assay outcomes and antibody contact residues from structural data, if available.

The starting point was SGA env (DNA) sequences, from a minimum of roughly 20 clones sampled longitudinally, beginning early (3-6 weeks) after infection, with 3-5 years of clinical follow-up. It is common for SGA from homogeneous infections to yield multiple identical sequences, all of which were kept. A naming convention for clones was used to ensure consistency and so sample time-point labels could be parsed from sequence names. To study variant dynamics, the number of elapsed days after the earliest sample from sample dates was computed, and the number of days post-infection estimated from the earliest sample was added. For homogeneous infections sampled before the onset of immune selection, a simple model of sequence evolution provides the estimate (Keele B F, Giorgi E E, Salazar-Gonzalez J F, Decker J M, Pham K T, et al. (2008) Identification and characterization of transmitted and early founder virus envelopes in primary HIV-1 infection. Proc Natl Acad Sci (USA) 105: 7552-7557; Giorgi E E, Funkhouser B, Athreya G, Perelson A S, Korber B T, Bhattacharya T (2010) Estimating time since infection in early homogeneous HIV-1 samples using a Poisson model. BMC Bioinformatics 11: 532. doi: 10.1186/1471-2105-11-532).

The HXB2 reference sequence was added to facilitate numbering positions, codon-aligned the sequences, translated them, and inferred a phylogeny. Though no algorithm aligns the HIV envelope perfectly, a useful starting point for manual alignment uses an HIV-specific hidden Markov model [GeneCutter]. Aligning all but the hypervariable loops is trivial given such a preliminary alignment. Because hypervariable loops evolve rapidly by tandem duplications, a useful alignment criterion is self-consistency, rather than identification of homologous sites. For example, a putative N-linked glycosylation motif could be placed at either the N- or C-terminal position of an otherwise gapped region. Uniform placement of such motifs, particularly where HXB2 has no corresponding sites, facilitates analysis because the variants appear more clearly as evolutionary signal if aligned consistently.

Maximum-likelihood trees were inferred from translated amino-acid sequences with PhyML and the HIVw (HIV-specific, within-host) substitution model. The phylogeny is used to order sequences and is an organizing principle for sequence evolution from the ancestral TF virus.

To identify potential N-linked glycosylation (PNG) sites, PNG sites were annotated by replacing asparagine sites that match the Nx[ST] motif to become Ox[ST]. (In the PNG motif, x indicates any amino acid except proline, and the third position is either serine or threonine).

For each aligned site, TF loss was computed per time-point sequenced, the maximum was identified, and this “peak” TF loss was compared with a threshold. The TF loss threshold determines the number of sites that are selected; a high TF loss threshold yields fewer sites than a low threshold. The threshold will depend on many variables, such as number of sequences sampled and time since infection. The threshold was adjusted and the resulting number of sites considered. This gave a list of sites in the alignment, which was considered as interesting evolutionary “hot spots” to be represented by a swarm of clones.

Clone Selection

Having used the TF-loss criterion to select sites from the alignment, a set of clones was identified to represent the variants that occur at these sites. Choosing k representatives from n clones gives at least 10¹⁰⁰ possibilities for n above 427 and k over 100. On the scale of the current example, choosing 50 clones from 250 candidates gives over 10⁵³ alternatives. To search such a vast space of possible solutions is intractable for even the fastest computers.

A simple, efficient algorithm was designed and implemented to select sequences to represent variants at sites selected by TF loss. The approach is greedy in that it adds clones iteratively, rather than refine the entire clone set for potentially better solutions. Such a greedy approach is unlikely to give the best possible solution, but can efficiently provide reasonably good solutions, and can be refined to include other criteria as needed. It works from the same alignment used to select sites, and assumes that the TF form and sample timepoints can be identified from clone names.

Clone selection works by initially tabulating amino acid variants among selected sites. This table of variant counts is used to monitor which remaining mutations need to be included in the swarm set. Variants that only ever appear once are disregarded. Candidates for clone selection must be functionally viable, by lacking long deletions and premature stop codons or incomplete codons, which typically result from frame-shift mutations. Starting with the TF form, the procedure iterates chronologically over timepoints sampled, and identifies a clone to represent each needed variant at each of the selected sites, should such a variant be present. The choice among multiple clones that carry a needed variant is resolved by a series of tie-breaking criteria, first to minimize distance (number of mutations, including gaps) to the TF form among selected sites, then for the full-length clone, and finally to minimize average distance to clones in the current swarm set. Any remaining ties would indicate a need for additional selection criteria. The clone selected to represent the needed variant is included in the swarm set, corresponding counts in the table of needed variants are set to zero, and iteration continues. Upon iterating over all sample timepoints, selected variants, and needed sites, the clone set is complete. A benefit of this approach is that it selects only as many clones are necessary to represent the variants in selected sites. However, the greedy approach errs towards inclusion of early point mutations that would be included among later variants.

### REFERENCES

Pissania F, Malherbe D C, Robins H, DeFilippis V R, Park B, et al. [Sellhorn G, Stamatatos L, Overbaugh J, Haigwood N L] (2012) Motif-optimized subtype A HIV envelope-based DNA vaccines rapidly elicit neutralizing antibodies when delivered sequentially. Vaccine 30: 5519-5526. dx.doi.org/10.1016/j.vaccine.2012.06.042

Malherbe D C, Doria-Rose N A, Misher L, Beckett T, Puryear W B, et al. [Schuman J T, Kraft Z, O′Malley J, Mori M, Srivastava I, Barnett S, Stamatatos L, Haigwood N L] (2011) Sequential immunization with a subtype B HIV-1 envelope quasispecies partially mimics the in vivo development of neutralizing antibodies. J Virol 85: 5262-5274. doi:10.1128/JVI.02419-10

Pissani F, D C Malherbe, Schuman J T, Robins H, Park B S, et al. [Krebs S J, Barnett S W, Haigwood N L] (2014) Improvement of antibody responses by HIV envelope DNA and protein co-immunization. Vaccine 32: 507-513. dx.doi.org/10.1016/j.vaccine.2013.11.022

Giorgi E E, Funkhouser B, Athreya G, Perelson A S, Korber B T, Bhattacharya T (2010) Estimating time since infection in early homogeneous HIV-1 samples using a Poisson model. BMC Bioinformatics 11: 532. doi: 10.1186/1471-2105-11-532

Haynes B F, Kelsoe G, Harrison S C, Kepler T B (2012) B-cell—lineage immunogen design in vaccine development with HIV-1 as a case study. Nature Biotechnol 30: 423-433. doi: 10.1038/nbt.2197

Kaufman L, Rousseew P J (2005) Finding groups in data: An introduction to cluster analysis. Hoboken: John Wiley and Sons. 342 p.

Keele B F, Giorgi E E, Salazar-Gonzalez J F, Decker J M, Pham K T, et al. (2008) Identification and characterization of transmitted and early founder virus envelopes in primary HIV-1 infection. Proc Natl Acad Sci (USA) 105: 7552-7557.

Kwong P D, Mascola J R, Nabel G J (2013) Broadly neutralizing antibodies and the search for an HIV -1 vaccine: the end of the beginning Nat Rev Immunol 13:693-701.

Liao H-X, Lynch R, Zhou T, Gao F, Alam S M, et al. (2013) Coevolution of a broadly neutralizing HIV-lantibody and founder virus. Nature 496: 469-476. doi: 10.1038/nature12053

Corti D, Lanzavecchia A (2013) Broadly neutralizing antiviral antibodies. Annu Rev Immunol 31: 705-742.

Burton D R, Desrosiers R C, Doms R W, Koff W C, Kwong P D, et al. (2004) HIV vaccine design and the neutralizing antibody problem. Nat Immunol 5: 233-236.

Burton D R, Ahmed R, Barouch D H, Butera S T, Crotty S, et al. (2012) A Blueprint for HIV vaccine discovery. Cell Host Microbe 12: 396-407. doi: 10.1016/j.chom.2012.09.008

Klein F, Mouquet H, Dosenovic P, Scheid J F, Scharf L, Nussenzweig M C (2013) Antibodies in HIV-1 vaccine development and therapy. Science 341: 1199-1204.

Korber B, Gnanakaran S (2009) The implications of patterns in HIV diversity for neutralizing antibody induction and susceptibility. Curr Opin HIV AIDS 4: 408-417. doi: 10.1097/COH.0b013e32832f129e

Kwong P D, Mascola J R (2012) Human antibodies that neutralize HIV: Identification, structures, and B cell ontogenies. Immunity 37: 412-425.

McGuire A T, Hoot S, Dreyer A M, Lippy A, Stuart A, et al. (2013) Engineering HIV envelope protein to activate germline B cell receptors of broadly neutralizing anti-CD4 binding site antibodies. J Exp Med 210: 655-633.

Murray J M, Moenne-Loccoz R, Velay A, Habersetzer F, Doffol M, et al. (2013) Genotype 1 hepatitis C virus envelope features that determine antiviral response assessed through optimal covariance networks. PLoS ONE 8(6): e67254. doi: 10.1371/journal.pone.0067254

### Example 9

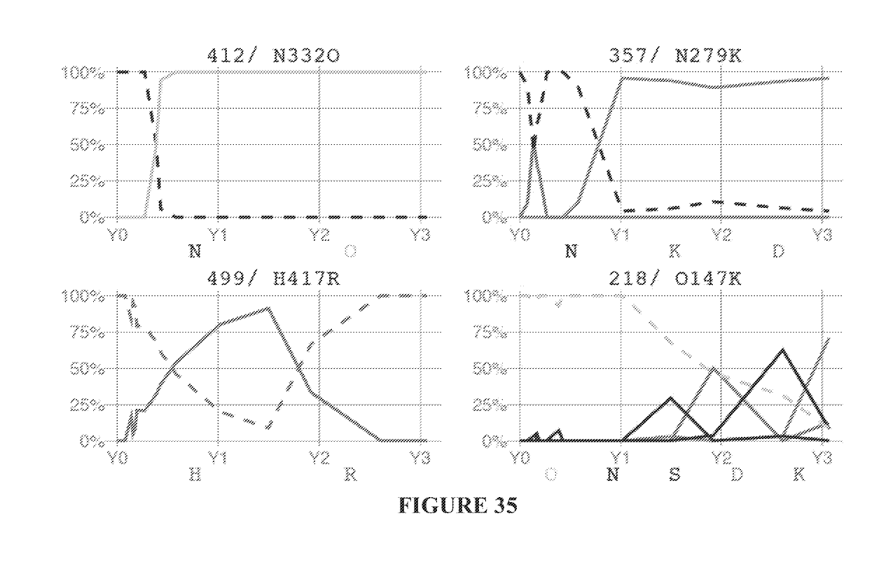

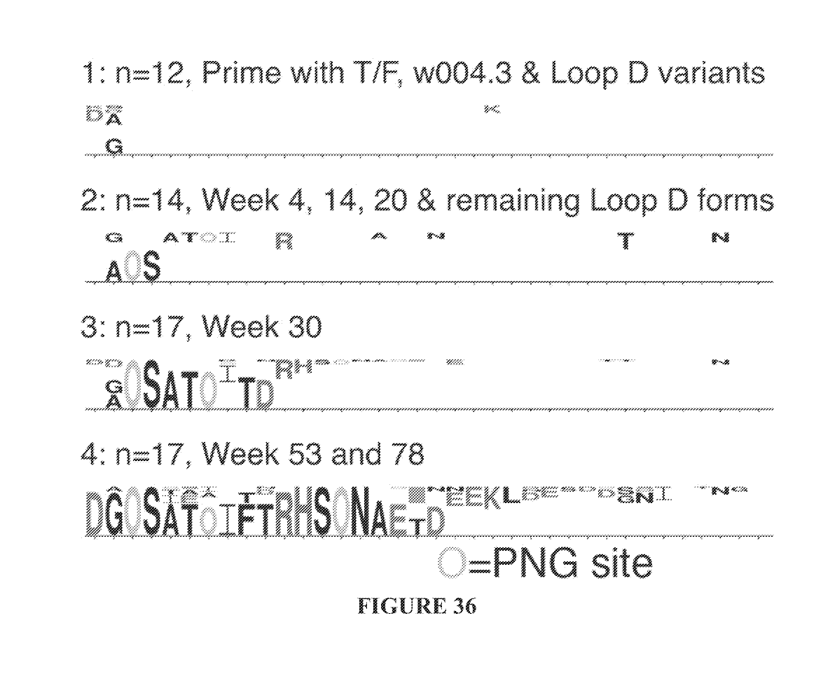

Example 9 describes swarm immunogen concept. Sites are identified by TF loss (FIG. 35), Clones with representative diversity are selected (FIG. 36).

### Example 10

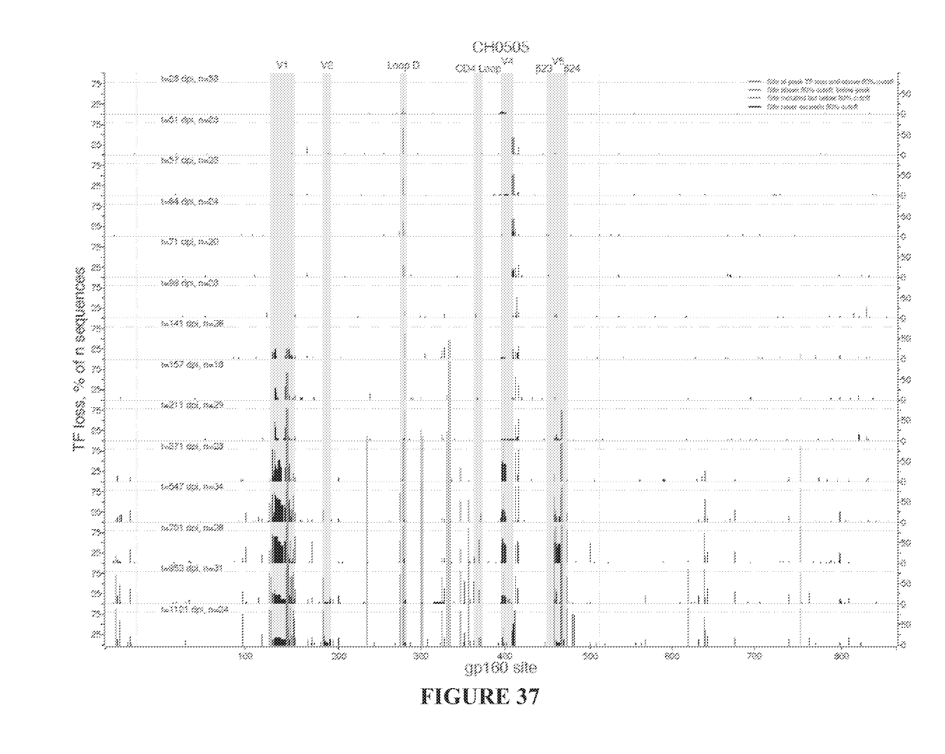

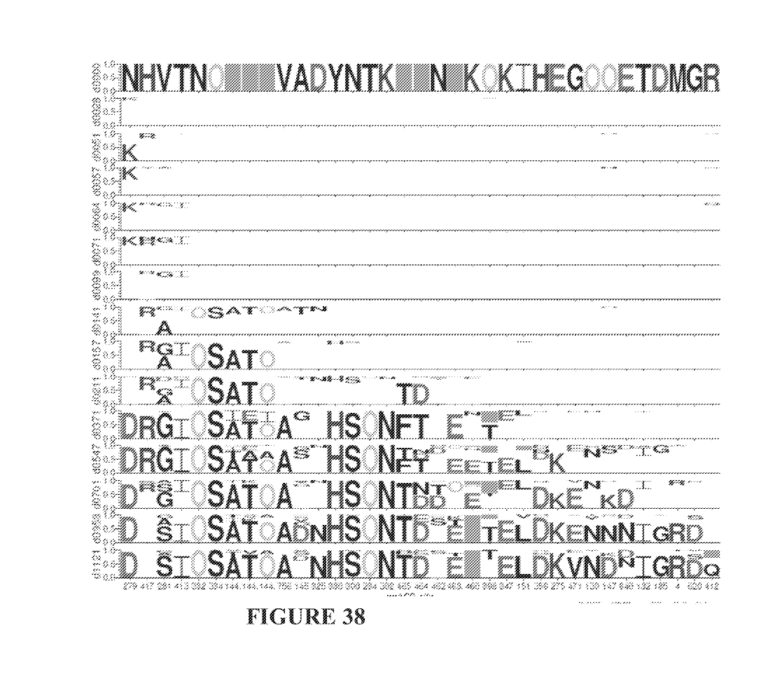

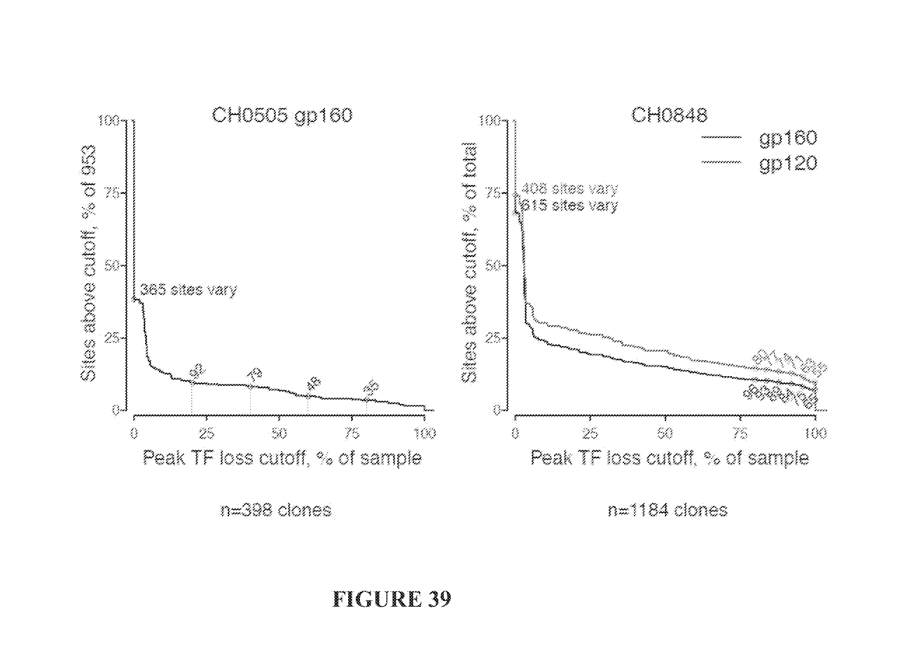

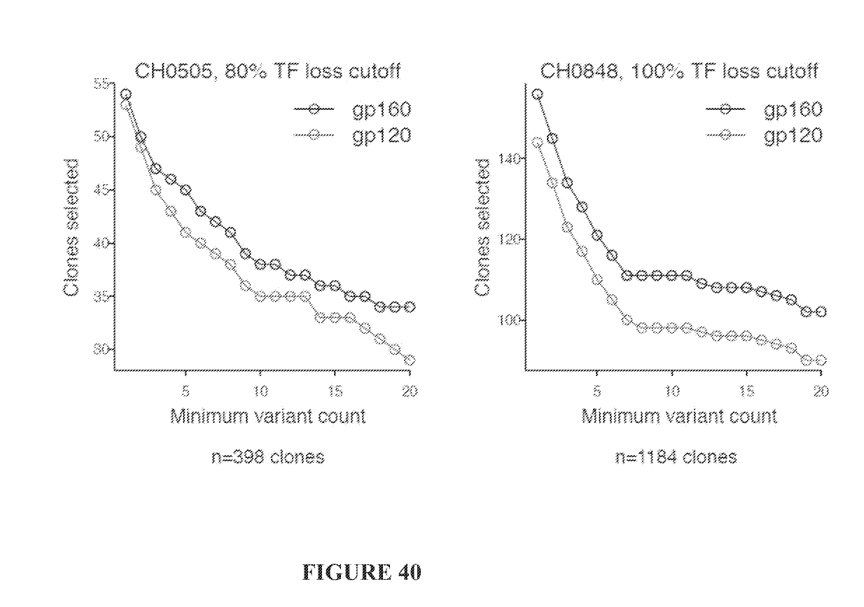

Example 10 describes swarm selection. Diversity in rapidly evolving sites are sampled as progression of mutations away from TF. Serially sampled sequences are aligned to TF or UA. For site selection, the number of sites selected depends on TF loss cutoff (FIG. 39). The loss of ancestral transmitted-founder (TF) amino acids in Envs from CH505 is shown in FIG. 37. The variant frequency across 35 sites selected from CH505 Env gp160 stratified by time is shown in FIG. 38. In some embodiments, CH505 sites with 80% TF loss formed two clusters on the gp120 outer domain. The outcome of clone selection depends on minimum variant count (FIG. 40).

### Example 11

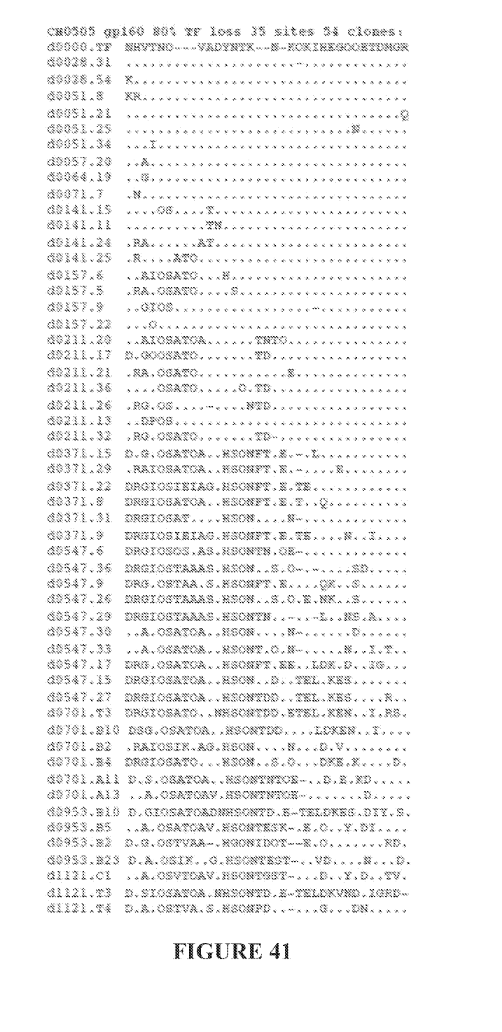

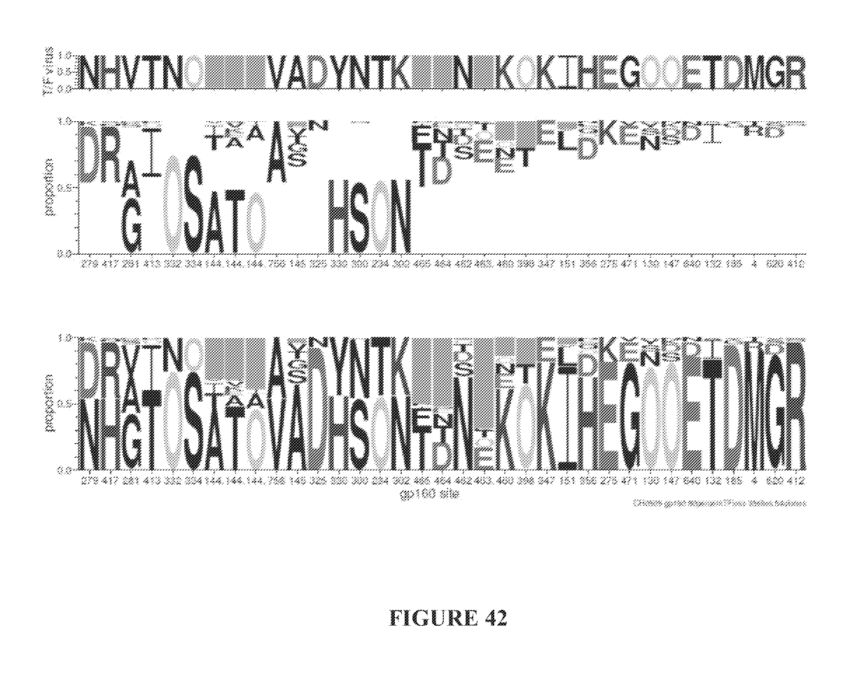

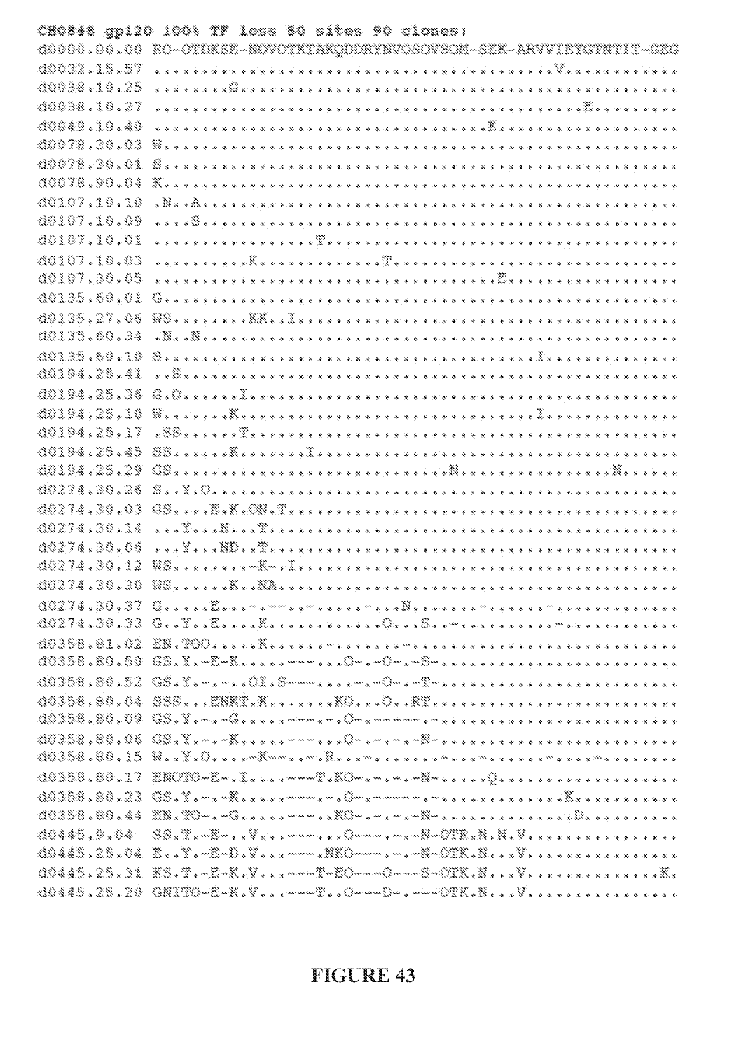

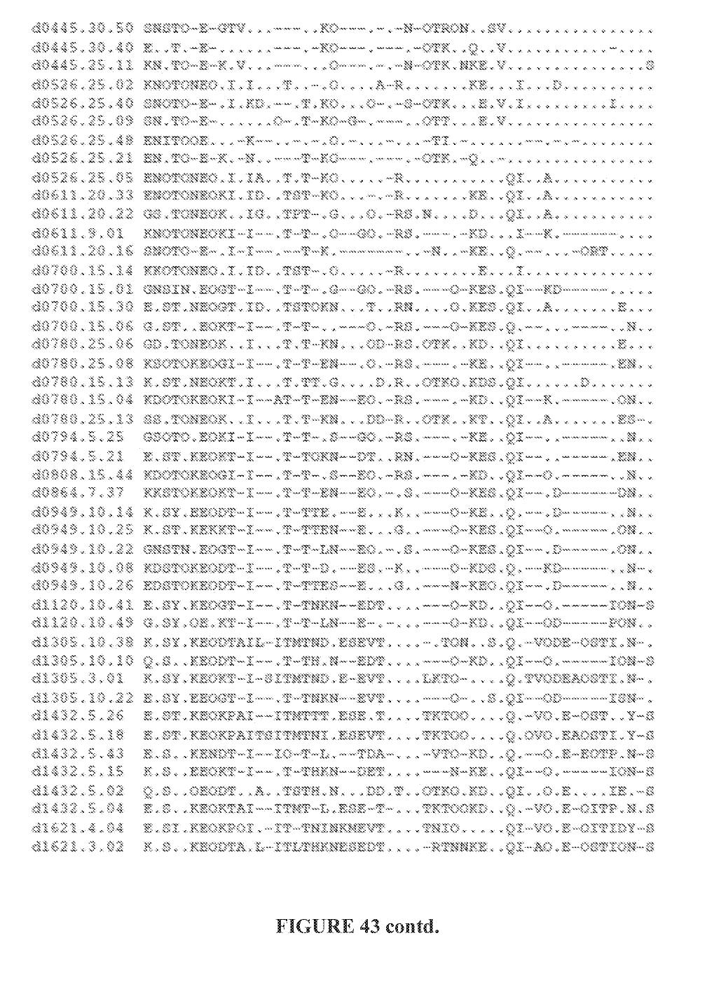

Example 11 describes selection procedure. The variants seen are tabulated across all sequences. Rare variants are excluded (<minimum variant count). Variant counts are updated while selecting sequences. For each time point sampled (1 . . . t), for each site selected (1 . . . s), and for each variant not yet included (1 . . . v), select the sequence that can uniquely minimize HD to TF among the selected sites, minimize HD to TF over full length, or minimize mean HD to current swarm. Concatamers from a swarm of 54 env clones that represent selected sites are shown in FIG. 41 and swarm variant frequency from 35 selected sites is shown in FIG. 42. Concatamers from a swarm of 90 env clones that represent selected sites are shown in FIG. 43.

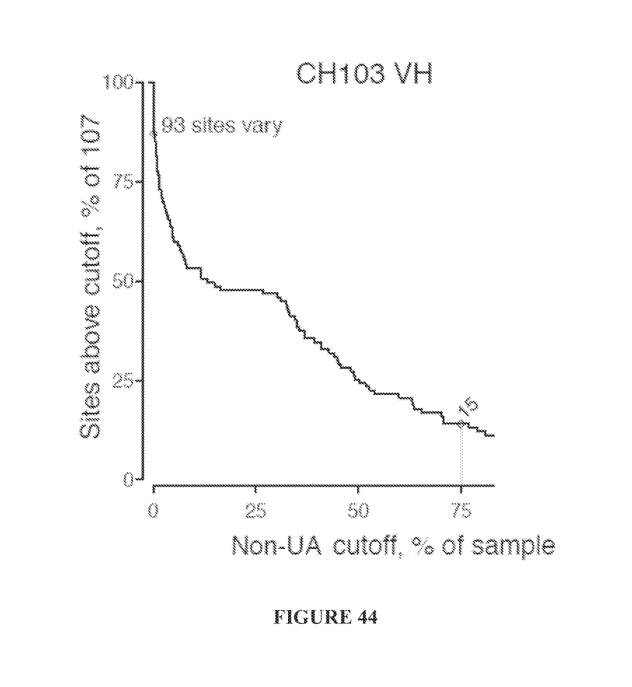

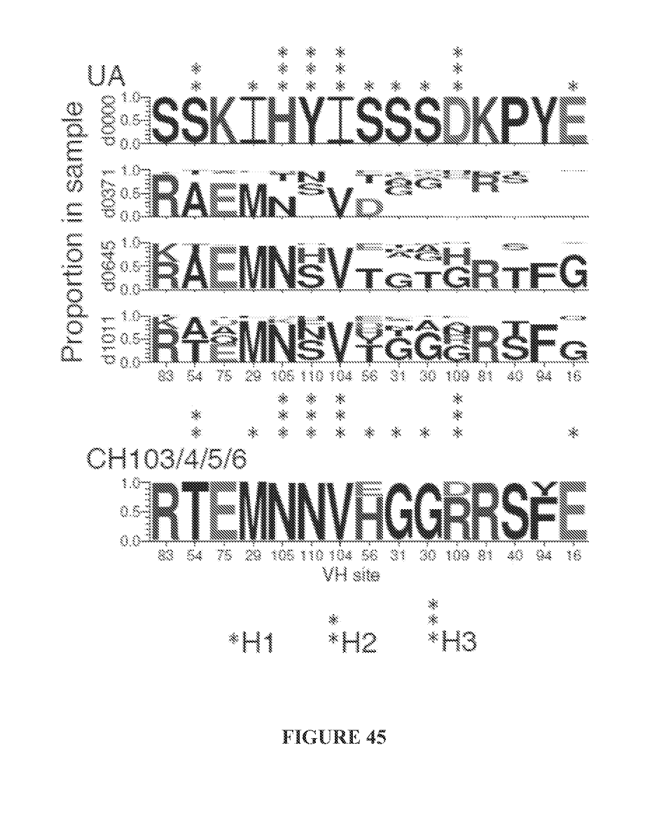

For CH103 VH, the sites above cutoff versus the non-UA cutoff is plotted in FIG. 44. The variant frequency across 15 V_{H} sites stratified by time is shown in FIG. 45.

Swarms are sequence sets that represent variant diversity from shared ancestor. Selected sites have highest peak TF loss. An algorithm selects clones that carry all but rare variants. Applications include reagent selection and immunogens for bnAb induction. R packages are in preparation (pixgramr and swarmtools).

### Example 12

Example 12 describes the structure of antibody CH103 in complex with the outer domain of HIV-1 gp120. Overall structure of the CH103-gp120 complex, with gp120 polypeptide depicted in ribbon and CH103 shown as a molecular surface.

### Example 13

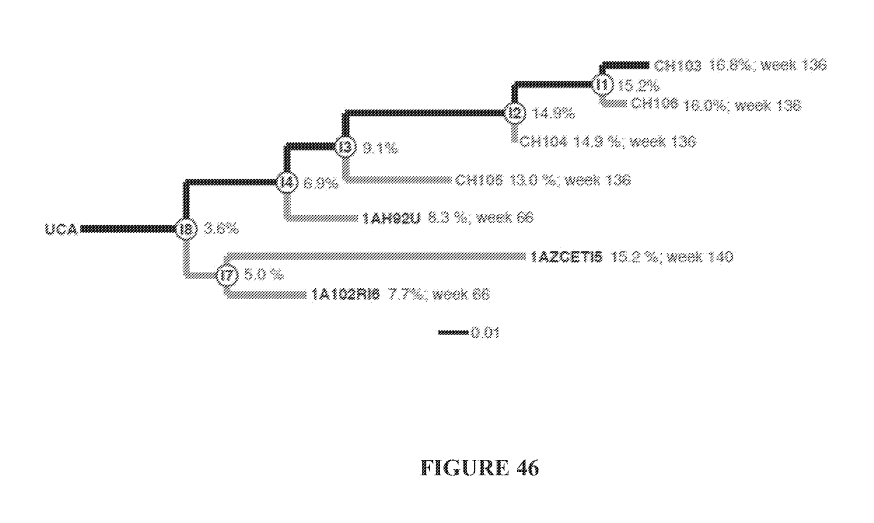

Example 13 describes the time of appearance and V_{H}DJ_{H} mutations in CH103 clonal family. Maximum likelihood phylogram showing the CH103 lineage with the inferred intermediates (circles, 11-4, 17 and 18), and percentage mutated V_{H} sites and timing indicated. Mutation frequency is 4-17% (FIG. 46).

### Example 14

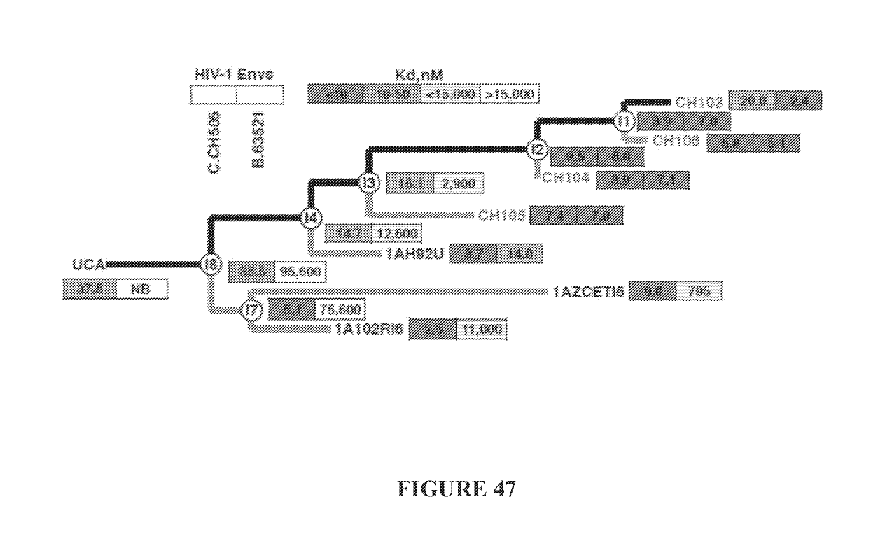

Example 14 describes the binding affinity maturation for the CH103 clonal family. Binding affinities (Kd, nM) of antibodies to autologous subtype C CH505 (C.CH505; left box) and heterologous B.63521 (right box) were measured by surface plasmon reasonance (FIG. 47).

### Example 15

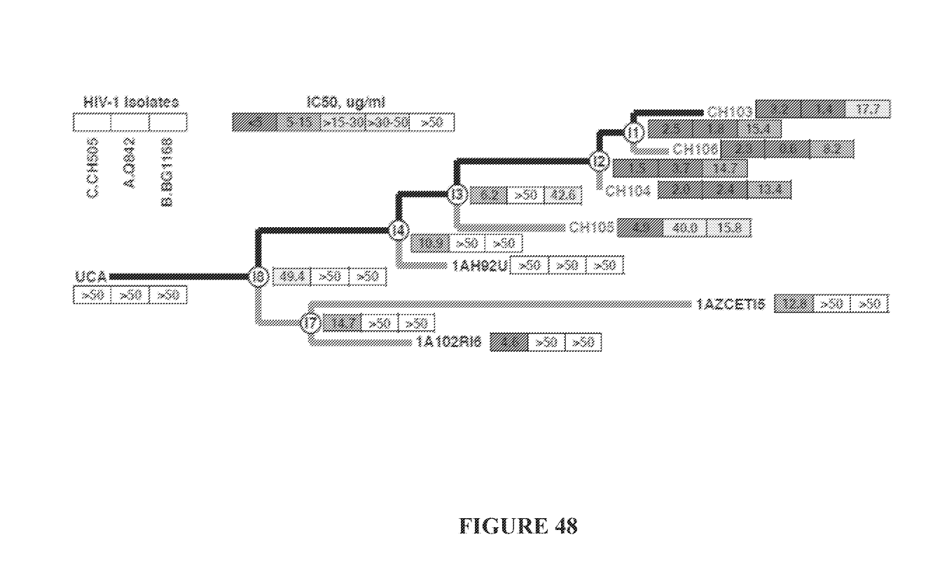

Example 15 describes the development of neutralization breadth in the CH103 clonal lineage. The phylogenetic CH103 clonal lineage tree showing the IC50 (mg ml21) of neutralization of the autologous transmitted/founder (C.CH505), heterologous tier clades A (A.Q842) and B (B.BG1168) viruses as indicated in FIG. 48. There is increasing neutralization potency and breadth (TZM-bl assay).

### Example 16

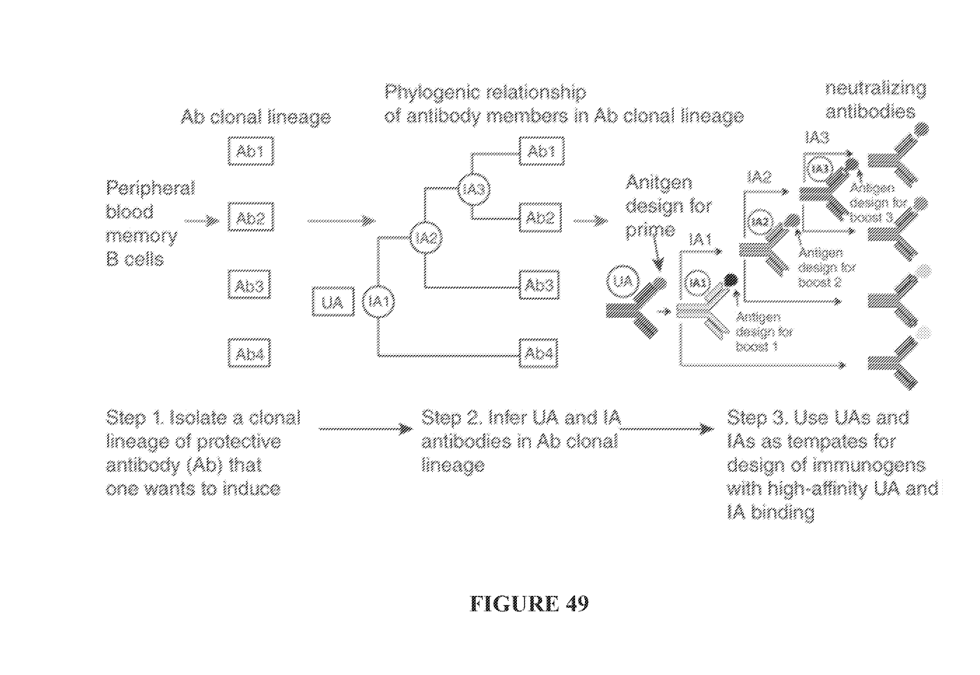

Example 16 describes the steps of a B-cell-lineage—based approach to vaccine design (FIG. 49). Step 1 is to isolate VH and VL chain members from the peripheral blood or tissues of patients containing BnAbs and to express these native Ig chain pairs as whole antibodies. Step 2 is to infer intermediate ancestor antibodies (IAs, labeled 1, 2 and 3) and the unmutated ancestor antibody (UA). Step 3 requires producing the unmutated and intermediate ancestors as recombinant mAbs and using structure-based alterations in the antigen (changes in Env constructs predicted to enhance binding to the unmutated or intermediate ancestors) or deriving altered antigens using a suitably designed selection strategy. Vaccine administration might prime with the antigen that binds the unmutated ancestor most tightly, and this is then followed by sequential boosts with antigens optimized for binding to each intermediate ancestor. Shown here is an actual clonal lineage of the V1/V2-directed BnAbs CH01-CH04. Targeting the unmutated ancestor with an immunogen that has enhanced binding may induce higher antibody responses. If high-affinity ligands for unmutated ancestors cannot be found, then high-affinity ligands targeting the intermediate ancestors may be equally useful for triggering a response.f

### Example 17

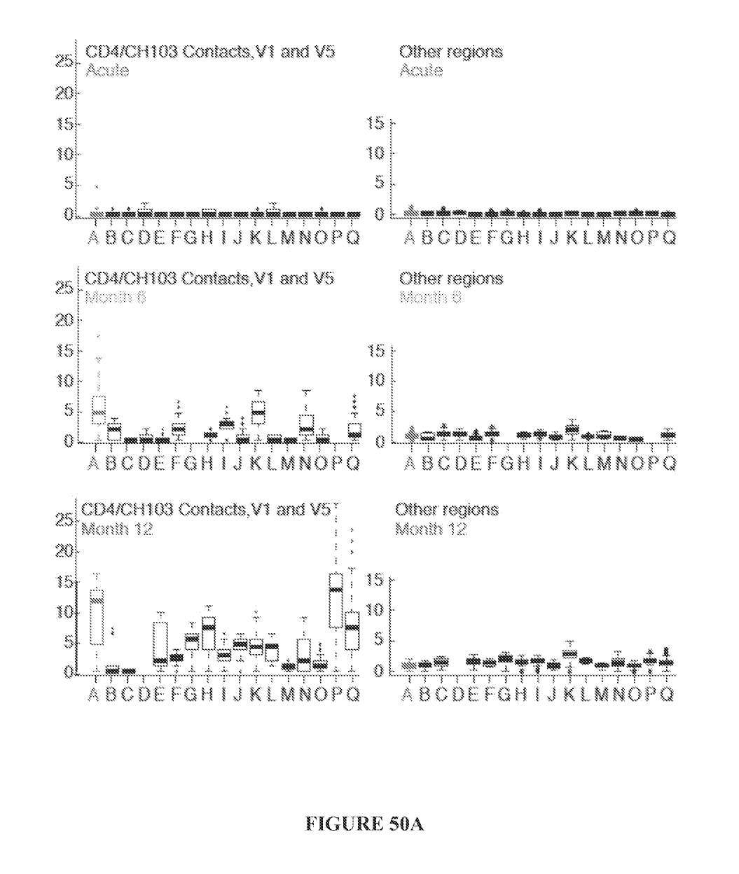

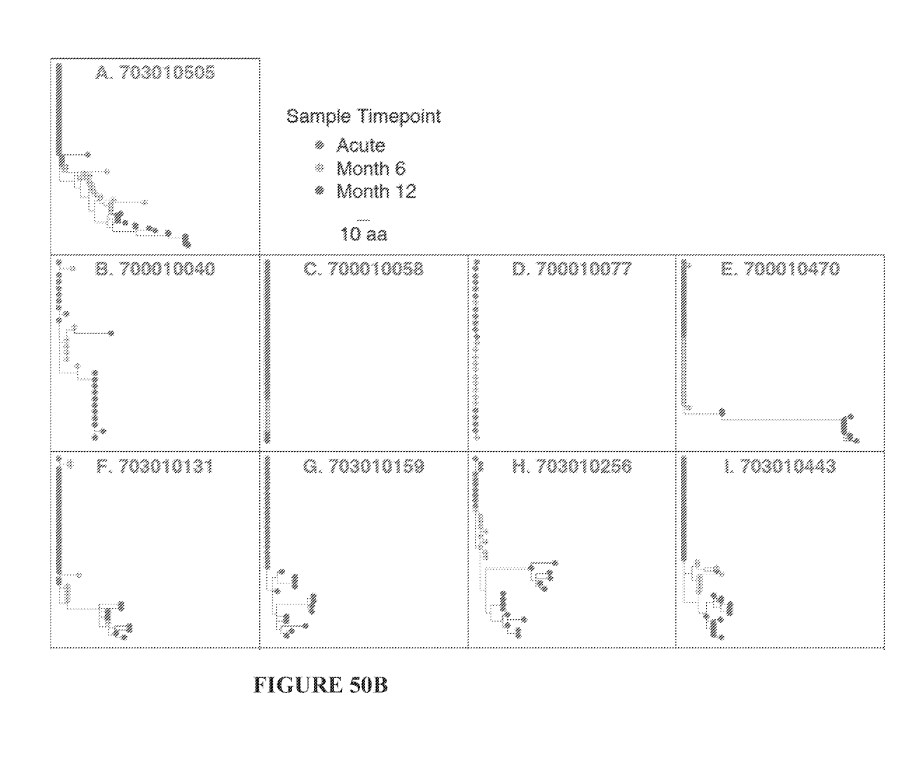

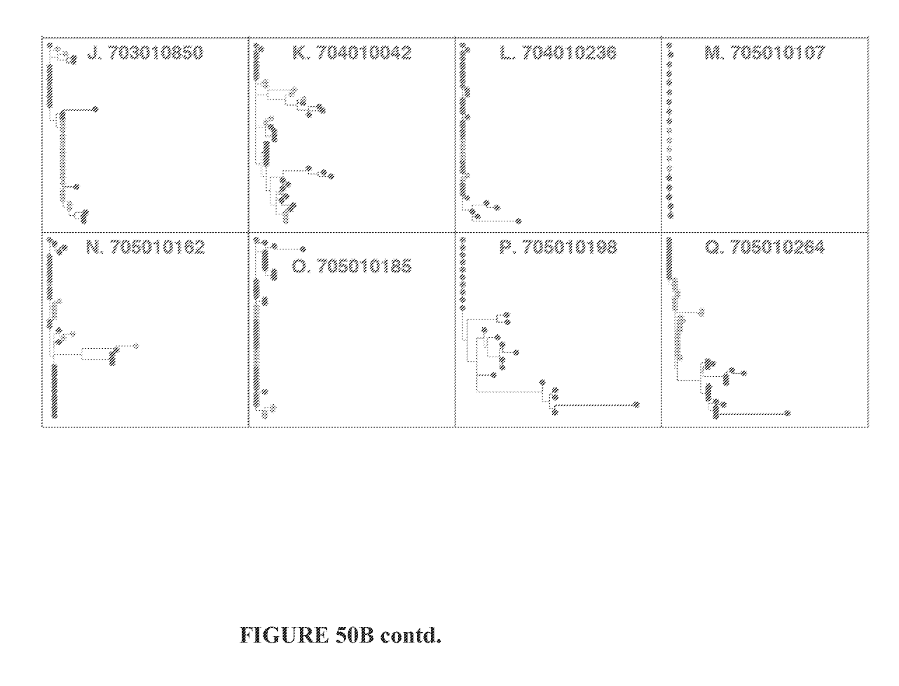

Example 17 describes how env diversification precedes breadth. At 6 months, divergence in contact resisues was greatest for CH505 among 17 subjects followed from acute infection. A comparison of the pace of viral sequence evolution in CH505 (indicated here by the 9-digit anonymous study-participant identifier 703010505) in regions relevant to the CH103 epitope with other subjects is shown in FIGS. 50A-50B. The regions of interest include the CH103 contacts defined by the structure in this paper, as well as VRC01 contacts and CD4bs contacts, and the V1 and V5 loops immediately adjacent to these contacts. (FIG. 50A) The distribution of sequence distances expressed as the percentage of amino acids that are different between two sequences, resulting from a pair-wise comparison of all sequences sampled in a given time point. Because these are all homogeneous (single-founder) infection cases, very few mutations appear in the CH103 relevant regions or other sites in the virus during acute infection (left hand panels). By 24 weeks after enrollment (week 30 from infection in (A) 703010505, labeled month 6 here as it is approximate), extensive mutations have begun to accrue, focused in CH103 relevant regions (top middle panel), but not in other regions of Env (bottom middle panel). Subject 703010505 has the highest ranked diversity among 15 subjects (B-Q) sampled in this time frame (p=0.067), indicating a focused selective pressure began unusually early in this subject. By 1 year (month 12 indicates samples taken between 10-14 months from enrollment, due to variation in timing of patient visits), this region has begun to evolve in many individuals, possibly due to autologous NAb responses active later in infection. (FIG. 50B) Phylogenetic trees based on concatenated CH103 relevant regions (HXB2 sites 124-127, 131, 132, 279-283, 364-371, 425-432, 455-465, 471-477) were created with PhyML3.0, using HIVw, a within-subject HIV protein substitution model, which was selected to be the optimum model for these sequences using ProtTest. Indels were treated as an additional character state, rather than as missing information. In this view, the extensive evolution away from the T/F virus by month 6, shown in gold, is particularly striking. Distances between sequences sampled in 703010505 (A) at month 6 and the T/F ancestral state were significantly greater than the sequences in the next most variable individual (L) designated by the 9-digit identifier 704010042 (Wilcoxon rank sum, p=0.0003: CH505, median=0.064, range=0.019-0.13, N=25, and 704010042, median=0.0271, range=0.009-0.056, N=26).

What is claimed is:

1. A composition comprising any one of a nucleic acid encoding HIV-1 envelope w000.TF, w004.31, w004.54, w007.8, w007.21, w007.25, w007.34, w008.20, w009.19, w010.7, w020.15, w020.11, w020.24, w020.25, w022.6, w022.5, w022.9, w022.22, w030.20, w030.17, w030.21, w030.36, w030.26, w030.13, w030.32, w053.15, w053.29, w053.22, w053.8, w053.31, w053.9, w078.6, w078.36, w078.9, w078.26, w078.29, w078.30, w078.33, w078.17, w078.15, w078.27, w100.T3, w100.B10, w100.B2, w100.B4, w100.A11, w100.A13, w136.B10, w136.B5, w136.B2, w136.B23, w160.C1, w160.T3, w160.T4, or any combination thereof.

2. A composition comprising an HIV-1 envelope polypeptide w000.TF, w004.31, w004.54, w007.8, w007.21, w007.25, w007.34, w008.20, w009.19, w010.7, w020.15, w020.11, w020.24, w020.25, w022.6, w022.5, w022.9, w022.22, w030.20, w030.17, w030.21, w030.36, w030.26, w030.13, w030.32, w053.15, w053.29, w053.22, w053.8, w053.31, w053.9, w078.6, w078.36, w078.9, w078.26, w078.29, w078.30, w078.33, w078.17, w078.15, w078.27, w100.T3, w100.B10, w100.B2, w100.B4, w100.A11, w100.A13, w136.B10, w136.B5, w136.B2, w136.B23, w160.C1, w160.T3, w160.T4, or any combination thereof.

3. A composition comprising any one of a nucleic acid encoding HIV-1 envelope w000.TF, w020.15, w030.13,w020.25, w004.54, w020.11, w078.15, w053.22, w136.B23, w053.31, w136.B2, w100.A13, w100.B4, w160.T4, w030.21, w053.15, w078.17, w136.B10, w053.29, w078.33, w136.B5, w030.36, w030.17, w078.9, w030.20, w100.B2, w078.6, or any combination thereof.

4. A composition comprising an HIV-1 envelope polypeptide w000.TF, w020.15, w030.13,w020.25, w004.54, w020.11, w078.15, w053.22, w136.B23, w053.31, w136.B2, w100.A13, w100.B4, w160.T4, w030.21, w053.15, w078.17, w136.B10, w053.29, w078.33, w136.B5, w030.36, w030.17, w078.9, w030.20, w100.B2, w078.6, or any combination thereof.

5. The composition of any of claims 1-4 further comprising an HIV-1 envelope polypeptide or a nucleic acid encoding an HIV-1 envelope selected from the group consisting of M5, M6 and M11, or any combination thereof, wherein the HIV-1 envelope is a loop D mutant envelope.

6. The composition of claim 1 or 3 wherein the nucleic acid encodes a gp120 envelope, a gp120D8 envelope, a gp140 envelope, a gp145 envelope, a gp150 envelope, or a transmembrane bound envelope.

7. The composition of claim 2 or 4 wherein the HIV-1 envelope is a gp120 or a gp120D8 variant.

8. The composition of any of claims 1-4 further comprising an adjuvant.

9. The composition of any one of claim 1, 3, or 6 wherein the nucleic acid is operably linked to a promoter inserted an expression vector.

10. A method of inducing an immune response in a subject comprising administering the composition of any one of claim 1-7 or 9 in an amount sufficient to induce an immune response.

11. The method of claim 10, wherein the composition is administered as a prime.

12. The method of claim 10, wherein the composition is administered as a boost.

13. The method of claim 10, wherein the composition is administered as multiple boosts.

14. The method of claim 10, wherein the composition further comprises an adjuvant.

15. The method of claim 10, further comprising administering an agent which modulates host immune tolerance.
